(19) United States
(12) Patent Application Publication
(10) Pub. No.: US 2025/0011887 A1
(43) Pub. Date: Jan. 9, 2025

(54) PROBES FOR IMPROVING CORONAVIRUS SAMPLE SURVEILLANCE

(71) Applicant: ILLUMINA, INC., San Diego, CA (US)

(72) Inventors: Brian Hawks, San Diego, CA (US); Stephen Gross, San Diego, CA (US); Gary Schroth, Danville, CA (US)

(21) Appl. No.: 18/898,408

(22) Filed: Sep. 26, 2024

Related U.S. Application Data
(63) Continuation of application No. PCT/US2023/076120, filed on Oct. 5, 2023.
(60) Provisional application No. 63/378,632, filed on Oct. 6, 2022.
(60) Provisional application No. 63/479,823, filed on Jan. 13, 2023.
(60) Provisional application No. 63/480,860, filed on Jan. 20, 2023.

(30) Foreign Application Priority Data
Oct. 5, 2023 (WO) PCT/US2023/076120

(51) Int. Cl. C12Q 1/70; C12Q 1/6806; C12Q 1/6832; C12Q 1/6874

(52) U.S. Cl. CPC C12Q 1/701; C12Q 1/6832; C12Q 1/6874; C12Q 1/6806

(57) ABSTRACT

Described herein are compositions and methods for enriching library fragments prepared for coronavirus sequences prepared from various samples. These methods may incorporate microfluidics and flowcells for greater case of use. Libraries enriched with the present methods may be used for sequencing. Also described are probes and methods for enzymatic depletion of unwanted RNA.

### CROSS-REFERENCE TO RELATED APPLICATIONS

This application is a bypass continuation claiming priority to PCT/2023/076120, filed Oct. 5, 2023, which claims the benefit of priority of U.S. Provisional Application Nos. 63/378,632, filed Oct. 6, 2022; 63/479,823, filed Jan. 13, 2023; and 63/480,860, filed Jan. 20, 2023; each of which is incorporated by reference herein in its entirety for any purpose.

### SEQUENCE LISTING

The application contains a Sequence Listing which has been submitted electronically in .XML format and is hereby incorporated by reference in its entirety. The sequence listing does not go beyond the disclosure of the PCT priority application as filed. Said .XML copy, is named “IP-2346-PCT_SL.xml” and is 31,630 kb in size. The sequence listing contained in this .XML file is part of the specification and is hereby incorporated by reference herein in its entirety.

### DESCRIPTION

### Field

This disclosure relates to probes for improving environmental sample (including wastewater samples and other samples) surveillance and surveillance of other samples for various coronaviruses. Libraries enriched with the present methods may be used to generate sequencing data. Also described are probes and methods for enzymatic depletion of unwanted RNA and cDNA from human wastewater and other samples.

### Background

Viruses continue to develop naturally resulting in new strains and diseases to human populations. For example, the World Health Organization (WHO) declared infection by the novel Severe Acute Respiratory Syndrome Coronavirus 2 (SARS-COV-2) as a pandemic and termed the related disease as coronavirus disease 2019 (COVID-19). Although a large percentage of persons infected with this novel virus experience mild to moderate respiratory, gastrointestinal, cardiovascular, and/or other discomforts without requiring medical care, infected persons with underlying medical problems and/or comorbidities, such as diabetes, cardiovascular disease, chronic respiratory disease, or cancer, are more likely to develop serious illness and/or die from COVID-19 or related secondary infections.

Transmission vectors of SARS-COV-2, and variants thereof, are under heavy investigation. Infected subjects, whether symptomatic or asymptomatic, shed virus and/or inactive viral particles thereof into community sewer systems through feces, nasal/sinus drainage/mucus, and phlegm. While this presents an opportunity to investigate wastewater for incidence of disease, sampling and measuring wastewater for a virus-of-interest such as SARS-COV-2 and/or variants thereof is problematic due to low concentrations of virus or particles thereof alone, or in combination with contaminants (e.g., other waterborne pathogens or human nucleic acids) in the wastewater. Non-limiting examples of waterborne pathogens include bacterial, viral, fungal, and parasitic pathogens, such as fecal coliforms. The mixture of contaminants and pathogens presents a difficult medium for viral DNA and RNA extraction therefrom, especially where concentrations of a virus-of-interest are low.

As such, public health officials need methods of enriching wastewater samples for coronavirus to quantify incidence of viral infection or disease in a community and to identify novel coronaviruses of interest in wastewater, such as from a sewer system. Public health officials also need methods of recovering nucleic acids from a virus-of-interest in wastewater. Investigations of other types of samples would also benefit from improved methods of recovering nucleic acids. Monitoring of other samples also provides valuable public health information and would benefit from improved methods of recovering nucleic acids.

Described herein is the development of a pan-coronavirus probe set for enrichment and detection of novel coronaviruses. Through an iterative design process, probes described herein are designed to have a broad diversity of targets in order to increase the odds of capturing genomic sequence from an as of yet undiscovered or novel variant coronavirus. The probe set described herein, simultaneously minimizes the overall number of oligonucleotides that are necessary to detect such a broad diversity of sequences and minimizes the amount of redundancy.

### SUMMARY

In accordance with the description, described herein are methods of enriching a sample for one or more target coronavirus nucleic acids and/or for improving environmental wastewater surveillance for various coronaviruses. These methods may be performed with standard lab equipment, such as flowcells comprised in sequencers. In some embodiments, standard sequencing consumables and platform (i.e., sequencer) can be used as a microfluidic device for enriching and/or depleting library fragments. In some embodiments, depleting abundant small noncoding RNAis performed after cDNA synthesis and amplification.

Embodiment 1. A method of enriching a sample for one or more target viral nucleic acids comprising the steps of: (a) providing a probe set comprising at least two nucleic acid probes complementary to one or more target viral nucleic acids, wherein the probe set comprises at least two of SEQ ID NOs: 1-22909; (b) allowing the probes in the probe set to hybridize to the target viral nucleic acids; (c) enriching the sample for the one or more target viral nucleic acids by amplifying the target viral nucleic acids and/or separating the target viral nucleic acids from the sample.

Embodiment 2. A method of enriching a sample for one or more target coronavirus nucleic acids comprising the steps of: (a) providing a probe set comprising at least two nucleic acid probes complementary to one or more target coronavirus nucleic acids, wherein the nucleic acid probes are affixed to a support; (b) capturing the one or more target coronavirus nucleic acids on the support; (c) using the one or more captured target coronavirus nucleic acids as a template strand to produce one or more nucleic acid duplexes immobilized on the support, wherein the one or more target coronavirus nucleic acids hybridize to one or more probes of the probe set on the support; (d) contacting a transposase and transposon with the one or more nucleic acid duplexes under conditions wherein the one or more nucleic acid duplexes and transposon composition undergo a transposition reaction to produce one or more tagged nucleic acid duplexes, wherein the transposon composition comprises a double stranded nucleic acid molecule comprising a transferred strand and a non-transferred strand; (e) contacting the one or more tagged nucleic acid duplexes with a nucleic acid modifying enzyme under conditions to extend the 3′ end of the immobilized strand to the 5′ end of the template strand to produce one or more end-extended tagged nucleic acid duplexes; (f) amplifying the one or more end-extended tagged nucleic acid duplexes to produce a plurality of tagged nucleic acid strands; (g) contacting the plurality of tagged nucleic acid strands with a probe set to create an enriched library; and (h) amplifying the enriched library.

Embodiment 3. The method of embodiment 1 or 2, wherein the sample comprises a sample from a mammal.

Embodiment 4. The method of embodiment 3, wherein the sample comprises a sample from a human, monkey, bat, dog, cat, horse, goat, sheep, cow, pig, rat and/or mouse.

Embodiment 5. The method of any one of embodiments 1-4, wherein the sample comprises a blood sample, a serum sample, and/or a whole blood sample.

Embodiment 6. The method of any one of embodiments 1-4, wherein the sample comprises a tissue sample.

Embodiment 7. The method of any one of embodiments 1-4, wherein the sample comprises a fecal sample, a urine sample, a mucus sample, a saliva sample, a lymph sample, a vaginal fluid sample, a semen sample, an amniotic sample, and/or a sweat sample.

Embodiment 8. The method of embodiment 1 or 2, comprises a freshwater sample, a wastewater sample, a saline water sample, or a combination thereof.

Embodiment 9. The method of embodiment 1 or 8, wherein the sample comprises a wastewater sample.

Embodiment 10. The method of any one of embodiments 1-9, wherein the probe set is biotinylated.

Embodiment 11. The method of any one of embodiments 1-10, wherein the one or more target coronavirus nucleic acids are coronavirus RNA molecules.

Embodiment 12. The method of any one of embodiments 1-11, wherein the one or more target coronavirus nucleic acids are genomic coronavirus RNA molecules.

Embodiment 13. The method of any one of embodiments 1-12, wherein the probe set further comprises at least two DNA probes that each hybridize to at least one target coronavirus molecule of the Alphacoronavirus, Betacoronavirus, Deltacoronavirus, Gammacoronavirus, and/or Bafinivirus genus.

Embodiment 14. The method of any one of embodiments 1-13, wherein the probe set further comprises at least two DNA probes that each hybridize to at least one target coronavirus molecule selected from Table 2.

Embodiment 15. The method of any one of embodiments 1-14, wherein at wherein the DNA probes further comprise any one of SEQ ID NOs 22917-23376.

Embodiment 16. The method of any one of embodiments 1-15, wherein at wherein the DNA probes further comprise two or more, or five or more, or 10 or more, or 25 or more sequences, or all of the sequences selected from SEQ ID NOs: 22917-23376.

Embodiment 17. The method of any one of embodiments 1-16, wherein the method further comprises depleting unwanted nucleic acid molecules from a nucleic acid sample.

Embodiment 18. The method of any one of embodiments 1-17, wherein the depleting unwanted nucleic acid molecules comprises depleting unwanted cDNA library fragments from a library of cDNA fragments prepared from RNA, wherein the unwanted library fragments comprise those prepared from unwanted RNA sequences, further comprising: (a) preparing a solid support comprising at least one immobilized oligonucleotide, wherein each immobilized oligonucleotide comprises a nucleic acid sequence corresponding to an unwanted RNA sequence or its complement; (b) adding the library of fragments to the solid support and hybridizing the library fragments to at least one immobilized oligonucleotide to allow binding of unwanted library fragments to at least one immobilized oligonucleotide; and (c) collecting library fragments not bound to at least one immobilized oligonucleotide.

Embodiment 19. The method of claim any one of embodiments 1-18 wherein the at least one immobilized oligonucleotide comprises a sequence comprising any one or more of SEQ ID NOs: 23377-24507 or its complement.

Embodiment 20. The method of any one of embodiments 1-19, wherein the depleting unwanted nucleic acid molecules comprises depleting off-target RNA nucleic acid molecules from a nucleic acid sample comprises: (a) contacting a nucleic acid sample comprising at least one RNA or DNA target sequence and at least one off-target RNA molecule from a first species with a probe set comprising at least two DNA probes complementary to discontiguous sequences along the full length of the at least one off-target RNA molecule from a second species, thereby hybridizing the DNA probes to the off-target RNA molecules to form DNA: RNA hybrids, wherein each DNA: RNA hybrid is at least 5 bases apart, or at least 10 bases apart, along a given off-target RNA molecule sequence from any other DNA: RNA hybrid, wherein the off-target DNA comprises at least one small noncoding RNA chosen from RN7SK, RN7SL1, RN7SL2, RN7SL5P, RPPH1, SNORD3A; (b) contacting the DNA: RNA hybrids with a ribonuclease that degrades the RNA from the DNA: RNA hybrids, thereby degrading the off-target RNA molecules in the nucleic acid sample to form a degraded mixture; (c) separating the degraded RNA from the degraded mixture; (d) sequencing the remaining RNA from the sample; (e) evaluating the remaining RNA sequences for the presence of off-target RNA molecules from the first species, thereby determining gap sequence regions; and (f) supplementing the probe set with additional DNA probes complementary to discontiguous sequences in one or more of the gap sequence regions.

Embodiment 21. The method of any one of embodiments 1-20, wherein the probe set comprises any one or more of SEQ ID NOs: 22917-23376.

Embodiment 22. The method of any one of embodiments 1-21, wherein the method further comprises depleting unwanted cDNA library fragments from a library of cDNA fragments prepared from RNA, wherein the unwanted library fragments comprise those prepared from unwanted RNA sequences.

Embodiment 23. A composition comprising a probe set comprising at least two DNA probes complementary to at least one target coronavirus RNA molecule in a nucleic acid sample wherein the target coronavirus RNA comprises at least one coronavirus molecule selected from Table 2.

Embodiment 24. A composition comprising a probe set comprising at least one DNA probe comprising at least one sequence of SEQ ID NOs: 1-22909.

Embodiment 25. The composition of embodiment 24, comprising at least 5, at least at least 10, at least 50, at least 100, at least 250, at least 500, at least 750, at least 1000, at least 1500, or at least 2000 sequences of SEQ ID NOs: 1-22909.

Embodiment 26. A kit comprising a probe set comprising: (a) at least one DNA probe comprising at least one sequence comprising at least one of SEQ ID NOs: 1-22909; and (b) a buffer.

Embodiment 27. The kit of any one of embodiment 26, wherein the buffer is a wash buffer and/or an elution buffer.

Embodiment 28. The kit of any one of embodiment 26 or 27, further comprising an RNA depletion buffer, a probe depletion buffer, and/or a probe removal buffer.

Embodiment 29. The kit of any one of embodiments 26-28, further comprising: (a) a ribonuclease; (b) a DNase; and (c) RNA purification beads.

Embodiment 30. The kit of any one of embodiments 26-29, wherein the ribonuclease is RNase H.

Embodiment 31. The kit of any one of embodiments 26-30, comprising a buffer and nucleic acid purification medium.

Embodiment 32. The kit of any one of embodiments 26-31, wherein the buffer is an RNA depletion buffer, a probe depletion buffer, and/or a probe removal buffer.

Embodiment 33. The kit of any one of embodiments 26-32. further comprising a nucleic acid destabilizing chemical.

Embodiment 34. The kit of embodiment 33, wherein the nucleic acid destabilizing chemical comprises betaine, DMSO, formamide, glycerol, or a derivative thereof, or a mixture thereof.

Embodiment 35. The kit of embodiment 33 or 34, wherein the nucleic acid destabilizing chemical comprises formamide.

Embodiment 36. The kit of any one of embodiments 26-35, wherein the at least one DNA probe comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, or 22909 probes comprising sequences selected from SEQ ID NOs: 1-22909.

Embodiment 37. The kit of any one of embodiments 26-36, wherein the at least one DNA probe comprises 500 or more, 600 or more, 700 or more, 800 or more, 900 or more, 1000 or more, 1100 or more, or 22909 probes comprising sequences selected from SEQ ID NOs: 1-22909.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

### BRIEF DESCRIPTION OF SELECT SEQUENCES

| | SEQ | |
|---|---|---|
| | ID | |
| Description | NO: | Sequence (3′ to 5′) |
| RN7SK | 22910 | GATGTGAGGGCGATCTGGCTGCGACATCTGTCACCCCATTGATCGCCAG |
| | | GGTTGATTCGGCTGATCTGGCTGGCTAGGCGGGTGTCCCCTTCCTCCCT |
| | | CACCGCTCCATGTGCGTCCCTCCCGAAGCTGCGCGCTCGGTCGAAGAGG |
| | | ACGACCATCCCCGATAGAGGAGGACCGGTCTTCGGTCAAGGGTATACGA |
| | | GTAGCTGCGCTCCCCTGCTAGAACCTCCAAACAAGCTCTCAAGGTCCAT |
| | | TTGTAGGAGAACGTAGGGTAGTCAAGCTTCCAAGACTCCAGACACATCC |
| | | AAATGAGGCGCTGCATGTGGCAGTCTGCCTTTCT |
| RN7SL1 | 22911 | GCCGGGCGCGGTGGCGCGTGCCTGTAGTCCCAGCTACTCGGGAGGCTGA |
| | | GGCTGGAGGATCGCTTGAGTCCAGGAGTTCTGGGCTGTAGTGCGCTATG |
| | | CCGATCGGGTGTCCGCACTAAGTTCGGCATCAATATGGTGACCTCCCGG |
| | | GAGCGGGGGACCACCAGGTTGCCTAAGGAGGGGTGAACCGGCCCAGGTC |
| | | GGAAACGGAGCAGGTCAAAACTCCCGTGCTGATCAGTAGTGGGATCGCG |
| | | CCTGTGAATAGCCACTGCACTCCAGCCTGGGCAACATAGCGAGACCCCG |
| | | TCTCT |
| RN7SL2 | 22912 | GCCGGGCGCGGTGGCGCGTGCCTGTAGTCCCAGCTACTCGGGAGGCTGA |
| | | GGTGGGAGGATCGCTTGAGCCCAGGAGTTCTGGGCTGTAGTGCGCTATG |
| | | CCGATCGGGTGTCCGCACTAAGTTCGGCATCAATATGGTGACCTCCCGG |
| | | GAGCGGGGGACCACCAGGTTGCCTAAGGAGGGGTGAACCGGCCCAGGTC |
| | | GGAAACGGAGCAGGTCAAAACTCCCGTGCTGATCAGTAGTGGGATCGCG |
| | | CCTGTGAATAGCCACTGCACTCCAGCCTGAGCAACATAGCGAGACCCCG |
| | | TCTCTT |
| RN7SL5P | 22913 | GCCGGGCGCGGTGGCGCGTGCCTGTGGTCCCAGCTACTCGGGAGGCTGA |
| | | GGCTGGAGGATCGCTTGAGTCCAGGAGTTCTGGGCTGTAGTGCGCTATG |
| | | CCGATCGGGTGTCCGCACTAAGTTCGGCATCAATATGGTGACCTCCCGG |
| | | GAGCGGGGGACCACCAGGTTGCCTAAGGAGGGGTGAACCGGCCCAGGTC |
| | | GGAAACGGAGCAGGTCAAAACTCCCGTGCTGATCAGTAGAAGTCTGTAA |
| | | TGCTACTGGTGTCCCCTAATTTTCTTATAGCCACAGTTCCTTTCGCCTG |
| | | AGCTCATTACAGAGACAAATATCCATT |
| RPPH1 | 22914 | GGCGGAGGGAAGCTCATCAGTGGGGCCACGAGCTGAGTGCGTCCTGTCA |
| | | CTCCACTCCCATGTCCCTTGGGAAGGTCTGAGACTAGGGCCAGAGGCGG |
| | | CCCTAACAGGGCTCTCCCTGAGCTTCGGGGAGGTGAGTTCCCAGAGAAC |
| | | GGGGCTCCGCGCGAGGTCAGACTGGGCAGGAGATGCCGTGGACCCCGCC |
| | | CTTCGGGGAGGGGCCCGGCGGATGCCTCCTTTGCCGGAGCTTGGAACAG |
| | | ACTCACGGCCAGCGAAGTGAGTTCAATGGCTGAGGTGAGGTACCCCGCA |
| | | GGGGACCTCATAACCCAATTCAGACTACTCTCCTCCGCC |
| SNORD3A with | 22915 | AAGACTATACTTTCAGGGATCATTTCTATAGTGTGTTACTAGAGAAGTT |
| the ALU region in | | TCTCTGAACGTGTAGAGCACCGAAAACCACGAGGAAGAGAGGTAGCGTT |
| bold and italics, in | | TTCTCCTGAGCGTGAAGCCGGCTTTCTGGCGTTGCTTGGCTGCAACTGC |
| some | | CGTCAGCCATTGATGATCGTTCTTCTCTCCGTATTGGGGAGTGAGAGGG |
| embodiments the | | AGAGAACGCGGTCTGAGTGGTTTTTCCTTCTTGATGGCTCAATGACAGA |
| ALU region was | | GACTAGCTCGTAAACTCCGGGGCGTTTCTGGGCTGTTCGCTCCTGCTTG |
| not used to | | GCATGTCGCGAGAAAGGTTTTCGCCTCCTGTTTCAGCGGTGACGGCTCT |
| generate probes | | TGGGTTTTCTCGGGGTGGCTTTTTAATTTTAGTCTTGGCGCGAGGCGGG |
| because it is a | | GGATGCTGTGTGGCACCTCCTATTGTCTCTTTTTGCGTTTTCTCCCATT |
| repetitive region in | | CTCGCTCCCTCTTTTGTCGCCGTTTCCCGCCCGCCACTCCCACCCCCAG |
| other areas of the | | ACGGGGTCTCCGGGTCTCTTGTTCTGTCTGCCGGCCCCGGCTGGATTGC |
| genome. | | AGTGGCGCGATCTCGGCTCCTAGCAACATCTGCCTCCCGGGCTCAAGCG |
| | | AGTCTCCCGCCTAAGCCCTCCCGAGTAGCCGGGGCTTAAAGGCGCACAC |
| | | GCCACTCCAGGCTTTTTTTTTTTTTTTTTTTTTTTTTTTGGCAGAAACG |
| | | GGGTGTCAGCATG |
| Reverse | 22916 | AGAAAGGCAGACTGCCACATGCAGCGCCTCATTTGGATGTGTCTGGAGT |
| complement of | | CTTGGAAGCTTGACTACCCTACGTTCTCCTACAAATGGACCTTGAGAGC |
| RN7SK with | | TTGTTTGGAGGTTCTAGCAGGGGAGCGCAGCTACTCGTATACCCTTGAC |
| probe sequences in | | CGAAGACCGGTCCTCCTCTATCGGGGATGGTCGTCCTCTTCGACCGAGC |
| bold and italics | | GCGCAGCTTCGGGAGGGACGCACATGGAGCGGTGAGGGAGGAAGGGGAC |
| (and with gaps | | ACCCGCCTAGCCAGCCAGATCAGCCGAATCAACCCTGGCGATCAATGGG |
| between the probes) | | GTGACAGATGTCGCAGCCAGATCGCCCTCACATC |
| Probe for | 22917 | AGAAAGGCAGACTGCCACATGCAGCGCCTCATTTGGATGTGTCTGGAGT |
| RN7SK | | C |
| Probe for | 22918 | CCCTACGTTCTCCTACAAATGGACCTTGAGAGCTTGTTTGGAGGTTCTA |
| RN7SK | | G |
| Probe for | 22919 | ACTCGTATACCCTTGACCGAAGACCGGTCCTCCTCTATCGGGGATGGTC |
| RN7SK | | G |
| Probe for | 22920 | CGCGCAGCTTCGGGAGGGACGCACATGGAGCGGTGAGGGAGGAAGGGGA |
| RN7SK | | C |
| Probe for | 22921 | CAGATCAGCCGAATCAACCCTGGCGATCAATGGGGTGACAGATGTCGCA |
| RN7SK | | G |
| Probe | 22922 | AGAGACGGGGTCTCGCTATGTTGCCCAGGCTGGAGTGCAGTGGCTATTC |
| for RN7SL1 | | A |
| Probe for | 22923 | TACTGATCAGCACGGGAGTTTTGACCTGCTCCGTTTCCGACCTGGGCCG |
| RN7SL1 | | G |
| Probe for | 22924 | GCAACCTGGTGGTCCCCCGCTCCCGGGAGGTCACCATATTGATGCCGAA |
| RN7SL1 | | C |
| Probe for | 22925 | GATCGGCATAGCGCACTACAGCCCAGAACTCCTGGACTCAAGCGATCCT |
| RN7SL1 | | C |
| Probe for RN7SL2 | 22926 | AAGAGACGGGGTCTCGCTATGTTGCTCAGGCTGGAGTGCAGTGGCTATT |
| | | C |
| Probe for RN7SL2 | 22927 | CTACTGATCAGCACGGGAGTTTTGACCTGCTCCGTTTCCGACCTGGGCC |
| | | G |
| Probe for RN7SL2 | 22928 | GGCAACCTGGTGGTCCCCCGCTCCCGGGAGGTCACCATATTGATGCCGA |
| | | A |
| Probe for RN7SL2 | 22929 | CGATCGGCATAGCGCACTACAGCCCAGAACTCCTGGGCTCAAGCGATCC |
| | | T |
| Probe | 22930 | AATGGATATTTGTCTCTGTAATGAGCTCAGGCGAAAGGAACTGTGGCTA |
| for RN7SL5P | | T |
| Probe | 22931 | CACCAGTAGCATTACAGACTTCTACTGATCAGCACGGGAGTTTTGACCT |
| for RN7SL5P | | G |
| Probe | 22932 | GGGCCGGTTCACCCCTCCTTAGGCAACCTGGTGGTCCCCCGCTCCCGGG |
| for RN7SL5P | | A |
| Probe | 22933 | GCCGAACTTAGTGCGGACACCCGATCGGCATAGCGCACTACAGCCCAGA |
| for RN7SL5P | | A |
| Probe | 22934 | GATCCTCCAGCCTCAGCCTCCCGAGTAGCTGGGACCACAGGCACGCGCC |
| for RN7SL5P | | A |
| Probe for RPPH1 | 22935 | GGCGGAGGAGAGTAGTCTGAATTGGGTTATGAGGTCCCCTGCGGGGTAC |
| | | C |
| Probe for RPPH1 | 22936 | AACTCACTTCGCTGGCCGTGAGTCTGTTCCAAGCTCCGGCAAAGGAGGC |
| | | A |
| Probe for RPPH1 | 22937 | CCCGAAGGGCGGGGTCCACGGCATCTCCTGCCCAGTCTGACCTCGCGCG |
| | | G |
| Probe for RPPH1 | 22938 | GAACTCACCTCCCCGAAGCTCAGGGAGAGCCCTGTTAGGGCCGCCTCTG |
| | | G |
| Probe for RPPH1 | 22939 | TTCCCAAGGGACATGGGAGTGGAGTGACAGGACGCACTCAGCTCGTGGC |
| | | C |
| Probe | 22940 | CCCGGAGACCCCGTCTGGGGGTGGGAGTGGCGGGCGGGAAACGGCGACA |
| for SNORD3A | | A |
| Probe | 22941 | TGGGAGAAAACGCAAAAAGAGACAATAGGAGGTGCCACACAGCATCCCC |
| for SNORD3A | | C |
| Probe | 22942 | TAAAATTAAAAAGCCACCCCGAGAAAACCCAAGAGCCGTCACCGCTGAA |
| for SNORD3A | | A |
| Probe | 22943 | TTTCTCGCGACATGCCAAGCAGGAGCGAACAGCCCAGAAACGCCCCGGA |
| for SNORD3A | | G |
| Probe | 22944 | CTGTCATTGAGCCATCAAGAAGGAAAAACCACTCAGACCGCGTTCTCTC |
| for SNORD3A | | C |
| Probe for | 22945 | ACGGAGAGAAGAACGATCATCAATGGCTGACGGCAGTTGCAGCCAAGCA |
| SNORD3A | | A |
| Probe for | 22946 | TTCACGCTCAGGAGAAAACGCTACCTCTCTTCCTCGTGGTTTTCGGTGC |
| SNORD3A | | T |
| Probe for | 22947 | AAACTTCTCTAGTAACACACTATAGAAATGATCCCTGAAAGTATAGTCT |
| SNORD3A | | T |
| (additional probe | | |
| added at start of | | |
| SNORD3A | | |
| transcript) | | |
| Probe for RN7SL1 | 22948 | CTCAGCCTCCCGAGTAGCTGGGACTACAGGCACGCGCCACCGCGCCCGG |
| and RN7SL2 | | C |
| (additional probe | | |
| added at start of | | |
| RN7SL1 and | | |
| RN7SL2 | | |
| transcript) | | |

Additional Probes

| | | |
|---|---|---|
| 12S_P1 | 22949 | GTTCGTCCAAGTGCACTTTCCAGTACACTTACCATGTTACGACTTGTCT |
| | | C |
| 12S_P2 | 22951 | TAGGGGTTTTAGTTAAATGTCCTTTGAAGTATACTTGAGGAGGGTGACG |
| | | G |
| 12S_P3 | 22951 | TTCAGGGCCCTGTTCAACTAAGCACTCTACTCTCAGTTTACTGCTAAAT |
| | | C |
| 12S_P4 | 22952 | AGTTTCATAAGGGCTATCGTAGTTTTCTGGGGTAGAAAATGTAGCCCAT |
| | | T |
| 12S_P5 | 22953 | GGCTACACCTTGACCTAACGTCTTTACGTGGGTACTTGCGCTTACTTTG |
| | | T |
| 12S_P6 | 22954 | TTGCTGAAGATGGCGGTATATAGGCTGAGCAAGAGGTGGTGAGGTTGAT |
| | | C |
| 12S_P7 | 22955 | CAGAACAGGCTCCTCTAGAGGGATATGAAGCACCGCCAGGTCCTTTGAG |
| | | T |
| 12S_P8 | 22956 | GTAGTGTTCTGGCGAGCAGTTTTGTTGATTTAACTGTTGAGGTTTAGGG |
| | | C |
| 12S_P9 | 22957 | ATCTAATCCCAGTTTGGGTCTTAGCTATTGTGTGTTCAGATATGTTAAA |
| | | G |
| 12S_P10 | 22958 | ATTTTGTGTCAACTGGAGTTTTTTACAACTCAGGTGAGTTTTAGCTTTA |
| | | T |
| 12S_P11 | 22959 | CTAAAACACTCTTTACGCCGGCTTCTATTGACTTGGGTTAATCGTGTGA |
| | | C |
| 12S_P12 | 22960 | GAAATTGACCAACCCTGGGGTTAGTATAGCTTAGTTAAACTTTCGTTTA |
| | | T |
| 12S_P13 | 22961 | ACTGCTGTTTCCCGTGGGGGTGTGGCTAGGCTAAGCGTTTTGAGCTGCA |
| | | T |
| 12S_P14 | 22962 | GCTTGTCCCTTTTGATCGTGGTGATTTAGAGGGTGAACTCACTGGAACG |
| | | G |
| 12S_P15 | 22963 | TAATCTTACTAAGAGCTAATAGAAAGGCTAGGACCAAACCTATTTGTTT |
| | | A |
| 16S_P1 | 22964 | AAACCCTGTTCTTGGGTGGGTGTGGGTATAATACTAAGTTGAGATGATA |
| | | T |
| 16S_P2 | 22965 | GCGCTTTGTGAAGTAGGCCTTATTTCTCTTGTCCTTTCGTACAGGGAGG |
| | | A |
| 16S_P3 | 22966 | AAACCGACCTGGATTACTCCGGTCTGAACTCAGATCACGTAGGACTTTA |
| | | A |
| 16S_P4 | 22967 | ACCTTTAATAGCGGCTGCACCATCGGGATGTCCTGATCCAACATCGAGG |
| | | T |
| 16S_P5 | 22968 | TGATATGGACTCTAGAATAGGATTGCGCTGTTATCCCTAGGGTAACTTG |
| | | T |
| 16S_P6 | 22969 | ATTGGATCAATTGAGTATAGTAGTTCGCTTTGACTGGTGAAGTCTTAGC |
| | | A |
| 16S_P7 | 22970 | TTGGGTTCTGCTCCGAGGTCGCCCCAACCGAAATTTTTAATGCAGGTTT |
| | | G |
| 16S_P8 | 22971 | TGGGTTTGTTAGGTACTGTTTGCATTAATAAATTAAAGCTCCATAGGGT |
| | | C |
| 16S_P9 | 22972 | GTCATGCCCGCCTCTTCACGGGCAGGTCAATTTCACTGGTTAAAAGTAA |
| | | G |
| 16S_P10 | 22973 | CGTGGAGCCATTCATACAGGTCCCTATTTAAGGAACAAGTGATTATGCT |
| | | A |
| 16S_P11 | 22974 | GGTACCGCGGCCGTTAAACATGTGTCACTGGGCAGGCGGTGCCTCTAAT |
| | | A |
| 16S_P12 | 22975 | GTGATGTTTTTGGTAAACAGGCGGGGTAAGGTTTGCCGAGTTCCTTTTA |
| | | C |
| 16S_P13 | 22976 | CTTATGAGCATGCCTGTGTTGGGTTGACAGTGAGGGTAATAATGACTTG |
| | | T |
| 16S_P14 | 22977 | ATTGGGCTGTTAATTGTCAGTTCAGTGTTTTGATCTGACGCAGGCTTAT |
| | | G |
| 16S_P15 | 22978 | TCATGTTACTTATACTAACATTAGTTCTTCTATAGGGTGATAGATTGGT |
| | | C |
| 16S_P16 | 22979 | AGTTCAGTTATATGTTTGGGATTTTTTAGGTAGTGGGTGTTGAGCTTGA |
| | | A |
| 16S_P17 | 22980 | TGGCTGCTTTTAGGCCTACTATGGGTGTTAAATTTTTTACTCTCTCTAC |
| | | A |
| 16S_P18 | 22981 | GTCCAAAGAGCTGTTCCTCTTTGGACTAACAGTTAAATTTACAAGGGGA |
| | | T |
| 16S_P19 | 22982 | GGCAAATTTAAAGTTGAACTAAGATTCTATCTTGGACAACCAGCTATCA |
| | | C |
| 16S_P20 | 22983 | TGTCGCCTCTACCTATAAATCTTCCCACTATTTTGCTACATAGACGGGT |
| | | G |
| 16S_P21 | 22984 | TCTTAGGTAGCTCGTCTGGTTTCGGGGGTCTTAGCTTTGGCTCTCCTTG |
| | | C |
| 16S_P22 | 22985 | TAATTCATTATGCAGAAGGTATAGGGGTTAGTCCTTGCTATATTATGCT |
| | | T |
| 16S_P23 | 22986 | TCTTTCCCTTGCGGTACTATATCTATTGCGCCAGGTTTCAATTTCTATC |
| | | G |
| 16S_P24 | 22987 | GGTAAATGGTTTGGCTAAGGTTGTCTGGTAGTAAGGTGGAGTGGGTTTG |
| | | G |
| 18S_P1 | 22988 | TAATGATCCTTCCGCAGGTTCACCTACGGAAACCTTGTTACGACTTTTA |
| | | C |
| 18S_P2 | 22989 | AAGTTCGACCGTCTTCTCAGCGCTCCGCCAGGGCCGTGGGCCGACCCCG |
| | | G |
| 18S_P3 | 22990 | GGCCTCACTAAACCATCCAATCGGTAGTAGCGACGGGCGGTGTGTACAA |
| | | A |
| 18S_P4 | 22991 | CAACGCAAGCTTATGACCCGCACTTACTCGGGAATTCCCTCGTTCATGG |
| | | G |
| 18S_P5 | 22992 | CCGATCCCCATCACGAATGGGGTTCAACGGGTTACCCGCGCCTGCCGGC |
| | | G |
| 18S_P6 | 22993 | CTGAGCCAGTCAGTGTAGCGCGCGTGCAGCCCCGGACATCTAAGGGCAT |
| | | C |
| 18S_P7 | 22994 | CTCAATCTCGGGTGGCTGAACGCCACTTGTCCCTCTAAGAAGTTGGGGG |
| | | A |
| 18S_P8 | 22995 | GGTCGCGTAACTAGTTAGCATGCCAGAGTCTCGTTCGTTATCGGAATTA |
| | | A |
| 18S_P9 | 22996 | CACCAACTAAGAACGGCCATGCACCACCACCCACGGAATCGAGAAAGAG |
| | | C |
| 18S_P10 | 22997 | CCTGTCCGTGTCCGGGCCGGGTGAGGTTTCCCGTGTTGAGTCAAATTAA |
| | | G |
| 18S_P11 | 22998 | CTGGTGGTGCCCTTCCGTCAATTCCTTTAAGTTTCAGCTTTGCAACCAT |
| | | A |
| 18S_P12 | 22999 | AAAGACTTTGGTTTCCCGGAAGCTGCCCGGCGGGTCATGGGAATAACGC |
| | | C |
| 18S_P13 | 23000 | GGCATCGTTTATGGTCGGAACTACGACGGTATCTGATCGTCTTCGAACC |
| | | T |
| 18S_P14 | 23001 | GATTAATGAAAACATTCTTGGCAAATGCTTTCGCTCTGGTCCGTCTTGC |
| | | G |
| 18S_P15 | 23002 | CACCTCTAGCGGCGCAATACGAATGCCCCCGGCCGTCCCTCTTAATCAT |
| | | G |
| 18S_P16 | 23003 | ACCAACAAAATAGAACCGCGGTCCTATTCCATTATTCCTAGCTGCGGTA |
| | | T |
| 18S_P17 | 23004 | CTGCTTTGAACACTCTAATTTTTTCAAAGTAAACGCTTCGGGCCCCGCG |
| | | G |
| 18S_P18 | 23005 | GCATCGAGGGGGCGCCGAGAGGCAAGGGGCGGGGACGGGCGGTGGCTCG |
| | | C |
| 18S_P19 | 23006 | CCGCCCGCTCCCAAGATCCAACTACGAGCTTTTTAACTGCAGCAACTTT |
| | | A |
| 18S_P20 | 23007 | GCTGGAATTACCGCGGCTGCTGGCACCAGACTTGCCCTCCAATGGATCC |
| | | T |
| 18S_P21 | 23008 | AGTGGACTCATTCCAATTACAGGGCCTCGAAAGAGTCCTGTATTGTTAT |
| | | T |
| 18S_P22 | 23009 | CCCGGGTCGGGAGTGGGTAATTTGCGCGCCTGCTGCCTTCCTTGGATGT |
| | | G |
| 18S_P23 | 23010 | GCTCCCTCTCCGGAATCGAACCCTGATTCCCCGTCACCCGTGGTCACCA |
| | | T |
| 18S_P24 | 23011 | TACCATCGAAAGTTGATAGGGCAGACGTTCGAATGGGTCGTCGCCGCCA |
| | | C |
| 18S_P25 | 23012 | GGCCCGAGGTTATCTAGAGTCACCAAAGCCGCCGGCGCCCGCCCCCCGG |
| | | C |
| 18S_P26 | 23013 | GCTGACCGGGTTGGTTTTGATCTGATAAATGCACGCATCCCCCCCGCGA |
| | | A |
| 18S_P27 | 23014 | TCGGCATGTATTAGCTCTAGAATTACCACAGTTATCCAAGTAGGAGAGG |
| | | A |
| 18S_P28 | 23015 | AACCATAACTGATTTAATGAGCCATTCGCAGTTTCACTGTACCGGCCGT |
| | | G |
| 18S_P29 | 23016 | ATGGCTTAATCTTTGAGACAAGCATATGCTACTGGCAGGATCAACCAGG |
| | | T |
| 28S_P1 | 23017 | GACAAACCCTTGTGTCGAGGGCTGACTTTCAATAGATCGCAGCGAGGGA |
| | | G |
| 28S_P2 | 23018 | CGAAACCCCGACCCAGAAGCAGGTCGTCTACGAATGGTTTAGCGCCAGG |
| | | T |
| 28S_P3 | 23019 | GGTGCGTGACGGGCGAGGGGGCGGCCGCCTTTCCGGCCGCGCCCCGTTT |
| | | C |
| 28S_P4 | 23020 | CTCCGCACCGGACCCCGGTCCCGGCGCGCGGCGGGGCACGCGCCCTCCC |
| | | G |
| 28S_P5 | 23021 | AGGGGGGGGCGGCCCGCCGGCGGGGACAGGCGGGGGACCGGCTATCCGA |
| | | G |
| 28S_P6 | 23022 | GCGGCGCTGCCGTATCGTTCGCCTGGGCGGGATTCTGACTTAGAGGCGT |
| | | T |
| 28S_P7 | 23023 | AGATGGTAGCTTCGCCCCATTGGCTCCTCAGCCAAGCACATACACCAAA |
| | | T |
| 28S_P8 | 23024 | TCCTCTCGTACTGAGCAGGATTACCATGGCAACAACACATCATCAGTAG |
| | | G |
| 28S_P9 | 23025 | CTCACGACGGTCTAAACCCAGCTCACGTTCCCTATTAGTGGGTGAACAA |
| | | T |
| 28S_P10 | 23026 | TTCTGCTTCACAATGATAGGAAGAGCCGACATCGAAGGATCAAAAAGCG |
| | | A |
| 28S_P11 | 23027 | TTGGCCGCCACAAGCCAGTTATCCCTGTGGTAACTTTTCTGACACCTCC |
| | | T |
| 28S_P12 | 23028 | GGTCAGAAGGATCGTGAGGCCCCGCTTTCACGGTCTGTATTCGTACTGA |
| | | A |
| 28S_P13 | 23029 | AGCTTTTGCCCTTCTGCTCCACGGGAGGTTTCTGTCCTCCCTGAGCTCG |
| | | C |
| 28S_P14 | 23030 | TTACCGTTTGACAGGTGTACCGCCCCAGTCAAACTCCCCACCTGGCACT |
| | | G |
| 28S_P15 | 23031 | GCGCCCGGCCGGGCGGGCGCTTGGCGCCAGAAGCGAGAGCCCCTCGGGC |
| | | T |
| 28S_P16 | 23032 | CCGGGTCAGTGAAAAAACGATCAGAGTAGTGGTATTTCACCGGCGGCCC |
| | | G |
| 28S_P17 | 23033 | CGCCCCGGGCCCCTCGCGGGGACACCGGGGGGGCGCCGGGGGCCTCCCA |
| | | C |
| 28S_P18 | 23034 | CATGTCTCTTCACCGTGCCAGACTAGAGTCAAGCTCAACAGGGTCTTCT |
| | | T |
| 28S_P19 | 23035 | CCAAGCCCGTTCCCTTGGCTGTGGTTTCGCTGGATAGTAGGTAGGGACA |
| | | G |
| 28S_P20 | 23036 | TCCATTCATGCGCGTCACTAATTAGATGACGAGGCATTTGGCTACCTTA |
| | | A |
| 28S_P21 | 23037 | TCCCGCCGTTTACCCGCGCTTCATTGAATTTCTTCACTTTGACATTCAG |
| | | A |
| 28S_P22 | 23038 | CACATCGCGTCAACACCCGCCGCGGGCCTTCGCGATGCTTTGTTTTAAT |
| | | T |
| 28S_P23 | 23039 | CCTGGTCCGCACCAGTTCTAAGTCGGCTGCTAGGCGCCGGCCGAGGCGA |
| | | G |
| 28S_P24 | 23040 | CGGCCCCGGGGGCGGACCCGGCGGGGGGGACCGGCCCGCGGCCCCTCCG |
| | | C |
| 28S_P25 | 23041 | CCGCCGCGCGCCGAGGAGGAGGGGGGAACGGGGGGCGGACGGGGCCGGG |
| | | G |
| 28S_P26 | 23042 | ACGAACCGCCCCGCCCCGCCGCCCGCCGACCGCCGCCGCCCGACCGCTC |
| | | C |
| 28S_P27 | 23043 | CGCGCGCGACCGAGACGTGGGGTGGGGGTGGGGGGCGCGCCGCGCCGCC |
| | | G |
| 28S_P28 | 23044 | GCGGCCGCGACGCCCGCCGCAGCTGGGGCGATCCACGGGAAGGGCCCGG |
| | | C |
| 28S_P29 | 23045 | GCGCCGCCGCCGGCCCCCCGGGTCCCCGGGGCCCCCCTCGCGGGGACCT |
| | | G |
| 28S_P30 | 23046 | CCGGCGGCCGCCGCGCGGCCCCTGCCGCCCCGACCCTTCTCCCCCCGCC |
| | | G |
| 28S_P31 | 23047 | CTCCCCCGGGGAGGGGGGAGGACGGGGAGCGGGGGAGAGAGAGAGAGAG |
| | | A |
| 28S_P32 | 23048 | AGGGAGCGAGCGGCGCGCGCGGGTGGGGCGGGGGAGGGCCGCGAGGGGG |
| | | G |
| 28S_P33 | 23049 | GGGGGCGCGCGCCTCGTCCAGCCGCGGCGCGCGCCCAGCCCCGCTTCGC |
| | | G |
| 28S_P34 | 23050 | CCCAGCCCTTAGAGCCAATCCTTATCCCGAAGTTACGGATCCGGCTTGC |
| | | C |
| 28S_P35 | 23051 | CATTGTTCCAACATGCCAGAGGCTGTTCACCTTGGAGACCTGCTGCGGA |
| | | T |
| 28S_P36 | 23052 | CGCGAGATTTACACCCTCTCCCCCGGATTTTCAAGGGCCAGCGAGAGCT |
| | | C |
| 28S_P37 | 23053 | AACCGCGACGCTTTCCAAGGCACGGGCCCCTCTCTCGGGGCGAACCCAT |
| | | T |
| 28S_P38 | 23054 | CTTCACAAAGAAAAGAGAACTCTCCCCGGGGCTCCCGCCGGCTTCTCCG |
| | | G |
| 28S_P39 | 23055 | CGCACTGGACGCCTCGCGGCGCCCATCTCCGCCACTCCGGATTCGGGGA |
| | | T |
| 28S_P40 | 23056 | TTTCGATCGGCCGAGGGCAACGGAGGCCATCGCCCGTCCCTTCGGAACG |
| | | G |
| 28S_P41 | 23057 | CAGGACCGACTGACCCATGTTCAACTGCTGTTCACATGGAACCCTTCTC |
| | | C |
| 28S_P42 | 23058 | GTTCTCGTTTGAATATTTGCTACTACCACCAAGATCTGCACCTGCGGCG |
| | | G |
| 28S_P43 | 23059 | CGCCCTAGGCTTCAAGGCTCACCGCAGCGGCCCTCCTACTCGTCGCGGC |
| | | G |
| 28S_P44 | 23060 | TCCGGGGGCGGGGAGCGGGGCGTGGGCGGGAGGAGGGGAGGAGGCGTGG |
| | | G |
| 28S_P45 | 23061 | AGGACCCCACACCCCCGCCGCCGCCGCCGCCGCCGCCCTCCGACGCACA |
| | | C |
| 28S_P46 | 23062 | GCGCGCCGCCCCCGCCGCTCCCGTCCACTCTCGACTGCCGGCGACGGCC |
| | | G |
| 28S_P47 | 23063 | CTCCAGCGCCATCCATTTTCAGGGCTAGTTGATTCGGCAGGTGAGTTGT |
| | | T |
| 28S_P48 | 23064 | GATTCCGACTTCCATGGCCACCGTCCTGCTGTCTATATCAACCAACACC |
| | | T |
| 28S_P49 | 23065 | GAGCGTCGGCATCGGGCGCCTTAACCCGGCGTTCGGTTCATCCCGCAGC |
| | | G |
| 28S_P50 | 23066 | AAAAGTGGCCCACTAGGCACTCGCATTCCACGCCCGGCTCCACGCCAGC |
| | | G |
| 28S_P51 | 23067 | CCATTTAAAGTTTGAGAATAGGTTGAGATCGTTTCGGCCCCAAGACCTC |
| | | T |
| 28S_P52 | 23068 | CGGATAAAACTGCGTGGCGGGGGTGCGTCGGGTCTGCGAGAGCGCCAGC |
| | | T |
| 28S_P53 | 23069 | TCGGAGGGAACCAGCTACTAGATGGTTCGATTAGTCTTTCGCCCCTATA |
| | | C |
| 28S_P54 | 23070 | GATTTGCACGTCAGGACCGCTACGGACCTCCACCAGAGTTTCCTCTGGC |
| | | T |
| 28S_P55 | 23071 | ATAGTTCACCATCTTTCGGGTCCTAACACGTGCGCTCGTGCTCCACCTC |
| | | C |
| 28S_P56 | 23072 | AGACGGGCCGGTGGTGCGCCCTCGGCGGACTGGAGAGGCCTCGGGATCC |
| | | C |
| 28S_P57 | 23073 | CGCGCCGGCCTTCACCTTCATTGCGCCACGGCGGCTTTCGTGCGAGCCC |
| | | C |
| 28S_P58 | 23074 | TTAGACTCCTTGGTCCGTGTTTCAAGACGGGTCGGGTGGGTAGCCGACG |
| | | T |
| 28S_P59 | 23075 | GCGCTCGCTCCGCCGTCCCCCTCTTCGGGGGACGCGCGCGTGGCCCCGA |
| | | G |
| 28S_P60 | 23076 | CCCGACGGCGCGACCCGCCCGGGGCGCACTGGGGACAGTCCGCCCCGCC |
| | | C |
| 28S_P61 | 23077 | GCACCCCCCCCGTCGCCGGGGCGGGGGCGCGGGGAGGAGGGGTGGGAGA |
| | | G |
| 28S_P62 | 23078 | AGGGGTGGCCCGGCCCCCCCACGAGGAGACGCCGGCGCGCCCCCGCGGG |
| | | G |
| 28S_P63 | 23079 | GGGGATTCCCCGCGGGGGTGGGCGCCGGGAGGGGGGAGAGCGCGGCGAC |
| | | G |
| 28S_P64 | 23080 | GCCCCGGGATTCGGCGAGTGCTGCTGCCGGGGGGGCTGTAACACTCGGG |
| | | G |
| 28S_P65 | 23081 | CCGCCCCCGCCGCCGCCGCCACCGCCGCCGCCGCCGCCGCCCCGACCCG |
| | | C |
| 28S_P66 | 23082 | AGGACGCGGGGCCGGGGGGCGGAGACGGGGGAGGAGGAGGACGGACGGA |
| | | C |
| 28S_P67 | 23083 | AGCCACCTTCCCCGCCGGGCCTTCCCAGCCGTCCCGGAGCCGGTCGCGG |
| | | C |
| 28S_P68 | 23084 | AAATGCGCCCGGCGGCGGCCGGTCGCCGGTCGGGGGACGGTCCCCCGCC |
| | | G |
| 28S_P69 | 23085 | CCGCCCGCCCACCCCCGCACCCGCCGGAGCCCGCCCCCTCCGGGGAGGA |
| | | G |
| 28S_P70 | 23086 | GGGAAGGGAGGGCGGGTGGAGGGGTCGGGAGGAACGGGGGGCGGGAAAG |
| | | A |
| 28S_P71 | 23087 | ACACGGCCGGACCCGCCGCCGGGTTGAATCCTCCGGGCGGACTGCGCGG |
| | | A |
| 28S_P72 | 23088 | TCTTAACGGTTTCACGCCCTCTTGAACTCTCTCTTCAAAGTTCTTTTCA |
| | | A |
| 28S_P73 | 23089 | CTTGTTGACTATCGGTCTCGTGCCGGTATTTAGCCTTAGATGGAGTTTA |
| | | C |
| 28S_P74 | 23090 | GCATTCCCAAGCAACCCGACTCCGGGAAGACCCGGGCGCGCGCCGGCCG |
| | | C |
| 28S_P75 | 23091 | GTCCACGGGCTGGGCCTCGATCAGAAGGACTTGGGCCCCCCACGAGCGG |
| | | C |
| 28S_P76 | 23092 | TTCCGTACGCCACATGTCCCGCGCCCCGCGGGGCGGGGATTCGGCGCTG |
| | | G |
| 28S_P77 | 23093 | CTCGCCGTTACTGAGGGAATCCTGGTTAGTTTCTTTTCCTCCGCTGACT |
| | | A |
| 28S_P78 | 23094 | GCGGGTCGCCACGTCTGATCTGAGGTCGCGTCTCGGAGGGGGACGGGCC |
| | | G |
| 5.8S_P1 | 23095 | AAGCGACGCTCAGACAGGCGTAGCCCCGGGAGGAACCCGGGGCCGCAAG |
| | | T |
| 5.8S_P3 | 23096 | GCAGCTAGCTGCGTTCTTCATCGACGCACGAGCCGAGTGATCCACCGCT |
| | | A |
| 5S_P1 | 23097 | AAAGCCTACAGCACCCGGTATTCCCAGGCGGTCTCCCATCCAAGTACTA |
| | | A |
| 5S_P3 | 23098 | TTCCGAGATCAGACGAGATCGGGCGCGTTCAGGGTGGTATGGCCGTAGA |
| | | C |
| HBA1_P1 | 23099 | GCCGCCCACTCAGACTTTATTCAAAGACCACGGGGGTACGGGTGCAGGA |
| | | A |
| HBA1_P2 | 23100 | GGGGGAGGCCCAAGGGGCAAGAAGCATGGCCACCGAGGCTCCAGCTTAA |
| | | C |
| HBA1_P3 | 23101 | GCACGGTGCTCACAGAAGCCAGGAACTTGTCCAGGGAGGCGTGCACCGC |
| | | A |
| HBA1_P4 | 23102 | GGGAGGTGGGCGGCCAGGGTCACCAGCAGGCAGTGGCTTAGGAGCTTGA |
| | | A |
| HBA1_P5 | 23103 | CCGAAGCTTGTGCGCGTGCAGGTCGCTCAGGGCGGACAGCGCGTTGGGC |
| | | A |
| HBA1_P6 | 23104 | CCACGGCGTTGGTCAGCGCGTCGGCCACCTTCTTGCCGTGGCCCTTAAC |
| | | C |
| HBA1_P7 | 23105 | CTCAGGTCGAAGTGCGGGAAGTAGGTCTTGGTGGTGGGGAAGGACAGGA |
| | | A |
| HBA1_P8 | 23106 | CTCCGCACCATACTCGCCAGCGTGCGCGCCGACCTTACCCCAGGCGGCC |
| | | T |
| HBA1_P9 | 23107 | CGGCAGGAGACAGCACCATGGTGGGTTCTCTCTGAGTCTGTGGGGACCA |
| | | G |
| HBA2_P1 | 23108 | GAGGGGAGGAGGGCCCGTTGGGAGGCCCAGCGGGCAGGAGGAACGGCTA |
| | | C |
| HBA2_P2 | 23109 | ACGGTATTTGGAGGTCAGCACGGTGCTCACAGAAGCCAGGAACTTGTCC |
| | | A |
| HBA2_P3 | 23110 | CAGGGGTGAACTCGGCGGGGAGGTGGGCGGCCAGGGTCACCAGCAGGCA |
| | | G |
| HBA2_P4 | 23111 | AAGTTGACCGGGTCCACCCGAAGCTTGTGCGCGTGCAGGTCGCTCAGGG |
| | | C |
| HBA2_P5 | 23112 | CATGTCGTCCACGTGCGCCACGGCGTTGGTCAGCGCGTCGGCCACCTTC |
| | | T |
| HBA2_P6 | 23113 | CCTGGGCAGAGCCGTGGCTCAGGTCGAAGTGCGGGAAGTAGGTCTTGGT |
| | | G |
| HBA2_P7 | 23114 | AACATCCTCTCCAGGGCCTCCGCACCATACTCGCCAGCGTGCGCGCCGA |
| | | C |
| HBA2_P8 | 23115 | CTTGACGTTGGTCTTGTCGGCAGGAGACAGCACCATGGTGGGTTCTCTC |
| | | T |
| HBB_P1 | 23116 | GCAATGAAAATAAATGTTTTTTATTAGGCAGAATCCAGATGCTCAAGGC |
| | | C |
| HBB_P2 | 23117 | CAGTTTAGTAGTTGGACTTAGGGAACAAAGGAACCTTTAATAGAAATTG |
| | | G |
| HBB_P3 | 23118 | GCTTAGTGATACTTGTGGGCCAGGGCATTAGCCACACCAGCCACCACTT |
| | | T |
| HBB_P4 | 23119 | CACTGGTGGGGTGAATTCTTTGCCAAAGTGATGGGCCAGCACACAGACC |
| | | A |
| HBB_P5 | 23120 | GCCTGAAGTTCTCAGGATCCACGTGCAGCTTGTCACAGTGCAGCTCACT |
| | | C |
| HBB_P6 | 23121 | CCCTTGAGGTTGTCCAGGTGAGCCAGGCCATCACTAAAGGCACCGAGCA |
| | | C |
| HBB_P7 | 23122 | CTTCACCTTAGGGTTGCCCATAACAGCATCAGGAGTGGACAGATCCCCA |
| | | A |
| HBB_P8 | 23123 | TCTGGGTCCAAGGGTAGACCACCAGCAGCCTGCCCAGGGCCTCACCACC |
| | | A |
| HBB_P9 | 23124 | ACCTTGCCCCACAGGGCAGTAACGGCAGACTTCTCCTCAGGAGTCAGAT |
| | | G |
| HBG1_P1 | 23125 | GTGATCTCTCAGCAGAATAGATTTATTATTTGTATTGCTTGCAGAATAA |
| | | A |
| HBG1_P2 | 23126 | CTCTGAATCATGGGCAGTGAGCTCAGTGGTATCTGGAGGACAGGGCACT |
| | | G |
| HBG1_P3 | 23127 | ATCTTCTGCCAGGAAGCCTGCACCTCAGGGGTGAATTCTTTGCCGAAAT |
| | | G |
| HBG1_P4 | 23128 | CACCAGCACATTTCCCAGGAGCTTGAAGTTCTCAGGATCCACATGCAGC |
| | | T |
| HBG1_P5 | 23129 | CACTCAGCTGGGCAAAGGTGCCCTTGAGATCATCCAGGTGCTTTGTGGC |
| | | A |
| HBG1_P6 | 23130 | AGCACCTTCTTGCCATGTGCCTTGACTTTGGGGTTGCCCATGATGGCAG |
| | | A |
| HBG1_P7 | 23131 | GCCAAAGCTGTCAAAGAACCTCTGGGTCCATGGGTAGACAACCAGGAGC |
| | | C |
| HBG1_P8 | 23132 | CTCCAGCATCTTCCACATTCACCTTGCCCCACAGGCTTGTGATAGTAGC |
| | | C |
| HBG1_P9 | 23133 | AAATGACCCATGGCGTCTGGACTAGGAGCTTATTGATAACCTCAGACGT |
| | | T |
| HBG2_P1 | 23134 | GTGATCTCTTAGCAGAATAGATTTATTATTTGATTGCTTGCAGAATAAA |
| | | G |
| HBG2_P2 | 23135 | TCTGCATCATGGGCAGTGAGCTCAGTGGTATCTGGAGGACAGGGCACTG |
| | | G |
| HBG2_P3 | 23136 | TCTTCTGCCAGGAAGCCTGCACCTCAGGGGTGAATTCTTTGCCGAAATG |
| | | G |
| HBG2_P4 | 23137 | ACCAGCACATTTCCCAGGAGCTTGAAGTTCTCAGGATCCACATGCAGCT |
| | | T |
| HBG2_P5 | 23138 | ACTCAGCTGGGCAAAGGTGCCCTTGAGATCATCCAGGTGCTTTATGGCA |
| | | T |
| HBG2_P6 | 23139 | GCACCTTCTTGCCATGTGCCTTGACTTTGGGGTTGCCCATGATGGCAGA |
| | | G |
| HBG2_P7 | 23140 | CCAAAGCTGTCAAAGAACCTCTGGGTCCATGGGTAGACAACCAGGAGCC |
| | | T |
| HBG2_P8 | 23141 | TCCAGCATCTTCCACATTCACCTTGCCCCACAGGCTTGTGATAGTAGCC |
| | | T |
| HBG2_P9 | 23142 | AATGACCCATGGCGTCTGGACTAGGAGCTTATTGATAACCTCAGACGTT |
| | | C |
| 5S_GNbac_P1 | 23143 | ATGCCTGGCAGTTCCCTACTCTCGCATGGGGAGACCCCACACTACCATC |
| | | G |
| 5S_GNbac_P2 | 23144 | ACTTCTGAGTTCGGCATGGGGTCAGGTGGGACCACCGCGCTACGGCCGC |
| | | C |
| 16S_GNbac_P1 | 23145 | GGTTACCTTGTTACGACTTCACCCCAGTCATGAATCACAAAGTGGTAAG |
| | | T |
| 16S_GNbac_P2 | 23146 | AAGCTACCTACTTCTTTTGCAACCCACTCCCATGGTGTGACGGGCGGTG |
| | | T |
| 16S_GNbac_P3 | 23147 | ACGTATTCACCGTGGCATTCTGATCCACGATTACTAGCGATTCCGACTT |
| | | C |
| 16S_GNbac_P4 | 23148 | AGACTCCAATCCGGACTACGACGCACTTTATGAGGTCCGCTTGCTCTCG |
| | | C |
| 16S_GNbac_P5 | 23149 | TGTATGCGCCATTGTAGCACGTGTGTAGCCCTGGTCGTAAGGGCCATGA |
| | | T |
| 16S_GNbac_P6 | 23150 | CCACCTTCCTCCAGTTTATCACTGGCAGTCTCCTTTGAGTTCCCGGCCG |
| | | G |
| 16S_GNbac_P7 | 23151 | GGATAAGGGTTGCGCTCGTTGCGGGACTTAACCCAACATTTCACAACAC |
| | | G |
| 16S_GNbac_P8 | 23152 | TGCAGCACCTGTCTCACGGTTCCCGAAGGCACATTCTCATCTCTGAAAA |
| | | C |
| 16S_GNbac_P9 | 23153 | GACCAGGTAAGGTTCTTCGCGTTGCATCGAATTAAACCACATGCTCCAC |
| | | C |
| 16S_GNbac_P10 | 23154 | CGTCAATTCATTTGAGTTTTAACCTTGCGGCCGTACTCCCCAGGCGGTC |
| | | G |
| 16S_GNbac_P11 | 23155 | TCCGGAAGCCACGCCTCAAGGGCACAACCTCCAAGTCGACATCGTTTAC |
| | | G |
| 16S_GNbac_P12 | 23156 | GTATCTAATCCTGTTTGCTCCCCACGCTTTCGCACTGAGCGTCAGTCTT |
| | | C |
| 16S_GNbac_P13 | 23157 | TTCGCCACCGGTATTCCTCCAGATCTCTACGCATTTCACCGCTACACCT |
| | | G |
| 16S_GNbac_P14 | 23158 | CTACGAGACTCAAGCTTGCCAGTATCAGATGCAGTTCCCAGGTTGAGCC |
| | | C |
| 16S_GNbac_P15 | 23159 | GACTTAACAAACCGCCTGCGTGCGCTTTACGCCCAGTAATTCCGATTAA |
| | | C |
| 16S_GNbac_P16 | 23160 | ATTACCGCGGCTGCTGGCACGGAGTTAGCCGGTGCTTCTTCTGCGGGTA |
| | | A |
| 16S_GNbac_P17 | 23161 | GTATTAACTTTACTCCCTTCCTCCCCGCTGAAAGTACTTTACAACCCGA |
| | | A |
| 16S_GNbac_P18 | 23162 | CGCGGCATGGCTGCATCAGGCTTGCGCCCATTGTGCAGTATTCCCCACT |
| | | G |
| 16S_GNbac_P19 | 23163 | GTCTGGACCGTGTCTCAGTTCCAGTGTGGCTGGTCATCCTCTCAGACCA |
| | | G |
| 16S_GNbac_P20 | 23164 | TAGGTGAGCCGTTACCCCACCTACTAGCTAATCCCATCTGGGCACATCC |
| | | G |
| 16S_GNbac_P21 | 23165 | AAGGTCCCCCTCTTTGGTCTTGCGACGTTATGCGGTATTAGCTACCGTT |
| | | T |
| 16S_GNbac_P22 | 23166 | CTCCATCAGGCAGTTTCCCAGACATTACTCACCCGTCCGCCACTCGTCA |
| | | G |
| 23S_GNbac_P1 | 23167 | AAGGTTAAGCCTCACGGTTCATTAGTACCGGTTAGCTCAACGCATCGCT |
| | | G |
| 23S_GNbac_P2 | 23168 | CCTATCAACGTCGTCGTCTTCAACGTTCCTTCAGGACCCTTAAAGGGTC |
| | | A |
| 23S_GNbac_P3 | 23169 | GGGGCAAGTTTCGTGCTTAGATGCTTTCAGCACTTATCTCTTCCGCATT |
| | | T |
| 23S_GNbac_P4 | 23170 | CCATTGGCATGACAACCCGAACACCAGTGATGCGTCCACTCCGGTCCTC |
| | | T |
| 23S_GNbac_P5 | 23171 | CCCCCTCAGTTCTCCAGCGCCCACGGCAGATAGGGACCGAACTGTCTCA |
| | | C |
| 23S_GNbac_P6 | 23172 | GCTCGCGTACCACTTTAAATGGCGAACAGCCATACCCTTGGGACCTACT |
| | | T |
| 23S_GNbac_P7 | 23173 | ATGAGCCGACATCGAGGTGCCAAACACCGCCGTCGATATGAACTCTTGG |
| | | G |
| 23S_GNbac_P8 | 23174 | ATCCCCGGAGTACCTTTTATCCGTTGAGCGATGGCCCTTCCATTCAGAA |
| | | C |
| 23S_GNbac_P9 | 23175 | ACCTGCTTTCGCACCTGCTCGCGCCGTCACGCTCGCAGTCAAGCTGGCT |
| | | T |
| 23S_GNbac_P10 | 23176 | CCTCCTGATGTCCGACCAGGATTAGCCAACCTTCGTGCTCCTCCGTTAC |
| | | T |
| 23S_GNbac_P11 | 23177 | GCCCCAGTCAAACTACCCACCAGACACTGTCCGCAACCCGGATTACGGG |
| | | T |
| 23S_GNbac_P12 | 23178 | AAACATTAAAGGGTGGTATTTCAAGGTCGGCTCCATGCAGACTGGCGTC |
| | | C |
| 23S_GNbac_P13 | 23179 | CCACCTATCCTACACATCAAGGCTCAATGTTCAGTGTCAAGCTATAGTA |
| | | A |
| 23S_GNbac_P14 | 23180 | TTCCGTCTTGCCGCGGGTACACTGCATCTTCACAGCGAGTTCAATTTCA |
| | | C |
| 23S_GNbac_P15 | 23181 | GACAGCCTGGCCATCATTACGCCATTCGTGCAGGTCGGAACTTACCCGA |
| | | C |
| 23S_GNbac_P16 | 23182 | CTTAGGACCGTTATAGTTACGGCCGCCGTTTACCGGGGCTTCGATCAAG |
| | | A |
| 23S_GNbac_P17 | 23183 | ACCCCATCAATTAACCTTCCGGCACCGGGCAGGCGTCACACCGTATACG |
| | | T |
| 23S_GNbac_P18 | 23184 | CACAGTGCTGTGTTTTTAATAAACAGTTGCAGCCAGCTGGTATCTTCGA |
| | | C |
| 23S_GNbac_P19 | 23185 | CCGCGAGGGACCTCACCTACATATCAGCGTGCCTTCTCCCGAAGTTACG |
| | | G |
| 23S_GNbac_P20 | 23186 | TTCCTTCACCCGAGTTCTCTCAAGCGCCTTGGTATTCTCTACCTGACCA |
| | | C |
| 23S_GNbac_P21 | 23187 | GTACGATTTGATGTTACCTGATGCTTAGAGGCTTTTCCTGGAAGCAGGG |
| | | C |
| 23S_GNbac_P22 | 23188 | ACCGTAGTGCCTCGTCATCACGCCTCAGCCTTGATTTTCCGGATTTGCC |
| | | T |
| 23S_GNbac_P23 | 23189 | ACGCTTAAACCGGGACAACCGTCGCCCGGCCAACATAGCCTTCTCCGTC |
| | | C |
| 23S_GNbac_P24 | 23190 | ACCAAGTACAGGAATATTAACCTGTTTCCCATCGACTACGCCTTTCGGC |
| | | C |
| 23S_GNbac_P25 | 23191 | ACTCACCCTGCCCCGATTAACGTTGGACAGGAACCCTTGGTCTTCCGGC |
| | | G |
| 23S_GNbac_P26 | 23192 | CGCTTTATCGTTACTTATGTCAGCATTCGCACTTCTGATACCTCCAGCA |
| | | T |
| 23S_GNbac_P27 | 23193 | TTCGCAGGCTTACAGAACGCTCCCCTACCCAACAACGCATAAGCGTCGC |
| | | T |
| 23S_GNbac_P28 | 23194 | CATGGTTTAGCCCCGTTACATCTTCCGCGCAGGCCGACTCGACCAGTGA |
| | | G |
| 23S_GNbac_P29 | 23195 | TAAATGATGGCTGCTTCTAAGCCAACATCCTGGCTGTCTGGGCCTTCCC |
| | | A |
| 23S_GNbac_P30 | 23196 | AACCATGACTTTGGGACCTTAGCTGGCGGTCTGGGTTGTTTCCCTCTTC |
| | | A |
| 23S_GNbac_P31 | 23197 | CCCGCCGTGTGTCTCCCGTGATAACATTCTCCGGTATTCGCAGTTTGCA |
| | | T |
| 23S_GNbac_P32 | 23198 | GGATGACCCCCTTGCCGAAACAGTGCTCTACCCCCGGAGATGAATTCAC |
| | | G |
| 23S_GNbac_P33 | 23199 | AGCTTTCGGGGAGAACCAGCTATCTCCCGGTTTGATTGGCCTTTCACCC |
| | | C |
| 23S_GNbac_P34 | 23200 | CGCTAATTTTTCAACATTAGTCGGTTCGGTCCTCCAGTTAGTGTTACCC |
| | | A |
| 23S_GNbac_P35 | 23201 | ATGGCTAGATCACCGGGTTTCGGGTCTATACCCTGCAACTTAACGCCCA |
| | | G |
| 23S_GNbac_P36 | 23202 | CCTTCGGCTCCCCTATTCGGTTAACCTTGCTACAGAATATAAGTCGCTG |
| | | A |
| 23S_GNbac_P37 | 23203 | GTACGCAGTCACACGCCTAAGCGTGCTCCCACTGCTTGTACGTACACGG |
| | | T |
| 23S_GNbac_P38 | 23204 | ACTCCCCTCGCCGGGGTTCTTTTCGCCTTTCCCTCACGGTACTGGTTCA |
| | | C |
| 23S_GNbac_P39 | 23205 | AGTATTTAGCCTTGGAGGATGGTCCCCCCATATTCAGACAGGATACCAC |
| | | G |
| 23S_GNbac_P40 | 23206 | ATCGAGCTCACAGCATGTGCATTTTTGTGTACGGGGCTGTCACCCTGTA |
| | | T |
| 23S_GNbac_P41 | 23207 | ACGCTTCCACTAACACACACACTGATTCAGGCTCTGGGCTGCTCCCCGT |
| | | T |
| 23S_GNbac_P42 | 23208 | GGGGAATCTCGGTTGATTTCTTTTCCTCGGGGTACTTAGATGTTTCAGT |
| | | T |
| 23S_GNbac_P43 | 23209 | ATTAACCTATGGATTCAGTTAATGATAGTGTGTCGAAACACACTGGGTT |
| | | T |
| 23S_GNbac_P44 | 23210 | GCCGGTTATAACGGTTCATATCACCTTACCGACGCTTATCGCAGATTAG |
| | | C |
| 5S_GPbac_P1 | 23211 | GCTTGGCGGCGTCCTACTCTCACAGGGGGAAACCCCCGACTACCATCGG |
| | | C |
| 5S_GPbac_P2 | 23212 | TTCCGTGTTCGGTATGGGAACGGGTGTGACCTCTTCGCTATCGCCACCA |
| | | A |
| 16S_GPbac_P1 | 23213 | TAGAAAGGAGGTGATCCAGCCGCACCTTCCGATACGGCTACCTTGTTAC |
| | | G |
| 16S_GPbac_P2 | 23214 | TCTGTCCCACCTTCGGCGGCTGGCTCCTAAAAGGTTACCTCACCGACTT |
| | | C |
| 16S_GPbac_P3 | 23215 | TCGTGGTGTGACGGGCGGTGTGTACAAGGCCCGGGAACGTATTCACCGC |
| | | G |
| 16S_GPbac_P4 | 23216 | ATTACTAGCGATTCCAGCTTCACGCAGTCGAGTTGCAGACTGCGATCCG |
| | | A |
| 16S_GPbac_P5 | 23217 | GTGGGATTGGCTTAACCTCGCGGTTTCGCTGCCCTTTGTTCTGTCCATT |
| | | G |
| 16S_GPbac_P6 | 23218 | CCAGGTCATAAGGGGCATGATGATTTGACGTCATCCCCACCTTCCTCCG |
| | | G |
| 16S_GPbac_P7 | 23219 | CACCTTAGAGTGCCCAACTGAATGCTGGCAACTAAGATCAAGGGTTGCG |
| | | C |
| 16S_GPbac_P8 | 23220 | ACCCAACATCTCACGACACGAGCTGACGACAACCATGCACCACCTGTCA |
| | | C |
| 16S_GPbac_P9 | 23221 | GACGTCCTATCTCTAGGATTGTCAGAGGATGTCAAGACCTGGTAAGGTT |
| | | C |
| 16S_GPbac_P10 | 23222 | ATTAAACCACATGCTCCACCGCTTGTGCGGGCCCCCGTCAATTCCTTTG |
| | | A |
| 16S_GPbac_P11 | 23223 | CCGTACTCCCCAGGCGGAGTGCTTAATGCGTTAGCTGCAGCACTAAGGG |
| | | G |
| 16S_GPbac_P12 | 23224 | ACTTAGCACTCATCGTTTACGGCGTGGACTACCAGGGTATCTAATCCTG |
| | | T |
| 16S_GPbac_P13 | 23225 | TCGCTCCTCAGCGTCAGTTACAGACCAGAGAGTCGCCTTCGCCACTGGT |
| | | G |
| 16S_GPbac_P14 | 23226 | ACGCATTTCACCGCTACACGTGGAATTCCACTCTCCTCTTCTGCACTCA |
| | | A |
| 16S_GPbac_P15 | 23227 | ATGACCCTCCCCGGTTGAGCCGGGGGCTTTCACATCAGACTTAAGAAAC |
| | | C |
| 16S_GPbac_P16 | 23228 | ACGCCCAATAATTCCGGACAACGCTTGCCACCTACGTATTACCGCGGCT |
| | | G |
| 16S_GPbac_P17 | 23229 | CCGTGGCTTTCTGGTTAGGTACCGTCAAGGTACCGCCCTATTCGAACGG |
| | | T |
| 16S_GPbac_P18 | 23230 | ACAACAGAGCTTTACGATCCGAAAACCTTCATCACTCACGCGGCGTTGC |
| | | T |
| 16S_GPbac_P19 | 23231 | CCATTGCGGAAGATTCCCTACTGCTGCCTCCCGTAGGAGTCTGGGCCGT |
| | | G |
| 16S_GPbac_P20 | 23232 | GGCCGATCACCCTCTCAGGTCGGCTACGCATCGTCGCCTTGGTGAGCCG |
| | | T |
| 16S_GPbac_P21 | 23233 | CTAATGCGCCGCGGGTCCATCTGTAAGTGGTAGCCGAAGCCACCTTTTA |
| | | T |
| 16S_GPbac_P22 | 23234 | TTCAAACAACCATCCGGTATTAGCCCCGGTTTCCCGGAGTTATCCCAGT |
| | | C |
| 16S_GPbac_P23 | 23235 | CCACGTGTTACTCACCCGTCCGCCGCTAACATCAGGGAGCAAGCTCCCA |
| | | T |
| 16S_GPbac_P24 | 23236 | GCATGTATTAGGCACGCCGCCAGCGTTCGTCCTGAGCCAGGATCAAACT |
| | | C |
| 23S_GPbac_P1 | 23237 | TGGTTAAGTCCTCGATCGATTAGTATCTGTCAGCTCCATGTGTCGCCAC |
| | | A |
| 23S_GPbac_P2 | 23238 | TATCAACCTGATCATCTTTCAGGGATCTTACTTCCTTGCGGAATGGGAA |
| | | A |
| 23S_GPbac_P3 | 23239 | GGCTTCATGCTTAGATGCTTTCAGCACTTATCCCGTCCGCACATAGCTA |
| | | C |
| 23S_GPbac_P4 | 23240 | GCAGAACAACTGGTACACCAGCGGTGCGTCCATCCCGGTCCTCTCGTAC |
| | | T |
| 23S_GPbac_P5 | 23241 | CAAATTTCCTGCGCCCGCGACGGATAGGGACCGAACTGTCTCACGACGT |
| | | T |
| 23S_GPbac_P6 | 23242 | GTACCGCTTTAATGGGCGAACAGCCCAACCCTTGGGACTGACTACAGCC |
| | | C |
| 23S_GPbac_P7 | 23243 | CGACATCGAGGTGCCAAACCTCCCCGTCGATGTGGACTCTTGGGGGAGA |
| | | T |
| 23S_GPbac_P8 | 23244 | GGGGTAGCTTTTATCCGTTGAGCGATGGCCCTTCCATGCGGAACCACCG |
| | | G |
| 23S_GPbac_P9 | 23245 | TTTCGTCCCTGCTCGACTTGTAGGTCTCGCAGTCAAGCTCCCTTGTGCC |
| | | T |
| 23S_GPbac_P10 | 23246 | GATTTCCAACCATTCTGAGGGAACCTTTGGGCGCCTCCGTTACCTTTTA |
| | | G |
| 23S_GPbac_P11 | 23247 | GTCAAACTGCCCACCTGACACTGTCTCCCCGCCCGATAAGGGCGGCGGG |
| | | T |
| 23S_GPbac_P12 | 23248 | GCCAGGGTAGTATCCCACCGATGCCTCCACCGAAGCTGGCGCTCCGGTT |
| | | T |
| 23S_GPbac_P13 | 23249 | ATCCTGTACAAGCTGTACCAACATTCAATATCAGGCTGCAGTAAAGCTC |
| | | C |
| 23S_GPbac_P14 | 23250 | CCTGTCGCGGGTAACCTGCATCTTCACAGGTACTATAATTTCACCGAGT |
| | | C |
| 23S_GPbac_P15 | 23251 | GCCCAGATCGTTGCGCCTTTCGTGCGGGTCGGAACTTACCCGACAAGGA |
| | | A |
| 23S_GPbac_P16 | 23252 | ACCGTTATAGTTACGGCCGCCGTTTACTGGGGCTTCAATTCGCACCTTC |
| | | G |
| 23S_GPbac_P17 | 23253 | CCTCTTAACCTTCCAGCACCGGGCAGGCGTCAGCCCCTATACTTCGCCT |
| | | T |
| 23S_GPbac_P18 | 23254 | CCTGTGTTTTTGCTAAACAGTCGCCTGGGCCTATTCACTGCGGCTCTCT |
| | | C |
| 23S_GPbac_P19 | 23255 | CAGAGCACCCCTTCTCCCGAAGTTACGGGGTCATTTTGCCGAGTTCCTT |
| | | A |
| 23S_GPbac_P20 | 23256 | ATCACCTTAGGATTCTCTCCTCGCCTACCTGTGTCGGTTTGCGGTACGG |
| | | G |
| 23S_GPbac_P21 | 23257 | TAGAGGCTTTTCTTGGCAGTGTGGAATCAGGAACTTCGCTACTATATTT |
| | | C |
| 23S_GPbac_P22 | 23258 | TCAGCCTTATGGGAAACGGATTTGCCTATTTCCCAGCCTAACTGCTTGG |
| | | A |
| 23S_GPbac_P23 | 23259 | CCGCGCTTACCCTATCCTCCTGCGTCCCCCCATTGCTCAAATGGTGAGG |
| | | A |
| 23S_GPbac_P24 | 23260 | TCAACCTGTTGTCCATCGCCTACGCCTTTCGGCCTCGGCTTAGGTCCCG |
| | | A |
| 23S_GPbac_P25 | 23261 | CGAGCCTTCCTCAGGAAACCTTAGGCATTCGGTGGAGGGGATTCTCACC |
| | | C |
| 23S_GPbac_P26 | 23262 | TACCGGCATTCTCACTTCTAAGCGCTCCACCAGTCCTTCCGGTCTGGCT |
| | | T |
| 23S_GPbac_P27 | 23263 | GCTCTCCTACCACTGTTCGAAGAACAGTCCGCAGCTTCGGTGATACGTT |
| | | T |
| 23S_GPbac_P28 | 23264 | TCGGCGCAGAGTCACTCGACCAGTGAGCTATTACGCACTCTTTAAATGG |
| | | T |
| 23S_GPbac_P29 | 23265 | AACATCCTGGTTGTCTAAGCAACTCCACATCCTTTTCCACTTAACGTAT |
| | | A |
| 23S_GPbac_P30 | 23266 | TGGCGGTCTGGGCTGTTTCCCTTTCGACTACGGATCTTATCACTCGCAG |
| | | T |
| 23S_GPbac_P31 | 23267 | AAGTCATTGGCATTCGGAGTTTGACTGAATTCGGTAACCCGGTAGGGGC |
| | | C |
| 23S_GPbac_P32 | 23268 | GCTCTACCTCCAAGACTCTTACCTTGAGGCTAGCCCTAAAGCTATTTCG |
| | | G |
| 23S_GPbac_P33 | 23269 | TCCAGGTTCGATTGGCATTTCACCCCTACCCACACCTCATCCCCGCACT |
| | | T |
| 23S_GPbac_P34 | 23270 | TTCGGGCCTCCATTCAGTGTTACCTGAACTTCACCCTGGACATGGGTAG |
| | | A |
| 23S_GPbac_P35 | 23271 | TCTACGACCACGTACTCATGCGCCCTATTCAGACTCGCTTTCGCTGCGG |
| | | C |
| 23S_GPbac_P36 | 23272 | TAACCTTGCACGGGATCGTAACTCGCCGGTTCATTCTACAAAAGGCACG |
| | | C |
| 23S_GPbac_P37 | 23273 | GGCTCTGACTACTTGTAGGCACACGGTTTCAGGATCTCTTTCACTCCCC |
| | | T |
| 23S_GPbac_P38 | 23274 | ACCTTTCCCTCACGGTACTGGTTCACTATCGGTCACTAGGGAGTATTTA |
| | | G |
| 23S_GPbac_P39 | 23275 | CTCCCGGATTCCGACGGAATTTCACGTGTTCCGCCGTACTCAGGATCCA |
| | | C |
| 23S_GPbac_P40 | 23276 | GTTTTGACTACAGGGCTGTTACCTCCTATGGCGGGCCTTTCCAGACCTC |
| | | T |
| 23S_GPbac_P41 | 23277 | CTTTGTAACTCCGTACAGAGTGTCCTACAACCCCAAGAGGCAAGCCTCT |
| | | T |
| 23S_GPbac_P42 | 23278 | CGTTTCGCTCGCCGCTACTCAGGGAATCGCATTTGCTTTCTCTTCCTCC |
| | | G |
| 23S_GPbac_P43 | 23279 | CAGTTCCCCGGGTCTGCCTTCTCATATCCTATGAATTCAGATATGGATA |
| | | C |
| 23S_GPbac_P44 | 23280 | GGTGGGTTTCCCCATTCGGAAATCTCCGGATCAAAGCTTGCTTACAGCT |
| | | C |
| 23S_GPbac_P45 | 23281 | TGTTCGTCCCGTCCTTCATCGGCTCCTAGTGCCAAGGCATCCACCGTGC |
| | | G |
| 16S:A1 | 23282 | AAACTAGATTCGAATATAACAAAACATTACATCCTCATCCAATCCCTTT |
| | | T |
| 16S:A2 | 23283 | GCGGTGTGTGCAAGGAGCAGGGACGTATTCACCGCGCGATTGTGACACG |
| | | C |
| 16S:A3 | 23284 | GCCTTTCGGCGTCGGAACCCATTGTCTCAGCCATTGTAGCCCGCGTGTT |
| | | G |
| 16S:A4 | 23285 | GCATACGGACCTACCGTCGTCCACTCCTTCCTCCTATTTATCATAGGCG |
| | | G |
| 16S:A5 | 23286 | CGGCATCCAAAAAAGGATCCGCTGGTAACTAAGAGCGTGGGTCTCGCTC |
| | | G |
| 16S:A6 | 23287 | CAACCTGGCTATCATACAGCTGTCGCCTCTGGTGAGATGTCCGGCGTTG |
| | | A |
| 16S:A7 | 23288 | AGGCTCCACGCGTTGTGGTGCTCCCCCGCCAATTCCTTTAAGTTTCAGT |
| | | C |
| 16S:A8 | 23289 | CCAGGCGGCGGACTTAACAGCTTCCCTTCGGCACTGGGACAGCTCAAAG |
| | | C |
| 16S:A9 | 23290 | TCCGCATCGTTTACAGCTAGGACTACCCGGGTATCTAATCCGGTTCGCG |
| | | C |
| 16S:A10 | 23291 | TTCCCACAGTTAAGCTGCAGGATTTCACCAGAGACTTATTAAACCGGCT |
| | | A |
| 16S:A12 | 23292 | CTCTTATTCCAAAAGCTCTTTACACTAATGAAAAGCCATCCCGTTAAGA |
| | | A |
| 16S:A13 | 23293 | CCCCCGTCGCGATTTCTCACATTGCGGAGGTTTCGCGCCTGCTGCACCC |
| | | C |
| 16S:A14 | 23294 | TTGTCTCAGGTTCCATCTCCGGGCTCTTGCTCTCACAACCCGTACCGAT |
| | | C |
| 16S:A16 | 23295 | CATTACCTAACCAACTACCTAATCGGCCGCAGACCCATCCTTAGGCGAA |
| | | A |
| 16S:A17 | 23296 | AAACCATTACAGGAATAATTGCCTATCCAGTATTATCCCCAGTTTCCCA |
| | | G |
| 16S:A18 | 23297 | AAGGGTAGGTTATCCACGTGTTACTGAGCCGTACGCCACGAGCCTAAAC |
| | | T |
| 23S:A1 | 23298 | ACCTAGCGCGTAGCTGCCCGGCACTGCCTTATCAGACAACCGGTCGACC |
| | | A |
| 23S:A2 | 23299 | CGTTCCTCTCGTACTGGAGCCACCTTCCCCTCAGACTACTAACACATCC |
| | | A |
| 23S:A3 | 23300 | CCTGTCTCACGACGGTCTAAACCCAGCTCACGTTCCCCTTTAATGGGCG |
| | | A |
| 23S:A4 | 23301 | GGTGCTGCTGCACACCCAGGATGGAAAGAACCGACATCGAAGTAGCAAG |
| | | C |
| 23S:A5 | 23302 | GGCTCTTGCCTGCGACCACCCAGTTATCCCCGAGGTAGTTTTTCTGTCA |
| | | T |
| 23S:A6 | 23303 | AGGAGGACTCTGAGGTTCGCTAGGCCCGGCTTTCGCCTCTGGATTTCTT |
| | | G |
| 23S:A7 | 23304 | CAAAGTAAGTTAGAAACACAGTCATAAGAAAGTGGTGTCTCAAGAACGA |
| | | A |
| 23S:A8 | 23305 | GACTTATAATCGAATTCTCCCACTTACACTGCATACCTATAACCAAGCT |
| | | T |
| 23S:A9 | 23306 | GTAAAACTCTACGGGGTCTTCGCTTCCCAATGGAAGACTCTGGCTTGTG |
| | | C |
| 23S:A10 | 23307 | TCACTAAGTTCTAGCTAGGGACAGTGGGGACCTCGTTCTACCATTCATG |
| | | C |
| 23S:A11 | 23308 | CGACAAGGCATTTCGCTACCTTAAGAGGGTTATAGTTACCCCCGCCGTT |
| | | T |
| 23S:A12 | 23309 | AACTGAACTCCAGCTTCACGTGCCAGCACTGGGCAGGTGTCGCCCTCTG |
| | | T |
| 23S:A13 | 23310 | CTAGCAGAGAGCTATGTTTTTATTAAACAGTCGGGCCCCCCTAGTCACT |
| | | G |
| 23S:A14 | 23311 | TTAAAACGCCTTAGCCTACTCAGCTAGGGGCACCTGTGACGGATCTCGG |
| | | T |
| 23S:A15 | 23312 | ACAAAACTAACTCCCTTTTCAAGGACTCCATGAATCAGTTAAACCAGTA |
| | | C |
| 23S:A16 | 23313 | ATAATGCCTACACCTGGTTCTCGCTATTACACCTCTCCCCAGGCTTAAA |
| | | C |
| 23S:A17 | 23314 | CAATCCTACAAAACATATCTCGAAGTGTCAGAAATTAGCCCTCAACGTC |
| | | A |
| 23S:A18 | 23315 | CTTTGCTGCTACTACTACCAGGATCCACATACCTGCAAGGTCCAAAGGA |
| | | A |
| 23S:A19 | 23316 | CAACCCACACAGGTCGCCACTCTACACAATCACCAAAAAAAAGGTGTTC |
| | | C |
| 23S:A20 | 23317 | GGATTAATTCCCGTCCATTTTAGGTGCCTCTGACCTCGATGGGTGATCT |
| | | G |
| 23S:A21 | 23318 | AGGGTGGCTGCTTCTAAGCCCACCTTCCCATTGTCTTGGGCCAAAGACT |
| | | C |
| 23S:A22 | 23319 | GTATTTAGGGGCCTTAACCATAGTCTGAGTTGTTTCTCTTTCGGGACAC |
| | | A |
| 23S:A23 | 23320 | CCTCACTCCAACCTTCTACGACGGTGACGAGTTCGGAGTTTTACAGTAC |
| | | G |
| 23S:A24 | 23321 | CCCTAAACGTCCAATTAGTGCTCTACCCCGCCACCAACCTCCAGTCAGG |
| | | C |
| 23S:A25 | 23322 | AATAGATCGACCGGCTTCGGGTTTCAATGCTGTGATTCCAGGCCCTATT |
| | | A |
| 23S:A26 | 23323 | ACAACGCTGCGGGCATATCGGTTTCCCTACGACTACAAGGATAAAAACC |
| | | T |
| 23S:A27 | 23324 | ACAAAGAACTCCCTGGCCCGTGTTTCAAGACGGACGATGCAACACTAGT |
| | | C |
| 23S:A28 | 23325 | ACAATGTTACCACTGATTCTTTCGGAAGAATTCATTCCTTACGCGCCAC |
| | | A |
| 23S:A29 | 23326 | CTGGTTTCAGGTACTTTTCACCCCCCTATAGGGGTACTTTTCAGCATTC |
| | | C |
| 23S:A30 | 23327 | CTCTATCGGTCTTGAGACGTATTTAGAATTGGAAGTTGATGCCTCCCAC |
| | | A |
| 23S:A31 | 23328 | ATCACCCTCTACGGTTCTAAAATTCCAAATAAAATTCGATTTATCCCAC |
| | | G |
| 23S:A32 | 23329 | TCTATACACCACATCTCCCTAATATTACTAAAAGGGATTCAGTTTGTTC |
| | | T |
| 23S:A33 | 23330 | GCCGTTACTAACGACATCGCATATTGCTTTCTTTTCCTCCGCCTACTAA |
| | | G |
| 23S:A34 | 23331 | GGGTTCCCAATCCTACACGGATCAACACAAAAAAAATGTGCTAGGAAGT |
| | | C |
| 5S:A1 | 23332 | ACTACTGGGATCGAAACGAGACCAGGTATAACCCCCATGCTATGACCGC |
| | | A |
| MM_16S_P10 | 23333 | GCGTATGCCTGGAGAATTGGAATTCTTGTTACTCATACTAACAGTGTTG |
| | | C |
| MM_16S_P11 | 23334 | GATTAACCCAATTTTAAGTTTAGGAAGTTGGTGTAAATTATGGAATTAA |
| | | T |
| MM_16S_P12 | 23335 | AGCTTGAACGCTTTCTTTATTGGTGGCTGCTTTTAGGCCTACAATGGTT |
| | | A |
| MM_16S_P13 | 23336 | ATTATTCACTATTAAAGGTTTTTTCCGTTCCAGAAGAGCTGTCCCTCTT |
| | | T |
| MM_16S_P14 | 23337 | CTTACTTTTTGATTTTGTTGTTTTTTTAGCAAGTTTAAAATTGAACTTA |
| | | A |
| MM_16S_P15 | 23338 | AACCAGCTATCACCAAGCTCGTTAGGCTTTTCACCTCTACCTAAAAATC |
| | | T |
| MM_16S_P7 | 23339 | AATACTTGTAATGCTAGAGGTGATGTTTTTGGTAAACAGGCGGGGTTCT |
| | | T |
| MM_16S_P8 | 23340 | TTTATCTTTTTGGATCTTTCCTTTAGGCATTCCGGTGTTGGGTTAACAG |
| | | A |
| MM_16S_P9 | 23341 | TTATTTATAGTGTGATTATTGCCTATAGTCTGATTAACTAACAATGGTT |
| | | A |
| RN_16S_P4 | 23342 | AGTGATTGTAGTTGTTTATTCACTATTTAAGGTTTTTTCCTTTTCCTAA |
| | | A |
| RN_16S_P5 | 23343 | TGGCTATATTTTAAGTTTACATTTTGATTTGTTGTTCTGATGGTAAGCT |
| | | T |
| RN_16S_P6 | 23344 | TTTTTTTAATCTTTCCTTAAAGCACGCCTGTGTTGGGCTAACGAGTTAG |
| | | G |
| RN_16S_P7 | 23345 | TGTTGGGTTAGTACCTATGATTCGATAATTGACAATGGTTATCCGGGTT |
| | | G |
| RN_16S_P8 | 23346 | AGGAGAATTGGTTCTTGTTACTCATATTAACAGTATTTCATCTATGGAT |
| | | C |
| RN_16S_P9 | 23347 | TTTGTGATATAGGAATTTATTGAGGTTTGTGGAATTAGTGTGTGTAAGT |
| | | A |
| MM_28S_P1 | 23348 | GCCGGGGAGTGGGTCTTCCGTACGCCACATTTCCCACGCCGCGACGCGC |
| | | G |
| MM_28S_P10 | 23349 | ACCTCGGGCCCCCGGGCGGGGCCCTTCACCTTCATTGCGCCACGGCGGC |
| | | T |
| MM_28S_P14 | 23350 | TCGCGTCCAGAGTCGCCGCCGCCGCCGGCCCCCCGAGTGTCCGGGCCCC |
| | | C |
| MM_28S_P15 | 23351 | CGCTGGTTCCTCCCGCTCCGGAACCCCCGCGGGGTTGGACCCGCCGCCC |
| | | C |
| MM_28S_P16 | 23352 | CGCCGACCCCCGACCCGCCCCCCGACGGGAAGAAGGAGGGGGGAAGAGA |
| | | G |
| MM_28S_P17 | 23353 | GGGACGACGGGGCCCCGCGGGGAAGAGGGGAGGGCGGGCCCGGGCGGAA |
| | | A |
| MM_28S_P18 | 23354 | GGCGCCGCGCGGAAAACCGCGGCCCGGGGGGCGGACCCGGCGGGGGAAC |
| | | A |
| MM_28S_P19 | 23355 | CCCCCACACGCGCGGGACACGCCCGCCCGCCCCCGCCACGCACCTCGGG |
| | | A |
| MM_28S_P2 | 23356 | CACCCGCTTTGGGCTGCATTCCCAAGCAACCCGACTCCGGGAAGACCCG |
| | | A |
| MM_28S_P20 | 23357 | TGGAGCGAGGCCCCGCGGGGAGGGGACCCGCGCCGGCACCCGCCGGGCT |
| | | C |
| MM_28S_P21 | 23358 | CGAGGCCGGCGTGCCCCGACCCCGACGCGAGGACGGGGCCGGGCGCCGG |
| | | G |
| MM_28S_P22 | 23359 | TCCCCGGAGCGGGTCGCGCCCGCCCGCACGCGCGGGACGGACGCTTGGC |
| | | G |
| MM_28S_P23 | 23360 | TCCACACGAACGTGCGTTCAACGTGACGGGCGAGAGGGCGGCCCCCTTT |
| | | C |
| MM_28S_P24 | 23361 | TCCCAAGACGAACGGCTCTCCGCACCGGACCCCGGTCCCGACGCCCGGC |
| | | G |
| MM_28S_P25 | 23362 | CCGCCGCGGGGACGACGCGGGGACCCCGCCGAGCGGGGACGGACGGGGA |
| | | C |
| MM_28S_P3 | 23363 | GCACCGCCACGGTGGAAGTGCGCCCGGCGGCGGCCGGTCGCCGGCCGGG |
| | | G |
| MM_28S_P6 | 23364 | CCCACCGGGCCCCGAGAGAGGCGACGGAGGGGGGTGGGAGAGCGGTCGC |
| | | G |
| MM_28S_P7 | 23365 | CCCGGCCCCCACCCCCACGCCCGCCCGGGAGGCGGACGGGGGGAGAGGG |
| | | A |
| MM_28S_P8 | 23366 | TATCTGGCTTCCTCGGCCCCGGGATTCGGCGAAAGCGCGGCCGGAGGGC |
| | | T |
| MM_28S_P9 | 23367 | CGCCGCCGACCCCGTGCGCTCGGCTTCGTCGGGAGACGCGTGACCGACG |
| | | G |
| RN_28S_P12 | 23368 | GCGCCCCCCCGCACCCGCCCCGTCCCCCCCGCGGACGGGGAAGAAGGGA |
| | | G |
| RN_28S_P14 | 23369 | CGAACCCCGGGAACCCCCGACCCCGCGGAGGGGGAAGGGGGAGGACGAG |
| | | G |
| RN_28S_P16 | 23370 | CACCCGGGGGGGCGACGAGGCGGGGACCCGCCGGACGGGGACGGACGGG |
| | | G |
| RN_28S_P17 | 23371 | GCCAACCGAGGCTCCTTCGGCGCTGCCGTATCGTTCCGCTTGGGCGGAT |
| | | T |
| RN_28S_P4 | 23372 | CCCGGGCCCCCGGACCCCCGAGAGGGACGACGGAGGCGACGGGGGGTGG |
| | | G |
| RN_28S_P5 | 23373 | TGGGAGGGGCGGCCCGGCCCCCGCGACCGCCCCCCTTTCCGCCACCCCA |
| | | C |
| RN_28S_P6 | 23374 | GGGAGAGGCCGGGGGGAGAGCGCGGCGACGGGTATCCGGCTCCCTCGGC |
| | | C |
| RN_28S_P7 | 23375 | CGCTGCTGCCGGGGGGCTGTAACACTCGGGGGGGGGTGGTCCGGCGCCC |
| | | A |
| RN_28S_P8 | 23376 | CGCCGCCGACCCCGTGCGCTCGGCTTCGCTCCCCCCCACCCCGAGAAGG |
| | | G |
| | 23377 | CTCATCCCCACCCTTTTCAACGGATGTGGGTTCGGTCCTCCACTGCCTC |
| | | T |
| | 23378 | AGCCGGGGCTTCTTAGTCAGGTACCGTCATTTTTTCTTCCCTGCTGATA |
| | | G |
| | 23379 | TAGATGATCAACCTACCGGGTTAGAGTAGCCATCACACAAGGGTAGTAT |
| | | C |
| | 23380 | CAGATGGCGGCATTGTCACTGCTCCGTCTCCACGTCACTCCTGAAGGTA |
| | | G |
| | 23381 | GGGAAGCAGGGTGGACCACCACCCAAGGCTAAATACTACCTGATGACCG |
| | | A |
| | 23382 | ACTAAACTTCACTCCGCATCACGTCTTCCCATTGCCGCACGGTTTTTCC |
| | | A |
| | 23383 | GTTCCTCCGCTTGTGCGGGCCCCCGTCAATTCCTTTGAGTTTCACCGTT |
| | | G |
| | 23384 | GCCCCAGACAACCATCGCTGGGGTTGAGCTACCTCACTGCGTCCCTCCG |
| | | C |
| | 23385 | CTTTCGTGCGGGTCGGAACTTACCCGACAAGGAATTTCGCTACCTTAGG |
| | | A |
| | 23386 | CAGGCGTCAGCTCGTATACGTCATCTTTCGATTTAGCACAAACCTGTGT |
| | | T |
| | 23387 | GGCTTCATGCTTAGATGCTTTCAGCACTTATCCCGTCCGCACATAGCTA |
| | | C |
| | 23388 | ATTACCGCGGCTGCTGGCACGTAGTTAGCCGGGGCTTCTTAGTCAGGTA |
| | | C |
| | 23389 | TTCACGCAAGATTTCTCGTGTCCCGCGCTACTCAGGATACCACTACGCT |
| | | T |
| | 23390 | ATCTAAAGTCTTCTCGTTTAAAATACTGGGCTGTTACCATCTGTGGCGG |
| | | A |
| | 23391 | GGGCTCTGACTTCTTGTAGGCATACGGTTTCAGGTTCTCTTTCACTCCG |
| | | C |
| | 23392 | GCTATGGATCGTCGGTTTGGTGGGCCGTTACCCCGCCAACTGCCTAATC |
| | | C |
| | 23393 | ATGACTTCAGCATGGGCGGTCATAACGCGGTACCAGAATATCAACTGGT |
| | | T |
| | 23394 | TTTCAGTTCAGGCGGTTCCCCTCATATACCTATGTATTCAGTATATGAT |
| | | G |
| | 23395 | CGAAAGGGGAGACGGCACGGGCCCGGAGGTTAGCGCCCCAGGCCTCGGT |
| | | T |
| | 23396 | TTTCGTCCCTGCTCGACTTGTAGGTCTCGCAGTCAAGCTCCCTTGTGCC |
| | | T |
| | 23397 | CTCTTATCGATGACATCTCCTCTTAACCTTCCAGCACCGGGCAGGTGTC |
| | | A |
| | 23398 | TCGTCCCTGACAACAGAGCTTTACGATCCGAAAACCTTCTTCACTCACG |
| | | C |
| | 23399 | ACCCAACATCTCACGACACGAGCTGACGACAACCATGCACCACCTGTCA |
| | | C |
| | 23400 | GTCCTCTCGTACTAAGGACAGAGCTCCTCAAATATCCTGCGCCCACGAC |
| | | A |
| | 23401 | TTATAGTTACGGCCGCCGTTTACCGGGGCTTCAATTCAGAGCTCTCACT |
| | | C |
| | 23402 | CGTTTCTACGAGTTAGAACTCAAATAATCAAAGGGCCGTATTTCAACAG |
| | | C |
| | 23403 | CACCAGTGTCGGTTTAGGGTACGGGCGGACCCGCCACCTCGCTCACGAA |
| | | G |
| | 23404 | CGTCCATCCCGGTCCTCTCGTACTAGGGACAGCTCCTCTCAAATATCCT |
| | | G |
| | 23405 | AGCTGACGCTCATGTTTCCAAGTCTCCCGCCTATCCTGTACATAGATTT |
| | | C |
| | 23406 | CTCTTTTAATGAGTGGCTGCTTCTAAGCCAACATCCTGGTTGTCTAAGC |
| | | A |
| | 23407 | ACAGCTTTTCTCGCCATCTTCCATCCCAGACTTCGGTACTAACTTCCCT |
| | | C |
| | 23408 | CATAGACCTGTGTTTTTGCTAAACAGTTGCTTGAGCCTATTCTCTGCGG |
| | | C |
| | 23409 | TCACGGTACTGGTTCACTATCGCTCACTCGTTTATATTTAGCCTTGGCG |
| | | G |
| | 23410 | ACTCACCCTGCCCCGATTAACGTTGGACAGGAACCCTTGGTCTTCCGGC |
| | | G |
| | 23411 | GGCTACAGTAAAGCTCCATGGGGTCTTTCCGTCTTGTCGCGGGTAACCG |
| | | G |
| | 23412 | GTACGATTTGATGTTACCTGATGCTTAGAGGCTTTTCCTGGAAGCAGGG |
| | | C |
| | 23413 | AAGTCATTGGCATTCGGAGTTTGACTGAATTCGGTAACCCGGTAGGGGC |
| | | C |
| | 23414 | GGTTACCTTGTTACGACTTCACCCCAGTCATGAATCACAAAGTGGTAAG |
| | | T |
| | 23415 | CCCTTCTCCCGTTGGCCTTAGAATCTTCTTCCTACCTACCTGTGTCGGT |
| | | T |
| | 23416 | TACCTTCACTAAGGTTCTTTCCGACGCTAGCCCTAAAGCTATTTCGGGG |
| | | A |
| | 23417 | CCCCCCTGCTTCCCACAGGGTTTCACGTGTCCCGTGGTACTCTGGATCA |
| | | C |
| | 23418 | GACCGGCCTTCCCATGCCGTTCGGTTAACAGATTAAGTCTTAAAAGCAG |
| | | T |
| | 23419 | TTCCTTTGACCCCCCCCCCCCCCCTCCCTATCCCCCCCCGCCCCCCCCC |
| | | A |
| | 23420 | CCCCCTCAGTTCTCCAGCGCCCACGGCAGATAGGGACCGAACTGTCTCA |
| | | C |
| | 23421 | CTTTGGGAGGCAACCGCCCCAGTTAAACTACCCGCCAGGCACTCTCCCC |
| | | G |
| | 23422 | ACATGATCGGTTCACACACTCACCACCACACAAGACCTCAAAGAGACCC |
| | | C |
| | 23423 | CCAGCACCGGGCAGGTGTCACCCCCTATACTTCGTCTTGCGACTTCGCA |
| | | G |
| | 23424 | GTACCGCTTTAATGGGCGAACAGCCCAACCCTTGGGACTGACTACAGCC |
| | | C |
| | 23425 | CCATTGCGGAAGATTCCCTACTGCTGCCTCCCGTAGGAGTCTGGGCCGT |
| | | G |
| | 23426 | TTCTCTGCGGCTCATGTTTCCATGAGCACCCCTTATCCCTAAGTTACGG |
| | | G |
| | 23427 | TTTGACTCATATCACACCTCACTGCTTAGACGTGCACTTCCAATCGCAC |
| | | G |
| | 23428 | CCGGTTTGCCCTCTTCCGCGTTCGCTCGCCACTACTTACGGAATCTCGT |
| | | T |
| | 23429 | TACCTGATCGACTTGTCAGTCTCCCAGTCAAGCGCCCTTATGCCATTAC |
| | | A |
| | 23430 | TCCCAAGCTTCGGTGTATGATTTAGCCCCGTTAAATTTTCGGCGCAGGG |
| | | T |
| | 23431 | CCTAGTCTTTTCAGTGCTCTACAAGCCGTGGTCATGGTTCGAGGCTGTA |
| | | C |
| | 23432 | TCGGGGTGCTTTTCACCTTTCCTTCACAGTACTCGTACGCTATCGGTCT |
| | | C |
| | 23433 | GGTCTGGGCTCTTTCCCTTTCGACTGCCCAACTTATCTCGTGCAGTCTG |
| | | A |
| | 23434 | GCACTCCACAGCTCCTTCCGGTACTGCTTCTTCGCGTTAAGAATGCTCC |
| | | T |
| | 23435 | GACTGCGAACCGTGAGCATTCGGAGTTCGTCAGGACTCGATAGGCGGTG |
| | | A |
| | 23436 | GTAAACAGTCGCTTGGGTCTATTCTCTGCGGCCCATTCCTGGGCACTCC |
| | | T |
| | 23437 | CCCACTTTCGTGCCTGCTCGACGTGTCTGTCTCGCAGTCAAGCCACCTT |
| | | G |
| | 23438 | TTTCCCTGCGGCTCCGGGACTTTATCCCTTAACCTTGCCAGTATGCACA |
| | | A |
| | 23439 | GGGCGCCTTCGCTTCGTAGCAGCTTTTCTCGCCAGCGTGAATTCAGCAG |
| | | C |
| | 23440 | TTCCGCCTGACCTTAGCTCCCGACTAACCCTGAGCGGACGAACCTTCCT |
| | | C |
| | 23441 | CTCTCAGGTCGGCTACTGATCGTCGGCTTGGTAGGCCGTTACCCCACCA |
| | | A |
| | 23442 | CTTCCTCCGGCTACTTAGATGTTTCAGTTCACCGGGTTCCCCTCCATAC |
| | | G |
| | 23443 | TACCTGATCGACTTGTTAGTCTCCCAGTCAAGCGCCCTTATGCCATTAC |
| | | A |
| | 23444 | GCAACCGCCCCAGTTAAACTACCCGCCAGGCACTGTCCCTGAACAGGAT |
| | | G |
| | 23445 | TTCCTCGTGTCTCGCCGTACTCAGGATCCCATTAGGCTTCGATCGGATT |
| | | T |
| | 23446 | ACGGATCGTCGCCTTGGTAGGCCTTTACCCCACCAACTAGCTAATGCAC |
| | | C |
| | 23447 | TGTCGGTTTGGGGTACGGGCGGCAACGCGCCTGACGCCGGGGCTTTTCT |
| | | C |
| | 23448 | CGGTTTCCGTTCGCGCTGAGGGAACCTTTGGGCGCCTCCGTTACATTTT |
| | | G |
| | 23449 | TTATAGTTACGGCCGCCGTTTACTGGGGCTTCAATTCAATGCTCTCACA |
| | | T |
| | 23450 | TGTAGCATGCGTGAAGCCCTGGACGTAAGGGGCATGATGATCTGACGTC |
| | | A |
| | 23451 | AGCACCGGGCAGGTGTCAGCACCTATACGTCAGCTCTCGCTTTCGCAGA |
| | | T |
| | 23452 | GCTGATAGGACGCGACCCCATCCCACGCCGATAGAATCTTTCCCACAAT |
| | | C |
| | 23453 | GTTTCAGGTTCTATTTCACTCCCCTCCCGGGGTGCTTTTCACCTTTCCC |
| | | T |
| | 23454 | CGGCTCCCATTCCGTGTCACCCCTGCGCTCACCTACCACGGCTACGCTC |
| | | C |
| | 23455 | TAGAGGCTTTTCTTGGCAGTGTGGAATCAGGAACTTCGCTACTATATTT |
| | | C |
| | 23456 | GGGGAATCTCGGTTGATTTCTTTTCCTCGGGGTACTTAGATGTTTCAGT |
| | | T |
| | 23457 | CATACCAGAGGTTCGTCCACCCAGGTCCTCTCGTACTATGGGCAGGCCT |
| | | C |
| | 23458 | CGCGGGTCCATCTTATACCACCGGAGTTTTTCACACTGAGCCATGCAGC |
| | | T |
| | 23459 | CTCCCGCAACCCCGGCCACGCAACCCCCGACGGGTATCGCGCGCGGCCG |
| | | G |
| | 23460 | TTCTCTGCGGCTCCATCTCTGGAGCACCCCTTCTCCCGAAGTTACGGGG |
| | | T |
| | 23461 | GAACATCCGGCATTACCACCCGTTTCCAGGAGCTATTCCGGAGCATGGG |
| | | G |
| | 23462 | AGGTCCCGGGGTCTTTTCGTCCTTCTGCGCTTAACGAGCATCTTTACTC |
| | | G |
| | 23463 | GCTTCGGTGGCATGTTTTAGCCCCGGACATTTTCGGCGCAGGACCTCTC |
| | | G |
| | 23464 | GCTTCAAAGCCTCCGACCTATCCTACACATCACGTGCCCAGATTCAATG |
| | | A |
| | 23465 | TACTTTATTTCGCTCCACATCACGGCTTCGTCTCATGCACAGCGGATTT |
| | | G |
| | 23466 | CATGGGGTCTTTCCGTCCTGTCGCGGGTAACCTGCATCTTCACAGGTAC |
| | | T |
| | 23467 | GACCTTCCTCTCAGAACCCCTACTGATCGTTGCCTTGGTGGGCCGTTAC |
| | | C |
| | 23468 | ATGTTTCAGTTCCCCGGGTTCCCCTCCATACGTTATGGATTGGCGTATG |
| | | G |
| | 23469 | TTAACGCTTTCGCTTGGCCGCTTACTGTATATCGCAAACAGCGAGTATT |
| | | C |
| | 23470 | CCACGGAAAACCACCTCCGCGGCCGGCTCCCATTCCGTGTCACCCCTGC |
| | | G |
| | 23471 | TCGTAACTCGCCGGTTCATTCTACAAAAGGCACGCTCTCACCCATTAAC |
| | | G |
| | 23472 | AGGATGCGACGAGCCGACATCGAGGTGCCAAACCTCCCCGCCGATATGG |
| | | A |
| | 23473 | TCCCCGGAGTACCTTTTATCCTTTGAGCGATGTCCCTTCCATACGGAAA |
| | | C |
| | 23474 | CGGCTTCCCTACTTTAATTTCGGTCCCTTACGCCCGGGTCAACCAACGC |
| | | C |
| | 23475 | CTGCTTCCAAGCCAACATCCTAGCTGTCTTAGCAGTCAGACTTCGTTAG |
| | | T |
| | 23476 | GCTACTCATACCGGCATTCTCACTTCTATGCGTTCCAGCGCTCCTCACG |
| | | G |
| | 23477 | GCCTTCGGTGTCTGCCTTATACCCGATTATTATCCATGCCCGGACCCTC |
| | | G |
| | 23478 | CCGGCTTTCCCAAAACCGTTCCACTAACATTGCAGAATCTTAAATGCAG |
| | | T |
| | 23479 | TACCTGTGTCGGTTTGCGGTACGGGCACCTTAGTATACACATAAGCTTT |
| | | T |
| | 23480 | TGTTACGCACTCTTTCAAGGGTGGCTGCTTCTGAGCCAACCTCCTGGCT |
| | | G |
| | 23481 | CTGGAGACCTTGGATATTCGGCCACAAGGATTCTCACCTTGTTCTCGCT |
| | | A |
| | 23482 | CAGTAACCCGCAAGGCTGCACCTAAATGCATTTCGGGGAGTACGAGCTA |
| | | T |
| | 23483 | AAACCTTGGATATTCGGCCTAGAGGATTCTCACCTCTATCTCGCTACTC |
| | | A |
| | 23484 | CGCTTGTGCGGGCCCCCGTCAATTTCTTTGAGTTTTAGCCTTGCGACCG |
| | | T |
| | 23485 | ACCGGGACACGTGATCCCACAACACCGGCAACGCAACCCCCGACGGGTA |
| | | T |
| | 23486 | GCTTTTCTCGCCTTCAGCCAAGTGTGCTTCCCTACTCTAATTTCGGTCC |
| | | C |
| | 23487 | CACTACTCACGGAGTATCCCTTCCTGCAGGTACTGAGATGTTTCACTTC |
| | | C |
| | 23488 | GATTGGAATTTCTCCGCTACCCACAGTTCATCCGCTACCATTTCAACGG |
| | | G |
| | 23489 | TTCCACGAGTCCCGCGCTACTCGGGAGACACCATCCATGGTGCACGCGC |
| | | A |
| | 23490 | GTCTTTTCGTCCCATCGCGGGTAATCGGCATCTTCACCGATACTACAAT |
| | | T |
| | 23491 | CCGTACATCATCTCGATGGCATTCGGAGTTTGATATTCTTTGGTAAGCT |
| | | T |
| | 23492 | GGGCTTGGCTACCCGGCTATAGACTTGGCAGTCTAACCGGTGCACCAGC |
| | | G |
| | 23493 | ACTTTCGTTACTGCTCGACCCGTCAGTCTCGCAGTTAGGCTCGCTTCTG |
| | | C |
| | 23494 | CTACTGTTTCTCCGCGTATACAACGCTCCCCTACCCAATCCATTACTGG |
| | | A |
| | 23495 | ACTTATAGTCAGCGCCCCTTCTCCCGAAGTTACGGGGCCATTTTGCCGA |
| | | G |
| | 23496 | CTTCCAAGCCAACATCCTAGCTGTCTTAGCAATCTGACTTCGTTAGTTC |
| | | A |
| | 23497 | CCTCGGCAACTGGCGTTACCGATTCTCAGCCTCCCACCTATCCTGTACA |
| | | T |
| | 23498 | CCATAACGGCTCCCATCATCACACCTCGCCATGCATGCCATGCGGATTT |
| | | G |
| | 23499 | CGTGCAGGTCGGAACTTACCCGACAAGGAATTTCGCTACCTTAGGACCG |
| | | T |
| | 23500 | CATCCAAACACTTTTCAACGTGTCCTGGTTCGGTCCTCCAGTGCGTTTT |
| | | A |
| | 23501 | GCCCTAAAGCTATTTCGGGGAGAACCAGCTATATCCGGGTTCGATTGGA |
| | | A |
| | 23502 | CAGTAAAGCTCTACGGGGTCTCTCCGTCCAGTCGCGGGTAATGGGCATC |
| | | T |
| | 23503 | GGAACCTTTGGGCGCCTCCGTTACGCTTTAGGAGGCGACCGCCCCAGTC |
| | | A |
| | 23504 | CCCGCCGTGTGTCTCCCGTGATAACATTCTCCGGTATTCGCAGTTTGCA |
| | | T |
| | 23505 | CAGGTGTCAGCCCCTATACTTCATCTTTCGATTTGGCAGAGACCTGTGT |
| | | T |
| | 23506 | GACTCTTCCCAGAGTCTTCTTCTATTCCCTTGGCTGCTTTATCGCAGTC |
| | | C |
| | 23507 | GGCAACCCAACAACCCACACACCATCATCTTCAGCTACAGGACTATCAC |
| | | C |
| | 23508 | AGCACCGGGCAGGTGTCAGGCTATATACCTCATGTTTCCATTTCGCATA |
| | | G |
| | 23509 | TTGCATACTATTAAGTTCAGCTCGGAAGGTGGATTTGCCTGCCTTCCTC |
| | | A |
| | 23510 | CCGGCGGATTTGCCAACCGGACACCCTACACCCTTGGACCAGGTCAATT |
| | | C |
| | 23511 | GCCGGTTATAACGGTTCATATCACCTTACCGACGCTTATCGCAGATTAG |
| | | C |
| | 23512 | CTGATACAACCAGTATCGCTCCGTCCATTTGCGCAGCACCAGTAATCAT |
| | | G |
| | 23513 | TCTTTGAATGTATGGCTGCTTCTGAGCCAACATCCTAGTTGTCTTCGAG |
| | | A |
| | 23514 | TGGATTCTCGCCCTCTTGTACTCATTTCGACTACGGGACTGTTACCCTC |
| | | T |
| | 23515 | CAGTATCAACTGCAATTTTACGGTTGAGCCGCAAACTTTCACAACTGAC |
| | | T |
| | 23516 | TTCTCTGCGGCTTACCTTCGTAAGCACCCCTTCTCCCGAAGTTACGGGG |
| | | T |
| | 23517 | ATTACTAGCGATTCCAGCTTCACGCAGTCGAGTTGCAGACTGCGATCCG |
| | | A |
| | 23518 | CATAGACCTGTGTTTTTGCTAAACAGTTGCTTGAGCCTATTCTCTGCGG |
| | | C |
| | 23519 | TATAAGTCGAGGCTGCACCTAAATGCATTTCGGGGAGTACGAGCTATCT |
| | | C |
| | 23520 | TCAACCTGTTGTCCATCGCCTACGCCTTTCGGCCTCGGCTTAGGTCCCG |
| | | A |
| | 23521 | GGGGTAGCTTTTATCCGTTGAGCGATGGCCCTTCCATGCGGAACCACCG |
| | | G |
| | 23522 | ATTAACCTATGGATTCAGTTAATGATAGTGTGTCGAAACACACTGGGTT |
| | | T |
| | 23523 | CCTCTTAACCTTCCAGCACCGGGCAGGCGTCAGCCCCTATACTTCGCCT |
| | | T |
| | 23524 | AAAAAGCAAGCTCTCTCAAGTTCCGTTCGACTTGCATGTGTTAGGCGCG |
| | | C |
| | 23525 | GGGCCCGTGTCTCAGTGCCCATGTGGGGGACCCTCCTCAGGCCGGCTAT |
| | | C |
| | 23526 | GACTTAACAAACCGCCTGCGTGCGCTTTACGCCCAGTAATTCCGATTAA |
| | | C |
| | 23527 | CAACCTGTTGTCCATCGGCTACGCTTTTCAGCCTCACCTTAGGTCCCGA |
| | | C |
| | 23528 | CACACACCACCACCACCCGAAAGCGGAGGCGGGGCGCGGGCAGATTGGT |
| | | T |
| | 23529 | CCGTTCGACTTGCATGTGTTAAGCACGCCGCCAGCGTTCATCCTGAGCC |
| | | A |
| | 23530 | GGCACCCTCTACGGCCAGGCCTTCAAGCCTGTTCCCCTGGCAAGCCGTT |
| | | T |
| | 23531 | GCCCTTCAAAAGCGTCCCTGTGTTTAAATCTTCGGAGGTTACGGAATTT |
| | | C |
| | 23532 | TCGTGGTGTGACGGGCGGTGTGTACAAGGCCCGGGAACGTATTCACCGC |
| | | G |
| | 23533 | TCCCGGGGTTCTTTTCACCGTTCCTTCACAGTACTATGCGCTATCGGTC |
| | | A |
| | 23534 | GACTGTTCGAGGTTAGACATCAAACGAGAACAGAGCGGTATTTCACCTT |
| | | G |
| | 23535 | CACCTTAGAGTGCCCAACTGAATGCTGGCAACTAAGATCAAGGGTTGCG |
| | | C |
| | 23536 | TATGGCACTTAAGCCGACACCTCACGGCACGAGCTGACGACAACCATGC |
| | | A |
| | 23537 | TCTCGTCCATTGACCAATATTCCTCACTGCTGCCTCCCGTAGGAGTTTG |
| | | G |
| | 23538 | TTTTCACCTTTCCCTCACGGTACTGGTTCGCTATCGGTCTCTCGGGAGT |
| | | A |
| | 23539 | TTCCCCATTCAGAGATCTCCGGATCAATGGATATTTGCTCCTCCCCGAA |
| | | G |
| | 23540 | TGAGCCAACATCCTGGTTGTCTGCGTATCTTCACATCGTTTTCCACTTA |
| | | A |
| | 23541 | TCGGAGTTTGATATTCTTCGGTAGGCTTTGACGCCCCCTAGGAAATTCA |
| | | G |
| | 23542 | CCTTCGGCTCCCCTATTCGGTTAACCTTGCTACAGAATATAAGTCGCTG |
| | | A |
| | 23543 | GTCTGGACCGTGTCTCAGTTCCAGTGTGGCTGGTCATCCTCTCAGACCA |
| | | G |
| | 23544 | TTATCCGTTCCGTACATAGCTGCCCAGCCGTGCCATTGGCATGACAACT |
| | | G |
| | 23545 | TTCACAGTACTATGCGCTATCGGTCACTAAGGAGTATTTAGCCTTGCGG |
| | | G |
| | 23546 | GACTCACCCGGGGACGACGAACGTGGCCCCGGAACCCTTGGTCATCCAG |
| | | C |
| | 23547 | GGCAACTTCAACCTGCACATGGATAGATCACCCGGTTTCGGGTCTACGT |
| | | A |
| | 23548 | ACCACGAATTCCGCCTGCCTCAACTGCACTCAAGATATCCAGTATCAAC |
| | | T |
| | 23549 | ACCACGCATTGCTGCATCCCAAGCTTCGGTTACATGCTTAGCCCCGTTA |
| | | C |
| | 23550 | CCAGAGCTTTTCTCGCCTCCGTCCAAGCATGCTTCCCTACTAAATTTCA |
| | | G |
| | 23551 | GCTGCACCTAAATGCATTTCGGAGAGAACCAGCTATCACGGAATTTGAT |
| | | T |
| | 23552 | CCTGGTTCGGGCCTCCAGTGAGTTTTACCTCACCTTCACCCTGCTCATG |
| | | G |
| | 23553 | ACTCACCCGGGGACGACGAACGTGGCCCCGGAACCCTTGGTCATCCAGC |
| | | G |
| | 23554 | AACATCCTGGTTGTCTGTGCAATTCCACATCCTTCTCCACTTAACGTGA |
| | | A |
| | 23555 | CTACGACTTCTCCCCATACAGAACGCTCTCCTACCATACATTAGATGTA |
| | | T |
| | 23556 | CACACTTAGCCCCGGACAACCATCACCGGGGATGAGCTACCTCACTGCG |
| | | T |
| | 23557 | GGGCGACCCTCCAACAGCGGCGGAACACATTTCGACTACGGGACTCTCA |
| | | C |
| | 23558 | CTCCGGTGCTTAACCTTGCCAGTGAGCGCAACTCGCCGGACCGTTCTAC |
| | | A |
| | 23559 | TTCGCAGGCTTACAGAACGCTCCCCTACCCAACAACGCATAAGCGTCGC |
| | | T |
| | 23560 | CCGTCAAGCCATGGGAGCCGGGTGTACCTAAAGTCGGTAACCGCAAGGA |
| | | G |
| | 23561 | TTACCTACACCATCACCTACACGCTTACACCAACAATCCACTAAGCGGC |
| | | A |
| | 23562 | GCGTACACCTGCAGCCTATCTACCTCGTAGTCTTCAAGGGGTCTTACCT |
| | | G |
| | 23563 | GCCGTCGCCCGTTAGTACCGGTCGGCTCCACCCCTCGCGGGGCTTCCAC |
| | | C |
| | 23564 | CACAGTGCTGTGTTTTTAATAAACAGTTGCAGCCAGCTGGTATCTTCGA |
| | | C |
| | 23565 | CTGTTATCCCCAGGGTAGCTTTTATCCGTTGAGCGACGGCATTTCCACT |
| | | C |
| | 23566 | ACTTAGATGCTTTCAGCACTTATCCAATCCCGACTTAGATACCCGGCAA |
| | | T |
| | 23567 | GCTTGCGCTAACCTCTCCTCTTAACCTTCCAGCACCGGGCAGGCGTCAG |
| | | C |
| | 23568 | ACCTATCCTGTACATGTGGTACAGATACTCAATATCAAACTGCAGTAAA |
| | | G |
| | 23569 | CTCCACCAGACTAAAACGAGGCTAGCCCTAAAGCTATTTCGAGGAGAAC |
| | | C |
| | 23570 | CCCGGCTTACCTTGGGCGGACGAACCTTCCCCAAGAAACCTTAGATTTT |
| | | C |
| | 23571 | GCAGAACAACTGGTACACCAGCGGTGCGTCCATCCCGGTCCTCTCGTAC |
| | | T |
| | 23572 | GACCAGGTCGATTCCATTGCCTGGCCCGGCTACCTTCCTGCGTCACACC |
| | | T |
| | 23573 | CTCTGAGACTTCAAATGTGTCCCTGTGCTTAACTCTTTTGGTGGTGACG |
| | | G |
| | 23574 | ACCTCGCGGTACGCCTTCGACGCTGACTGGAATGCTCCCCTACCGATCA |
| | | T |
| | 23575 | CGTCCATCCTGAGGGAACCTTTGGGCGCCTCCGATACCCTTTCGGAGGC |
| | | G |
| | 23576 | CACCTATCGGTCTCTCCTTAGGTCCCGACTAACCCAGGGCGGACGAGCC |
| | | T |
| | 23577 | CGCTCGCCGCTACTAAGGAAATCGATGTTTCTTTCTCTTCCTCCGGCTA |
| | | C |
| | 23578 | CGCGAGTCCATCTTCAAGCGATAAAATCTTTGATATCAAAACCATGTGG |
| | | T |
| | 23579 | TGACTGGAGTTTGTCCAGCCGGGTTTCCCCATTCAGAGATCTGCGGATC |
| | | A |
| | 23580 | CCTACTTAGCTACCCGGCTATGCCCCTGGCGGAACAACCGGTGCACCAG |
| | | C |
| | 23581 | ACGCTTAAACCGGGACAACCGTCGCCCGGCCAACATAGCCTTCTCCGTC |
| | | C |
| | 23582 | GATTTGCCTGGGATAATCAACATCTACACCCTTTAACGGACTATTCCGT |
| | | C |
| | 23583 | CTAATGCGCCGCGGGTCCATCTGTAAGTGGTAGCCGAAGCCACCTTTTA |
| | | T |
| | 23584 | GGATCTTAGCACTCGCAGTCTGACTGCCGACCATAAATCAATGGCATTC |
| | | G |
| | 23585 | ACCTATCCTGTACATGTGGTACAGGTACTCAATATCAAACTGCAGTAAA |
| | | G |
| | 23586 | TCACCGGGGATGAGCTACCTCACTGCGTCCCTCCGCAGCTTGCCTACTA |
| | | C |
| | 23587 | GCCATGCAGATTCTCACTGCATTCGCGCTACTCATTCCGGCATTCTCAC |
| | | T |
| | 23588 | CTTCACCTCACATACGACGCTCCCCTACCCCTGACAATTACTTGTCAAG |
| | | C |
| | 23589 | CCCTACTGATCGTCGCCTTGGTGGGCCGTTACCCCGCCAACAAGCTAAT |
| | | C |
| | 23590 | ACGCATTCGGAGTTTGTCAAGACTTGATAGGCGGTGAAGCCCTCGCATC |
| | | T |
| | 23591 | ACATTTTAGGAGGCGACCGCCCCAGTCAAACTGCCCGTCAGACACTGTC |
| | | T |
| | 23592 | GGTGGGTTTCCCCATTCGGAAATCTCCGGATCAAAGCTTGCTTACAGCT |
| | | C |
| | 23593 | CTCATCCCCACCCTTTTCAACGGATGTGGGTTCGGTCCTCCATTGCCTT |
| | | T |
| | 23594 | AGGTCACTTGGTTTCGGGTCTACATCTACGTACTTAACCGCCCTTTTCA |
| | | G |
| | 23595 | ACACACTCACCACACCACCACAACATCAAAGACATCACAATGGCAGGCT |
| | | C |
| | 23596 | TGACAACTGGTGCACCAGAGGTGCGTCCATCCCGGTCCTCTCGTACTAG |
| | | G |
| | 23597 | TCTGCCTCTGCACATTGCTCCTCTACCGCGCATCTTCTTCAGACGCACC |
| | | C |
| | 23598 | CTTTTCTCGACAGTACGGGATCACCAACTTCACCAATTAAGGCTACGCA |
| | | T |
| | 23599 | CCCTCATGTCACTATTTATTCATGACATGATGACACGCTGTTAACGTGC |
| | | C |
| | 23600 | GTACGCAGTCACACGCCTAAGCGTGCTCCCACTGCTTGTACGTACACGG |
| | | T |
| | 23601 | GGCGACCACCCCAGTCAAACTACCCACCAAGCAATGTCCGCGCATAGCG |
| | | C |
| | 23602 | GACTTAGTCCCAATCACGAGCCTCACCTTAGACGGCTCCATCCCACAAG |
| | | G |
| | 23603 | GCGCTTATGCGGTATTAGCAGTCATTTCTAACTGTTATCCCCCTGTATA |
| | | A |
| | 23604 | CGCTTTCACTGCGGCTACGTGTCTCGTGACACTCAACCTCGCCAGTGAC |
| | | G |
| | 23605 | ATGCTTTTCGCTTACAGGACTATAACCTTCTTTGGTGTGCCTTCCCATA |
| | | C |
| | 23606 | CGACTAACCCAGGGCGGACGAGCCTTCCCCTGGAAACCTTAGTCTTACG |
| | | G |
| | 23607 | TAGGACCCGACTAACCCTGATCCGATTAGCGTTGATCAGGAAACCTTAG |
| | | T |
| | 23608 | ACAGCTTTTCTCGTCTCTTTCCAAACTGACTTCCGCTTACGCGTCCCTT |
| | | A |
| | 23609 | TAAGACTTGCTCTCGCTGCGGCTTCAGACCTTAAGTCCTTAACCTTGCC |
| | | A |
| | 23610 | CTCTCAAACCAGCTATGGATCGTCGGCTTGGTAGGCCATTACCCCACCA |
| | | A |
| | 23611 | GGAATTTCTCCCCTATCCACACGTCATCTCCACCCTTTTCAACGGATGT |
| | | G |
| | 23612 | CCGGTCCATGGTCGGTACGGGAATATCCACCCGTTCATCCATTCGACTA |
| | | C |
| | 23613 | CCCCCGACCGGTTTCACGGCCGCAGGTTAGAATTCCAGAAACCTAAGGG |
| | | C |
| | 23614 | AAGTTTCGGTGGCTACGGAATTTCAACCGTATGTGCATCGACTACGCCT |
| | | C |
| | 23615 | TGCGCTCCCTTTACACCCAGTAAATCCGGATAACGCTTGCCCCCTACGT |
| | | A |
| | 23616 | ATTTCGCCTACGGGACTGTCACCCTCTATGGTCCACCTTTCCAGGTGAG |
| | | T |
| | 23617 | GCTTCGGTGGCATGTTTTAGCCCCGGACATTTTCGGCGCAGGACCTCTC |
| | | G |
| | 23618 | GACATGTCTCCACATCATTCAGTTGCAATTCAAGCCCGGGTAAGGTTCC |
| | | T |
| | 23619 | CGATAACTGGCACACCAGAGGTGCGTCCTTCCCGGTCCTCTCGTACTAG |
| | | G |
| | 23620 | AACGCTTATCGGTGCGGACCTCCATCCCGTGTTACCGGGACTTCATCCT |
| | | G |
| | 23621 | CCACTCCGTCGATGTGAACTCTTGGGAGTGATAAGCCTGTTATCCCCAG |
| | | G |
| | 23622 | GCCGCCTTTTCAACGGAGGTCGGTTCGGCCCTCCATGGAGTTTTACCTC |
| | | C |
| | 23623 | ACCGTTATAGTTACGGCCGCCGTTTACTGGGGCTTCAATTCGCACCTTC |
| | | G |
| | 23624 | AGGTGTTCTCATGTGGGTTTCCCCATTCAGAGATCTGCGGGTCAATGGA |
| | | T |
| | 23625 | AGCCTGTTCCCCTGGCAAGCCGTTTTATGACTCCCGCCCGGTCCGTCGG |
| | | A |
| | 23626 | GCTGACCTACTACGAGGGGGGATCCCAACGCGCCCGCGCCGCGACCCCC |
| | | C |
| | 23627 | GTTATCCCCCTGTATGAGGCAGGTTACCCACGCGTTACTCACCCGTCCG |
| | | C |
| | 23628 | CGGACATCTTCGGCGCACAATCACTCGACCAGTGAGCTATTACGCACTC |
| | | T |
| | 23629 | TGCTTGATGCCCGATTATTATCCACGCCAAACTCCTCGACTAGTGAGCT |
| | | G |
| | 23630 | CTCCATTCGGAAATCTGCGGATCAAAGCCTACTTACGGCTCCCCGCAGC |
| | | T |
| | 23631 | GCTGTTGGTCCGGATTGTTCTCCTTTAGGACATGGACCTTAGCACCCAT |
| | | G |
| | 23632 | TGCTGGCACGGAGTTAGCCGTCACTTCCTTGTTGAGTACCGTCATTATC |
| | | T |
| | 23633 | GCTATCGGTCAGACAGGTATGCTTAGACTTACCCAACGGTCTGGGCTGA |
| | | T |
| | 23634 | TATTCCTCACTGCTGCCTCCCGTAGGAGTTTGGACCGTGTCTCAGTTCC |
| | | A |
| | 23635 | TCCCGCTGGCCTTAGAATTCTCTTCCTGTCCACCTGTGTCGGTTTGCGG |
| | | T |
| | 23636 | CGACTATTGTCCTCGGCTTAGGTCCCGACTTACCCTGAGAGGACGAGCC |
| | | T |
| | 23637 | GGTCCTTTTCACCTTTCCTTCACAGTACTATGCGCTATCGGTCACTAAG |
| | | T |
| | 23638 | TCGGCTACTGATCGTCGCCTTGGTAGGCCGTTGCCCTGCCAACTAGCTA |
| | | A |
| | 23639 | CTTGGGAGTATGTTTACACGCACTATTACCGTTTTCCGAGGAAATTGGT |
| | | A |
| | 23640 | CACACAACCCCTACCAGGTATCACATGCACACGGTTTAGCCTCATCCAC |
| | | G |
| | 23641 | CCACGGCTTCGGTGTTGTGTTTTAGCCCCGGACATTTTCGGCGCAGGGC |
| | | C |
| | 23642 | CCACCTTCCTCCAGTTTATCACTGGCAGTCTCCTTTGAGTTCCCGGCCG |
| | | G |
| | 23643 | AGCTTTCGGGGAGAACCAGCTATCTCCCGGTTTGATTGGCCTTTCACCC |
| | | C |
| | 23644 | CGAGCCTTCCTCAGGAAACCTTAGGCATTCGGTGGAGGGGATTCTCACC |
| | | C |
| | 23645 | CCCAGGGCTAGATCATCCCGCTTCGGGTCCAGGACAAGCGACTGAAAAC |
| | | G |
| | 23646 | AAAATCATGGGAAATCTCATCTTGAGGGGGGCTTCGCACTTAGATGCTT |
| | | T |
| | 23647 | ATCCTGTACAAGCTGTACCAACATTCAATATCAGGCTGCAGTAAAGCTC |
| | | C |
| | 23648 | TTAGCAGGTGGTCCGGATTCTTCTCCTCTCGGGCACGGACCTTAGCACC |
| | | C |
| | 23649 | GTCCGTTTACGGTACGGGTACCTCAAGGATAAGTTTAGCGGGTTTTCTA |
| | | G |
| | 23650 | CACTGGCGTGCTGCCTTCTCTGCCTCCCACCTATCCTGTACATGAAATA |
| | | C |
| | 23651 | TGCGGTATTAGCAGTCATTTCTAACTGTTATCCCCCTGTATAAGGCAGG |
| | | T |
| | 23652 | GCTATCGGTCAGACAGGTATGCTTAGACTTACACCACGGTCGGTGCGGA |
| | | T |
| | 23653 | TTTACTCCTTTCGGATGGGATATCTCATCTTGAGGGGGGCTTCACGCTT |
| | | A |
| | 23654 | TGGCCGGTCGCCCTCTCAGGCCGGCTACCCGTCGAAGCCTTGGTGAGCC |
| | | G |
| | 23655 | AAGCCTGTTCCCCTGGCAAGCCGTTTTATGACTCCCGCCCGGCCCGTCG |
| | | G |
| | 23656 | AAGGTTAAGCCTCACGGTTCATTAGTACCGGTTAGCTCAACGCATCGCT |
| | | G |
| | 23657 | GACATCATACTAACGCGCCCTATTAAGACTCGGTTTCCCTACGGCTCCG |
| | | T |
| | 23658 | TGTGTTTTTGTTAAACAGTTGCCTGGACCGATTCTCTGCGCCTCAAGTC |
| | | G |
| | 23659 | GCCCCAGTCAAACTACCCACCAGACACTGTCCGCAACCCGGATTACGGG |
| | | T |
| | 23660 | GCGTCACACCTGTTAATGCGCTTGCCTTACCGGTTCAGGTCCCGCGCTC |
| | | C |
| | 23661 | GCGATGGCCCTTCCATGCGGAACCACCGGATCACTAAGCCCGACTTTCG |
| | | T |
| | 23662 | AAGCTCCATGGGGTCTTTCCGTCTAGTCGCGGGTAACCGGCATCTTCAC |
| | | C |
| | 23663 | CGCTAGCCCTAAAGCTATTTCGGAGAGAACCAGCTATCTCCAAGTTCGT |
| | | T |
| | 23664 | TCCCATCCGCACTTCGCTTCCCTGCTATGCCGTTGGCACGACAACAGTT |
| | | G |
| | 23665 | TTTCACTCCCCTCCCGGGGTCCTTTTCACCTTTCCTTCACAGTACTCTG |
| | | C |
| | 23666 | CGTCCTCGGCTTAGGCCCCGACTTACCCTGGGCGGATGAACCTTCCCCA |
| | | G |
| | 23667 | CGACATCGAGGTGCCAAACCTCCCCGTCGATGTGGACTCTTGGGGGAGA |
| | | T |
| | 23668 | TACCTGATCGACTTGTCAGTCTCCCAGTCAAGCGCCCTTATGCCATTAC |
| | | A |
| | 23669 | CTTCCAAGCCAACATCCTAGCTGTCTTAGCAATCTGACTTCGTTAGTTC |
| | | A |
| | 23670 | ACGCCTTAACCATGTGAAGGGTAGATTTTCTGACCCCTTCGGCCTGAAC |
| | | G |
| | 23671 | CTCAAGGATTAAGTTTAGCGGATTTTCTCGGGAGTATGTTTACACGCAC |
| | | T |
| | 23672 | CCCCATCCATCACCGATAAATCTTTAATCTCTTTCAGATGTCTTCTAGA |
| | | G |
| | 23673 | ATACTTTGGGACCTTAGCTGTGGGTCTGGGCTGTTTCCCTTTTGACAAT |
| | | G |
| | 23674 | CGCCCATAGGCGGTGCCGGCCCATGACGGCCGGCGGGTTCCCCCATTCG |
| | | G |
| | 23675 | AAAATCATGGGAAATCTCATCTTGAGGTGGGCTTCGCACTTAGATGCTT |
| | | T |
| | 23676 | ACAACTTGATACCCGATTATTATCCACGCCCGACTCCTCGACTAGTGAG |
| | | C |
| | 23677 | CTGAGTTTGATAAGCTTCGCTAACCTCTCGGCCGCTAGGCTATTCAGTG |
| | | C |
| | 23678 | GCCCAGATCGTTGCGCCTTTCGTGCGGGTCGGAACTTACCCGACAAGGA |
| | | A |
| | 23679 | TTATAGTTACGGCCGCCGTTCACTGGGGCTTCGGATCACTGCTTCAGAT |
| | | C |
| | 23680 | GGCATTGTCCCACCGCCGGGTCACGGCGGCTGGTTAGAAACCCAATACT |
| | | G |
| | 23681 | GTCCACACATTTAGCCCCAGACAACCATCGCTGGGGTTGAGCTACCTCA |
| | | C |
| | 23682 | TCTCACGACGTTCTGAACCCAGCTCGCGTGCCGCTTTAATGGGCGAACA |
| | | G |
| | 23683 | ATGCGACGAGCCGACATCGAGGTGCCAAACCTCCCCGTCGATGTGAACT |
| | | C |
| | 23684 | CCTGTGTCGGTTTAGGGTACGGGCAGTTTGAACCTCGCGCCGATGCTTT |
| | | T |
| | 23685 | CGATATTGCAAGGGTGGTATCCCAACAGCGCCTCCTCAGAGACTGGCGT |
| | | C |
| | 23686 | CCCCCGACCGGATTCACGGCCGCAGGTTAGAATTTCAGCACCTCAAGAG |
| | | T |
| | 23687 | TCAGATGGCGGCATTGTCACTACTGCGTCTCCACATCACTCCTGGAGGT |
| | | A |
| | 23688 | CTTTTCGTCCCATCGCGGGTAATCGGCATCTTCACCGATACTACAATTT |
| | | C |
| | 23689 | ACAACGAATTCCGCCAACTTCCCGCGCACTCAAGCCCTCCAGTTCGCGC |
| | | T |
| | 23690 | CCCGAAGTTACGGGGCCAATTTGCCGAGTTCCTTAACAACCCTTCTCCC |
| | | G |
| | 23691 | TCAAGGGGGTTTACTTCTTTCGAATGGGATATCTCATCTTAAGGGGGGC |
| | | T |
| | 23692 | CTTCACAGTACTATACGCTATCGGTCACTGGGTAGTATTTAGGGTTGGA |
| | | G |
| | 23693 | ATTCCGTCAGACGGCCGGACTGTCACTTCTCCGTCACCACATCGCTCTC |
| | | T |
| | 23694 | CGGTACTGGTTCACTATCGGTCACTAGGGAGTATTTAGGGTTGGGAGAT |
| | | G |
| | 23695 | AGCTGATGGTCCGGATTCTTCTCCTTTAGGACATGGACCTTAGCACCCA |
| | | T |
| | 23696 | CGTATTACCGCGGCTGCTGGCACGGAATTAGCCGGTCCTTATTCATAAG |
| | | G |
| | 23697 | ACGGGTTAGCCTCGCCACGCACCACTGACTCGCAGACTCATTTTTCGAT |
| | | A |
| | 23698 | ACGGCGTGGACTACCAGGGTATCTAATCCTGTTCGCTCCCCACGCTTTC |
| | | G |
| | 23699 | TGCGCATTCGGAGTTTATCAAGACTTGATAGGCGGTGAAGCCCTCGCAT |
| | | C |
| | 23700 | CTGTTGTCCATCGGCTACGACTCTCGTCCTCACCTTAGGCCCCGACTTA |
| | | C |
| | 23701 | GGCTCACGCCTCACCTTCGACGCGGAGTGGAATGCTCCCCTACCGATGT |
| | | T |
| | 23702 | GATGTTTCAGTTCAGGCGGTTCCCTCGATATACCTATTTTTAAGTTCAG |
| | | T |
| | 23703 | CATTGTCTAAGATTCCCCACTGCTGCCTCCCGTAGGAGTCTGGGCCGTG |
| | | T |
| | 23704 | TCACAGTACTATGCGCTATCGGTCACTAAGTGGTATTTAGCCTTAGGGG |
| | | G |
| | 23705 | GTAGTATTTAGGCTTGGAGGATGGTCCCTCCTGCTTCCCACAGGGTTTC |
| | | A |
| | 23706 | TTGGGACCTTAGCTGCGGGTCTGGGCTCTTTCCCTTTTGACTATCCAAC |
| | | T |
| | 23707 | CAGCTTGGTGGCGCAGAACTAAGCATTTGACTCAGTCCTCACCTCACTG |
| | | C |
| | 23708 | ACCAAGTACAGGAATATTAACCTGTTTCCCATCGACTACGCCTTTCGGC |
| | | C |
| | 23709 | AAGCCCGCTTGTGCGATTACACTCGACACCCGATTGCCAACCGGGCCGA |
| | | G |
| | 23710 | CCTTAAATACGCACAACCATCGGCGCACTGCAGCTACCTGTCTGCGTCA |
| | | C |
| | 23711 | CTACCCAGCGATGCCTTTGGCAAGACAACTGGTACACCAGCGGTAAGTC |
| | | C |
| | 23712 | CCTGTGTCGGTTTACGGTACGGGCGCATGGCAAACAATAGCGGCTTTTC |
| | | T |
| | 23713 | CCGCGCTTACCCTATCCTCCTGCGTCCCCCCATTGCTCAAATGGTGAGG |
| | | A |
| | 23714 | GGCTCTCTGTACTGTCAGGTTTCAGCAAGGACTAACTCTTAATCTGCCC |
| | | C |
| | 23715 | GGATCACCGGATTCGGGCCGTAAGGCCCCCATCATCGCGCCTCGCCCCG |
| | | A |
| | 23716 | TGGTCTCCGCTCGTTCAGACAAGGTTTCACGTGTCTCGTCCTACTCTGG |
| | | A |
| | 23717 | CAATCCCACTTTATGCCACCGGATCACTAAGTCCTACTTTCGTACCTGC |
| | | T |
| | 23718 | GTCACCAAGTAGTATTTAGCCTTGGGGGGTGGGCCCCCCGTCTTCCCAC |
| | | C |
| | 23719 | ATCCCCGGAGTACCTTTTATCCGTTGAGCGATGGCCCTTCCATTCAGAA |
| | | C |
| | 23720 | TACCTCTCACGGTGACCATCCGACGCGGCACCTAAATGCCTTTCGGGGA |
| | | G |
| | 23721 | CCGTACTCCCCAGGCGGAGTGCTTAATGCGTTAGCTGCAGCACTAAGGG |
| | | G |
| | 23722 | ATCACCAGTTTTACCCTAGGGCGCTCCTTGCGGTTACGCACTTCAGGTA |
| | | C |
| | 23723 | GGAGGGCACCTTTAGAAGCCTCCGTTACGCTTTTGGAGGCGACCACCCC |
| | | A |
| | 23724 | CTGGAGACCTTGGATATTCGGCCACAAGGATTCTCACCTTGTTCTCGCT |
| | | A |
| | 23725 | GGGCTTTCACCCTCTTTGGCTGGCTTTCCCAAAACCATTCTGCTAGGAT |
| | | C |
| | 23726 | GTGGGATTGGCTTAACCTCGCGGTTTCGCTGCCCTTTGTTCTGTCCATT |
| | | G |
| | 23727 | ATGCTACGCAGAGAAGTCCGGATATCAATGCCAGACTAGAGTAAAGCTC |
| | | C |
| | 23728 | TCCGTATACTCTCAGGTTCGACTCTCCCCGCGGATTTGCCTACGGGAAT |
| | | C |
| | 23729 | CTGGACCTATTCTCTGCGCCTCACATTGCTGTGAGGACCCTTTATCCCG |
| | | A |
| | 23730 | TTAGCAGGTGGTCCGGATTCTTCTCCTCTCGGGCACGGACCTTAGCACC |
| | | C |
| | 23731 | GCCTGTACACCTGCATCCTATCAACGTCATAGTCTTTGACGACCCTGAG |
| | | A |
| | 23732 | AGACTCCAATCCGGACTACGACGCACTTTATGAGGTCCGCTTGCTCTCG |
| | | C |
| | 23733 | GGTTTGCCCTCCTGCCTCTTCGCTCGCCGCTACTGAGGCAATCGCTCTT |
| | | G |
| | 23734 | ACCTTTCCCTCACGGTACTGGTACGCTATCGGTCAGACAGGTATGCTTA |
| | | G |
| | 23735 | CCGGTCCTCTCGTACTAGGGACAGCTCCCATCAAATATCCTGCGCCCAC |
| | | G |
| | 23736 | CCATTGGCATGACAACCCGAACACCAGTGATGCGTCCACTCCGGTCCTC |
| | | T |
| | 23737 | ATGTGCTTGTAAGCACAGAGTTTCAGGTTCTTTTCACTCCCCTCCCGGG |
| | | G |
| | 23738 | CCCTTCTCCCGAAGTTACGGGGTAATTTTGCCGAGTTCCTTAACAACCC |
| | | T |
| | 23739 | CCTGAGTCGGTTTAGGGTACGGGCGCGTTATGCCCTCACGTCGAGGCTT |
| | | T |
| | 23740 | ATCTGGGCTGTTTCCCTTTCGACAATGAAACTTATCTCACACTGTCTGA |
| | | C |
| | 23741 | CGTATTTCAAGGATGGCTCCACAAACACTGGCGTGCCTGCTTCAAAGCC |
| | | T |
| | 23742 | GGTCATTGCCTGCTTGCGGCTGACCATGGCTTATCGCAGCTGACCACGT |
| | | C |
| | 23743 | CCTGGCGCGGGTAACCAGCATCTTCACTGGTACTTCAATTTCACCGGGT |
| | | G |
| | 23744 | GTAACTCACAAGGCTGCACCTAAATGCATTTCGGGGAGTACGAGCTATC |
| | | T |
| | 23745 | GTCGGTTTGGGGTACGGGCGGCCATAGCCCTCACGCCGAGGCTTTTCTC |
| | | G |
| | 23746 | CACCGTCTATGGTCCCATTTTCCAAAGGGTTCTACTCATGAAATGTCTT |
| | | G |
| | 23747 | CCGGCAACGCAACCCCCGACGGGTATCACGCGCAACCGGTTTGGTCTGA |
| | | T |
| | 23748 | TTATCCTTCTGTGTCACTGCTTCATTCCATCGGTAGTGCAGGAATCTAC |
| | | A |
| | 23749 | CAGAGCACCCCTTCTCCCGAAGTTACGGGGTCATTTTGCCGAGTTCCTT |
| | | A |
| | 23750 | ATACTATCAGGTTCGATTCTCATGGTGGATTTGCCTGCCAAGATCAACA |
| | | T |
| | 23751 | CTTACGGGGCTTTCACCCTCTCTGGCCGGCTTTCCCAAAACCGTTCTGC |
| | | T |
| | 23752 | GACCGGCCTTCCCATGCCGTTCGGTTAACAACTTAAGTCCTAAATGCGG |
| | | T |
| | 23753 | CGTTTATCCGATCCGTACGTAGTTGCCCAGCTATGCTCCTGGCGGAACA |
| | | A |
| | 23754 | GTATCTAATCCTGTTTGATACCCACACTTTCGAGCATCAGCGTCAGTTA |
| | | C |
| | 23755 | GGTGCTTGTAAACACAAGGTTTCAGGTTCTTTTTCACTCCCCGTCAGGG |
| | | G |
| | 23756 | GTAGGCGCACGGTTTCAGGAACTCTTTCACTCCCCTCCCGGGGTGCTTT |
| | | T |
| | 23757 | ACTTCTGAGTTCGGCATGGGGTCAGGTGGGACCACCGCGCTACGGCCGC |
| | | C |
| | 23758 | TTCCGTGTTCGGTATGGGAACGGGTGTGACCTCTTCGCTATCGCCACCA |
| | | A |
| | 23759 | TCGCCTTAGGACCCGACTCACCCGGGGACGTTAACCGTGGCCCCGGAAC |
| | | C |
| | 23760 | CACTCACCCACAACCATGGGCTCCCCATCATGCCTCAACCTTCACGCCC |
| | | A |
| | 23761 | CTCCGAGACTTCATATGTGTCCCTGTGTTTAACTCTTTTGGTGGTGACG |
| | | G |
| | 23762 | AAAATTCCCTACTGCTGCCTCCCGTAGGAGTTTGGGCCGTGTCTCAGTC |
| | | C |
| | 23763 | GACCAGGTAAGGTTCTTCGCGTTGCATCGAATTAAACCACATGCTCCAC |
| | | C |
| | 23764 | CGAAGTTTGATAGGGTTCGGTAAGCTTTGTGGCCCCCTAGCCCATTCAG |
| | | T |
| | 23765 | AGGCTTGCGCCGCCGCTTCGCCCCGATGGGGACGCTCTCCTACCCAGCG |
| | | T |
| | 23766 | CGAACAGAGCGGTATTTCACCTTACGGCTCCGCGCGATCTGGCGACCGC |
| | | G |
| | 23767 | ACCGTTCTACAAAAAGTACGCGGTTGTACTCGTATGGTACTTCCACAGT |
| | | T |
| | 23768 | CGTTTCGCTCGCCGCTACTCAGGGAATCGCATTTGCTTTCTCTTCCTCC |
| | | G |
| | 23769 | GCTACTTGGGACAACACGATCGGAAGACGGCTCACGTCCAGGTACGGGG |
| | | C |
| | 23770 | AAGGTCCCCCTCTTTGGTCTTGCGACGTTATGCGGTATTAGCTACCGTT |
| | | T |
| | 23771 | GTTCTGAACCCAGCTCGCGTACCACTTTAATCGGCGAACAGCCGAACCC |
| | | T |
| | 23772 | TGATTCAAAGCCTCCGGCCTATCCTACACATCAATCACCCAAATTCAAT |
| | | G |
| | 23773 | GTCTTTTCGTCCCATCGCGGGTAATCGGCATCTTCACCGATACTACAAT |
| | | T |
| | 23774 | CCCCCCCCCCCCTTCCCCCCTCTCCTCCCCCTTCCCCCTTTCGCGCCCC |
| | | C |
| | 23775 | CAGGTGTCACCCCATATACGTCATCTTTCGATTTAGCATAGAGCTGTGT |
| | | T |
| | 23776 | CTCCACCAGACTAAAACGAGGCTAGCCCTAAAGCTATTTCGAGGAGAAC |
| | | C |
| | 23777 | TTCCGTCAGCCGGCAGGACTGTCACTTCTCCGTCTCCACGTCACTCCAT |
| | | G |
| | 23778 | CGCTAATTTTTCAACATTAGTCGGTTCGGTCCTCCAGTTAGTGTTACCC |
| | | A |
| | 23779 | CTTGGCAGTGTGACATCACTAACTTCGCTACTAAACTTCGCTCCCCATC |
| | | A |
| | 23780 | CCCGTTAAATTTTCGGCGCAGAGTCACTCGACCAGTGAGCTATTACGCA |
| | | C |
| | 23781 | CCCGGAGTACCTTTTATCCTTTGAGCGATGTCCCTTCCATGCGGAAACA |
| | | C |
| | 23782 | TTCTCTGCGGCTCCATCGCTGCAGCACCCCTTCTCCCGAAGTTACGGGG |
| | | T |
| | 23783 | AAGCTACCTACTTCTTTTGCAACCCACTCCCATGGTGTGACGGGCGGTG |
| | | T |
| | 23784 | GCACAGCCATGTGTTTTTGTTAAACAGTTGCCTGGACCTATTCTCTGCG |
| | | C |
| | 23785 | GCCAACATCCTGGTTGTCTGTGCAATTCCACATCCTTTTCCACTTAACT |
| | | A |
| | 23786 | GGTCACCCGGTTTCGGGCCCATTATATGCAACTTAACGCCCTTTTCAAA |
| | | C |
| | 23787 | TTATAGTTACGGCCGCCGTTCACTGGGGCTTCGATTCAATGCTTGCACA |
| | | T |
| | 23788 | GTTTATCTGAGATTGGTAATCCGGGATGGACCCCTCAATCAAACAGTGC |
| | | T |
| | 23789 | CGAAGTTACGGGGTCATTTTGCCGAGTTCCTTGACAATGCTTCTTCCGC |
| | | C |
| | 23790 | GTCCACACACGCGTGTGTCCCTCATCAGTTCTCACCCTCCATGCCCCCC |
| | | G |
| | 23791 | CCGGCCCGTCGGGGCCGGGACACACGCTCCCGCAACCCCGGCCACGCAA |
| | | C |
| | 23792 | CCGGTACATTTTCGGCGCAGGGTCACTCGACTAGTGAGCTATTACGCAC |
| | | T |
| | 23793 | CTCGAACTTCTTGTAAGCACACGGTTTCAGGTTCTCTTTCACTCCCCTT |
| | | C |
| | 23794 | TTTCAGTTCAGGCGGTTCCCCCCGTATCCCTATGGATTCAGAATACGGT |
| | | G |
| | 23795 | TCCGTTACATTTTGGGAGGCGACCGCCCCAGTCAAACTGCCTACCTGAC |
| | | A |
| | 23796 | CCGCTCCTTCCATCAAGGTTCCACGTGTCTCGATGTACTCTGGATCCTG |
| | | C |
| | 23797 | CCACGTGTTACTCACCCGTCCGCCGCTAACATCAGGGAGCAAGCTCCCA |
| | | T |
| | 23798 | GACTCCGTACTGTCAGGTTCGGCTCAACGGGTGGATTTGCCTGCCCATC |
| | | T |
| | 23799 | ACGTGTCCGGCGGTACTCTGGATACAGATGGCTGTTCAGGCTTTTCGTG |
| | | T |
| | 23800 | TGGGCTGTTTCCCTTTGGACAATGAAACTTATCTCCCACTGTCTGACTC |
| | | C |
| | 23801 | ACATAGCTACCCAGCCATGCCCTTGGCAGAACAACTGGTACACCAGCGG |
| | | T |
| | 23802 | CAGAGGTCAGTCCAACACGGTCCTCTCGTACTAGTGTCAGAGCCACGCA |
| | | A |
| | 23803 | GTTTGATAGGGTTCAGTAACTTCTCAGCCCCTAGCCCATTCAGTGCTTT |
| | | A |
| | 23804 | CGGCACCGGGCAGGCGTCACACCCTATACGTCCACTGTTCGTGTTGGCA |
| | | G |
| | 23805 | AACCCAATAAATCCGGATAACGCTTGCCCCCTACGTATTACCGCGGCTG |
| | | C |
| | 23806 | CCATACATCAATTATCTGGCATTCTGAGTTTGATAGGGTTCAGTAACCT |
| | | C |
| | 23807 | CCTCCGTTACACTTTGGGAGGCGACCGCCCCAGTCAAACTGCCCGCCAA |
| | | G |
| | 23808 | CTGTTATCCCCGAGGTAGCTTTTATCCGTTAAGCGACGGCTTTTCCACT |
| | | C |
| | 23809 | TAGCCCATTCAGTGCTTTACCTCCGGTAATCTAAATCAACGCTAGCCCT |
| | | A |
| | 23810 | TCCACAGCTCCTTACGGTACTGCTTCGTCCCGCATGCAATGCTCCTCTA |
| | | C |
| | 23811 | CCATCGCGGGTAATCGGCATCTTCACCGATACTACAATTTCACCGAGCT |
| | | C |
| | 23812 | CTGGACCTATTCTCTGCGCCCAACTCTCGTTGGGACCCTTTATCCCGAA |
| | | G |
| | 23813 | CTTTTACCTTTACACTCTACGATTGATTTCCAACCAATCTGAGCCAACC |
| | | T |
| | 23814 | TTATAGTTACGGCCGCCGTTTACCGGGGCTTCAATTCAAAGCTTCATAT |
| | | T |
| | 23815 | GCCATTAAGATTCTCACTTAATTCTCGCTACTTATTCCGGCATTCTCAC |
| | | T |
| | 23816 | GGCCGATCACCCTCTCAGGTCGGCTACGCATCGTCGCCTTGGTGAGCCG |
| | | T |
| | 23817 | CTTCTCCCGCTGGCCTTAGAATCTTCTTCCTATCTACCTGTGTCGGTTT |
| | | G |
| | 23818 | TTCCTTCACCCGAGTTCTCTCAAGCGCCTTGGTATTCTCTACCTGACCA |
| | | C |
| | 23819 | GCTAGTCCTAAAACTATTTCGGGGAGAACCAGCTATCTCCGGGTTCGAT |
| | | T |
| | 23820 | CCTCCGGCCGGTTTCACGGCCGCAAGTTAGAATTCCAGCACTACAAGAG |
| | | T |
| | 23821 | TGTTCGTCCCGTCCTTCATCGGCTCCTAGTGCCAAGGCATCCACCGTGC |
| | | G |
| | 23822 | GCCAGGCCTTCAAGCCTGTTCCCCTGGCTAGCCGCTTTATGACTCCCGC |
| | | C |
| | 23823 | CTTTCTTTTCCTCCGGCTACTTAGATGTTTCAGTTCACCGGGTTCCCTT |
| | | C |
| | 23824 | ATGATTCTCACATAATTCTCGCTACTCATTCCGGCATTCTCACTCGTAT |
| | | G |
| | 23825 | CGGGCACGGACCTTAGCACCCATGCCCTTACTGCCGGACTGCAGACCGT |
| | | G |
| | 23826 | GTGAGTTTCCTCATTCAGAGATCTCCGGATCAATGCTTATTTGCAGCTC |
| | | C |
| | 23827 | TAAATGCAGTCCGAACCCCGGAGTGCACGCACTCCGGTTTGGGCTCTTT |
| | | C |
| | 23828 | GCCCAAGGGTAGATCACTTGGTTTCGCGTCTACTCCTTCCGACTATACG |
| | | C |
| | 23829 | AGCTTAGCGGATTTTCTCGGGAGTCTGATTACCGGCGCTATTGGATTCC |
| | | A |
| | 23830 | CTCGCAGTCAAGCTCCCTTCTGCCTTTGCACTCTCCGAATGATTTCCAA |
| | | C |
| | 23831 | GTCTAGTCCCACGTACTTGTGCGCCCTGTTCAGACTCGCTTTCGCTCCG |
| | | C |
| | 23832 | TTCTCCGCTATCCACACCTCATCGCCACCCTTTTCAACGGATGTGCGTT |
| | | C |
| | 23833 | GCCGGCTCCCATTCCGTGTCACCCCTGCGCTCACCTACCACGGCTACGC |
| | | T |
| | 23834 | TCCCGGGGTCCTTTTCACCTTTCCTTCACAGTACTATGCGCTATCGGTC |
| | | A |
| | 23835 | CCAACATCCTGGTTGTCTGTGCAATTCCACATCCTTTTCCACTTAAATC |
| | | C |
| | 23836 | GCTGGCGCCGCGGCTTCGAAGCCTCCCGCCTATGCTACACAATCCGCAC |
| | | C |
| | 23837 | ACGCCCAATAATTCCGGACAACGCTTGCCACCTACGTATTACCGCGGCT |
| | | G |
| | 23838 | CCCTACCAGGTATCACATGCACACGGTTTAGCCTCATCCACGTTCGTTC |
| | | G |
| | 23839 | AGCACCGGGCAGGTGTCAGGCTGTATACGTGATCTTTCAATTTGGCACA |
| | | G |
| | 23840 | CTCCCCATCATGCCTCAACCTTCACGCCCAGCGGATTTACCTACCAGAC |
| | | A |
| | 23841 | CTTCAACTTAACCTCGCACGTAAACGTAACTCGCCGGTTCATTCTACAA |
| | | A |
| | 23842 | AGAGTAGCCATAACACAAGGGTAGTATCCCAACAACGCCTCAGTCGAAA |
| | | C |
| | 23843 | GCTCGCGTACCACTTTAAATGGCGAACAGCCATACCCTTGGGACCTACT |
| | | T |
| | 23844 | CATAGACCTGTGTTTTTGCTAAACAGTTGCTTGAGCCTATTCTCTGCGG |
| | | C |
| | 23845 | ACACACAACCCCTACCAAGTATCACATGCACACGGTTTAGCCTCATCCA |
| | | C |
| | 23846 | TCTACGACCACGTACTCATGCGCCCTATTCAGACTCGCTTTCGCTGCGG |
| | | C |
| | 23847 | CATTCGGATATCTCTGGATCAAGGCTTACTTACAGCTCCCCAAAGCATG |
| | | T |
| | 23848 | GCTCTCCTACCACTGTTCGAAGAACAGTCCGCAGCTTCGGTGATACGTT |
| | | T |
| | 23849 | TCTTTTCGTCCCATCGCGGGTAATCGGCATCTTCACCGATACTACAATT |
| | | T |
| | 23850 | TGTACCCCCCATTGTAACACGTGTGTAGCCCCGGACGTAAGGGCCGTGC |
| | | T |
| | 23851 | TCCCCGGAGTACCTTTTATCCTTTGAGCGATGTCCCTTCCATACGGAAA |
| | | C |
| | 23852 | CGTTGAGCGATGGCCCTTCCTTTCGGTACCACCGGATCACTAAGCCCGA |
| | | C |
| | 23853 | TTCAAGGGGTCTTACTCGTTATACGATGGGATATCTAATCTTGGAGTCG |
| | | G |
| | 23854 | CCTCCTGATGTCCGACCAGGATTAGCCAACCTTCGTGCTCCTCCGTTAC |
| | | T |
| | 23855 | ACCTTGGTCTTACGGCGGGAGGGAATCTCACCCTCCTTATCGTTACTTA |
| | | T |
| | 23856 | CGTGCCCCGCCCTACTCAGGATACTGCTAGCCACGATCAACTTTTAGGT |
| | | A |
| | 23857 | CACCCTCAGTTCATCCGGAAGCTTTTCAACGCTTATCGGTTCGGTCCTC |
| | | C |
| | 23858 | TCTACCTCCATGAGACTAATACGAGGCTAGCCCTAAAGCTATTTCGAGG |
| | | A |
| | 23859 | TACCTGTGTCGGTTTGCGGTACGGGCACCTTAGCATACACTAGAACTTT |
| | | T |
| | 23860 | AGCGGTTCCACAGCTTGTAAACATATGGTTTCAGGTTCTCTTTCACTCC |
| | | C |
| | 23861 | TTATAGTTACGGCCGCCGTTCACTGGGGCTTCGGGTCAAAGCTTGCACT |
| | | C |
| | 23862 | TTATAGTTACGGCCGCCGTTTACTGGGGCTTCGGTTCGATGCTTCGATT |
| | | G |
| | 23863 | GCCTTACGGGGTGGTCCCCGCTCATTCCCACAAGGTTTCTCGTGTCTCG |
| | | T |
| | 23864 | CCGGAGTTTTTCACACTGAGCCATGCAGCTCTGTGCGCTTATGCGGTAT |
| | | T |
| | 23865 | CTTCTCCCGTTGGCCTTAGAATCTTCTTCCTACCTACCTGTGTCGGTTT |
| | | G |
| | 23866 | TGCCGCTTTAATGGGCGAACAGCCCAACCCTTGGGACCGACTACAGCCC |
| | | C |
| | 23867 | GGAGTTCTTCGTGATATCTAAGCATTTCACCGCTACACCACGAATTCCG |
| | | C |
| | 23868 | AGTGATGGGCAGGTTGGATACGCGTTACTCACCCGTGCGCCGGTCGACG |
| | | C |
| | 23869 | TCACGGTACTCGTACGCTATCGGTCAGACAGGTATACTCAGGCTTACCC |
| | | G |
| | 23870 | ACGCATTCGGAGTTTGTCAAGACTTGATAGGCGGTGAAGCCCTCGCATC |
| | | T |
| | 23871 | CATCATCTGTATGGCATTCGGAGTTTGATATCCCTTAGTAAGCTTTGAC |
| | | G |
| | 23872 | TTCTCCGCTATCCACACCTCATCGCCACCCTTTTCAACGGATGTGCGTT |
| | | C |
| | 23873 | AAGCACTTTGGTTTGGGCTGTTCCCCGTTCGCTCGCCGCTACTTAGGGA |
| | | A |
| | 23874 | CACTTATGCCCGATTATTATCCACGCCAAACTCCTCGACTAGTGAGCTG |
| | | T |
| | 23875 | CTTAGGACCCGACTCACCCAGGGCAGACAAACTTGACCCTGGAACCCTT |
| | | G |
| | 23876 | CTCATCAGTTCTCACCCCCAATGTCCCCCGGATTTACCTGAGGGACGGG |
| | | C |
| | 23877 | CCCATGGTGCACGCACCATGGTTTGGGCTCTTCCGCGTTCGCTCGCCGC |
| | | T |
| | 23878 | GCTAGTCCTAAAACTATTTCGGGGAGAACCAGCTATCTCCGGGTTCGAT |
| | | T |
| | 23879 | ACCCCATCAATTAACCTTCCGGCACCGGGCAGGCGTCACACCGTATACG |
| | | T |
| | 23880 | CATTCCGGCATTCTCACTCGAATACAATCCACCGCTGCTTCCGCTACGA |
| | | C |
| | 23881 | GTTTCAGTTCGCCGGGTACCTCTCTTGCAGGCCATGTATTCACCTGCAG |
| | | A |
| | 23882 | ACCTGAGGCTACTCGCCTCGACTACCTGTGTCGGTTTGCGGTACGGGTA |
| | | G |
| | 23883 | AAGGCTAGCCCTAAAGCTATTTCGAGGAGAACCAGCTATCTCCGGGTTC |
| | | G |
| | 23884 | ATTATTATTTTCTCCTCCTACGGGTACTGAGATGTTTCACTTCCCCGCG |
| | | T |
| | 23885 | GCTTGCGCTAACCTCTCCTCTTAACCTTCCAGCACCGGGCAGGCGTCAG |
| | | C |
| | 23886 | CAGAGGTCTGTCCAACACGGTCCTCTCGTACTAGTGTCAGAGCCACGCA |
| | | A |
| | 23887 | ATCCTCTCAGACCAGTTACGGATCGTCGCCTTGGTAGGCCTTTACCCCA |
| | | C |
| | 23888 | TCACGCAGAATTCCTCGTGCTCCGCGCTACTCAGGATACCACTAGGCTT |
| | | C |
| | 23889 | CGCGTCTTCGGTGGCGTGCTTGAGCCCCGCTACATTGTCGGCGCGGAAC |
| | | C |
| | 23890 | TACTTATGCCCGATTATTATCCACGCCAAACTCCTCGACTAGTGAGCTG |
| | | T |
| | 23891 | ACCGTAGTGCCTCGTCATCACGCCTCAGCCTTGATTTTCCGGATTTGCC |
| | | T |
| | 23892 | AGCTGACGCCTGTATTTCCCAGTCTCCCACCTATCCTGTACATGAAATA |
| | | C |
| | 23893 | GGCGTTGCTGATCCGCGATTACTAGCGACTCCGCCTTCACGGAGCCGGG |
| | | T |
| | 23894 | GGGTGCCGCATGGGTTAAGCTTAGCGGATTTTCTCGGGAGTATGGTTAC |
| | | C |
| | 23895 | TCTTCAGCCCCAGGATGCGATGAGCCGACATCGAGGTGCCAAACTTCCT |
| | | C |
| | 23896 | CGCCGGCACCGGATCACTATCTCCGACTTTCGTCCCTGCTCGATCCGTC |
| | | G |
| | 23897 | CACACTATCCGTCTCCGTCACTCCTTCGCTCCATATACGGGTGCAGGAA |
| | | T |
| | 23898 | ACTGTCAGGTTCGACTCTTCCTGCGGATTTGCCTGCAGGAATCAACATC |
| | | T |
| | 23899 | TCTTTCGGCGAGGGGGTTTCCCACCCCCTTTATCGTTACTTATACCTAC |
| | | A |
| | 23900 | CTTTTCAGTGCTCTACAGGACACATCCATCACCTGAGGCTGTACCTCAA |
| | | T |
| | 23901 | ATGACCCTCCCCGGTTGAGCCGGGGGCTTTCACATCAGACTTAAGAAAC |
| | | C |
| | 23902 | TTTCACAACTGACTTAAATATCCATCTACGCTCCCTTTAAACCCAATAA |
| | | A |
| | 23903 | CTACTTATTTTCGGTCCCTTACGCCCGGGTCAACCAACGCCCGGGTCCA |
| | | G |
| | 23904 | GTATTTAGGCTTACCGGGTGGTCCCGGCAGATTCACAGCAGATTCCACG |
| | | A |
| | 23905 | CTTCAACCTGGACATGGATAGGTCACCCGGTTTCGGGTCTGCACACACT |
| | | G |
| | 23906 | TCCGGAAGCCACGCCTCAAGGGCACAACCTCCAAGTCGACATCGTTTAC |
| | | G |
| | 23907 | GGTCACCCGGTTTCGGGCCCATTGTATGCAACTTAACGCCCTTTTCAAA |
| | | C |
| | 23908 | GGCTACACATTTTAAAATGCTTAACCTTGCCGGAAAAAGTAACTCGTAG |
| | | G |
| | 23909 | CAAATTTCCTGCGCCCGCGACGGATAGGGACCGAACTGTCTCACGACGT |
| | | T |
| | 23910 | GCCAGGGTAGTATCCCACCGATGCCTCCACCGAAGCTGGCGCTCCGGTT |
| | | T |
| | 23911 | TTCACTGAAGGGTAACACCCCATAACAGGTGCCAGGTTTCCCCATTCGG |
| | | A |
| | 23912 | TCCAGCTAATCAGACGCGGGTCCATCTTATACCACCGGAGTTTTTCACA |
| | | C |
| | 23913 | CTTTATGAATATGCTTAGCGGATTTTCTTGGGAGCCTGATTACGTCCAT |
| | | T |
| | 23914 | CATCAGGTAGTATTCAGGCTTACCAGGTGGTCCTGGCAGATTCACACGA |
| | | A |
| | 23915 | CATGCACCACGGATTTGCCTATGATGCGCGCTGCGTGCTTGACCACGGA |
| | | A |
| | 23916 | GACAGCCTGGCCATCATTACGCCATTCGTGCAGGTCGGAACTTACCCGA |
| | | C |
| | 23917 | TCACTGCTTTAAGCAGCTCCGACCGCTTGTAGGCGCACGGTTTCAGGAA |
| | | C |
| | 23918 | GCTCCCAACACCACGCGGCGATACCAACCCGAAGGAAGGAACCACCACG |
| | | A |
| | 23919 | GACTTCCCATTCCATTCCACTAAACCTTTACAATACCGTTTTCTGTCCG |
| | | A |
| | 23920 | ACTTAACGACCCGTCTGCGCTCCCTTTAAACCCAATAAATCCGGATAAC |
| | | G |
| | 23921 | GGGGTGGGTTTCATACTTAGATGCTTTCAGCAGTTATCCGCTCCGCACT |
| | | T |
| | 23922 | GAAATCCTCGGATCAAAGCCCTGCTGGCGGCTCCCCGAGGCATATCGCA |
| | | G |
| | 23923 | CTTTCATGGCCCCTACTGATCATCGCCTTGGTAGGCCATTACCCTACCA |
| | | A |
| | 23924 | CTGTTATCCCCAGGGTAACTTTTATCCGTTGAGCGATGGCATTTCCACT |
| | | C |
| | 23925 | CCTACCCTCAGCTCATCCAGAAGCTTTTCAACGCTTATTGGTGCGGTCC |
| | | T |
| | 23926 | ACCAAGAAGGTGCTCCGACCGCTTGTAGGCACATGGTTTCAGGAACTAT |
| | | T |
| | 23927 | CTTCTCCCGTTGGCCTTAGAATCTTCTTCCTACCTACCTGTGTCGGTTT |
| | | G |
| | 23928 | CCTGGCCAAGGGTAGATCACTTGGTTTCGCGTCTGCCACTGCCGACTAT |
| | | A |
| | 23929 | GGGGGTCTCCCTTATGCCGAAGGCACGGGAGCAATTTGCCGAGTTCCTT |
| | | G |
| | 23930 | CATGGTTTAGCCCCGTTACATCTTCCGCGCAGGCCGACTCGACCAGTGA |
| | | G |
| | 23931 | ATCCGCCGCCTTTTCAACGGAGGTCGGTTCGGTCCTCCATGGAATTTTA |
| | | C |
| | 23932 | CCAAAGTCAATGCTAAGCTGTAGTAAAGGTTCACGGGGTCTTTTCGTCC |
| | | C |
| | 23933 | AAAGTTCGGTGGTTACGGAATTTCTACCGTATGTGCATCGACTACGCCG |
| | | T |
| | 23934 | CAGGTGTCAGCCCCTATACTTCATCTTTCGATTTAGCAGAGACCTGTGT |
| | | T |
| | 23935 | ACTTAAAGCCAGCGCCCCTTCTCCCGAAGTTACGGGGCCATTTTGCCGA |
| | | G |
| | 23936 | ACTTAGATGCTTTCAGCACTTATCCGATCCAGACTTAGATACCCGGCAA |
| | | T |
| | 23937 | CTACAGGATTTAGTTTAGCGGATTTTCTTGGCAGCATGATTACATGCAC |
| | | T |
| | 23938 | CCTTAACCTTCCGGCACTGGGCAGGTGTCAGCCCGTATACGTCGTATCT |
| | | C |
| | 23939 | TGAGCCAACATCCTAGTTGTCTTCGAAATCCCACATCCTTTTCCACTTA |
| | | A |
| | 23940 | CAGGATGTGACGAGCCGACATCGAGGTGCCAAACCCCTCCGTCGATATG |
| | | A |
| | 23941 | GGTTTTGCCGGTCCATGGTCGGTACGGGAATATCCACCCGTTCATCCAT |
| | | T |
| | 23942 | CTTTACGCTATCGGTCATTGGGTAGTATTTAGGCTTGGAGGGTGGTCCC |
| | | C |
| | 23943 | GCATGGATTAAGTTTAGCGGATTTTCTAGGAAGTATGATTACCTACGCT |
| | | A |
| | 23944 | ACTGTCCATCCTCTGGTTTCACAGAGCTATGTTAGAATTTCAGTAACCG |
| | | A |
| | 23945 | ACCTCGCGGTACGCCTTCGACGCCGACTGGAATGCTCCCCTACCGATCA |
| | | T |
| | 23946 | CTCTTGCGATGAGCTCTCCTCTTAACCTTCCAGCACCGGGCAGGTGTCA |
| | | G |
| | 23947 | AGCTGACGCCTTGGCTTCCCAGTCTCCCACCTATCCTGTACATGTAATA |
| | | C |
| | 23948 | GAATGAATGGCTGCTTCCAAGCCAACATCCTAGCTGTCACTGGGACCAG |
| | | A |
| | 23949 | TGAGCCAACATCCTGGTTGTCTACGTATCTTCACATCGTTTTCCACTTA |
| | | A |
| | 23950 | TGAGGGCACCTTTAGAAGCCTCCGTTACGCTTTTGGAGGCGACCACCCC |
| | | A |
| | 23951 | TTAAATCGACCGAAGTTTCAATAAAGTAATTCCCGTTCGACTTGCATGT |
| | | G |
| | 23952 | AGTCGGGTTGCAGACTCCAATCCGAACTGAGAGAGGCTTTAGGGATTAG |
| | | C |
| | 23953 | CCTGTGTCGGTTTACGGTACGGGTATGGTATGAACAATAGCGGCTTTTC |
| | | T |
| | 23954 | CTCCCGGATTCCGACGGAATTTCACGTGTTCCGCCGTACTCAGGATCCA |
| | | C |
| | 23955 | AAACATTAAAGGGTGGTATTTCAAGGTCGGCTCCATGCAGACTGGCGTC |
| | | C |
| | 23956 | CCTGAGTATATTCAACCCGACTACGTGTGTCCGTTTACGGTACGGGTAC |
| | | C |
| | 23957 | ACCACGAATTCCGCCTGCCTCAACTGCACTCAAGATATCCAGTATCAAC |
| | | T |
| | 23958 | AGTGAGCTATTACGCACTCTTTTAATGAGTGGCTGCTTCTAAGCCAACA |
| | | T |
| | 23959 | GGCTCACGCCCCGCCTTCAACGCCGAGTGGAATGCTCCCCTACCGATGA |
| | | T |
| | 23960 | AGGGCACCTTTAGAAGCCTCCGTTACACTTTTGGAGGCGACCACCCCAG |
| | | T |
| | 23961 | CTCTGCCATCGCCATCGCCGTTCGGCTTAGACTTAGGACCCGACTGACC |
| | | C |
| | 23962 | GCCGAGTTCCTTAACAAGGGTTCTCCCGCTCGTCTTAGGATTCTCTCCT |
| | | C |
| | 23963 | CTCCCCCCCCCCCCTTCCCCTCCGCGGCCACCTTTCCCCCCCCCTCCCC |
| | | A |
| | 23964 | CCCATATACACGGGTTAGAATCCAAACAAATGAAGGGTCGTATTTCAAC |
| | | A |
| | 23965 | CCCGCATCAGCGGGTTAGAACTCAAATAATCAAAGGGCCGTATTTCAAC |
| | | A |
| | 23966 | CTTCACAGTACTATACGCTATCGGTCACTGGGTAGTATTTAGGGTTGGA |
| | | G |
| | 23967 | CATTCCCACTTAATACCACCGGATCACTAAGCCCTACTTTCGTACCTGC |
| | | T |
| | 23968 | CTTCCGTCGCCCCGCGGTGGTTTCACTGCTCCGTCTCCACGTCGCCCCA |
| | | T |
| | 23969 | GCGGGTAACCTGCATCTTCACAGGTACTAAAATTTCACCGAGTCTCTCG |
| | | T |
| | 23970 | AAAAGTACGCGGTTGAGCTAATAATGCTCTTCCACAGCTTGTAAACACA |
| | | G |
| | 23971 | CGGTACGGGAATATCAACCCGTTCATCCATTCGACTACGCCTGTCGGCC |
| | | T |
| | 23972 | CCTCATCTACCTGTGTCGGTTTGCGGTACGGGCGCCTTAGTATACCTCA |
| | | T |
| | 23973 | GTAGTATTTAGCCTTGGAGGGTGGTCCCTCCTGCTTCCCACAGGGTTTC |
| | | A |
| | 23974 | TTCCGTCAGGTGGCGGCACTTACGTTCCTTCGTCTCTCCATCGAGGTAT |
| | | A |
| | 23975 | CTTCAAAGTCTCCGGCCTATCCTACACATCAATTACCCAAATTCAATGT |
| | | T |
| | 23976 | CTCTCAGGGCTCTTACTAACTGAACGTTATGGGAAATCTCATCTTGAGG |
| | | G |
| | 23977 | AAGTCCTCGAGCGATTAGTATTGGTCCGCTTCACGTCTCACAACGCTTC |
| | | C |
| | 23978 | ACGCCTTTCGTGCAGGTCGGAACTTACCCGACAAGGAATTTCGCTACCT |
| | | T |
| | 23979 | CCTGATCGACTTGTATGTCTCCCAGTCAAGCGCCCTTATGCCATTACAC |
| | | T |
| | 23980 | CGTTTTCCACTTAGCATGTATTAGGGACCTTAGCTGTGGGTCTGGGCTG |
| | | T |
| | 23981 | TAGTCAAGTATCGTCTCTCTTCTTCCTTGCTGATAGACCTTTACATACC |
| | | G |
| | 23982 | GACACATGGTTTTCTGCAACTGCCGGCCGGCCCGTCGGAGCCGGCGCAC |
| | | G |
| | 23983 | TTTCTCGTGTCTCGTGGTACTCTGGATCCCGCCTTGCCGCTCCCGGTTT |
| | | C |
| | 23984 | CTAATGAGATGTTTCAGTTCACAGCGTTTACCTCCAACTAGACTATGAA |
| | | T |
| | 23985 | ATCCTTTCCCACTTAGCACGCGCTTGGGGACCTTAGACGACGATCTGGG |
| | | C |
| | 23986 | GTTTCACGTGTCTGGCCGTACTCTGGAACTCGCTCAGCTCTTGTCGTTT |
| | | T |
| | 23987 | ATGGTTATAGTTACCACCGCCGTTTACCGGGGCTTGAATTCACCGCTTC |
| | | G |
| | 23988 | CCGCACGGAATGGCCGTCTCGTCTCGGGGGGGGCTTCCCGCTTAGATGC |
| | | T |
| | 23989 | TGCTCGACTTGTCTGTCTCGCAGTCAAGCTCCCTTATACCTTTACACTC |
| | | T |
| | 23990 | ATGCATTGCCAGAAGCTTTTCCTGGAAGCCGTCATCATGTGCTTCGCTA |
| | | C |
| | 23991 | TCTTGCGGCGAGCAGGTTTCTCACCTGCTTTATCGTTACTTATACCTAC |
| | | A |
| | 23992 | CGCGCACGCAACCCCCGACGGGTATCACGCGCACGCGGTTTGGTCTGAT |
| | | C |
| | 23993 | CGCTTTATCGTTACTTATGTCAGCATTCGCACTTCTGATACCTCCAGCA |
| | | T |
| | 23994 | GACAGTGCCCAAATCATTACGCCTTTCGTGCGGGTCGGAACTTACCCGA |
| | | C |
| | 23995 | TCCCATCTATCCTGTGCATGCAACACCGAAACCCAATATTAGGCTACAG |
| | | T |
| | 23996 | CCCGGGTCATGCCCTTTCAGAGTGTCCCTCTGCTTAAAACTTTCGGTGG |
| | | T |
| | 23997 | GGGATCCCATTCCCGGCTTCCGCTCTCTGCACGTGTCCCCACAGTTCTG |
| | | T |
| | 23998 | CACCTCGCCATACACGCCGCACGGATTTGCCTATGCGACTGGCTGCGTG |
| | | C |
| | 23999 | TCGCTCCTCAGCGTCAGTTACAGACCAGAGAGTCGCCTTCGCCACTGGT |
| | | G |
| | 24000 | TATCGAACCATAACGGCTCCCATCATCACACCTCGCCATGCATGCCATG |
| | | C |
| | 24001 | TTCACCGGGGCTTCAATTCGGAGCTTGCACCCCTCCTCTTGACCTTCCG |
| | | G |
| | 24002 | CTGCAGGATTAAGTTTAGCGGATTTTCTCGGCAGCATGCTTACGCGCAC |
| | | T |
| | 24003 | TCTCCTACCATACCTATAAAGGTATCCACAGCTTCGGTAATATGTTTTA |
| | | G |
| | 24004 | GGGCGCGTCATGCCCTCACGTCGAGGCTTTTCTCGGCAGCATAGGATCA |
| | | C |
| | 24005 | CTCCGACGGATTGTAGGCGCACGGTTTCAGGAACTCTTTCACTCCCCTC |
| | | C |
| | 24006 | CACTCGACTAGTGAGCTATTACGCACTCTTTGAATGAATAGCTGCTTCT |
| | | A |
| | 24007 | ACTCCCCTCGCCGGGGTTCTTTTCGCCTTTCCCTCACGGTACTGGTTCA |
| | | C |
| | 24008 | CCCTCCCGGGGTTCTTTTCACCTTTCCCTCACGGTACTATGCGCTATCG |
| | | G |
| | 24009 | CTGGTCCTCTCGTACTAGGAGCAGATCCTCTCAAATTTCCTTCGCCCGC |
| | | G |
| | 24010 | ACTTTCGTTACTGCTCGGGCCGTCACCCTCGCAGTTAGGCTAGCTTTTG |
| | | C |
| | 24011 | TGTAATAGCCACGTAATTTAAAACTGAAATTGAGAGAGACTTACCCAGA |
| | | G |
| | 24012 | GGTGGTCTACCGGGAGACTTACCCTCATGTGAGGTGGGAATACTCATCT |
| | | T |
| | 24013 | TGGCGGTCTGGGCTGTTTCCCTTTCGACTACGGATCTTATCACTCGCAG |
| | | T |
| | 24014 | TCTCCACATCACTCTTATAGGTAGTACAGGAATATTAACCTGTTCTGCC |
| | | A |
| | 24015 | CCATTCTGAGGGTACCTTTGGGCGCCTCCGTTACTCTTTCGGAGGCGAC |
| | | C |
| | 24016 | GATGGCAGGACTGTCACTTCTCCGTCTCCACATCGCTCCATAAAGTAGT |
| | | A |
| | 24017 | TCGGCGCAGAGTCACTCGACCAGTGAGCTATTACGCACTCTTTAAATGG |
| | | T |
| | 24018 | CGCGGCATGGCTGCATCAGGCTTGCGCCCATTGTGCAGTATTCCCCACT |
| | | G |
| | 24019 | CGGACATCCTTAATGACATTCGCAGTTTGATTGTATTCAGTACCCCGGG |
| | | A |
| | 24020 | TACCGGCATTCTCACTTCTAAGCGCTCCACCAGTCCTTCCGGTCTGGCT |
| | | T |
| | 24021 | TTCGGGCCTCCATTCAGTGTTACCTGAACTTCACCCTGGACATGGGTAG |
| | | A |
| | 24022 | CGGAGGCGACCGCCCCAGTCAAACTCCCCGCCTGGCATTGTCCCACCGC |
| | | C |
| | 24023 | ACCTTTTAGGAGGCGACCGCCCCAGTCAAACTGCCCGTCAGACACTGTC |
| | | T |
| | 24024 | ACAGCCCAGCCTTCCGTTGTGCGTACTTCACTACACAACAGCCTCACTG |
| | | C |
| | 24025 | TCATACCACCGGAGTTTTTACCCCTGCACCATGCGGTGCTGTGGTCTTA |
| | | T |
| | 24026 | CACTCACCCGAAGGCTTGCTCCCAAACAAAAGAGGTTTACAACCCGAAG |
| | | G |
| | 24027 | CGTCAATTCATTTGAGTTTTAACCTTGCGGCCGTACTCCCCAGGCGGTC |
| | | G |
| | 24028 | ACTTTCGTTCCTGCTCGACTTGTCAGTCTCGCAGTCAGGCTGGCTTGTG |
| | | C |
| | 24029 | CCACCAGGGAGGCTCCGACGGTTTGTGGGCGCACGGTTTCAGGAACTGT |
| | | T |
| | 24030 | ACTGGCGTGCACGTCTCTTTGTCTCCCACCTATCCTGTACATGTATGAC |
| | | C |
| | 24031 | TGATAGCGTGAGGTCCGAAGATCCCCCACTTTCTCCCTCAGGACGTATG |
| | | C |
| | 24032 | AAATCTTTAATCTCTTTCAGATGTCTTCTAGAGACGTCATTGGGTATTA |
| | | G |
| | 24033 | CACCGGGGCCCCAAGACCCACACACACCAACAAACCCGAAGGCTTAGTG |
| | | G |
| | 24034 | TACTTTTCCAATTTTTTTTTTTTTTTTTTTTTTTTTTTTCTTCCAATAA |
| | | A |
| | 24035 | CTCTGCCTATCCTTCTGTGTCACTGCATCCGGTTGCTCGGCGGTATCGG |
| | | A |
| | 24036 | ATGCCTGGCAGTTCCCTACTCTCGCATGGGGAGACCCCACACTACCATC |
| | | G |
| | 24037 | AACATCCTGGTTGTCTAAGCAACTCCACATCCTTTTCCACTTAACGTAT |
| | | A |
| | 24038 | CTCCGGCCGGGCCCGCCAGGACCCGGACACACGCTCCCTCAACACCACG |
| | | C |
| | 24039 | TTCTCTGCGGCTCTTTCGAGCACTCCTTATTCCGAAGTTACGGAGTCAA |
| | | T |
| | 24040 | GGCACAGCCCTGTGTTTTTGTTAAACAGTTGCCTGGACCGATTCTCTGC |
| | | G |
| | 24041 | TGCTCCCCACGCTTTCGAGCCTCAACGTCAGTTACTGTCCAGTAAGCCG |
| | | C |
| | 24042 | ATGCGTCCCACGGATTTGCCTATGGGACGGGCTGCGTGCTTGACCACGG |
| | | A |
| | 24043 | CCCAGACAACCATCGCTGGGGTTGAGCTACCTCCCTGCGTCCCTCCGCA |
| | | G |
| | 24044 | ACGCCGTTAGGCCTCACCTTAGCTCCCGACTGACCTGGAGCGGACGAAC |
| | | C |
| | 24045 | GCCTTTAGCCTTAACCTTGCCAGCCGGCGTAACTCGCCGGACCGTTCTA |
| | | C |
| | 24046 | TGGCCGTTCAACCTCTCAGTCCGGCTACTGATCGTCGCCATGGTGAGCC |
| | | G |
| | 24047 | CGCTTTCGCTCGCCACTACTCACGGAGTATCCCTTCCTGCAGGTACTGA |
| | | G |
| | 24048 | AGGACCCGACTCACCCGGGGACGACGAACGTGGCCCCGGAACCCTTGGT |
| | | C |
| | 24049 | CATTGCGGAAGATTCCCCACTGCTGCCTCCCGTAGGAGTCTGGACCGTG |
| | | T |
| | 24050 | GCATGTATTAGGCACGCCGCCAGCGTTCGTCCTGAGCCAGGATCAAACT |
| | | C |
| | 24051 | CCCGTTACCCATCATCGCCATGGTAGGCCTTTACCCTACCATCTAGCTA |
| | | A |
| | 24052 | GCCCTCACCCGATTAGTAACAGTCAGCTCCATGTGTTGCCACACTTCCA |
| | | C |
| | 24053 | ACCCCAAGTCATCCCCCGGTTTTCAACCCAGGTGGGTTCGGTCCTCCAC |
| | | G |
| | 24054 | CGCCTTAGGACCCGACTAACCCAGGGCGGATAAACCTAGCCCTGGAACC |
| | | C |
| | 24055 | TTCCGTCTTGCCGCGGGTACACTGCATCTTCACAGCGAGTTCAATTTCA |
| | | C |
| | 24056 | GTACGGGTAACACAGAAATATGCTTAGCGGGTTTTCTTGGGAGCCGGTT |
| | | T |
| | 24057 | AAGCTCCATGGGGTCTTTCCGTCTTGTCGCGGGTAACCGGCATCTTCAC |
| | | C |
| | 24058 | AACTTTATTCCCTTATAGAAGCAGTTTACAACCCATAGGGCCGTCTTCG |
| | | T |
| | 24059 | GGGCGGGATTCGCACCCGCCTCTCGCTACTCATGTCTGCATTCTCACTC |
| | | C |
| | 24060 | ATACTATCAGGTTCGGATCTCATGGTGGATTTGCCTGCCATGATCGACT |
| | | C |
| | 24061 | ACGCCGTCGGGCATATAAAGCCCTCCGACAGTTTGTAAACACAGGGTTT |
| | | C |
| | 24062 | GCCTATCGACCACGTGTTCTGCATGGGGTCTTCAGCGGCTCGGGGCCGC |
| | | A |
| | 24063 | GGATAAGGGTTGCGCTCGTTGCGGGACTTAACCCAACATTTCACAACAC |
| | | G |
| | 24064 | GCCCCCGAGCCTTGGCAGTGCTCTACACGGCGTGAGGTTCATCCGAGGC |
| | | T |
| | 24065 | TTCCTTAACCAAGAATCTCTCAACGCCTTAGTATGTTCTACCCGACCAC |
| | | G |
| | 24066 | TTTCCCTGCGGCTCCGGGACTTTATCCCTTAACCTTGCCAGTATGCACA |
| | | A |
| | 24067 | TACTGTCAGGTTCGACTCTTGCACCGGATTTGCCTGGCACAATCAACAT |
| | | C |
| | 24068 | GCCTTCCCATGCCATTCTGCTAGATACCTTCCATACCGTGCGCTGTCCG |
| | | A |
| | 24069 | ATGAGCCGACATCGAGGTGCCAAACACCGCCGTCGATATGAACTCTTGG |
| | | G |
| | 24070 | TTCGGCTCAAAGTCCGGATTTGCCTGGACCTCTCATCACCTACACTCTT |
| | | C |
| | 24071 | ACGCATTTCACCGCTACACGTGGAATTCCACTCTCCTCTTCTGCACTCA |
| | | A |
| | 24072 | TTTCCGTTTCGCCTACGGGGCTCTCACCCTCTCTGGCCGGTCTTTCCAG |
| | | A |
| | 24073 | GCCCCGGACAACCATCGCCGGGGATGAGCTACCTCCCTGCGTCCCTCCG |
| | | C |
| | 24074 | TGTCGCGGGTAACCGGCATCTTCACCGGTACTACAATTTCGCCGGGCGG |
| | | G |
| | 24075 | AAGCCCTCGATCTATTAGTACACACTTGCTGAATGGATCGCTCCACTTA |
| | | C |
| | 24076 | CCTTGGCAACAGTTCTCTCGCTCACCTCGGGATACTCTCCCTGCCCACC |
| | | T |
| | 24077 | TCTCCGCCAAAGCCAAAGCCTTGGTTTCCCAGAGTCCCATCTATCCTGT |
| | | G |
| | 24078 | AGGAGTATTCAGGCTTACCAGGTGGTCCTGGCAGATTCACACGAGATTT |
| | | C |
| | 24079 | CAGGATGTGACGAGCCGACATCGAGGTGCCAAACCACTCCGTCGATATG |
| | | A |
| | 24080 | CAACCTGTTGTCCATCGGCTACGCTTTTCAGCCTCACCTTAGGTCCCGA |
| | | C |
| | 24081 | TCAGATGGCGGCACTGCCACGACTCCGTCTCCACGTCACTCCCCAAGGT |
| | | A |
| | 24082 | CTACGGGGCCATCACCCTCTGCGGCCCGGCATTCAATCCGGTTCGCCTC |
| | | A |
| | 24083 | CCAGGTCATAAGGGGCATGATGATTTGACGTCATCCCCACCTTCCTCCG |
| | | G |
| | 24084 | CCTTTAATCATGTGAACATGCGGACTCATGATGCCATCTTGTATTAATC |
| | | T |
| | 24085 | TTTTCACACCTGACTTAAGATCCCGCCTTAAGCTTCCCTTTACACCCAG |
| | | T |
| | 24086 | CCTACCCTCAGCTCATCCAGAAGCTTTTCAACGCTTATTGGTGCGGTCC |
| | | T |
| | 24087 | GTCACACTGAGTATTTAGGCTTACCGGGTGGTCCCGGCAGATTCACAGC |
| | | A |
| | 24088 | CCAGGATAACTTACGTACACCATTCGACGCCGTGAGTATGCTCCCCTAC |
| | | C |
| | 24089 | AGAGAACCAGCTATCTCCAAGTTCGTTTGGAATTTCTCCGCTACCCACA |
| | | A |
| | 24090 | CCCGAAGTTACGGGGTAATTTTGCCGAGTTCCTTAACAACCCTTCTCCC |
| | | G |
| | 24091 | GGCTCACGCCCCACCTTCGACGCGGAGTGGAATGCTCCCCTACCGATGT |
| | | T |
| | 24092 | GTATCTAATCCTGTTTGCTCCCCACGCTTTCGCACTGAGCGTCAGTCTT |
| | | C |
| | 24093 | CGCGAGTCCATCCTGAAGCGAATAAATCCTTTTCCCTCAGCACCATGCG |
| | | G |
| | 24094 | TTATCGCAGCTTATCACGTCTTTCTTCGGCTCTTAGTGCCAAGGCATCC |
| | | A |
| | 24095 | CGGCAAAGATTCTCACTTTGCTCTCGCTACTCATGCCGGCATTCTCTCT |
| | | C |
| | 24096 | CCGGCAGACCGATCAAGAAAAAACCCACAACCCCGCACGCGCAACCCCT |
| | | G |
| | 24097 | GGGCTGTTTCCCTTTTGACTATGAGACTTATCTCACATAGTCTGACTGC |
| | | T |
| | 24098 | CCCCACTGCTGCCTCCCGTAGGAGTCTGGACCGTGTCTCAGTTCCAGTG |
| | | T |
| | 24099 | TTGTGACTATTCTCTGCGGCCTGCTCTCGCAGGCACCCCTTATCCCGAA |
| | | G |
| | 24100 | TTACCTCCACTTCAACCTGGACATGGGTAGGTCACCCGGTTTCGGGTCG |
| | | A |
| | 24101 | TCGCAAGGTTATCCCCAAGTGAAGGGCAGGTTGGATACGCGTTACTCAC |
| | | C |
| | 24102 | CGCGATCGGCAGACCATGCGCGTTCAGGTACGGGGCCCTCACCCTCTGC |
| | | G |
| | 24103 | GCCTTTCACTCCTACACTCGGCTCATCCAGAAGCTTTTCAACGCTTATT |
| | | G |
| | 24104 | AGTTTGATAAGGTTCAGTAACCTCTCGGCCCCTAGCCAATTCAGTGCTT |
| | | T |
| | 24105 | GGCTGCAACACGGTGACGTGAAGCGAATCCCAAAAACCATCTCTCAGTT |
| | | C |
| | 24106 | CCGGTCTCTCGACTAGTGAGCTGTTACGCACTCTTTGAATGAATGGCTG |
| | | C |
| | 24107 | GGATCACTAACTCCAACTTTCGTTACTGCTCGAACTGTCGCTCTCGCAG |
| | | T |
| | 24108 | CTCGCGTACCGCTTTAATGGGCGAACAGCCCAACCCTTGGGACCGACTA |
| | | C |
| | 24109 | CGGCTACGCCTTTCGGCCTCACCTTAGCTCCCGACTAACTTGGAGCGGA |
| | | C |
| | 24110 | ACCTTTCCCTCACGGTACTGGTTCACTATCGGTCACTAGGGAGTATTTA |
| | | G |
| | 24111 | ATACTGTCAGGTTCGACTCTTGCACCGGATTTGCCTGGCGCAATCAGCA |
| | | T |
| | 24112 | TGTCATGCTCTATGGTCTTTCTTTCCAGAAAGTTCTTCTCCGATGTCTT |
| | | C |
| | 24113 | ATCACCTTAGGATTCTCTCCTCGCCTACCTGTGTCGGTTTGCGGTACGG |
| | | G |
| | 24114 | ACGTATTCACCGTGGCATTCTGATCCACGATTACTAGCGATTCCGACTT |
| | | C |
| | 24115 | TAGAGCATTTTCTTGGAAGCAGGATTACCCACACTATTGGTTTACTCCG |
| | | A |
| | 24116 | CATTGACCAATATTCCTCACTGCTGCCTCCCGTAGGAGTTTGGGCCGTG |
| | | T |
| | 24117 | ATCCGCCGCCTTTTCAACGGAGGTCGGTTCGGTCCTCCATGGAATTTTA |
| | | C |
| | 24118 | CCTGTGTCGGTTTACGGTACGGGCGCATGGCAAACGATAGCGGCTTTTC |
| | | T |
| | 24119 | GCCCAAGGGTAGATCACTTGGTTTCGCGTCTACTCCTTCCGACTATACG |
| | | C |
| | 24120 | GGCGGATTTTCCCAAATCCTTCGACTATCAAGTTCTTTGGTAACTCAAA |
| | | T |
| | 24121 | CTTTCGGGGAGTACGAGCTATCTCCGAGTTTGATTGGCCTTTCACTCCT |
| | | A |
| | 24122 | CTCTAGTTAGCCTGCTGCGTCCCTCCTTCACTCAATACTCTAGTACAGG |
| | | A |
| | 24123 | CGCCGTCGATGTGAACTCTTGGGCGAGATCAGCCTGTTATCCCCAGGGT |
| | | A |
| | 24124 | AGTCGTTTCCAACTGTTGTCCCCCACTCCAGGGCAGGTTACTCACGCGT |
| | | T |
| | 24125 | GCATGCTTAAAGTTCGGCGGCTACGGAATTTCAACCGTATGTGCATCGA |
| | | C |
| | 24126 | ATTACCGCGGCTGCTGGCACGGAATTAGCCGGTCCTTATTCTTATGGTA |
| | | C |
| | 24127 | CGCACAGCCCTGTGTTTTTGTTAAACAGTTGCCTGGACCTATTCTCTGC |
| | | G |
| | 24128 | CATAATTTTATTTTCTTCTCCTACGGGTACTGAGATGTTTCACTTCCCC |
| | | G |
| | 24129 | ACCTTGGGCGGACGAACCTTCCCCAAGAAACCTTAGATTTTCGGCCATT |
| | | A |
| | 24130 | TACTATCAGGTTCGGCTCTCAAGGTGGATTTGCCTGCCTCGATCTGCGC |
| | | C |
| | 24131 | CTGTACATGCAATACCAAGCTCCAGTACCAAACTGGAGTAAAGCTCCAT |
| | | G |
| | 24132 | TGCTTGACCACGGAAAACCACCTCCGCGGCCGGCTCCCATTCCGTGTCA |
| | | C |
| | 24133 | CAGTAACCCGCAAGGCTGCACCTAAATGCATTTCGGGGAGTACGAGCTA |
| | | T |
| | 24134 | AAGCCAACATCCTGGTTGTCTACGCAATTGCACATCCTTTTCCACTTAA |
| | | C |
| | 24135 | CACATCTTACGACGGCAGTCTCGACAGAGTCCCCAGCATCACCTGATGG |
| | | T |
| | 24136 | TTATAGTTACGGCCGCCGTTCACTGGGGCTTCGATTCAATGCTTGCACA |
| | | T |
| | 24137 | CATCTTTACTCGTACTGCAATTTCGCCGAGCTCCTGGTCGAGACAGTGG |
| | | G |
| | 24138 | ACACCGAGCCATGCAGCTCTGTGCGCTTATGCGGTATTAGCAGTCATTT |
| | | C |
| | 24139 | AGGTCCCGCGCTCCCCACCACCGTCCCCGTCAAAGACGGGGTTCGGGAT |
| | | G |
| | 24140 | ATCGAGCTCACAGCATGTGCATTTTTGTGTACGGGGCTGTCACCCTGTA |
| | | T |
| | 24141 | GGAATTTCTCCCCTAGCCACAAGTCATCCGCTAACTTTTCAACGGTAGT |
| | | C |
| | 24142 | GCTCTACCTCCAAGACTCTTACCTTGAGGCTAGCCCTAAAGCTATTTCG |
| | | G |
| | 24143 | TTATAGTTACGGCCGCCGTTTACTGGGGCTTCAATTCAATGCTTCTCTT |
| | | G |
| | 24144 | CTTCAACCTGGACATGGATAGGTCACCCGGTTTCGGGTCTGCACACACT |
| | | G |
| | 24145 | GAGGCTAGCCCTAAAGCTATTTCGAGGAGAACCAGCTATCTCCGGGTTC |
| | | G |
| | 24146 | TGGGCTGTTTCCCTTTCGACTACGGATCTTAGCACTCGCAGTCTGACTG |
| | | C |
| | 24147 | CTCCGGCCTATCCTACACATCGATTGCCCAAATTCAATGTAAAGCTATA |
| | | G |
| | 24148 | CCACTTCACCTAACAACAATGCAAAAAGGGCGTGCCACTGGTAGATGAC |
| | | A |
| | 24149 | ACCCTCAGGTCATCCAGAAGCTTTTCAACGCTTATTGGTTCGGTCCTCC |
| | | A |
| | 24150 | AGTATCCCTTCCTGCAGGTACTGAGATGTTTCACTTCCCTGCGTACCCC |
| | | C |
| | 24151 | ACTTGGTATCCCTTCGGCTCCGCACCTTAAGTGCTTAACCTCGCCAGTA |
| | | T |
| | 24152 | TCGGATACGTGTGTCGTCACACTTAACCTTGCCGGCAAAGGCAACTCGT |
| | | A |
| | 24153 | GGATCACTAACTCCAACTTTCGTTACTGCTCGAACTGTCGCTCTCGCAG |
| | | T |
| | 24154 | CGAACGCCTTAGTATTTTCAACCTGACTACCTGTGTCGGTTTGGGGTAC |
| | | G |
| | 24155 | TTCTGCTTCTGCCCGTACACGTTGCTCCCCTACCCAGAAGTTTCCTTCT |
| | | G |
| | 24156 | TCACGGTACTAGTTCGCTATCGGTCAGACAGGTATATCTAGGCTTACCC |
| | | C |
| | 24157 | ACTTCTTACAAAGCTCCGACCGCTTGTAGGCGCATGGTTTCAGGGACTA |
| | | T |
| | 24158 | TCTTTAAAGGATGGCTGCTTCTGAGCCAACCTCCTAGTTGTCTGGGCAT |
| | | C |
| | 24159 | CCCCATTGGGGCCCACAACACCGCACACACAACCCCTACCAAGTATCAC |
| | | A |
| | 24160 | CTCAACTTCAACCTGCTCATGGCTAGATCACCCGGTTTCGGGTCTGCAA |
| | | C |
| | 24161 | GCATACGCCACACGGCTTATGCTCGCCACCCGCCACTGACTCGCAGACT |
| | | C |
| | 24162 | GTTCGTCTATATGCCCGCACCTCACTGCGCCATGCCGGCAGACATGACC |
| | | A |
| | 24163 | ATCTGGGCTGTTTCCCTTTTGACAATGACATTTATCTGACACTGTCTGA |
| | | C |
| | 24164 | CTATTAGTAGCAGTCAGCTCCATGTGTTACCACACTTCCACCCCTGCCC |
| | | T |
| | 24165 | TTTCACAACTGACTTAAACATCCATCTACGCTCCCTTTAAACCCAATAA |
| | | A |
| | 24166 | CCGTTGAATTTTCGGCGCAGAGTCACTCGACCAGTGAGCTATTACGCAC |
| | | T |
| | 24167 | TCCTTAACGAGAGTTCGCTCGCTCACCTGAGGCTACTCGCCTCGACTAC |
| | | C |
| | 24168 | CCACTCCGTCGATGTGAACTCTTGGGAGTGATAAGCCTGTTATCCCCAG |
| | | G |
| | 24169 | CAACAGGATGAAGTTTAGCGGATTTTCTCGGGAGTATGATTACATGCGC |
| | | T |
| | 24170 | GACGGGCTGCGTGCTTGACCACGGAAAACCACCTCCGCGGCCGGCTACC |
| | | C |
| | 24171 | CGGATTTGCCTATGATGCGCGCTGCGTGCTTGACCACGGAAAACCACCT |
| | | C |
| | 24172 | CTGAGTTTGATAAGCTTCGCTAACCTCTCGGCCGCTAGGCTATTCAGTG |
| | | C |
| | 24173 | TGCAGCACCTGTCTCACGGTTCCCGAAGGCACATTCTCATCTCTGAAAA |
| | | C |
| | 24174 | AGGCTAGCCCTAAAGCTATTTCGGGGAGAACCAGCTATCTCCGAGTTCG |
| | | A |
| | 24175 | GACGTCCTATCTCTAGGATTGTCAGAGGATGTCAAGACCTGGTAAGGTT |
| | | C |
| | 24176 | GTTTTGACTACAGGGCTGTTACCTCCTATGGCGGGCCTTTCCAGACCTC |
| | | T |
| | 24177 | CTGGGGCTTCAATTCAGATCTTCGCTAACGCTAAACCCTCCTCTTAACC |
| | | T |
| | 24178 | CCTTAGTATATTCAACCCGACTACGTGTGTCCGTTTACGGTACGGGTAC |
| | | C |
| | 24179 | CTATACATCATCTTACGATTTAGCAGAGAGCTGTGTTTTTGATAAACAG |
| | | T |
| | 24180 | CTAACAATGTCCCCCGACTCGATTCAGAGCCGCAGGTTAGAATTCCAAT |
| | | A |
| | 24181 | TTTGGCCTCTTCCGCGTTCGCTCGCCACTACTTACGGAATCTCAGTTGA |
| | | T |
| | 24182 | CCCGCCAACTGGCTAATCAGACGCGGGTCCATCTTATACCACCGGAGTT |
| | | T |
| | 24183 | GCTACTTGGGACACGCGATCGGAAGACGGCAAGCGTCCAGGTACGGGGC |
| | | T |
| | 24184 | CATCACCGGGGATGAGCTACCTCACTGCGTCCCTCCGCAGCTTGCCTAC |
| | | T |
| | 24185 | ACAACTTAATACCCGATTATTATCCACGCCAGACTCCTCGACTAGTGAG |
| | | C |
| | 24186 | CTCTCAGACCAGTTACGGATCGTCGCCTTGGTAGGCCTTTACCCCACCA |
| | | A |
| | 24187 | TCACGTAGTCTGACTGCTGATCATCAATTAGCCGGCATTCAGAGTTTGA |
| | | T |
| | 24188 | TAGGTCACCCGGTTTCGGGTGTACTGCATGCAACTTTACGCCCTTTTCA |
| | | G |
| | 24189 | TACTTTAGTTCGCTCCACATCACGGCTTCGTCTCATGCACAGCGGATTT |
| | | G |
| | 24190 | CTTACGGGGCTTTCACCCTCTCTGGCAGGCTTTCCCAAAAACCTTTCTG |
| | | C |
| | 24191 | GGCCGGGCTTTCGATCCCGTTCTTCTATCCTCTCTCTTGCCATATCATG |
| | | G |
| | 24192 | ACGGCTTCTACTCGTATACAACGCTCCCCTACCACTATAGTTTCCTACA |
| | | A |
| | 24193 | ATCGAGTTTTCTTTCTCTTCCTCCGGCTACTTAGATGTTTCAGTTCACC |
| | | G |
| | 24194 | GCTTTACATACCGAAATACTTCTTCACTCACGCGGCGTCGCTGCATCAG |
| | | G |
| | 24195 | TCCCTTCTGCCTTTGCACTCTTCTAATGGTTTCCGACCATTATGAGGGA |
| | | A |
| | 24196 | CTCCATCAGGCAGTTTCCCAGACATTACTCACCCGTCCGCCACTCGTCA |
| | | G |
| | 24197 | TGCCAAACCTCCCCGTCGATGTGAACTCTTGGGGGAGATAAGCCTGTTA |
| | | T |
| | 24198 | GCCTGGACCTATTCTCTGCGCCTCACATTACTGTGAGGACCCTTTATCC |
| | | C |
| | 24199 | ACCTTTACACCTGCATCCTATCAACGTCGTAGTCTACAACGACCCTCAG |
| | | A |
| | 24200 | GTATTCATTAACGCTAGAAGCTTTTCTTGGCAGAGTGACATCACTAGCT |
| | | T |
| | 24201 | GCTGTTGGTCCGGATTGTTCTCCTTTAGGACATGGACCTTAGCACCCAT |
| | | G |
| | 24202 | AAAAACCCTCCCCCCCCCCCCTTCCCCTCCGCGGCCACCTTTCCCCCCC |
| | | C |
| | 24203 | CTGTCGGTACCCGATACGGGCCCTCAAGCATCCAGTAGCTCTACCCCCC |
| | | G |
| | 24204 | ATCTACGCATTTCACCGCTACACTAGGAATTCCGCTTACCTCTGTTGCA |
| | | C |
| | 24205 | TCTGTCCCACCTTCGGCGGCTGGCTCCTAAAAGGTTACCTCACCGACTT |
| | | C |
| | 24206 | TGACCAAGGGTAGATCACTTGGTTTCGCGTCTACTCCTTCCGACTAATC |
| | | G |
| | 24207 | TGTGCACTTGCACTCGCCACCCGATTGCCAACCGGGCTGAGCGGACCTT |
| | | T |
| | 24208 | CAGCCTCACTCCCAGGCTGTAAAATATGCCCCTTCGGAGTTTGATAAGG |
| | | T |
| | 24209 | ACGCTTCCACTAACACACACACTGATTCAGGCTCTGGGCTGCTCCCCGT |
| | | T |
| | 24210 | CTGTCAAGGTCGACTCTCCCTGCGGATTTGCCTACAGGAATCTACATCT |
| | | A |
| | 24211 | CCTGTGTTTTTGGTAAACAGTCGCTACCCCCTGGCCTGTGCCACCCCCC |
| | | G |
| | 24212 | ATCTGATAGCGTGAGGTCCGAAGATCCCCCACTTTCTCCCTCAGGACGT |
| | | A |
| | 24213 | ACACTTTGGGACCTTAGCCGGTGGTCTGGGCTCTTTCCCTTTTGACTAC |
| | | C |
| | 24214 | CTACAAGGGATCTTACCTGATTGAATCAGTGGGATATCTTATCTTTGGG |
| | | T |
| | 24215 | CTGAAGGGTAACCCCACATAACCAGGGCCAGGTTTCCCCATTCGGACAT |
| | | C |
| | 24216 | TCAGTCCGCGGCGCTGTCACGCCTCCGTCTCCACGTCACTCCTTAAGGT |
| | | A |
| | 24217 | TTAACAAGGGTTCTCCCGTTCGTCTCAGGATTCTCTCCTCGCCCACCTG |
| | | C |
| | 24218 | CTAACATCCTAGTTGTCTGTGCAACCCCACATCCTTTTCCACTTAACAA |
| | | T |
| | 24219 | GATAAATCTTTCCCCCGTAGGGCACATTCGGTATTACTCCCAGTTTCCC |
| | | G |
| | 24220 | GTTTACAATCCGAAGACCTTCTTCCCACACGCGGCGTTGCTGCATCAGG |
| | | G |
| | 24221 | CGGCGCACTGCAGCTACCTGTCTGCGTCACCCCTGTTAACACGCTTGCC |
| | | T |
| | 24222 | ATGAAGCTGGAATCGCTAGTAATCGTATATCAGCAATGATACGGTGAAT |
| | | A |
| | 24223 | CGGATTTGCCTATGGGACGGGCTGCGTGCTTGACCACGGAAAACCACCT |
| | | C |
| | 24224 | GGATGACCCCCTTGCCGAAACAGTGCTCTACCCCCGGAGATGAATTCAC |
| | | G |
| | 24225 | GGTACGGGTAACATATACTATAACTTAGAAGATTTTCTCGGAAGTCGAC |
| | | T |
| | 24226 | CTTTGTAACTCCGTACAGAGTGTCCTACAACCCCAAGAGGCAAGCCTCT |
| | | T |
| | 24227 | TCTTACTTCTTGCGAATGGGAGATCTCATCTTGGAGTAGGCTTCGTGCT |
| | | T |
| | 24228 | GTCAAGCTCCCTTATACCTTTACACTCTGCGATTGATTTCCAACCAATC |
| | | T |
| | 24229 | CCACCTATCCTACACATCAAGGCTCAATGTTCAGTGTCAAGCTATAGTA |
| | | A |
| | 24230 | AAAAGCAGTTTACAACCCATAGGGCCGTCATCCTGCACGCTACTTGGCT |
| | | G |
| | 24231 | TGAGGGCACCTTTAGAAGCCTCCGTTACACTTTTGGAGGCGACCACCCC |
| | | A |
| | 24232 | ACGCTCTAACCTTATGGTAACCGGATTTGCCTGGTAACCAGCCGCTTCG |
| | | C |
| | 24233 | GCTTCCAAGCCAACATCCTAGCTGTCTTAGCAATCTGACTTCGTTAGTT |
| | | C |
| | 24234 | TGGCCGTTCACCCTCTCAGGCCGGCTATGGATCGTCGCCTTGGTAGGCC |
| | | G |
| | 24235 | TGAGCCAACATCCTGGTTGTCTTCGAAATCCCACATCCTTTTCCACTTA |
| | | A |
| | 24236 | CTAGAGAGTATTTAGGGTTAGGAGATGGTCCTCCCAGATTCCGACGAGA |
| | | T |
| | 24237 | GCCTTTCGGCCTCGCGTTAGGTCCCGACTTACCCAGGGCGGACGAACCT |
| | | T |
| | 24238 | GTCAAACTGCCCACCTGACACTGTCTCCCCGCCCGATAAGGGCGGCGGG |
| | | T |
| | 24239 | TGGAGTAAAGCTCCATGGGGTCTTTCCGTCCTGGCGCAGGTAACCAGCA |
| | | T |
| | 24240 | TTTCTTCTCCTACGGGTACTGAGATGTTTCACTTCCCCGCGTAACCCCC |
| | | A |
| | 24241 | ACCAGCTATGGATCGTCGGCTTGGTAGGCCATTACCCCACCAACTACCT |
| | | A |
| | 24242 | GGGGCAAGTTTCGTGCTTAGATGCTTTCAGCACTTATCTCTTCCGCATT |
| | | T |
| | 24243 | CACCAGTGTCGGTTTGGGGTACGGGCGGCCATAGCCCTCACGCCGAGGC |
| | | T |
| | 24244 | GACGTTCTGAACCCAGCTCGCGTGCCGCTTTAATGGGCGAACAGCCCAA |
| | | C |
| | 24245 | GGTTAGAATTCCAATATCGCAAGGATGGTATCCCAACGGCCTCTCCGCC |
| | | A |
| | 24246 | AGGTTACCCACGCGTTACTCACCCGTCCGCCACTAGAAACAATCTAAAT |
| | | C |
| | 24247 | CAGGTGTCACCCCATATACGTCATCTTTCGATTTAGCATAGAGCTGTGT |
| | | T |
| | 24248 | TCTTTCGGCGAGGGGGTTTCCCACCCCCTTTATCGTTACTTATACCTAC |
| | | A |
| | 24249 | CTTAGGACCGTTATAGTTACGGCCGCCGTTTACCGGGGCTTCGATCAAG |
| | | A |
| | 24250 | CCACTTAGTGATGATTTGGGGACCTTAGCTGGCGGTCTGGGTTGTTTCC |
| | | C |
| | 24251 | TCCCCCATTCGGACACCTCCGCTTCTTCGCTTCCTTACAGCTTCACGGA |
| | | G |
| | 24252 | ATAGATCACCCGGTTTCGGGTCTGCCCCCACTGACTCTGGCCCTCTTAA |
| | | G |
| | 24253 | GCCTATCAAACACGTGTTCCACATGCGGGCTTCAGGACCCCGAAGGGCC |
| | | C |
| | 24254 | CCATTTCTGACTGTTATCCCCCTGTATAAGGCAGGTTGCCCACGCGTTA |
| | | C |
| | 24255 | CATCATCTGTATGGCATTCGGAGTTTGATATCCCTTAGTAAGCTTTGAC |
| | | G |
| | 24256 | GTTTGGGGTACGGGCGGCTAAAACCTCGCGCCGATGCTTTTCTAGGCAG |
| | | C |
| | 24257 | GCGATGGCCCTTCCATACGGTACCACCGGATCACTAAGCCCGACTTTCG |
| | | T |
| | 24258 | GAGTTAACCCCGGCGGTCCCCCGTGAGTTCCCACCATAACGTGCTGGCA |
| | | A |
| | 24259 | GGATAATCGGCGGACGGGATTCCCACCCGTCACACGCTACTCATGCCTG |
| | | C |
| | 24260 | TACCTCTTCGTTATGATATGTCCGCAACCCCAATAAAGAAAACTTTATT |
| | | G |
| | 24261 | ACGTGTCCGGCGGTACTCTGGATTCAGCTGGCGGATCTTCTCTTTCGCA |
| | | T |
| | 24262 | TCGAGACCAGACTTCGTTAGACTAACTCAGACAGGATTCCGGGACCTTA |
| | | G |
| | 24263 | TGGCCGTTCAACCTCTCAGTCCGGCTACCAATCGTCGCCTTGGTGGGCC |
| | | G |
| | 24264 | TATAAGTCAAGGCTGCACCTAAATGCATTTCGGGGAGTACGAGCTATCT |
| | | C |
| | 24265 | CTACTGTTTCACCGCGTATACAACGCTCCCCTACCCAGCATGTAAACAT |
| | | G |
| | 24266 | TTATAGTTACGGCCGCCGTTTACTGGGGCTTCAATTCACACCTTCGACA |
| | | A |
| | 24267 | GGATGGACCCCTCACCCAAACAGTGCTCTACCTCCATGATTCTTAATGT |
| | | C |
| | 24268 | TTGGGACCTTAGCTGCGGGTCTGGGCTCTTTCCCTTTTGACTATCCAAC |
| | | T |
| | 24269 | GGCTCTGACTACTTGTAGGCACACGGTTTCAGGATCTCTTTCACTCCCC |
| | | T |
| | 24270 | TCGCTACTCATTCCGGCATTCTCACTCGTGTACAGTCCACCGCTGCTTT |
| | | C |
| | 24271 | CCTCCCCCCCCCCCCCCCCCCCCCCCCCTTCCCCCCTCTCCTCCCCCTT |
| | | C |
| | 24272 | TAACACCCCATAACAGGTGCCAGGTTTCCCCATTCGGACATCCTCGGAT |
| | | C |
| | 24273 | ACCTCGACACGGACGGTGACAAGCCGGTACCAGAATATCAACTGGTTAC |
| | | C |
| | 24274 | ATAGATCACCCGGTTTCGGGTCTACTCCGGCTGACTCGCTCGCCCTATT |
| | | C |
| | 24275 | TAAATGATGGCTGCTTCTAAGCCAACATCCTGGCTGTCTGGGCCTTCCC |
| | | A |
| | 24276 | CAGCTTATAGGGTTGCGTACTTCACTACAACCCAACCTTGATGCTTGCA |
| | | C |
| | 24277 | GCTTGGGCCTTTTCACTGCGGCTGACTTATCGCCAGCGCCCCTTCTCCC |
| | | G |
| | 24278 | TGAGGTCGGCTTCACGCTTAGATGCTTTCAGCGTTTATCCGTTCCGCAC |
| | | T |
| | 24279 | CTCCGGGTACTGTCAGGTTCGACTCTCAGGGCGGATTTGCCTACCCCGA |
| | | T |
| | 24280 | GCTTGGGCCTCTTCACTGCGGCTTAATTGCTTAAGCACTCCTTCTCGCT |
| | | A |
| | 24281 | TTTATCCCGAAGTTACAGGGTCAGTTTGCCTAGTTCCTTAACCGTGAAT |
| | | C |
| | 24282 | GTAGTTAGCCGGAGCTTCCTCCTAAAGTACCGTCATTATCGTCCTTTAA |
| | | G |
| | 24283 | TCTTTCGGCGAGGGGGTTTCCCGCCCCCTTTATCGTTACTTATACCTAC |
| | | A |
| | 24284 | GGATGTACTAGCAGCTTTTCTCGCCAGCGTGAACTCACTCGCTTCCCTA |
| | | C |
| | 24285 | TTAGTATCAGTGCTTTATCAGGGGCGCATATACTCGGGTACCAGAATAT |
| | | C |
| | 24286 | GCTTGGCGGCGTCCTACTCTCACAGGGGGAAACCCCCGACTACCATCGG |
| | | C |
| | 24287 | AGATTCACGCAGAATTCCTCGTGCTCCGCGCTACTCAGGATACTACTAT |
| | | G |
| | 24288 | TATCAACCTGATCATCTTTCAGGGATCTTACTTCCTTGCGGAATGGGAA |
| | | A |
| | 24289 | TCAATAGGCACGCCACCACACTCTTATGGAGCGGTGACTGCTTGTAAGT |
| | | C |
| | 24290 | CTACTATATTTCGGTCCCTTACGCCCGGGGCAACCATCGCCCGGGATAA |
| | | C |
| | 24291 | TGCCATGACTGCTTGTAAGTCCACGGTTTCAGGTTCTCTTTCACTCCCC |
| | | T |
| | 24292 | TCCATTTGCGCAGCACCAGTAATCATGTTCTTAACATAGTCAGCATGTC |
| | | C |
| | 24293 | TCTCAGTCCCAATGTGGCCGGTCACCCTCTCAGGTCGGCTACTGATCGT |
| | | C |
| | 24294 | TGGCCGTTCAACCTCTCAGTCCGGCTACTGATCGTCGCCTTGGTGGGCC |
| | | T |
| | 24295 | TTATAGTTACGGCCGCCGTTTACCGGGGCTTCAATTCGGAGCTCTCACT |
| | | C |
| | 24296 | TAGTGAAAGGTAGATTTTCTGACCCTTTCGACCTGAACGTACCAACCAG |
| | | C |
| | 24297 | TCTTGGCAGTGTGACATCACTAACTTCGCTACTAAACTTCGCTCCCCAT |
| | | C |
| | 24298 | ACCTGCTTTCGCACCTGCTCGCGCCGTCACGCTCGCAGTCAAGCTGGCT |
| | | T |
| | 24299 | TCGGAGTTTGATATTCTTCGGTAAGCTTTGACGCCCCCTAGGAAATTCA |
| | | G |
| | 24300 | ACCCACCGAGTGGGCGCCCATCAGGTCTCAAGCACATAGCCGGCGGATT |
| | | T |
| | 24301 | TACGGGTGCCGCATGGATAAGTTTAGCGGATTTTCTCGGGAGCATGGTT |
| | | A |
| | 24302 | TTCAAACAACCATCCGGTATTAGCCCCGGTTTCCCGGAGTTATCCCAGT |
| | | C |
| | 24303 | TCCTTAACCACGCTGCATACCATAACTCGCCGGACCATTCTACAAAAGG |
| | | T |
| | 24304 | CCGGCACCGGGCAGGTGTCAGGCTGTATACGTCATCTTTCGAGTTTGCA |
| | | C |
| | 24305 | CAGGAATATTCAGGCTTACCCAACGGTCTGGGCGGATTCGCACGGGGTT |
| | | C |
| | 24306 | TTTATCCCGAAGTTACAGGGTCAGTTTGCCTAGTTCCTTAACCGTGAAT |
| | | C |
| | 24307 | CTTCTGCAATTGCACTCGTCGATTGGTTTCCATCCAATCTGAGCGTACC |
| | | T |
| | 24308 | TCGGTTTGCCCTCTTCCGCGTTCGCTCGCCACTACTTACGGAATCTCGT |
| | | T |
| | 24309 | AAGCTCCATGGGGTCTTTCCGTCTTGTCGCGGGTAACCGGCATCTTCAC |
| | | C |
| | 24310 | CATCGGCCTCACCGTTCGGCTGAGCCTTAGGACCCGACTAACCCTGATC |
| | | C |
| | 24311 | CCTCGCCATACACGCCGCACGGATTTGCCTATGCGACTGGCTGCGTGCT |
| | | T |
| | 24312 | CCTGTCGCGGGTAACCTGCATCTTCACAGGTACTATAATTTCACCGAGT |
| | | C |
| | 24313 | TCAGCCTTATGGGAAACGGATTTGCCTATTTCCCAGCCTAACTGCTTGG |
| | | A |
| | 24314 | TTTCACAACACGCTTAAAAGGCGGCCTACGCTCCCTTTAAACCCAATAA |
| | | A |
| | 24315 | CCCCGCGGTACTCTGGATCCTGCTAGCTCTCGCTCCTTTTCGTCTACGT |
| | | G |
| | 24316 | ATCGGTTCACACACTCACCCACCCCAGAAGCATCAAAAACACTCCCAAG |
| | | A |
| | 24317 | TAGAAAGGAGGTGATCCAGCCGCACCTTCCGATACGGCTACCTTGTTAC |
| | | G |
| | 24318 | GCCCATTGTCCAATATTCCCCACTGCTGCCTCCCGTAGGAGTCTGGACC |
| | | G |
| | 24319 | TCACCTTTCCCTCACGGTACTGGTTCGCTATCGGTCTCTCGGGAGTATT |
| | | T |
| | 24320 | CGAAGTTACGGGGTCATTTTGCCGAGTTCCTTGACAATGCTTCTCCCGC |
| | | C |
| | 24321 | AGATCCTCTCAAATTTCCTACGCCCGCGACGGATAGGGACCGAACTGTC |
| | | T |
| | 24322 | TCTCAGTCCCAATGTGGCCGGTCACCCTCTCAGGTCGGCTACTGATCGT |
| | | C |
| | 24323 | GGCAACCCAACAACCCACACATCATCATCTTCAGCTACAGGACTCTCAC |
| | | C |
| | 24324 | GCACTATTGCCTTGTCCCGGAGGACGCGGCATACTGTCAGGTTCGAATC |
| | | A |
| | 24325 | CCGTGGCTTTCTGGTTAGGTACCGTCAAGGTACCGCCCTATTCGAACGG |
| | | T |
| | 24326 | ATACTATCAGGTTCGACTCTTATCCCGGATTTGCCTGGGATAATCAACA |
| | | T |
| | 24327 | TAAGTCCTTAACCTTGCTGCATACAATCGCTCGCCGGACCGTTCTACAA |
| | | A |
| | 24328 | ATCTGGGCTGTTTCCCTTTTGACAATGACATTTATCTGACACTGTCTGA |
| | | C |
| | 24329 | AGAGTAACCATAACACAAGGGTAGTATCCCAACAACGCCTCCTCCGAAA |
| | | C |
| | 24330 | TGGACAGGATTCTCACCTGTCTTACGCTACTCATACCGGCATTCTCACT |
| | | T |
| | 24331 | GCCCGGCTACCTTCCTGCGTCACACCTGTTAATACGCTTGGCTCCCCAG |
| | | T |
| | 24332 | GTCAAGCTCCCTTATACCTTTACACTCTGCGAATGATTTCCAACCATTC |
| | | T |
| | 24333 | CCCAACCCTTGGAACATACTACAGCCCCAGGTGGCGAAGAGCCGACATC |
| | | G |
| | 24334 | TCTTTCGGCGAGGGGGTTTCCCACCCCCTTTATCGTTACTTATACCTAC |
| | | A |
| | 24335 | GGGTGTTCCCCTTTTGCCCGCGGAACTTATCTCTCGCGGACTGACTCCC |
| | | A |
| | 24336 | ACCCGGTTTCGGGTCTATGGCATACAACTTCTCGCCCTTGTCAGACTCG |
| | | C |
| | 24337 | CTGCCTGGCTTACGCCTACGGGGCTTTCACCCTCTCCGGCGCCGGCATT |
| | | C |
| | 24338 | GCTGCGGGGCTGAGCCCCTTAACCTCGCCGGAAAAAGTAACTCGTAGGT |
| | | T |
| | 24339 | AAGGATGGCTCTCTTCAAATCTCCTGCGCCCGCGACGGATAGGGACCGA |
| | | A |
| | 24340 | CAGGCCCCACAACACCGCACACACAACCCCCGCCGGGTATCACATGCAC |
| | | A |
| | 24341 | CCCCTACGGATCCATGCCTTGGTGGGCCATTACCCCACCAACTAGCTAA |
| | | T |
| | 24342 | ACTTAGCACTCATCGTTTACGGCGTGGACTACCAGGGTATCTAATCCTG |
| | | T |
| | 24343 | TATCCATCGAAGACTAGGTGGGCCGTTACCCCGCCTACTATCTAATGGA |
| | | A |
| | 24344 | CAGGCGTCAGCTCGTATACGTCATCTTTCGATTTAGCACAAACCTGTGT |
| | | T |
| | 24345 | TGGCCGTTCAACCTCTCAGTCCGGCTACCGATCGCGGTCTTGGTGAGCC |
| | | G |
| | 24346 | CCTGTGTTTTTGCTAAACAGTCGCCTGGGCCTATTCACTGCGGCTCTCT |
| | | C |
| | 24347 | ACGCCTTTCGGCCTGACCTTAGCTCCCGACTTACTTGGAGCGGACGAAC |
| | | C |
| | 24348 | GGTCTGGGCTCTTTCCCTTTTGACTGCCCAACTTATCTCGTGCAGTCTG |
| | | A |
| | 24349 | GAATGAATGGCTGCTTCTGAGCCAACATCCTAGTTGTCTTAGAGATCCC |
| | | A |
| | 24350 | CCCCATCATGCCTCAACCTTCACGCCCAGCGGATTTACCTACCAGACAG |
| | | T |
| | 24351 | AAAAGTACGCGGTTCATCATATAAAGATGTTCCACAGCTTGTAAACACA |
| | | G |
| | 24352 | ATCTGAAGTCTTCTCGTTTAACATACAGGACTATTACCTTCTGTGGTGA |
| | | G |
| | 24353 | GGTCACACCCTTTTGAAGTGTCCCTTTGCTTAAATTACAGATGGTTACG |
| | | G |
| | 24354 | CAGCTTATCACGTCTTTCATCGGCTCTTAGTGCCAAGGCATCCACCCTG |
| | | C |
| | 24355 | TTCCATTCGGCACCGCCGGATCACTATTCCCGACTTTCGTCCCTGTTCG |
| | | A |
| | 24356 | TCCAGGTTCGATTGGCATTTCACCCCTACCCACACCTCATCCCCGCACT |
| | | T |
| | 24357 | TACACCTTCTGCGTACATAGAACGCTCTCCTACCATCCCCTAAGGGATC |
| | | C |
| | 24358 | GCTTGCGCTAACCTCTCCTCTTAACCTTCCAGCACCGGGCAGGCGTCAG |
| | | C |
| | 24359 | CGCCCGTTAGTACCGGTCGGCTCCACCCCTCGCGGGGCTTCCACCTCCG |
| | | G |
| | 24360 | CTCCGGGACCTTAGACGGCGGTCTGGATTCTTCTCCTCTCGGGGACGGA |
| | | C |
| | 24361 | TGGTTAAGTCCTCGATCGATTAGTATCTGTCAGCTCCATGTGTCGCCAC |
| | | A |
| | 24362 | TAAGTCCTTAACCTTGCTGCATACAATCGCTCGCCGGACCGTTCTACAA |
| | | A |
| | 24363 | ACCGGACTTTCCATTTCCGGCCCATGTTTCCCTCCCGTGTCCCCACAGT |
| | | T |
| | 24364 | CGGCTCCCACCTATGCTACGCAGAAGAATCCGGATATCAATGCCAGACT |
| | | A |
| | 24365 | ACCCCACATCCTTTTCCACTTAACATATATTTGGGGACCTTAGCTGGTG |
| | | G |
| | 24366 | CCACACCACTTCACCTAACAACAACACACAAGCACGATGATGGTAGTCA |
| | | C |
| | 24367 | TCATCCCCGCACTTTTCACGTACGTGTGGTTCGGACCTCCACGACGTCT |
| | | T |
| | 24368 | CCCTTCAAAGCCTCCGACCTATCCTACACATCACGTGCCCAGATTCAAT |
| | | G |
| | 24369 | CTTCACCTAACAACAATGCGCAAGCAGGACGTCAGTAGCCATCCTCATC |
| | | A |
| | 24370 | GGGGTACGGGCGGCAACGCGCCTGACGCCGAAGCTTTTCTCGGCACCAC |
| | | G |
| | 24371 | ATGGCTAGATCACCGGGTTTCGGGTCTATACCCTGCAACTTAACGCCCA |
| | | G |
| | 24372 | ATTAAACCACATGCTCCACCGCTTGTGCGGGCCCCCGTCAATTCCTTTG |
| | | A |
| | 24373 | GCCGGCTTTCCCAAAGCCGTTCTGCTACCTCTCGCGGATCAATTATGCG |
| | | G |
| | 24374 | ACGCCTTCCGGCCTCACCTTAGCTCCCGACTAACTTGGAGCGGACGAAC |
| | | C |
| | 24375 | ACACCACGCGGCGATACCAACCCGAAGGAAGGAACCACCACGAGGCGGA |
| | | G |
| | 24376 | CCGAACCCCGAGATGCACGCATCTCGGTTTGGCCTCTTTCGCGTTCGCT |
| | | C |
| | 24377 | GGGACTTCATCCTGGCCAAGTGTAGATCACTTGGTTTCGCGTCTACCCC |
| | | C |
| | 24378 | AGCCCTCGACCTATTAGTACTGCCAAGCTGAATGCCTCACGGCACTTAC |
| | | A |
| | 24379 | GGGAGCGGGATTACCTTCACTATCAATCCACCCGAAGGTTTCATGTACT |
| | | A |
| | 24380 | CACGCGGGATTCCACGAGGCCCGCGCTACTTGGGACAACACGATCGGAA |
| | | G |
| | 24381 | CCTACACCCTTCAACCATCTATTCCGTCAGATGGCGGCACTGTCACTAC |
| | | T |
| | 24382 | CCCCGTACCTGTTCTCGATACCAGGTTAGAACCCCGGTCACACAAGAGT |
| | | G |
| | 24383 | GTTTCACGTGTCTGGCCGTACTCTGGATCCTGCGCAGCTCTCTCCGTTT |
| | | T |
| | 24384 | TTCCCGCTTAGATGCTTTCAGCGGTTATCCCTCCCGAACGTAGCCAACC |
| | | G |
| | 24385 | GCACTCCCACAGCTTGTAGACACAGGGTTTCAGGTTCTCTTTCACTCCC |
| | | C |
| | 24386 | CCTGGCCAAGGGTAGATCACTTGGTTTCGCGTCTGCCACTGCCGACTAT |
| | | A |
| | 24387 | CCGCGAGGGACCTCACCTACATATCAGCGTGCCTTCTCCCGAAGTTACG |
| | | G |
| | 24388 | AAGCTCCATGGGGTCTTTCCGTCTTGCCGCAGGTAACCGGCATCTTCAC |
| | | C |
| | 24389 | CGTCGGCTTGGTGGGCCGTTACCTCACCAACTACCTAATCCAACGCGGG |
| | | T |
| | 24390 | GCTCCCACCTATCCTGTACATGCAATACCAAGCTCCAGTACCAAACTGG |
| | | A |
| | 24391 | ACCGGACTTTCCATTTCCGGCCCATGTTTCCCTCCCGTGTCCCCACAGT |
| | | T |
| | 24392 | CAGTTCCCCGGGTCTGCCTTCTCATATCCTATGAATTCAGATATGGATA |
| | | C |
| | 24393 | GGTCCCGGCAGATTCGCGCAGGATTCCTCGTGTCCCGCGTTACTCAGGA |
| | | T |
| | 24394 | GTATTAACTTTACTCCCTTCCTCCCCGCTGAAAGTACTTTACAACCCGA |
| | | A |
| | 24395 | GGGGGCGGGGAGCGGGGCGTGGGCGGGAGGAGGGGAGGAGGCGTGGGGG |
| | | G |
| | 24396 | CACGAGGCCCGCGCTACTTGGGACACGCGATCGGGAGACGGCAAGCGTC |
| | | C |
| | 24397 | CGTTTATCCCCTCCCTACTTAGCTACCCAGCGATGCTCTTGGCAGAACA |
| | | A |
| | 24398 | CCTCTTAACCTTCCGGCACCGGGCAGGCGTCAGAGCGTATACAGCGGCT |
| | | T |
| | 24399 | ACCTTGGGCGGACGAACCTTCCCCAAGAAACCTTAGATTTTCGGCCATT |
| | | A |
| | 24400 | TTCGTTCGCCACTACTAGCAGAATCATAATTTTATTTTCTTCTCCTACG |
| | | G |
| | 24401 | GTTTCTCGCATGCCTCTCGCTACTCATACCGGCATTCTCTCTTGTGCAG |
| | | T |
| | 24402 | CCTATCAACGTCGTCGTCTTCAACGTTCCTTCAGGACCCTTAAAGGGTC |
| | | A |
| | 24403 | CTGTTATCCCCAGGGTAGCTTTTATCCGTTGAGCGACGGCATTTCCATT |
| | | C |
| | 24404 | CAACAATATATGGAACACCTACCTGGCGAGACAATAGAATGTGTTCCCT |
| | | C |
| | 24405 | TTATAGTTACGGCCGCCGTTTACTGGGGCTTCAATTCAATGCTTCTCTT |
| | | G |
| | 24406 | ACAACAGAGCTTTACGATCCGAAAACCTTCATCACTCACGCGGCGTTGC |
| | | T |
| | 24407 | CCCGTTCCACGGGTTAGAATCCAAACAAATAAAGGGTCGTATTTCAACA |
| | | G |
| | 24408 | CCCCCTTCCCCCCTCTCCTCCCCCTTCCCCCTTTCGCGCCCCCTTTTCC |
| | | C |
| | 24409 | TGGTGTTCCAACCAATTCGGCTTGGGGGGATGGATCTTAAAAACTGGTC |
| | | C |
| | 24410 | CTCGTGTCCCGCCGTACTCAGGATCCTGCTTGGCATCAAGTGAATTTCA |
| | | A |
| | 24411 | AGCTTCTACACCCTTCAACCATCTATTCCGTCAGATGGCGGCACTGTCA |
| | | C |
| | 24412 | CCGATTAGTACCAGTCAACTCCGTACATCACTGCACTTCCATCCCTGGC |
| | | C |
| | 24413 | CGCTTGAACCACACATCAGGCCCCACGGCTTGCCACCATGTTAACCCGA |
| | | A |
| | 24414 | TGGCGAGACAATAGAATGTGTTCCCTCGTTTGTGGCATAGGACCATCAG |
| | | C |
| | 24415 | CGTCCATCCCGGTCCTCTCGTACTAGGGACAGCTCCTCTCAAATATCCT |
| | | G |
| | 24416 | TCGAGGTGCCAAACCTCCCCGTCGATGTGAACTCTTGGGGGAGATAAGC |
| | | C |
| | 24417 | CTTAACAACTTAACCTCGCTGCACACAGTAACTCGCCGGCCCGTTCTAC |
| | | A |
| | 24418 | GTCAACAGGTAGTATTCAGGCTTACCAGGTGGTCCTGGCAGATTCACAC |
| | | G |
| | 24419 | AGGCACGCCGTCACACATTGCTGTGCTCCGACCGCTTGTAGGCGTATGG |
| | | T |
| | 24420 | TCCCTTTCCCCCTTCCCCCCCCCCCCCCCCCCCCCCCCCTTTCCCCCCC |
| | | C |
| | 24421 | AACCATGACTTTGGGACCTTAGCTGGCGGTCTGGGTTGTTTCCCTCTTC |
| | | A |
| | 24422 | TGCCATTACACTCTATGAGACCGGTTACCAATCGGTCCGAAGGGCACCT |
| | | T |
| | 24423 | GATTGGAATTTCTCCGCTACCCACACCTCATCCGCTACCATTTCAACGG |
| | | G |
| | 24424 | TTCTCGTGTCCCGCGGTACTCTGGATCCTGCTCAGTCTGCTCTGTTTTC |
| | | G |
| | 24425 | GTAAACCCCCACAACAGCTATGAATTCACTGAAGGGTAACACCCCATAA |
| | | C |
| | 24426 | TCCCGAAGTTACAGGGTCAATTTGCCTAGTTCCTTAACCGTGAATCACT |
| | | C |
| | 24427 | CCCCCGACGGGTATCACACGCGCAAGGTTTGGCCATCATCCGCTTTCGC |
| | | T |
| | 24428 | CCCTTGTCTCAGTGCCCATCTCCGGGCTCCTCCTTCCAGAGCCCGTACC |
| | | C |
| | 24429 | TCAGACTTGCTCTCGCTGCGGCTTCACACCTTAAGTGCTTAACCTCGCC |
| | | G |
| | 24430 | CTCCATTCGGAAATCCACGGATCAATGCCTACTTACGGCTCCCCGTGGC |
| | | T |
| | 24431 | TTTTACGGTTGAGCCGCAAACTTTCACAACTGACTTAACAACCCGCCTA |
| | | C |
| | 24432 | CGGTTTAGGCTCTTCCGCGTTCGCTCGCCGCTACTTACGGAATCGAGTT |
| | | T |
| | 24433 | CTTCACTATATACTCTAGTACAGGAATATCAACCTGTTGGCCATCGGAT |
| | | A |
| | 24434 | TGTTTCAGTTCACTGCGTCTTCCTTCTCATAACCTTAACAGTTATGGAT |
| | | A |
| | 24435 | GACGGAGCTTATCCCCCGCCGACTCACTGCCGGGATACGCGTCACGGGT |
| | | A |
| | 24436 | CCGAACTGTCTCACGACGTTCTGAACCCAGCTCGCGTACCGCTTTAATG |
| | | G |
| | 24437 | GACGGTGACAAGCCGGTACCAGAATATCAACTGGTTACCCATCGACTAC |
| | | G |
| | 24438 | GATGCGCATTCGGAGTTTGTCAAGACTTGATAGGCGGTGAAGCCCTCGC |
| | | A |
| | 24439 | TAGGTGAGCCGTTACCCCACCTACTAGCTAATCCCATCTGGGCACATCC |
| | | G |
| | 24440 | TGGTCCCCGCTCATTCCATCAAGGTTTCTCGTGTCTCGATGTACTCTGG |
| | | A |
| | 24441 | ATGCTCCCCTACCGATACTTTTTAATGCTATCCCGCGCCTTCGGTACCT |
| | | G |
| | 24442 | TTACCTTTACTTCAACCTGACCATGGGTAGGTCACCCGGTTTCGGGTCG |
| | | A |
| | 24443 | GTAGTATTTAGCCTTGGAGGATGGTCCCTCCTGCTTCCCACAGGGTTTC |
| | | A |
| | 24444 | GATTTCCAACCATTCTGAGGGAACCTTTGGGCGCCTCCGTTACCTTTTA |
| | | G |
| | 24445 | ATCCCTTCCGGGCTTGGCTACTCGGCCGTAGACTTGGCAGTCTAACCGA |
| | | T |
| | 24446 | GATGCGCATTCGGAGTTTGTCAAGACTTGATAGGCGGTGAAGCCCTCGC |
| | | A |
| | 24447 | GTAATCGCCTTGGTGGGCCATTACCCCACCAACAAGCTGATAGGCCGCA |
| | | G |
| | 24448 | ACCCTCAGGTCATCCAGAAGCTTTTCAACGCTTATTGGTTCGGTCCTCC |
| | | A |
| | 24449 | AGCTCCATGGGGTCTTTCCGTCTAGTTGCGGGTAACCTGCATTTTCACA |
| | | G |
| | 24450 | CGTGGGGATTAAGTTTAGCGGATTTTCTCGGGAGTATGATTACGTGCGC |
| | | T |
| | 24451 | TATTTTGGGACCTTAACTGGCGGTCTGGGCTGTTTCCCTCTTGACCATG |
| | | G |
| | 24452 | TAACCTTGCACGGGATCGTAACTCGCCGGTTCATTCTACAAAAGGCACG |
| | | C |
| | 24453 | GACGGCCCAGAGACCTGCCTTCGCCATCGGTGTTCTTCCCGATATCTAC |
| | | A |
| | 24454 | TCACACGGGATTCCACGAGTCCCGCGCTACTTGGGAGACACGATCCGGA |
| | | G |
| | 24455 | AGTATTTAGCCTTGGAGGATGGTCCCCCCATATTCAGACAGGATACCAC |
| | | G |
| | 24456 | TTTGGCCTCTTCCGCGTTCGCTCGCCACTACTAGCGGAATCTCGGTTGA |
| | | T |
| | 24457 | CTGCTTCCAAGCCAACATCCTAGCTGTCTTAGCAGTCAGACTTCGTTAG |
| | | T |
| | 24458 | CTGGGGCTTCAATTCACACCTTCGCTTACGCTAAGCGCTCCTCTTAACC |
| | | T |
| | 24459 | GTTTGGGCTTCTCCCCTTTCGCTCGCCGCTACTCAGGGAATCACTGTTG |
| | | T |
| | 24460 | ACAATCCACACCGAATGCCAATACCAAGGTATAGTAAAGGTCCCGGGGT |
| | | C |
| | 24461 | CAGGGTAGCTTTTATCCGTTGAGCGATGGCCCTTCCATACGGTACCACC |
| | | G |
| | 24462 | ATAGGCGGTGAAGCCCTCTTGACCTATCGGTCGCTCTACCTCTCACGGT |
| | | G |
| | 24463 | GCCATGCAGATTCTCACTGCATTCGCGCTACTCATTCCGGCATTCTCAC |
| | | T |
| | 24464 | CGGTACGCCGCCGGTACGGGAATATCCACCCGTTCATCCATTCGACTAC |
| | | G |
| | 24465 | GCACTCCACAGCTCCTTCCGGTACTGCTTCTTCGCGTTAAGAATGCTCC |
| | | T |
| | 24466 | CGTTCACTCTTCCTTGGCTCCTACCTATCCTGTACATGTGTAACAGATA |
| | | C |
| | 24467 | CCCCTGACCTGATTCAAGGCCACAGGTTAGAATTTCAGCACTTCAAGAG |
| | | T |
| | 24468 | CTACCCAGCAATGCCTTTGGCAAGACAACTGGTACACCAGCGGTAAGTC |
| | | C |
| | 24469 | CCAGCACCGGGCAGGCGTCACCCCCTATACTTCATCTTACGATTTCGCA |
| | | G |
| | 24470 | ATTCCTCACTGCTGCCTCCCGTAGGAGTTTGGACCGTGTCTCAGTCCCA |
| | | A |
| | 24471 | CTACGAGACTCAAGCTTGCCAGTATCAGATGCAGTTCCCAGGTTGAGCC |
| | | C |
| | 24472 | CTCTCAACGATGACGTCTCCTCTTAACCTTCCAGCACCGGGCAGGTGTC |
| | | A |
| | 24473 | ATTACCGCGGCTGCTGGCACGGAGTTAGCCGGTGCTTCTTCTGCGGGTA |
| | | A |
| | 24474 | GCGATGGACTTTCACACCGGACGCGACGAGCCGCCTACGAGCCCTTTAC |
| | | G |
| | 24475 | CCCACACCGGATATGGACCGAACTGTCTCACGACGTTCTGAACCCAGCT |
| | | C |
| | 24476 | GAATGAATGGCTGCTTCTGAGCCAACATCCTAGTTGTCTTAGAGATCCC |
| | | A |
| | 24477 | TCCCCGGAGTACCTTTTATCCTTTGAGCGATGTCCCTTCCATACGGAAA |
| | | C |
| | 24478 | GTAAAGCCACCTTATACCCTTGCATTCTACAGGAGATTTCTGACCTCCT |
| | | T |
| | 24479 | TCCGCCTGCGCACCCTTTAAACCCAATAAATCCGGATAACGCTCGTATC |
| | | C |
| | 24480 | AGGAAGTATTCAGGCTTACCAGGTGGTCCTGGCAGATTCACACACGATT |
| | | C |
| | 24481 | GTGTAGGATTCTCACCTACATCTCGCTACTCACACCGGCATTCTCACTT |
| | | C |
| | 24482 | GAACTGAGACCGGTTTTCAGGGATCCGCTCCATGTCGCCATGTCGCATC |
| | | C |
| | 24483 | TTCCTGAAGTTGATTCTTCGGGTTAGACAGCCAAACTTCTCAGGGTGGT |
| | | A |
| | 24484 | CGGTACTGGTACGCTATCGGTCAGACAGGTATGCTTAGACTTACGCCAC |
| | | G |
| | 24485 | GTTTCCCCTCGACTTGCATGTGTTAAGCCTGTAGCTAGCGTTCATCCTG |
| | | A |
| | 24486 | CGAAGTTACGGGGTCATTTTGCCGAGTTCCTTGACAATGCTTCTCCCGC |
| | | C |
| | 24487 | CTTGGGAATGATCAGCCTGTTATCCCCGGGGTACCTTTTATCCGTTGAG |
| | | C |
| | 24488 | GTCTATAAGTACTTCGATTTTTGCAAGTCCGAACCCCGAACGTCCGTAG |
| | | A |
| | 24489 | CACCTTTCCTTCACAGTACTGGTTCACTATCGGTCTCTCGGGAGTATTT |
| | | A |
| | 24490 | CCGGGAATTCCAGTCTCCCCTACCGCACTCCAGCCCGCCCGTACCCGGC |
| | | G |
| | 24491 | ACAGCTTTTCTCGCCATCTTCCATCTCGGACTTCGGTACTAATTTCCCT |
| | | C |
| | 24492 | TCTTTCGGCGAGGGGGGTTCCCGCCCCCTTTATCGTTACTTATACCTAC |
| | | A |
| | 24493 | TGTATGCGCCATTGTAGCACGTGTGTAGCCCTGGTCGTAAGGGCCATGA |
| | | T |
| | 24494 | CTTTCGTCTCTGATCGAGTTGTCACTCTCGCAGTCAGGCACCCTTCTGC |
| | | C |
| | 24495 | GATACTACAATTTCACTGAGCTCTTGGTTGAGACAGCGTCCGGATCATT |
| | | A |
| | 24496 | GATGTTTCAGTTCAGGCGGTTCCCTCAATACACCTATTTTAAATTTCAG |
| | | T |
| | 24497 | AAAAAAAAACAAAAAAAAAAACCCTCCCCCCCCCCCCTTCCCCTCCGCG |
| | | G |
| | 24498 | GCCCTGTTAAGACTTGGTATCCCTTCGGCTCCGCACCTTAAGTGCTTAA |
| | | C |
| | 24499 | ACCACGAATTCCGCCTGCCTCAACTGCACTCAAGATATCCAGTATCAAC |
| | | T |
| | 24500 | GAGTTTTTCACACTGTGCCATGCAGCACTGTGCGCTTATGCGGTATTAG |
| | | C |
| | 24501 | TGCCTAGTTCCTTAACCATGAATCTCTCAACGCCTCAGTATGTTCTACC |
| | | C |
| | 24502 | GGTGTGTACAAGGCCCGGGAACGTATTCACCGCGCCGTGGCTGATGCGC |
| | | G |
| | 24503 | TTCGCCACCGGTATTCCTCCAGATCTCTACGCATTTCACCGCTACACCT |
| | | G |
| | 24504 | CGCTTAACGCGTTAGCTCCGACACGGAACACGTGGAACGTGCCCCACAT |
| | | C |
| | 24505 | ACACGAGCCGAAACCCGTGTCTCTCAGACTCCCACCTATCCTGTGCATC |
| | | A |
| | 24506 | ACTCGATTTCTCTTCGGCTCCACACCTTAAGTGCTTAACCTTGCCGGCA |
| | | C |
| | 24507 | TGAACCCGCCCCGAAGGGAAACGCCATCTCTGGCGTCGTCGGGAACATG |
| | | T |

### DESCRIPTION OF THE EMBODIMENTS

This application discloses method for enriching viral molecules from a nucleic acid sample, particularly coronavirus molecules. In some embodiments, the viral molecules are viral RNA molecules. In some embodiments, the viral molecules are genomic viral DNA or RNA molecules. In some embodiments, solid supports can be prepared for enriching desired library fragments or depleting unwanted library fragments, wherein oligonucleotides are immobilized to the solid support. In some embodiments, the solid support is a flowcell.

Also disclosed herein are compositions comprising a probe set comprising at least two DNA probes complementary to at least one target viral nucleic acid molecules in a nucleic acid sample.

Disclosed herein are also kits for depleting or enriching libraries. In some embodiments, the kit comprises probe compositions disclosed herein and instructions for using the probe set. Such a kit may further comprise reagents for preparing a cDNA library from RNA, such as reagents for a stranded method of cDNA preparation from a sample comprising RNA, as described below.

### I. Target and Off-Target Nucleic Acids

### A. Coronavirus Targets

Described herein are methods for enriching viral molecules from a nucleic acid sample. In some embodiments, the viral molecule is a coronavirus molecule. In some embodiments, at least one coronavirus molecule is of the Alphacoronavirus, Betacoronavirus, Deltacoronavirus, Gammacoronavirus, and/or Bafinivirus genus.

As used herein, the term “nucleic acid” is intended to be consistent with its use in the art and includes naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g., found in deoxyribonucleic acid (DNA)) or a ribose sugar (e.g., found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from the group consisting of adenine, thymine, cytosine or guanine and a ribonucleic acid can have one or more bases selected from the group consisting of uracil, adenine, cytosine, or guanine. Useful non-native bases that can be included in a nucleic acid are known in the art. The term “target,” when used in reference to a nucleic acid, is intended as a semantic identifier for the nucleic acid in the context of a method or composition set forth herein and does not necessarily limit the structure or function of the nucleic acid beyond what is otherwise explicitly indicated.

In some embodiments, the present methods decrease library preparation costs and hands-on-time, as compared to prior art methods of enrichment, followed by library preparation.

As used herein, “desired RNA” or “a desired RNA sequence” refers to any RNA that a user wants to analyze. As used herein, a desired RNA includes the complement of a desired RNA sequence. Desired RNA may be RNA from which a user would like to collect sequencing data, after cDNA and library preparation. In some instances, the desired RNA is mRNA (or messenger RNA). In some instances, the desired RNA is a portion of the mRNA in a sample. For example, a user may want to analyze RNA transcribed from cancer-related genes, and thus this is the desired RNA.

As used herein, “desired library fragments” refers to library fragments prepared from cDNA prepared from desired RNA.

In some embodiments, the desired RNA sequence is a coronavirus sequence.

Also disclosed herein are compositions comprising a library fragment bound to an immobilized oligonucleotide on a solid support. In some embodiments, a single-stranded library fragment comprising cDNA prepared from a sample comprising RNA is hybridized to a solid support comprising immobilized oligonucleotides. In some embodiments, the cDNA comprised in the composition is complementary to RNA comprised in the sample.

### B. Off Target RNA

Also described herein are methods for depleting off-target RNA molecules from a nucleic acid sample.

As used herein, “off-target RNA,” “an off-target RNA sequence”, “unwanted RNA,” or “an unwanted RNA sequence” refers to any RNA that a user does not wish to analyze. As used herein, an unwanted RNA includes the complement of an unwanted RNA sequence. When RNA is converted into cDNA and this cDNA is prepared into a library, a user would sequence library fragments that were prepared from all RNA transcripts in the absence of depletion. Methods described herein for depleting library fragments prepared from unwanted RNA can thus save the user time and consumables related to sequencing and analyzing sequencing data prepared from unwanted RNA. In some embodiments, off-target RNA relates to small non-coding RNA (sncRNA). In some embodiments, the off-target RNA comprises sncRNA with MALAT 1. In some embodiments, off-target RNA comprises at least one small noncoding RNA chosen from RN7SK, RN7SL1, RN7SL2, RN7SL5P, RPPH1, SNORD3A. In some embodiments the off-target RNA is not MALAT1.Small noncoding RNAs are highly abundant as reads during the sequencing process and can lead to noise when analyzing sequencing data. MALAT1 is also highly abundant in the genome. MALAT1 is a highly conserved large, infrequently spliced non-coding RNA which is highly expressed in the nucleus. Trying to remove these reads after sequencing results in wasted sequencing.

As used herein, “off-target RNA,” “unwanted RNA” or “unwanted RNA sequence” also includes fragments of such RNA. For example, an unwanted RNA may comprise part of the sequence of an unwanted RNA. In some embodiments, unwanted RNA sequence is from human, rat, mouse, or bacteria. In some embodiments, the bacteria are Archaca species, E. Coli, or B. subtilis.

As used herein, “off-target library fragments” or “unwanted library fragments” also includes library fragments prepared from cDNA prepared from unwanted RNA.

Also described herein are compositions comprising a probe set comprising at least two DNA probes complementary to discontiguous sequences at least 5, or at least 10, or 15 bases apart along the full length of at least one off-target RNA molecule in a nucleic acid sample and a ribonuclease capable of degrading RNA in a DNA: RNA hybrid, wherein the off-target RNA comprises at least one small noncoding RNA chosen from RN7SK, RN7SL1, RN7SL2, RN7SL5P, RPPH1, SNORD3A

In some embodiments, the off-target RNA is high-abundance RNA. High-abundance RNA is RNA that is very abundant in many samples and which users do not wish to sequence, but it may or may not be present in a given sample. In some embodiments, the high-abundance RNA sequence is a ribosomal RNA (rRNA) sequence. Exemplary high-abundance RNAs are disclosed in WO2021/127191 and WO 2020/132304, each of which is incorporated by reference herein in its entirety.

In some embodiments, the high-abundance RNA sequences are the most abundant RNA sequences determined to be in a sample. In some embodiments, the high-abundance RNA sequences are the most abundant RNA sequences across a plurality of samples even though they may not be the most abundant in a given sample. In some embodiments, a user utilizes a method of determining the most abundant RNA sequences in a sample, as described herein.

In a given sample, the most abundant sequences are the 100 most abundant sequences. In some embodiments, in addition to depleting the 100 most abundant sequences, the method also is capable of depleting the 1,000 most abundant sequences, or the 10,000 most abundant sequences in a sample. In some embodiments, the off-target RNA sequence comprises a sequence with homology of at least 90%, at least 95%, or at least 99% to a most abundant sequence in a sample comprising RNA. In some embodiments, the off-target RNA sequence comprises a sequence with homology of at least 90%, at least 95%, or at least 99% to a most abundant sequence in a sample comprising RNA, wherein the most abundant sequences comprise the 100 most abundant sequences. In some embodiments, homology is measured against the 1,000 most abundant sequences, or the 10,000 most abundant sequences.

In some embodiments, the high-abundance RNA sequences are comprised in RNA known to be highly abundant in a range of samples.

In some embodiments, the off-target RNA sequence is globin mRNA or 28S, 23S, 18S, 5.8S, 5S, 16S, 12S, HBA-A1, HBA-A2, HBB, HBB-B1, HBB-B2, HBG1, or HBG2 RNA, or a fragment thereof.

In some embodiments, the off-target RNA sequence is 28S, 18S, 5.8S, 5S, 16S, or 12S RNA from humans, or a fragment thereof. In some embodiments, the off-target RNA sequence is rat 16S, rat 28S, mouse 16S, or mouse 28S RNA.

In some embodiments, the off-target RNA sequence is comprised in mRNA related to one or more “housekeeping” genes. For example, a housekeeping gene may be one that is commonly expressed in a sample from a tumor or other oncology-related sample, but that is not implicated in tumor genesis or progression. Housekeeping genes are typically constitutive genes that are required for the maintenance of basal cellular functions that are essential for the existence of a cell, regardless of its specific role in the tissue or organism. In some embodiments, the off-target RNA sequence is comprised in 23S, 16S, or 5S RNA from Gram-positive or Gram-negative bacteria.

### II. Compositions

Described herein are compositions comprising a probe set comprising at least one DNA probe comprising at least one sequence of SEQ ID NOs: 1-22909.

Also described herein are compositions comprising a probe set comprising at least two DNA probes complementary to at least one target coronavirus nucleic acid molecules in a nucleic acid sample wherein the target coronavirus nucleic comprises at least one coronavirus molecule selected from Table 2.

In some embodiments, the one or more target coronavirus nucleic acids are coronavirus RNA molecules. In some embodiments, the one or more target coronavirus nucleic acids are genomic coronavirus RNA molecules.

In some embodiments, the probe set further comprises at least two DNA probes that each hybridize to at least one target coronavirus molecule of the Alphacoronavirus, Betacoronavirus, Deltacoronavirus, Gammacoronavirus, and/or Bafinivirus genus.

In some embodiments, the probe set further comprises at least two DNA probes that each hybridize to at least one target coronavirus molecule selected from Table 2.

Also described herein are compositions comprising a probe set comprising at least one DNA probe comprising at least one sequence of SEQ ID NOs: 1-22909. In some embodiments, the composition comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, or 22909sequences selected from SEQ ID NOs: 1-22909. In some embodiments, the at least one DNA probe comprises 500 or more, 600 or more, 700 or more, 800 or more, 900 or more, 1000 or more, 1100 or more, or 22909sequences selected from SEQ ID NOs: 1-22909.

In some embodiments, the composition comprises at least 5, at least at least 10, at least 50, at least 100, at least 250, at least 500, at least 750, at least 1000, at least 1500, or at least 2000 sequences of SEQ ID NOs: 1-22909. In some embodiments, the composition comprises two or more, five or more, 10 or more, or 25 or more sequences selected from SEQ ID NOs: 1-22909.

In some embodiments the probe set comprises any one or more of SEQ ID NOs: 22910-24507.

In some embodiments the probe set is biotinylated.

### III. Methods of Use

### A. Methods of Enriching for Viral Nucleic Acids

Described herein are methods of enriching a sample for one or more target viral nucleic acids.

In some embodiments, the present methods decrease library preparation costs and hands-on-time, as compared to prior art methods of enriching for vial nucleic acids, followed by library preparation.

In some embodiments, the method comprises providing any of the compositions described herein, in Section II (Compositions) above.

In some embodiments, the method comprises providing a probe set comprising at least two nucleic acid probes complementary to one or more target viral nucleic acids, wherein the probe set comprises at least two of SEQ ID NOs: 1-22909; allowing the probes in the probe set to hybridize to the target viral nucleic acids; and enriching the sample for the one or more target viral nucleic acids by amplifying the target viral nucleic acids and/or separating the target viral nucleic acids from the sample.

Also described herein are methods of enriching a sample for one or more target coronavirus nucleic acids. In some embodiments, the present methods detect or enrich for new or unknown viral pathogens, including coronaviruses, or new or unknown strains of viral pathogens, including coronaviruses. This may include analysis of patient samples. In some embodiments, the present methods detect co-infections with one or more additional pathogens, including viruses or bacteria. In some embodiments, the present methods detect or enrich for specific viral pathogen strains. In some embodiments, the present methods can be used to perform strain typing and/or strain characterization for monitoring viral pathogen evolution and epidemiology (e.g., coronavirus evolution and epidemiology). In some embodiments, the present methods detect or enrich for viral nucleic acids that exhibit resistance. Resistance can include resistance to anti-viral therapies (whether small molecule therapy or other therapies including treatment with antibodies (including antigen-binding fragments thereof or other biologics with CDRs responsible for specific binding), viral entry inhibitors, viral assembly inhibitors, viral DNA and RNA polymerase inhibitors, viral reverse transcriptase inhibitors, viral protease inhibitors, viral integrase inhibitors, and inhibitors of viral shedding. In some embodiments, the present methods are used to identify hospital-associated viral infections (e.g., hospital-associated coronavirus infections). As used herein, a hospital-associated viral infection refers to an infection whose development spread through and/or is favored by a hospital environment, nursing home, rehabilitation facility, group home, residential facility, medical office, clinic, or other clinical settings. This infection is spread to a subject in the clinical setting by a number of means, for example through contaminated equipment, bed linens, or air droplets. In some embodiments, the present methods are used for viral resequencing. In some embodiments, resequencing allows for testing for known mutations or scanning for one or more mutations in a given target region. Such methods may be used in a panel used for detection of and/or typing of viral pathogens (e.g., coronaviruses).

In some embodiments, the method comprises providing a probe set comprising at least two nucleic acid probes complementary to one or more target coronavirus nucleic acids, wherein the nucleic acid probes are affixed to a support; capturing one or more target coronavirus nucleic acids on a support; using the one or more captured target coronavirus nucleic acids as a template strand to produce one or more nucleic acid duplexes immobilized on the support, wherein the at least one target coronavirus nucleic acids hybridize to one or more probes in a probe set on the support; contacting a transposase and transposon with the one or more nucleic acid duplexes under conditions wherein the one or more nucleic acid duplexes and transposon composition undergo a transposition reaction to produce one or more tagged nucleic acid duplexes, wherein the transposon composition comprises a double stranded nucleic acid molecule comprising a transferred strand and a non-transferred strand; contacting the one or more tagged nucleic acid duplexes with a nucleic acid modifying enzyme under conditions to extend the 3′ end of the immobilized strand to the 5′ end of the template strand to produce one or more end-extended tagged nucleic acid duplexes; amplifying the one or more end-extended tagged nucleic acid duplexes to produce a plurality of tagged nucleic acid strands; contacting the plurality of tagged nucleic acid strands with a probe set to create an enriched library; and amplifying the enriched library. A wide variety of solid supports may be used to immobilize oligonucleotides for depleting or enriching as described herein, including those described in WO 2014/108810, which is incorporated in its entirety herein.

The composition and geometry of the solid support can vary with its use. In some embodiments, the solid support is a planar structure such as a slide, chip, microchip and/or array. As such, the surface of a substrate can be in the form of a planar layer. In some embodiments, the solid support comprises one or more surfaces of a flowcell. The term “flowcell” as used herein refers to a chamber comprising a solid surface across which one or more fluid reagents can be flowed. Examples of flowcells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and U.S. 2008/0108082, each of which is incorporated herein by reference.

In some embodiments, a flowcell is comprised within an apparatus or device for sequencing nucleic acids, which may be referred to as a sequencer. In some embodiments, a sequence may also comprise reservoirs for collection of samples or tubing (such as for collecting samples in a reservoir of for exiting of waste). In some embodiments, one or more reservoirs are separate from the flowcell and are comprised in the sequencer. In some embodiments, modifications are made to standard sequencers to improve fluidics system recipes and/or hardware for use of reservoirs in the present methods.

As used herein, a “flowcell” may comprise a flowcell-like device that is not intended to be imaged. While standard flowcells used for imaging may be employed in the present methods, flowcells can also be engineered differently than flowcells intended for imaging. In some embodiments, a flowcell may have a high density of immobilized oligonucleotides, wherein imaging infrastructure would have difficulty separating out into different bridge-amplified clusters associated with different immobilized oligonucleotides. In some embodiments, a high density of immobilized oligonucleotides improves hybridization efficiency. In some embodiments, standard clear glass may be used in a flowcell. In other embodiments, hard plastic may be used in the flowcell. Use of glass in a flowcell may allow use of a standard flowcell without further optimization, whereas use of hard plastic may reduce the cost of manufacturing the flowcell and/or improve stability of a flowcell. Depending on the advantages desired, different materials may be used. In some embodiments, immobilized oligonucleotides are embedded in a substrate other than that of a standard flowcell (i.e., embedded in a substrate other than PAZAM) to improve immobilization of oligonucleotides of longer length.

### B. Methods of Supplementing a Probe Set for Use in Enriching for Viral Nucleic Acids

Also described herein are methods of supplementing a probe set for use in enriching for viral nucleic acid molecules from a nucleic acid sample.

In some embodiments, the methods of enriching for viral nucleic acids described herein can be supplemented with or used in conjunction with other enrichment panels. In some embodiments, the method also targets genitourinary pathogens, Antimicrobial Resistance (AMR) markers, respiratory viruses, respiratory pathogens (e.g., viruses, bacteria, fungi, and/or parasites), and/or exonic content. In some embodiments, the method is used with, supplemented with, or used in conjunction with the Urinary Pathogen ID/AMR Panel or Enrichment Kit (UPIP; Illumina). In some embodiments, the method is used with, supplemented with, or used in conjunction with the Respiratory Virus Oligos Panel or Enrichment Kit (RVOP; Illumina). In some embodiments, the method is used with the Illumina Exome Panel (Illumina). In some embodiments, the method is used with, supplemented with, or used in conjunction with the Virus Surveillance Panel or Enrichment Kit (VSP; Illumina) In some embodiments, the method is used with, supplemented with, or used in conjunction with the Respiratory Pathogen ID/Antimicrobial Resistance (AMR) Panel or Enrichment Kit (Illumina). In some embodiments, the method is used with, supplemented with, or used in conjunction with the Pan-CoV Panel or Enrichment Kit (Illumina). In some embodiments, the method is supplemented with or used in conjunction with the Illumina Exome Panel (Illumina). In some embodiments, the method targets and enriches for coding RNA sequences. In some embodiments, the method is used with the Illumina RNA Prep with Enrichment (Illumina).

Examples of supplemental probe sets that can be readily used in the methods of the present disclosure are described, for example, in U.S. Provisional Application No. 63/250,563, filed Sep. 30, 2021, U.S. Provisional Application No. 63/351,170, filed Jun. 10, 2022, and U.S. Provisional Application No. 63/378,610, filed Oct. 6, 2022, each of which is incorporated by reference herein in its entirety.

In some embodiments the method comprises depleting unwanted nucleic acid molecules from a nucleic acid sample.

In some embodiments, the depleting unwanted nucleic acid molecules comprises depleting unwanted cDNA library fragments from a library of cDNA fragments prepared from RNA, wherein the unwanted library fragments comprise those prepared from unwanted RNA sequences, further comprising: preparing a solid support comprising at least one immobilized oligonucleotide, wherein each immobilized oligonucleotide comprises a nucleic acid sequence corresponding to an unwanted RNA sequence or its complement, adding the library of fragments to the solid support and hybridizing the library fragments to at least one immobilized oligonucleotide to allow binding of unwanted library fragments to at least one immobilized oligonucleotide, and collecting library fragments not bound to at least one immobilized oligonucleotide.

In some embodiments, the at least one immobilized oligonucleotide comprises a sequence comprising any one or more of SEQ ID NOs: 23377-24507 or its complement.

In some embodiments, the depleting unwanted nucleic acid molecules comprises depleting off-target RNA nucleic acid molecules from a nucleic acid sample comprises contacting a nucleic acid sample comprising at least one RNA or DNA target sequence and at least one off-target RNA molecule from a first species with a probe set comprising at least two DNA probes complementary to discontiguous sequences along the full length of the at least one off-target RNA molecule from a second species, thereby hybridizing the DNA probes to the off-target RNA molecules to form DNA: RNA hybrids, wherein each DNA: RNA hybrid is at least 5 bases apart, or at least 10 bases apart, along a given off-target RNA molecule sequence from any other DNA: RNA hybrid, wherein the off-target DNA comprises at least one small noncoding RNA chosen from RN7SK, RN7SL1, RN7SL2, RN7SL5P, RPPH1, SNORD3A; contacting the DNA: RNA hybrids with a ribonuclease that degrades the RNA from the DNA: RNA hybrids, thereby degrading the off-target RNA molecules in the nucleic acid sample to form a degraded mixture; separating the degraded RNA from the degraded mixture; sequencing the remaining RNA from the sample; evaluating the remaining RNA sequences for the presence of off-target RNA molecules from the first species, thereby determining gap sequence regions; and supplementing the probe set with additional DNA probes complementary to discontiguous sequences in one or more of the gap sequence regions.

In some embodiments, the probe set comprises any one or more of SEQ ID NOs: 22917-23376.

In some embodiments, the method further comprises depleting unwanted cDNA library fragments from a library of cDNA fragments prepared from RNA, wherein the unwanted library fragments comprise those prepared from unwanted RNA sequences. In some embodiments, a solid support comprises more than one pool of immobilized oligonucleotides on its surface.

For example, a solid support may comprise a first pool of immobilized oligonucleotides for depleting and a second pool of immobilized oligonucleotides for enriching. In some embodiments, one pool of immobilized oligonucleotides may be blocked (such as with complementary nucleic acid sequences) to avoid binding to complementary library fragments during certain steps of methods using the solid support.

In some embodiments, a solid support has two pools of immobilized oligonucleotides on its surface, wherein the first pool comprises immobilized oligonucleotides each comprising an unwanted RNA sequence and the second pool comprises immobilized oligonucleotides each comprising a solid support adapter sequence that can bind to a library adapter comprised in library fragments. In some embodiments, solid support adapter sequences are bound by adapter complements, wherein the adapter complements can be denatured during a method to allow binding of solid support adapter sequences to library adapters in library fragments. Such a solid support can be used for methods of preparing a depleted library and amplifying the depleted library on the same solid support.

In some embodiments, at least one unwanted RNA sequence has at least 90%, at least 95%, or at least 99% homology to a high-abundance RNA sequence in a sample used to prepare the library of fragments. In some embodiments, all unwanted sequences have at least 90%, at least 95%, or at least 99% homology to a high-abundance RNA sequence in a sample used to prepare the library of fragments.

### C. Samples

In some embodiments, the sample comprises a microbe sample, a microbiome sample, a bacteria sample, a yeast sample, a plant sample, an animal sample, a patient sample, an epidemiology sample, an environmental sample, a soil sample, a water sample, a metatranscriptomics sample, or a combination thereof. In some embodiments, samples are from mixed populations of microbes such as microbial populations or viral populations from patients.

In some embodiments the sample is a water sample. In some embodiments, the water sample is a freshwater sample, a wastewater sample, a saline water sample, or a combination thereof. In some embodiments, the sample comprises a wastewater sample.

In some embodiments, the sample may be from a mammal. In some embodiments the sample may be from a human, monkey, bat, dog, cat, horse, goat, sheep, cow, pig, rat and/or mouse. In some instances, reservoirs of coronaviruses or other microbes in animal populations can serve as samples to predict what diseases or strains of diseases may become human pathogens or to compare sequences in animal reservoirs to sequences of pathogens infecting humans.

In some embodiments, samples may be from a patient. In some embodiments, samples may be from a patient with cancer (i.e., an oncology sample). In some embodiments, samples may be from a patient with a rare disease. In some embodiments, samples may be from a patient with coronavirus SARS-COV2 (COVID-19).

In some embodiments, the sample may be a tumor sample. In some embodiments, the sample may be a blood sample, a serum sample, and/or a whole blood sample. In some embodiments the sample may be a tissue sample. In some embodiments the sample may be a fecal sample, a urine sample, a mucus sample, a saliva sample, a lymph sample, a vaginal fluid sample, a semen sample, an amniotic sample, and/or a sweat sample.

### D. Library Preparation

Libraries prepared by any method can be used together with the present methods of enriching and/or depleting. In some embodiments, probes are single-stranded to allow for hybridizing and capturing of single-stranded library fragments that are complementary. In some embodiments, specific binding of a single-stranded library fragment to a probe generates a double-stranded oligonucleotide. In some embodiments, the double-stranded oligonucleotide forms a DNA: RNA hybrid. The probe specifically bound to the library fragment may be bound with a high-enough affinity to be recognized for degradation with a ribonuclease. In some embodiments, the off-target RNA molecules are degraded after contacting the sample with a ribonuclease to form a degraded mixture.

As used herein, the term “library” refers to a collection of members. In one embodiment, the library includes a collection of nucleic acid members, for example, a collection of whole genomic, subgenomic fragments, cDNA, cDNA fragments, RNA, RNA fragments, or a combination thereof. In some embodiments, a portion or all library members include a non-target adaptor sequence. The adaptor sequence can be located at one or both ends. The adaptor sequence can be used in, for example, a sequencing method (for example, an NGS method), for amplification, for reverse transcription, or for cloning into a vector.

In some embodiments, this DNA: RNA hybrid-specific cleavage comprises use of RNase H. This methodology is implemented as part of the current Illumina Total RNA Stranded Library Prep workflow and New England Biolabs NEBNext rRNA Depletion Kit and RNA depletion methods as described in U.S. Pat. Nos. 9,745,570 and 9,005,891.

### E. Amplification

In some embodiments, methods described herein comprise one or more amplification step. In some embodiments, library fragments are amplified before being added to a solid support. In some embodiments library fragments are amplified after a method of depleting described herein. In some embodiments, amplifying is by PCR amplification.

As used herein, “amplify,” “amplifying,” or “amplification reaction” and their derivatives, refer generally to any action or process whereby at least a portion of a nucleic acid molecule is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. Amplification optionally includes linear or exponential replication of a nucleic acid molecule. In some embodiments, such amplification can be performed using isothermal conditions; in other embodiments, such amplification can include thermocycling. In some embodiments, the amplification is a multiplex amplification that includes the simultaneous amplification of a plurality of target sequences in a single amplification reaction. In some embodiments, “amplification” includes amplification of at least some portion of DNA and RNA based nucleic acids alone, or in combination. The amplification reaction can include any of the amplification processes known to one of ordinary skill in the art. In some embodiments, the amplification reaction includes polymerase chain reaction (PCR).

1. Amplification after Enriching

In some embodiments, collected library fragments are amplified after a method of enriching. In some embodiments, an enriched library is amplified.

In some embodiments, the amplifying is performed with a thermocycler. In some embodiments, the amplifying is by PCR amplification.

As used herein, the term “polymerase chain reaction” (“PCR”) refers to the method as described in U.S. Pat. Nos. 4,683,195 and 4,683,202, which describe a method for increasing the concentration of a segment of a polynucleotide of interest in a mixture of genomic DNA without cloning or purification. This process for amplifying the polynucleotide of interest consists of introducing a large excess of two oligonucleotide primers to the DNA mixture containing the desired polynucleotide of interest, followed by a series of thermal cycling in the presence of a DNA polymerase. The two primers are complementary to their respective strands of the double stranded polynucleotide of interest. The mixture is denatured at a higher temperature first and the primers are then annealed to complementary sequences within the polynucleotide of interest molecule. Following annealing, the primers are extended with a polymerase to form a new pair of complementary strands. The steps of denaturation, primer annealing, and polymerase extension can be repeated many times (referred to as thermocycling) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as the “polymerase chain reaction” (hereinafter “PCR”). Because the desired amplified segments of the polynucleotide of interest become the predominant nucleic acid sequences (in terms of concentration) in the mixture, they are said to be “PCR amplified.” In a modification to the method discussed above, the target nucleic acid molecules can be PCR amplified using a plurality of different primer pairs, in some cases, one or more primer pairs per target nucleic acid molecule of interest, thereby forming a multiplex PCR reaction.

In some embodiments, the amplifying is performed without PCR amplification. In some embodiments, the amplifying does not require a thermocycler. In some embodiments, depleting and amplifying after the depleting is performed in a sequencer.

In some embodiments, the amplifying is performed without a thermocycler. In some embodiments, the amplifying is performed by bridge or cluster amplification

### F. Sequencing of Enriched Libraries

In some embodiments, a library enriched for enriching for target viral sequences library fragments is sequenced.

In some embodiments, sequencing data generated after enriching for target viral sequences is capable of capturing novel coronaviruses with homology to the sequence in the probe set. In some embodiments, sequencing data generated after enriching for target viral sequences is capable of capturing new or unknown viruses (e.g., new or unknown coronaviruses). In some embodiments, sequencing data generated after enriching for target viral sequences is capable of capturing co-infections. In some embodiments, sequencing data generated after enriching for target viral sequences is capable of capturing specific viral strains (e.g., specific coronavirus strains). In some embodiments, sequencing data generated after enriching for target viral sequences is capable of capturing viral nucleic acids that exhibit resistance. In some embodiments, sequencing data generated after enriching for target viral sequences provides unbiased viral pathogen detection. In some embodiments, sequencing data generated after enriching for target viral sequences is capable of capturing viral nucleic acids present in hospital-associated infection management.

Enriched libraries prepared by the present method can be used with any type of RNA sequencing, such as RNA-seq, small RNA sequencing, long non-coding RNA (lncRNA) sequencing, circular RNA (circRNA) sequencing, targeted RNA sequencing, exosomal RNA sequencing, and degradome sequencing.

Enriched libraries can be sequenced according to any suitable sequencing methodology, such as direct sequencing, including sequencing by synthesis, sequencing by ligation, sequencing by hybridization, nanopore sequencing and the like. In some embodiments, the enriched libraries are sequenced on a solid support. In some embodiments, the solid support for sequencing is the same solid support on which the enriching is performed. In some embodiments, the solid support for sequencing is the same solid support upon which amplification occurs after the enriching.

Flowcells provide a convenient solid support for performing sequencing. One or more library fragments (or amplicons produced from library fragments) in such a format can be subjected to an SBS or other detection technique that involves repeated delivery of reagents in cycles. For example, to initiate a first SBS cycle, one or more labeled nucleotides, DNA polymerase, etc., can be flowed into/through a flowcell that houses one or more amplified nucleic acid molecules. Those sites where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flowcell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with amplicons produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

The term “flow cell” as used herein refers to a chamber comprising a solid surface across which one or more fluid reagents can be flowed. Examples of flow cells and related fluidic systems and detection platforms that can be readily used in the methods of the present disclosure are described, for example, in Bentley et al., Nature 456:53-59 (2008); WO 04/018497; WO 91/06678; WO 07/123744; U.S. Pat. Nos. 7,057,026; 7,211,414; 7,315,019; 7,329,492; 7,405,281; and US Pat. Publication No. 2008/0108082.

### G. Whole Genome Sequencing, Amplicon Sequencing, Metagenomic Analysis, and Metatranscriptomic Analysis

In some embodiments, samples are sequenced using whole-genome sequencing and/or amplicon sequencing. Whole genome sequencing refers to sequencing the genome of any organism including viral pathogens (e.g., coronaviruses) and host organisms. For example, whole genome sequencing may be performed on a microbial isolate. Transmission dynamics may be evaluated by whole genome sequencing. Whole genome sequencing also provides useful information on strain characterization, resistance detection, and hospital-associated infection management.

In some embodiments, samples are sequenced using amplicon sequencing. The term “amplicon” refers to the resultant mixture of compounds after two or more cycles of the PCR steps of denaturation, annealing and extension. Thus, amplicon sequencing is the sequencing of amplicons and this can provide useful information on variant identification and characterization. In some embodiments, amplicon sequencing encompasses amplification of one or more segments of one or more target sequences, which can be performed by using probes to target and amplify regions of interest, followed by sequencing, such as next-generation sequencing. Amplicon sequencing may be performed on a variety of samples, including patient samples or microbial isolates, and is useful for strain characterization. It is also useful for viral resequencing and resistance detection.

In some embodiments, additional information may be obtained about samples using metagenomic and/or metatranscriptomic analyses. Metagenomic and/or metatranscriptomic analysis may be performed on patient samples and may provide unbiased viral pathogen detection. In some embodiments, metagenomic or metatranscriptomic analyses comprises sequencing the genomes of a plurality of individuals of different species in a given sample. In some embodiments, metagenomic or metatranscriptomic analyses is done without prior knowledge regarding the biological species in the sample, whether they be viral or human. In some embodiments, metagenomic or metatranscriptomic analyses enables determination of which species are present, and their relative abundances. Thus, metagenomic and/or metatranscriptomic analysis may be useful for unknown viral pathogen detection, co-infection detection, resistance detection, and/or strain characterization.

In some embodiments, whole genome sequencing, amplicon sequencing, metgenomic analysis, and/or metatranscriptomic analyses may be used in combination with each other.

### IV. Kits

Described herein is a kit comprising any of the compositions described herein in Section II, Compositions, above.

Disclosed herein are also kits for depleting or enriching libraries. In some embodiments, the kit comprises a solid support disclosed herein and instructions for using the solid support. Such a kit may further comprise reagents for preparing a cDNA library from RNA, such as reagents for a stranded method of cDNA preparation from a sample comprising RNA, as described below.

In some embodiments the kit comprises at least one DNA probe comprising at least one sequence comprising at least one of SEQ ID NOs: 1-2909 and a buffer.

In some embodiments, the buffer is a wash buffer and/or an elution buffer.

In some embodiments, the kit further comprises an RNA depletion buffer, a probe depletion buffer, and/or a probe removal buffer.

In some embodiments, the kit further comprises a ribonuclease; a DNase; and RNA purification beads. In some embodiments, the ribonuclease is RNase H.

In some embodiments, the kit comprises a buffer and nucleic acid purification medium. In some embodiments, the buffer is an RNA depletion buffer, a probe depletion buffer, and/or a probe removal buffer.

In some embodiments, the kit comprises a nucleic acid destabilizing chemical. In some embodiments, the nucleic acid destabilizing chemical comprises betaine, DMSO, formamide, glycerol, or a derivative thereof, or a mixture thereof. In some embodiments, the nucleic acid destabilizing chemical comprises formamide.

Throughout this application and claims, the term “and/or” means one or more of the listed elements or a combination of any two or more of the listed elements.

The term “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided. The term “consisting of” is limited to whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Unless otherwise specified, “a,” “an,” “the,” and “at least one” are used interchangeably and mean one or more than one.

As used herein, the term “each,” when used in reference to a collection of items, is intended to identify an individual term in the collection but does not necessarily refer to every term in the collection unless the context clearly dictates otherwise.

The recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

All headings are for the convenience of the reader and should not be used to limit the meaning of the text that follows the heading, unless so specified.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

### EXAMPLES

The following examples are illustrative only and are not intended to limit the scope of the application. Modifications will be apparent and understood by skilled artisans and are included within the spirit and under the disclosure of this application.

### Example 1. Preparation of Probes to Improve Enrichment of Coronaviruses of Interest in Wastewater Samples

### A. Probe Design

Probes were designed by a proprietary algorithm for enrichment probes running on a Linux server (first iteration probes). The weighting for spacing and probe scoring variables were set to 6 and 1 respectively. Probe spacing was set to ‘adjacent’, or 80 bp center to center.

The first iteration probes aimed to strike a balance between capturing the most important virus species/the ones most heavily represented as isolates in NCBI nucleotide database and capturing a diverse set of sequences from the four coronavirus genera. Choice of diverse sequences was based on phylogenetic trees generated by sequence alignments using the MAFFT alignment algorithm set to the FFT-NS-i iterative refinement method and generating trees using Jalview average distance method. See Table 1.

TABLE 1

| FASTA_ID | Name | Accession | Genus | Subgenus |
|---|---|---|---|---|
| 229E | Human coronavirus 229E | Consensus | Alphacoronavirus | Duvinacovirus |
| AlphaCoV1 | Phylogenetic grouping of various animal | Consensus | Alphacoronavirus | Tegacovirus |
| | transmissible gastroenteritis viruses | | | |
| AlphaSp | Phylogenetic grouping of various | Consensus | Alphacoronavirus | unclassified |
| | alphacoronavirus sp. strains | | | |
| Anlong | Bat coronavirus isolate Anlong | Consensus | unclassified | n/a |
| | | | Coronavirinae | |
| AvianCoV | Phylogenetic grouping of avian | Consensus | Gammacoronavirus | Igacovirus |
| | coronavirus (infectious bronchitis) strains | | | |
| Beluga | Beluga Whale coronavirus | Consensus | Gammacoronavirus | Cegacovirus |
| BetaCoV1 | Phylogenetic grouping of various | Consensus | Betacoronavirus | Embecovirus |
| | embecovirus isolates | | | |
| BetaSp | Phylogenetic grouping of rodent | Consensus | Betacoronavirus | Embecovirus |
| | embecovirus isolates | | | |
| CDPHE15 | Bat coronavirus CDPHE15 | Consensus | Alphacoronavirus | Colacovirus |
| Delta | Consensus sequences of NCBI | Consensus | Deltacoronavirus | multiple |
| | Deltacoronavirus sequences | | | |
| HKU1 | Human coronavirus HKU1 | Consensus | Betacoronavirus | Embecovirus |
| HKU2 | Rhinolophus bat coronavirus HKU2 | Consensus | Alphacoronavirus | Rhinacovirus |
| HKU4 | Tylonycteris bat coronavirus HKU4 | Consensus | Betacoronavirus | Merbecovirus |
| HKU5 | Pipistrellus bat coronavirus HKU5 | Consensus | Betacoronavirus | Merbecovirus |
| HKU8 | Miniopterus bat coronavirus HKU8 | Consensus | Alphacoronavirus | Minunacovirus |
| HKU9 | Rousettus bat coronavirus HKU9 | Consensus | Betacoronavirus | Nobecovirus |
| HKU10 | Bat coronavirus HKU10 | Consensus | Alphacoronavirus | Decacovirus |
| HKU14 | rabbit coronavirus HKU14 | Consensus | Betacoronavirus | Embecovirus |
| HKU15 | Porcine coronavirus HKU15 | Consensus | Deltacoronavirus | Buldecovirus |
| HKU25 | Hypsugo bat coronavirus HKU25 | Consensus | Betacoronavirus | Merbecovirus |
| HKU31 | Erinaceus hedgehog coronavirus HKU31 | Consensus | Betacoronavirus | Merbecovirus |
| MERS | Middle East respiratory syndrome | Consensus | Betacoronavirus | Merbecovirus |
| | coronavirus | | | |
| MurineCoV | Phylogenetic grouping of rodent | Consensus | Betacoronavirus | Embecovirus |
| | embecovirus isolates | | | |
| NL63 | Human Coronavirus NL63 | Consensus | Alphacoronavirus | Setracovirus |
| PEDV | Porcine epidemic diarrhea virus | Consensus | Alphacoronavirus | Pedacovirus |
| SARSCoV2 | Severe acute respiratory syndrome | Consensus | Betacoronavirus | Sarbecovirus |
| | coronavirus 2 | | | |
| SARSCoV | Severe acute respiratory syndrome | Consensus | Betacoronavirus | Sarbecovirus |
| | coronavirus | | | |
| Sparrow | Sparrow deltacoronavirus | Consensus | Deltacoronavirus | Buldecovirus |

A further round of probe design using the same methods was targeted to a larger and yet more diverse set of coronavirus isolates (second iteration probes). See Table 2. In addition to the publicly available sequences provided in Table 2, second iteration probes were also designed to a number of proprietary sequences.

TABLE 2

| FASTA_ID | Name | Accession | Genus | Subgenus |
|---|---|---|---|---|
| 229ER | Rousettus aegyptiacus | MN611517 | Alphacoronavirus | Duvinacovirus |
| | bat coronavirus | | | |
| | 229E_related | | | |
| AcCoV_JC34 | Coronavirus | KX964649 | Alphacoronavirus | Luchacovirus |
| | AcCoV_JC34 | | | |
| Alpha2 | Mink coronavirus strain | HM245926 | Alphacoronavirus | Minacovirus |
| | WD1133 | | | |
| Alpha3398 | Alphacoronavirus | HM245925 | Alphacoronavirus | Minacovirus |
| | Bat_CoV/P.kuhlii/Italy/ | | | |
| | 3398_19/2015 | | | |
| AlphaBat | Bat alphacoronavirus | MN065811 | Alphacoronavirus | unclassified |
| AlphaMink | Alphacoronavirus | MF113046 | Alphacoronavirus | Minacovirus |
| | Mink/China/1/2016 | | | |
| BatCoV | Bat coronavirus | MG916904 | unclassified | n/a |
| | BtCoV/Rh/YN2012 | | Coronavirinae | |
| | isolate | | | |
| | BtCoV/Rh/YN2012_Ra13591 | | | |
| BatCoV1A | Bat coronavirus 1A | EU420137 | Alphacoronavirus | Minunacovirus |
| BM48_31 | Bat coronavirus | GU190215 | Betacoronavirus | Sarbecovirus |
| | BM48_31/BGR/2008 | | | |
| BtKYNL63 | NL63_related bat | KY073746 | Alphacoronavirus | Setracovirus |
| | coronavirus strain | | | |
| | BtKYNL63_9b | | | |
| BtRf_AlphaCoV | BtRf_AlphaCoV/YN2012 | KJ473808 | Alphacoronavirus | Rhinacovirus |
| BtRl_BetaCoV | Coronavirus | MK211374 | Betacoronavirus | Sarbecovirus |
| | BtRl_BetaCoV/SC2018 | | | |
| BtSk_AlphaCoV | Coronavirus | MK211372 | Alphacoronavirus | Pedacovirus |
| | BtSk_AlphaCoV/GX2018D | | | |
| CHB25 | Hipposideros pomona | MN611525 | Alphacoronavirus | Decacovirus |
| | bat coronavirus CHB25 | | | |
| FelineAlpha1 | Feline alphacoronavirus 1 | MH817484 | Alphacoronavirus | Tegacovirus |
| FerretCoV | Ferret coronavirus | NC_030292 | Alphacoronavirus | Minacovirus |
| GCCDC1 | Rousettus bat | KU762338 | Betacoronavirus | Nobecovirus |
| | coronavirus GCCDC1 | | | |
| HghCoV1 | Hedgehog coronavirus 1 | NC_039207 | Betacoronavirus | Merbecovirus |
| HKU10R | Hipposideros pomona | MN611523 | Alphacoronavirus | Decacovirus |
| | bat coronavirus | | | |
| | HKU10_related | | | |
| HKU11 | Bulbul coronavirus | FJ376620 | Deltacoronavirus | Buldecovirus |
| | HKU11 | | | |
| HKU12 | Thrush coronavirus | FJ376621 | Deltacoronavirus | Buldecovirus |
| | HKU12 | | | |
| HKU16 | White_eye coronavirus | JQ065044 | Deltacoronavirus | Buldecovirus |
| | HKU16 | | | |
| HKU17 | Sparrow coronavirus | JQ065045 | Deltacoronavirus | Buldecovirus |
| | HKU17 | | | |
| HKU18 | Magpie_robin | JQ065046 | Deltacoronavirus | Buldecovirus |
| | coronavirus HKU18 | | | |
| HKU19 | Night heron coronavirus | JQ065047 | Deltacoronavirus | Herdecovirus |
| | HKU19 | | | |
| HKU20_FJ376622 | Munia coronavirus | FJ376622 | Deltacoronavirus | Buldecovirus |
| | HKU20 | | | |
| HKU20_JQ065048 | Wigeon coronavirus | JQ065048 | Deltacoronavirus | Andecovirus |
| | HKU20 | | | |
| HKU21 | Common moorhen | JQ065049 | Deltacoronavirus | Moordecovirus |
| | coronavirus HKU21 | | | |
| HKU23 | Camel coronavirus | KT368891 | Betacoronavirus | Embecovirus |
| | HKU23 | | | |
| HKU24 | China Rattus coronavirus | NC_026011 | Betacoronavirus | Embecovirus |
| | HKU24 | | | |
| HKU2R | Rhinolophus affinis bat | MN611522 | Alphacoronavirus | Rhinacovirus |
| | coronavirus | | | |
| | HKU2_related | | | |
| HKU32 | Rhinolophus bat | MK720945 | Alphacoronavirus | Decacovirus |
| | coronavirus HKU32 | | | |
| HKU33 | Tylonycteris bat | MK720944 | Alphacoronavirus | Nyctacovirus |
| | coronavirus HKU33 | | | |
| HKU4R | Tylonycteris pachypus | MN611519 | Betacoronavirus | Merbecovirus |
| | bat coronavirus | | | |
| | HKU4_related | | | |
| HKU5R | Pipistrellus abramus bat | MN611520 | Betacoronavirus | Merbecovirus |
| | coronavirus | | | |
| | HKU5_related | | | |
| HKU8R | Miniopterus pusillus bat | MN611518 | Alphacoronavirus | Minunacovirus |
| | coronavirus | | | |
| | HKU8_related | | | |
| Hp_Beta | Bat Hp_betacoronavirus | KF636752 | Betacoronavirus | Hibecovirus |
| | Zhejiang2013 | | | |
| HuB | Rhinolophus | KJ473810 | Alphacoronavirus | Decacovirus |
| | ferrumequinum | | | |
| | alphacoronavirus | | | |
| | HuB_2013 | | | |
| MbCoV1 | Miniopterus bat | EU420138 | Alphacoronavirus | Minunacovirus |
| | coronavirus 1 | | | |
| MinkCoV1 | Mink coronavirus 1 | HM245925 | Alphacoronavirus | Minacovirus |
| MrAlphaSax | Myotis ricketti | KJ473806 | Alphacoronavirus | Myotacovirus |
| | alphacoronavirus | | | |
| | Sax_2011 | | | |
| MsbCoV1 | Miniopterus schreibersii | MN611524 | Alphacoronavirus | Minunacovirus |
| | bat coronavirus 1_related | | | |
| MvAlphaSC | Nyctalus velutinus | KJ473809 | Alphacoronavirus | Nyctacovirus |
| | alphacoronavirus | | | |
| | SC_2013 | | | |
| Quail | Quail deltacoronavirus | MH532440 | Deltacoronavirus | Buldecovirus |
| SARSRC | Severe acute respiratory | DQ648856 | Betacoronavirus | Sarbecovirus |
| | syndrome_related | | | |
| | coronavirus | | | |
| Sb512 | Scotophilus bat | NC_009657 | Alphacoronavirus | Pedacovirus |
| | coronavirus 512 | | | |
| SEAC | Swine enteric | MK977618 | Alphacoronavirus | Rhinacovirus |
| | alphacoronavirus | | | |
| Sk512R | Scotophilus kuhlii bat | MN611521 | Alphacoronavirus | Pedacovirus |
| | coronavirus 512_related | | | |
| WIV16 | SARS_like coronavirus | KT444582 | Betacoronavirus | Sarbecovirus |
| | WIV16 | | | |
| BtCoV_008_16 | Bat alphacoronavirus | MN065811 | Alphacoronavirus | unclassified |
| | strain | | | |
| | BtCoV/008_16/M.bra/FIN/ | | | |
| | 2016 | | | |
| CanadaGoose | Canada goose | MK359255 | Gammacoronavirus | unclassified |
| | coronavirus strain | | | |
| | Cambridge_Bay_2017 | | | |
| Lucheng19 | Lucheng Rn rat | KF294380 | Alphacoronavirus | Luchacovirus |
| | coronavirus isolate | | | |
| | Lucheng-19 | | | |
| Shrew | Wencheng Sm shrew | NC_035191 | Alphacoronavirus | unclassified |
| | coronavirus isolate | | | |
| | Xingguo-101 | | | |
| M95169 | Avian-infectious- | M95169 | Gammacoronavirus | Igacovirus |
| | bronchitis-virus-pol- | | | |
| | protein,-spike-protein,- | | | |
| | small-virion-associated- | | | |
| | protein,-membrane- | | | |
| | protein,-and- | | | |
| | nucleocapsid-protein- | | | |
| | genes | | | |
| AF029248 | Mouse-hepatitis-virus- | AF029248 | Betacoronavirus | Embecovirus |
| | strain-MHV-A59-C12- | | | |
| | mutant | | | |
| AF201929 | Murine-hepatitis-virus- | AF201929 | Betacoronavirus | Embecovirus |
| | strain-2 | | | |
| AF208066 | Murine-hepatitis-virus- | AF208066 | Betacoronavirus | Embecovirus |
| | strain-Penn-97-1 | | | |
| AF208067 | Murine-hepatitis-virus- | AF208067 | Betacoronavirus | Embecovirus |
| | strain-ML-10 | | | |
| AJ271965 | Transmissible- | AJ271965 | Alphacoronavirus | Tegacovirus |
| | gastroenteritis-virus- | | | |
| | complete-genome,- | | | |
| | genomic-RNA | | | |
| AJ311317 | Avian-infectious- | AJ311317 | Gammacoronavirus | Igacovirus |
| | bronchitis-virus-(strain- | | | |
| | Beaudette-CK)- | | | |
| | complete-genomic-RNA | | | |
| AY338732 | Avian-infectious- | AY338732 | Gammacoronavirus | Igacovirus |
| | bronchitis-virus-1a- | | | |
| | protein-(1a),-1ab- | | | |
| | polyprotein-(1),-S- | | | |
| | protein-(S),-3a-protein- | | | |
| | (3a),-3b-protein-(3b),-E- | | | |
| | protein-(3c),-M-protein- | | | |
| | (M),-5a-protein-(5a),-5b- | | | |
| | protein-(5b),-and- | | | |
| | nucleocapsid-protein- | | | |
| | (N)-genes | | | |
| AY692454 | Avian-infectious- | AY692454 | Gammacoronavirus | Igacovirus |
| | bronchitis-virus- | | | |
| | polyprotein-1a,- | | | |
| | polyprotein-1b,-spike- | | | |
| | protein,-3a-protein,-3b- | | | |
| | protein,-small-envelope- | | | |
| | protein,-membrane- | | | |
| | protein,-5a-protein,-5b- | | | |
| | protein,-and- | | | |
| | nucleocapsid-protein- | | | |
| | mRNAs | | | |
| AY994055 | Feline-infectious- | AY994055 | Alphacoronavirus | Tegacovirus |
| | peritonitis-virus | | | |
| AY597011 | Human-coronavirus- | AY597011 | Betacoronavirus | Embecovirus |
| | HKU1-genotype-A | | | |
| NC_009021 | Rousettus-bat- | NC_009021 | Betacoronavirus | Nobecovirus |
| | coronavirus-HKU9 | | | |
| NC_010438 | Bat-coronavirus-HKU8 | NC_010438 | Alphacoronavirus | Minunacovirus |
| NC_010646 | Beluga-Whale- | NC_010646 | Gammacoronavirus | Cegacovirus |
| | coronavirus-SW1 | | | |
| NC_010800 | Turkey-coronavirus | NC_010800 | Gammacoronavirus | Igacovirus |
| NC_012936 | Rat-coronavirus-Parker | NC_012936 | Betacoronavirus | Embecovirus |
| FN430414 | Infectious-bronchitis- | FN430414 | Gammacoronavirus | Igacovirus |
| | virus-ITA/90254/2005 | | | |
| FN430415 | Infectious-bronchitis- | FN430415 | Gammacoronavirus | Igacovirus |
| | virus-NGA/A116E7/2006 | | | |
| AB551247 | Murine-hepatitis-virus- | AB551247 | Betacoronavirus | Embecovirus |
| | RNA,-strain:-MHV-MI | | | |
| HQ166910 | Zaria-bat-coronavirus- | HQ166910 | Betacoronavirus | Hibecovirus |
| | strain-ZBCoV | | | |
| GU002364 | Fathead-minnow- | GU002364 | Bafinivirus | Pimfabavirus |
| | nidovirus-replicase- | | | |
| | polyprotein-1ab- | | | |
| | (pp1ab),-replicase- | | | |
| | polyprotein-1a-(pp1a),- | | | |
| | spike-glycoprotein-(S),- | | | |
| | membrane-protein-(M),- | | | |
| | and-nucleocapsid- | | | |
| | protein-(N)-genes | | | |
| NC_017083 | Rabbit-coronavirus- | NC_017083 | Betacoronavirus | Embecovirus |
| | HKU14 | | | |
| NC_018871 | Rousettus-bat- | NC_018871 | Alphacoronavirus | Decacovirus |
| | coronavirus-HKU10 | | | |
| NC_022103 | Bat-coronavirus- | NC_022103 | Alphacoronavirus | Colacovirus |
| | CDPHE15/USA/2006 | | | |
| KF850449 | Rat-coronavirus-strain- | KF850449 | Betacoronavirus | Embecovirus |
| | mouse_lab/USA/ | | | |
| | MG6644_HOLMES10/1996 | | | |
| KF294370 | Longquan-R1-rat- | KF294370 | Betacoronavirus | Embecovirus |
| | coronavirus-isolate- | | | |
| | Longquan-189-orf1ab- | | | |
| | polyprotein-gene;-and- | | | |
| | hemagglutinin-esterase,- | | | |
| | spike-protein,-envelope- | | | |
| | protein,-membrane- | | | |
| | protein,-and- | | | |
| | nucleocapsid-protein- | | | |
| | genes | | | |
| KF294371 | Longquan-R1-rat- | KF294371 | Betacoronavirus | Embecovirus |
| | coronavirus-isolate- | | | |
| | Longquan-370-orf1ab- | | | |
| | polyprotein-gene;-and- | | | |
| | hemagglutinin-esterase,- | | | |
| | spike-protein,-envelope- | | | |
| | protein,-membrane- | | | |
| | protein,-and- | | | |
| | nucleocapsid-protein- | | | |
| | genes | | | |
| KF294372 | Longquan-R1-rat- | KF294372 | Betacoronavirus | Embecovirus |
| | coronavirus-isolate- | | | |
| | Longquan-708-orf1ab- | | | |
| | polyprotein-gene;-and- | | | |
| | hemagglutinin-esterase,- | | | |
| | spike-protein,-envelope- | | | |
| | protein,-membrane- | | | |
| | protein,-and- | | | |
| | nucleocapsid-protein- | | | |
| | genes | | | |
| LN610099 | Guinea-fowl-coronavirus- | LN610099 | Gammacoronavirus | Igacovirus |
| | GfCoV/FR/2011- | | | |
| | complete-genome | | | |
| LC061272 | Equine-coronavirus- | LC061272 | Betacoronavirus | Embecovirus |
| | RNA,-strain:-Tokachi09 | | | |
| LC061273 | Equine-coronavirus- | LC061273 | Betacoronavirus | Embecovirus |
| | RNA,-strain:-Obihiro 12-1 | | | |
| LC061274 | Equine-coronavirus- | LC061274 | Betacoronavirus | Embecovirus |
| | RNA,-strain:-Obihiro12-2 | | | |
| KT253269 | 229E-related-bat- | KT253269 | Alphacoronavirus | Duvinacovirus |
| | coronavirus-isolate- | | | |
| | BtCoV/KW2E- | | | |
| | F151/Hip_cf._rub/GHA/ | | | |
| | 2011-orf1ab,-orf1a,- | | | |
| | spike-glycoprotein,- | | | |
| | orf4,-E,-M,-N,-and-orf8- | | | |
| | genes | | | |
| KT253270 | 229E-related-bat- | KT253270 | Alphacoronavirus | Duvinacovirus |
| | coronavirus-isolate- | | | |
| | BtCoV/FO1A- | | | |
| | F2/Hip_aba/GHA/2010- | | | |
| | orf1ab,-orf1a,-spike- | | | |
| | glycoprotein,-orf4,-E,- | | | |
| | M,-N,-and-orf8-genes | | | |
| KT253271 | 229E-related-bat- | KT253271 | Alphacoronavirus | Duvinacovirus |
| | coronavirus-isolate- | | | |
| | BtCoV/KW2E- | | | |
| | F56/Hip_cf._rub/GHA/2 | | | |
| | 011-orf1ab,-orf1a,-spike- | | | |
| | glycoprotein,-orf4,-E,- | | | |
| | M,-N,-and-orf8-genes | | | |
| KT253272 | 229E-related-bat- | KT253272 | Alphacoronavirus | Duvinacovirus |
| | coronavirus-isolate- | | | |
| | BtCoV/AT1A- | | | |
| | F1/Hip_aba/GHA/2010- | | | |
| | orf1ab,-orf1a,-spike- | | | |
| | glycoprotein,-orf4,-E,- | | | |
| | M,-N,-and-orf8-genes | | | |
| NC_028752 | Camel-alphacoronavirus- | NC_028752 | Alphacoronavirus | Duvinacovirus |
| | isolate- | | | |
| | camel/Riyadh/Ry141/2015 | | | |
| NC_028806 | Swine-enteric- | NC_028806 | Alphacoronavirus | Tegacovirus |
| | coronavirus-strain- | | | |
| | Italy/213306/2009 | | | |
| LC119077 | Ferret-coronavirus- | LC119077 | Alphacoronavirus | Minacovirus |
| | genomic-RNA,-strain:- | | | |
| | FRCoV4370 | | | |
| NC_032107 | NL63-related-bat- | NC_032107 | Alphacoronavirus | Setracovirus |
| | coronavirus-strain- | | | |
| | BtKYNL63-9a | | | |
| KU556804 | Infectious-bronchitis- | KU556804 | Gammacoronavirus | Igacovirus |
| | virus-isolate- | | | |
| | Ck/Aus/N1/88 | | | |
| KU556805 | Infectious-bronchitis- | KU556805 | Gammacoronavirus | Igacovirus |
| | virus-isolate-Armidale-A3 | | | |
| KU556806 | Infectious-bronchitis- | KU556806 | Gammacoronavirus | Igacovirus |
| | virus-isolate- | | | |
| | Ck/Aus/N1/03 | | | |
| KU556807 | Infectious-bronchitis- | KU556807 | Gammacoronavirus | Igacovirus |
| | virus-isolate- | | | |
| | Ck/Aus/N1/08 | | | |
| NC_032730 | Lucheng-Rn-rat- | NC_032730 | Alphacoronavirus | Luchacovirus |
| | coronavirus-isolate- | | | |
| | Lucheng-19 | | | |
| KX721498 | Infectious-bronchitis- | KX721498 | Gammacoronavirus | Igacovirus |
| | virus-isolate-SCZJ-2 | | | |
| KY419111 | Porcine- | KY419111 | Betacoronavirus | Embecovirus |
| | hemagglutinating- | | | |
| | encephalomyelitis-virus- | | | |
| | strain-PHEV- | | | |
| | CoV/swine/USA/15TOSU1727/ | | | |
| | 2015 | | | |
| KY588134 | Infectious-bronchitis- | KY588134 | Gammacoronavirus | Igacovirus |
| | virus-isolate- | | | |
| | Pakistan/Mass/1003/2A/2015 | | | |
| KY588135 | Infectious-bronchitis- | KY588135 | Gammacoronavirus | Igacovirus |
| | virus-isolate- | | | |
| | Pakistan/Mass/1009/13A/2015 | | | |
| NC_034440 | Bat-coronavirus-isolate- | NC_034440 | unclassified | n/a |
| | PREDICT/PDF-2180 | | Coronavirinae | |
| KY983584 | Human-coronavirus- | KY983584 | Betacoronavirus | Embecovirus |
| | HKU1-strain-SC2628 | | | |
| NC_034976 | Goat-torovirus-strain-SZ | NC_034976 | Gammacoronavirus | Igacovirus |
| KY273667 | Infectious-bronchitis- | KY273667 | Gammacoronavirus | Igacovirus |
| | virus-isolate- | | | |
| | Jordan/Mass/15/2004 | | | |
| LC215871 | Ferret-coronavirus- | LC215871 | Alphacoronavirus | Minacovirus |
| | genomic-RNA,-strain:- | | | |
| | ferret063 | | | |
| KX722531 | Feline-coronavirus- | KX722531 | Alphacoronavirus | Tegacovirus |
| | isolate-Cat-2-Holstebro | | | |
| KY370043 | Rodent-coronavirus- | KY370043 | unclassified | n/a |
| | isolate-RtRn- | | Coronavirinae | |
| | CoV/YN2013-ORF1ab- | | | |
| | polyprotein,- | | | |
| | hemagglutinin-esterase- | | | |
| | protein,-spike- | | | |
| | glycoprotein,-envelope- | | | |
| | protein,-membrane- | | | |
| | protein,-and- | | | |
| | nucleocapsid-protein- | | | |
| | genes | | | |
| KY370044 | Rodent-coronavirus- | KY370044 | unclassified | n/a |
| | isolate-RtAs- | | Coronavirinae | |
| | CoV/IM2014-ORF1ab- | | | |
| | polyprotein,- | | | |
| | hemagglutinin-esterase- | | | |
| | protein,-spike- | | | |
| | glycoprotein,-envelope- | | | |
| | protein,-membrane- | | | |
| | protein,-and- | | | |
| | nucleocapsid-protein- | | | |
| | genes | | | |
| KY370045 | Rodent-coronavirus- | KY370045 | unclassified | n/a |
| | isolate-RtMruf-CoV- | | Coronavirinae | |
| | 1/JL2014-ORF1ab- | | | |
| | polyprotein,-spike- | | | |
| | glycoprotein,-envelope- | | | |
| | protein,-membrane- | | | |
| | protein,-and- | | | |
| | nucleocapsid-protein- | | | |
| | genes | | | |
| KY370046 | Rodent-coronavirus- | KY370046 | unclassified | n/a |
| | isolate-RtMruf-CoV- | | Coronavirinae | |
| | 2/JL2014-ORF1ab- | | | |
| | polyprotein,- | | | |
| | hemagglutinin-esterase- | | | |
| | protein,-spike- | | | |
| | glycoprotein,-envelope- | | | |
| | protein,-membrane- | | | |
| | protein,-and- | | | |
| | nucleocapsid-protein- | | | |
| | genes | | | |
| KY370048 | Rodent-coronavirus- | KY370048 | unclassified | n/a |
| | isolate-RtMm- | | Coronavirinae | |
| | CoV/GD2015-ORF1ab- | | | |
| | polyprotein,- | | | |
| | hemagglutinin-esterase- | | | |
| | protein,-spike- | | | |
| | glycoprotein,-envelope- | | | |
| | protein,-membrane- | | | |
| | protein,-and- | | | |
| | nucleocapsid-protein- | | | |
| | genes | | | |
| KY370049 | Rodent-coronavirus- | KY370049 | unclassified | n/a |
| | isolate-RtNn- | | Coronavirinae | |
| | CoV/SAX2015-ORF1ab- | | | |
| | polyprotein,- | | | |
| | hemagglutinin-esterase- | | | |
| | protein,-spike- | | | |
| | glycoprotein,-envelope- | | | |
| | protein,-membrane- | | | |
| | protein,-and- | | | |
| | nucleocapsid-protein- | | | |
| | genes | | | |
| KY370050 | Rodent-coronavirus- | KY370050 | unclassified | n/a |
| | isolate-RtRl- | | Coronavirinae | |
| | CoV/FJ2015-ORF1ab- | | | |
| | polyprotein,-spike- | | | |
| | glycoprotein,-envelope- | | | |
| | protein,-membrane- | | | |
| | protein,-and- | | | |
| | nucleocapsid-protein- | | | |
| | genes | | | |
| KY370051 | Rodent-coronavirus- | KY370051 | unclassified | n/a |
| | isolate-RtBi- | | Coronavirinae | |
| | CoV/FJ2015-ORF1ab- | | | |
| | polyprotein,- | | | |
| | hemagglutinin-esterase- | | | |
| | protein,-spike- | | | |
| | glycoprotein,-envelope- | | | |
| | protein,-membrane- | | | |
| | protein,-and- | | | |
| | nucleocapsid-protein- | | | |
| | genes | | | |
| KY370052 | Rodent-coronavirus- | KY370052 | unclassified | n/a |
| | isolate-RtMm-CoV- | | Coronavirinae | |
| | 1/IM2014-ORF1ab- | | | |
| | polyprotein,- | | | |
| | hemagglutinin-esterase- | | | |
| | protein,-spike- | | | |
| | glycoprotein,-envelope- | | | |
| | protein,-membrane- | | | |
| | protein,-and- | | | |
| | nucleocapsid-protein- | | | |
| | genes | | | |
| KY370053 | Shrew-coronavirus- | KY370053 | Alphacoronavirus | Soracovirus |
| | isolate-Shrew- | | | |
| | CoV/Tibet2014- | | | |
| | ORF1ab-polyprotein,- | | | |
| | spike-glycoprotein,- | | | |
| | envelope-protein,- | | | |
| | membrane-protein,-and- | | | |
| | nucleocapsid-protein- | | | |
| | genes | | | |
| KY370054 | Rodent-coronavirus- | KY370054 | unclassified | n/a |
| | isolate-RtClan- | | Coronavirinae | |
| | CoV/GZ2015-ORF1ab- | | | |
| | polyprotein-gene;-and- | | | |
| | spike-glycoprotein,- | | | |
| | envelope-protein,- | | | |
| | membrane-protein,-and- | | | |
| | nucleocapsid-protein- | | | |
| | genes | | | |
| MF416379 | Murine-hepatitis-virus- | MF416379 | Betacoronavirus | Embecovirus |
| | isolate- | | | |
| | MHV/NYC/Manhattan/poolF5 | | | |
| MG428700 | Human-coronavirus- | MG428700 | Alphacoronavirus | Setracovirus |
| | NL63-isolate- | | | |
| | Kilifi_HH_1602_01-Jun-2010 | | | |
| MG693168 | Bat-coronavirus-isolate- | MG693168 | unclassified | n/a |
| | CMR704-P12-ORF1a,- | | Coronavirinae | |
| | ORF1b,-spike-protein,- | | | |
| | ORF3,-Membrane- | | | |
| | protein,-capsid,- | | | |
| | hypothetical-protein- | | | |
| | ORFx,-and-hypothetical- | | | |
| | protein-ORFy-genes | | | |
| MG693169 | Bat-coronavirus-isolate- | MG693169 | unclassified | n/a |
| | CMR900-ORF1a,- | | Coronavirinae | |
| | ORF1b,-Spike-protein,- | | | |
| | ORF3,-Membrane- | | | |
| | protein,-capsid,- | | | |
| | hypothetical-protein- | | | |
| | ORFx,-and-hypothetical- | | | |
| | protein-ORFy-genes | | | |
| MG693170 | Bat-coronavirus-isolate- | MG693170 | unclassified | n/a |
| | CMR66-ORF1a,- | | Coronavirinae | |
| | ORF1b,-Spike-protein,- | | | |
| | ORF3,-Membrane- | | | |
| | protein,-capsid,- | | | |
| | hypothetical-protein- | | | |
| | ORFx,-and-hypothetical- | | | |
| | protein-ORFy-genes | | | |
| MG693171 | Bat-coronavirus-isolate- | MG693171 | unclassified | n/a |
| | CMR891-892-ORF1a- | | Coronavirinae | |
| | gene;-and-ORF1b,- | | | |
| | Spike-protein,-ORF3,- | | | |
| | Membrane-protein,- | | | |
| | capsid,-hypothetical- | | | |
| | protein-ORFx,-and- | | | |
| | hypothetical-protein- | | | |
| | ORFy-genes | | | |
| MG693172 | Bat-coronavirus-isolate- | MG693172 | unclassified | n/a |
| | CMR705-P13-ORF1a,- | | Coronavirinae | |
| | ORF1b,-Spike-protein,- | | | |
| | ORF3,-Membrane- | | | |
| | protein,-capsid,- | | | |
| | hypothetical-protein- | | | |
| | ORFx,-and-hypothetical- | | | |
| | protein-ORFy-genes | | | |
| NC_038294 | Betacoronavirus- | NC_038294 | Betacoronavirus | Merbecovirus |
| | England-1-isolate- | | | |
| | H123990006 | | | |
| MK163627 | Alphacoronavirus- | MK163627 | Alphacoronavirus | |
| | UKRn3-pp1a,-pp1ab,- | | | |
| | NS2,-spike,-NS4,-E- | | | |
| | protein,-M-protein,- | | | |
| | NS7a,-nucleocapsid,- | | | |
| | and-NS9-genes | | | |
| MG923574 | Bat-alphacoronavirus- | MG923574 | Alphacoronavirus | unclassified |
| | isolate- | | | |
| | BtCoV/020_16/M.dau/FIN/ | | | |
| | 2016-polyprotein,- | | | |
| | spike-protein,- | | | |
| | hypothetical-protein,- | | | |
| | envelope-protein,- | | | |
| | membrane-protein,-and- | | | |
| | nucleocapsid-protein- | | | |
| | genes | | | |
| MK953937 | Infectious-bronchitis- | MK953937 | Gammacoronavirus | Igacovirus |
| | virus-strain-Brazil/SP55 | | | |
| MK957244 | Infectious-bronchitis- | MK957244 | Gammacoronavirus | Igacovirus |
| | virus-strain-PR05 | | | |
| MK957245 | Infectious-bronchitis- | MK957245 | Gammacoronavirus | Igacovirus |
| | virus-strain-PR01 | | | |
| MK204393 | Avian-coronavirus-non- | MK204393 | Gammacoronavirus | Igacovirus |
| | structural-polyprotein- | | | |
| | (ORF1ab)-and-spike- | | | |
| | protein-(S)-genes | | | |
| MK204411 | Avian-coronavirus-non- | MK204411 | Gammacoronavirus | Igacovirus |
| | structural-polyprotein- | | | |
| | (ORF1ab),-spike- | | | |
| | protein-(S),-3a-(3a),-3b- | | | |
| | (3b),-envelope-protein- | | | |
| | (3c),-membrane- | | | |
| | glycoprotein,-5a-(5a),- | | | |
| | 5b-(5b),-and- | | | |
| | nucleocapsid-(N)-genes | | | |
| LC469308 | Bat-coronavirus-Vs- | LC469308 | unclassified | n/a |
| | CoV-1-genomic-RNA | | Coronavirinae | |
| MK618759 | Infectious-bronchitis- | MK618759 | Gammacoronavirus | Igacovirus |
| | virus-isolate-K047-12 | | | |
| MT040333 | Pangolin-coronavirus- | MT040333 | Betacoronavirus | Sarbecovirus |
| | isolate-PCoV_GX-P4L | | | |
| MT040334 | Pangolin-coronavirus- | MT040334 | Betacoronavirus | Sarbecovirus |
| | isolate-PCoV_GX-P1E | | | |
| MT040335 | Pangolin-coronavirus- | MT040335 | Betacoronavirus | Sarbecovirus |
| | isolate-PCoV_GX-P5L | | | |
| MT040336 | Pangolin-coronavirus- | MT040336 | Betacoronavirus | Sarbecovirus |
| | isolate-PCoV_GX-P5E | | | |
| MT084071 | Pangolin-coronavirus- | MT084071 | Betacoronavirus | Sarbecovirus |
| | isolate-MP789-genomic- | | | |
| | sequence | | | |
| MT121216 | Pangolin-coronavirus- | MT121216 | Betacoronavirus | Sarbecovirus |
| | isolate-MP789 | | | |
| MN690608 | Bottlenose-dolphin- | MN690608 | Gammacoronavirus | Cegacovirus |
| | coronavirus-strain- | | | |
| | 37112-1 | | | |
| MN690609 | Bottlenose-dolphin- | MN690609 | Gammacoronavirus | Cegacovirus |
| | coronavirus-strain- | | | |
| | 37112-2 | | | |
| MN690610 | Bottlenose-dolphin- | MN690610 | Gammacoronavirus | Cegacovirus |
| | coronavirus-strain- | | | |
| | 37112-3 | | | |
| MN690611 | Bottlenose-dolphin- | MN690611 | Gammacoronavirus | Cegacovirus |
| | coronavirus-strain- | | | |
| | 37112-4 | | | |
| MN262644 | Avian-coronavirus- | MN262644 | Gammacoronavirus | Igacovirus |
| | strain-CV10 | | | |
| MN711790 | Infectious-bronchitis- | MN711790 | Gammacoronavirus | Igacovirus |
| | virus-isolate- | | | |
| | GA/1472/2004 | | | |
| MN199462 | Infectious-bronchitis- | MN199462 | Gammacoronavirus | Igacovirus |
| | virus-isolate-R17/16 | | | |
| MN199463 | Infectious-bronchitis- | MN199463 | Gammacoronavirus | Igacovirus |
| | virus-isolate-R17/19 | | | |
| MN199464 | Infectious-bronchitis- | MN199464 | Gammacoronavirus | Igacovirus |
| | virus-isolate-R17/27 | | | |
| MN199465 | Infectious-bronchitis- | MN199465 | Gammacoronavirus | Igacovirus |
| | virus-isolate-R17/36 | | | |
| MN199466 | Infectious-bronchitis- | MN199466 | Gammacoronavirus | Igacovirus |
| | virus-isolate-R18/23 | | | |
| MT072864 | Pangolin-coronavirus- | MT072864 | Betacoronavirus | Sarbecovirus |
| | isolate-PCoV_GX-P2V | | | |
| MT072865 | Pangolin-coronavirus- | MT072865 | Betacoronavirus | Sarbecovirus |
| | isolate-PCoV_GX-P3B- | | | |
| | genomic-sequence | | | |
| MN128086 | Infectious-bronchitis- | MN128086 | Gammacoronavirus | Igacovirus |
| | virus-strain- | | | |
| | TW2296/95 vac | | | |
| MN128087 | Infectious-bronchitis- | MN128087 | Gammacoronavirus | Igacovirus |
| | virus-strain- | | | |
| | TW2575/98 vac | | | |
| MN128088 | Infectious-bronchitis- | MN128088 | Gammacoronavirus | Igacovirus |
| | virus-strain- | | | |
| | TW2296/95w | | | |
| NC_046954 | Rodent-coronavirus- | NC_046954 | unclassified | n/a |
| | isolate-RtMruf-CoV- | | Coronavirinae | |
| | 2/JL2014 | | | |
| NC_046955 | Shrew-coronavirus- | NC_046955 | unclassified | n/a |
| | isolate-Shrew- | | Coronavirinae | |
| | CoV/Tibet2014- | | | |
| | ORF1ab-polyprotein,- | | | |
| | spike-glycoprotein,- | | | |
| | envelope-protein,- | | | |
| | membrane-protein,-and- | | | |
| | nucleocapsid-protein- | | | |
| | genes | | | |
| NC_046965 | Canada-goose- | NC_046965 | Gammacoronavirus | unclassified |
| | coronavirus-strain- | | | |
| | Cambridge_Bay_2017 | | | |
| MN307884 | Infectious-bronchitis- | MN307884 | Gammacoronavirus | Igacovirus |
| | virus-isolate- | | | |
| | ck/CH/LJX/2017/07 | | | |
| MT114538 | Canine-coronavirus- | MT114538 | Alphacoronavirus | Tegacovirus |
| | strain-B639_ZJ_2019 | | | |
| MT114539 | Canine-coronavirus- | MT114539 | Alphacoronavirus | Tegacovirus |
| | strain-B600_ZJ_2019 | | | |
| MT114540 | Canine-coronavirus- | MT114540 | Alphacoronavirus | Tegacovirus |
| | strain-B447_ZJ_2019 | | | |
| MT114541 | Canine-coronavirus- | MT114541 | Alphacoronavirus | Tegacovirus |
| | strain-B363_ZJ_2019 | | | |
| MT114542 | Canine-coronavirus- | MT114542 | Alphacoronavirus | Tegacovirus |
| | strain-B203_GZ_2019 | | | |
| MT114543 | Canine-coronavirus- | MT114543 | Alphacoronavirus | Tegacovirus |
| | strain-B194_GZ_2019 | | | |
| MT114544 | Canine-coronavirus- | MT114544 | Alphacoronavirus | Tegacovirus |
| | strain-B135_JS_2018 | | | |
| MN794188 | Infectious-bronchitis- | MN794188 | Gammacoronavirus | Igacovirus |
| | virus-strain-I0305/19 | | | |
| MN509587 | Infectious-bronchitis- | MN509587 | Gammacoronavirus | Igacovirus |
| | virus-strain- | | | |
| | cK/CH/LDL/091021 | | | |
| MN509588 | Infectious-bronchitis- | MN509588 | Gammacoronavirus | Igacovirus |
| | virus-strain-I0730/17 | | | |
| MN509589 | Infectious-bronchitis- | MN509589 | Gammacoronavirus | Igacovirus |
| | virus-strain- | | | |
| | cK/CH/LHLJ/110664 | | | |
| MN517816 | Infectious-bronchitis- | MN517816 | Gammacoronavirus | Igacovirus |
| | virus-strain- | | | |
| | cK/CH/LJL/090608 | | | |
| MN517817 | Infectious-bronchitis- | MN517817 | Gammacoronavirus | Igacovirus |
| | virus-strain- | | | |
| | cK/CH/LSD/110723 | | | |
| MN531554 | Infectious-bronchitis- | MN531554 | Gammacoronavirus | Igacovirus |
| | virus-strain- | | | |
| | cK/CH/LLN/120611 | | | |
| MN531555 | Infectious-bronchitis- | MN531555 | Gammacoronavirus | Igacovirus |
| | virus-strain- | | | |
| | cK/CH/LSD/1112145 | | | |
| MN531556 | Infectious-bronchitis- | MN531556 | Gammacoronavirus | Igacovirus |
| | virus-strain- | | | |
| | cK/CH/LSD/110856 | | | |
| NC_048212 | Bat-coronavirus | NC_048212 | unclassified | n/a |
| | | | Coronavirinae | |
| NC_048213 | Infectious-bronchitis- | NC_048213 | Gammacoronavirus | Igacovirus |
| | virus-isolate-Ind-TN92-03 | | | |
| NC_048214 | Duck-coronavirus- | NC_048214 | Gammacoronavirus | Igacovirus |
| | isolate-DK/GD/27/2014 | | | |
| NC_048216 | NL63-related-bat- | NC_048216 | Alphacoronavirus | Setracovirus |
| | coronavirus-strain- | | | |
| | BtKYNL63-9b | | | |
| NC_048217 | Murine-hepatitis-virus- | NC_048217 | Betacoronavirus | Embecovirus |
| | strain-A59 | | | |
| MT337384 | Bat-coronavirus-HKU9- | MT337384 | Betacoronavirus | Nobecovirus |
| | 1-isolate- | | | |
| | MCL_20_Bat_76_10 | | | |
| MT337385 | Coronavirus-BtRt- | MT337385 | Betacoronavirus | Nobecovirus |
| | BetaCoV/GX2018- | | | |
| | isolate- | | | |
| | MCL_20_Bat_76_8 | | | |
| MT337386 | Coronavirus-BtRt- | MT337386 | Betacoronavirus | Nobecovirus |
| | BetaCoV/GX2018- | | | |
| | isolate- | | | |
| | MCL_19_Bat_606_2 | | | |
| MT337387 | Coronavirus-BtRt- | MT337387 | Betacoronavirus | Nobecovirus |
| | BetaCoV/GX2018- | | | |
| | isolate- | | | |
| | MCL_19_Bat_606_10 | | | |
| MN548286 | Infectious-bronchitis- | MN548286 | Gammacoronavirus | Igacovirus |
| | virus-strain-D1466 | | | |
| MN548287 | Infectious-bronchitis- | MN548287 | Gammacoronavirus | Igacovirus |
| | virus-strain-H120 | | | |
| MN548288 | Infectious-bronchitis- | MN548288 | Gammacoronavirus | Igacovirus |
| | virus-strain-Italy02 | | | |
| MN548289 | Infectious-bronchitis- | MN548289 | Gammacoronavirus | Igacovirus |
| | virus-strain-QX | | | |
| MN692770 | Swine-enteric- | MN692770 | Alphacoronavirus | Tegacovirus |
| | coronavirus-isolate- | | | |
| | SeCoV-1480-Murcia- | | | |
| | Lorca | | | |
| MT663548 | Bat-alphacoronavirus- | MT663548 | Alphacoronavirus | unclassified |
| | isolate-AMA_L_F | | | |
| MN548285 | Infectious-bronchitis- | MN548285 | Gammacoronavirus | Igacovirus |
| | virus-strain-CR88 | | | |
| MN987230 | Infectious-bronchitis- | MN987230 | Gammacoronavirus | Igacovirus |
| | virus-isolate- | | | |
| | IBV/ck/EGY- | | | |
| | Monuf/NR725/16 | | | |
| MN987231 | Infectious-bronchitis- | MN987231 | Gammacoronavirus | Igacovirus |
| | virus-isolate- | | | |
| | IBV/ck/EGY- | | | |
| | Monuf/USC-5/13 | | | |
| MW251308 | Bat-coronavirus- | MW251308 | Betacoronavirus | Sarbecovirus |
| | RacCS203 | | | |

### A. Deduplication of Probes

Because the second set of virus inputs for developing the second iteration probes represented a less curated set of sequences, it was expected that there would be a very large degree of sequence homology across the various genomes. Therefore, the present probe set (first iteration probes and second iteration probes) was subjected to deduplication using the dedupe algorithm from the Joint Genome Institute as part of their BBTools suite of bioinformatic software tools. (jgi.doe.gov/data-and-tools/software-tools/bbtools/) The probe set was deduplicated using the Clustering by overlap method. In short, highly homologous probes were removed from the probe set and only the ‘best representative’ identified by the clustering algorithm were retained.

### B. Specificity Check

The combination of probes comprising the first iteration probes and the second iteration probes is henceforth referred to as the v1 probe set. This probe set was then tested for theoretical pull-down efficacy against the entirety of the coronavirus isolate sequences that we had downloaded from NCBI in 2020, which contained ˜2700 sequences. Theoretical pulldown was calculated using both high and low stringency assumptions, which consisted of 70% minimum identity over 30 bp for low and 90% minimum identity over 70 bp for high stringency. Using the higher stringency requirements, a gap analysis of the sequences (i.e., the sequences expected *not* to be captured) was generated.

C. Probe Set backfill

From this analysis a BED file was generated, and all gaps in sequences were used as input regions for the proprietary algorithm to generate another set of probes to backfill regions that for any reason were not showing up as ‘covered’ in the analysis. In many cases, only 1 or a few extra probes were added per genome. See Table 3. This probe set was then subjected to the deduplication step as outlined above. SEQ ID NOs: 1-22909 resulted from this analysis.

TABLE 3

| Accession | Name | Accession | Name |
|---|---|---|---|
| KF636752 | Bat Hp- | KP202848 | Transmissible gastroenteritis virus |
| | betacoronavirus/Zhejiang2013 | | isolate SHXB |
| KM347965 | Ferret coronavirus isolate FRCoV- | KP780179 | Infectious bronchitis virus strain |
| | NL-2010 | | gammaCoV/Ck/Italy/I2022/13 |
| KX512809 | Ferret enteric coronavirus strain | KP849472 | Alphacoronavirus 1 strain 23/03 |
| | FECV1 | | |
| KX512810 | Ferret systemic coronavirus strain | KP886808 | Bat SARS-like coronavirus |
| | FSCV6 | | YNLF_31C |
| HM245925 | Mink coronavirus strain WD1127 | KP886809 | Bat SARS-like coronavirus |
| | | | YNLF_34C |
| KY073745 | NL63-related bat coronavirus | KR608272 | Infectious bronchitis virus isolate |
| | strain BtKYNL63-9b | | LDT3-A |
| KY073746 | NL63-related bat coronavirus | KR822424 | European turkey coronavirus 080385d |
| | strain BtKYNL63-15 | | |
| KJ473809 | BtNv-AlphaCoV/SC2013 | KT368891 | Camel coronavirus HKU23 isolate |
| | | | camel/Riyadh/Ry123/2015 |
| KU762338 | Rousettus bat coronavirus isolate | KT444582 | SARS-like coronavirus WIV16 |
| | GCCDC1 356 | | |
| MK720944 | Tylonycteris bat coronavirus | KT696544 | Transmissible gastroenteritis virus |
| | HKU33 strain GZ151867 | | strain JS2012 |
| MF113046 | Alphacoronavirus | KT852992 | Infectious bronchitis virus isolate |
| | Mink/China/1/2016 | | tl/CH/LDT3/03 |
| MH938449 | Alphacoronavirus Bat- | KU131570 | Human coronavirus OC43 strain |
| | CoV/P.kuhlii/Italy/3398-19/2015 | | HCoV-OC43/UK/London/2011 |
| MH938450 | Alphacoronavirus Bat- | KU182964 | Bat coronavirus isolate JTMC15 |
| | CoV/P.kuhlii/Italy/206679-3/2010 | | |
| MH938448 | Alphacoronavirus Bat- | KU215419 | Feline coronavirus isolate inoculum |
| | CoV/P.kuhlii/Italy/206645- | | |
| | 41/2011 | | |
| HM245926 | Mink coronavirus strain WD1133 | KU215420 | Feline coronavirus isolate Cat1_day7 |
| MG916901 | Bat coronavirus | KU215421 | Feline coronavirus isolate |
| | BtCoV/Rh/YN2012 isolate | | Cat2_day21_deletion |
| | BtCoV/Rh/YN2012_Rs3376 | | |
| MG916902 | Bat coronavirus | KU215422 | Feline coronavirus isolate |
| | BtCoV/Rh/YN2012 isolate | | Cat2_day21_withoutdeletion |
| | BtCoV/Rh/YN2012_Rs4125 | | |
| MG916903 | Bat coronavirus | KU215423 | Feline coronavirus isolate Cat3_day9 |
| | BtCoV/Rh/YN2012 isolate | | |
| | BtCoV/Rh/YN2012_Rs4259 | | |
| MG916904 | Bat coronavirus | KU215424 | Feline coronavirus isolate |
| | BtCoV/Rh/YN2012 isolate | | Cat1_day28_deletion |
| | BtCoV/Rh/YN2012_Ra13591 | | |
| MK492263 | Bat coronavirus strain BtCoV92 | KU215425 | Feline coronavirus isolate |
| | | | Cat1_day28_withoutdeletion |
| MN197549 | Infectious bronchitis virus isolate | KU215426 | Feline coronavirus isolate Cat2_day84 |
| | CK/CH/GD/GDTS13 | | |
| MN512434 | Infectious bronchitis virus isolate | KU215427 | Feline coronavirus isolate |
| | IBV/Ck/Can/17-035614 | | Cat3_day28_deletion |
| MN512435 | Infectious bronchitis virus isolate | KU215428 | Feline coronavirus isolate |
| | IBV/Ck/Can/17-036989 | | Cat3_day28_withoutdeletion |
| MN512436 | Infectious bronchitis virus isolate | KU291448 | Human coronavirus 229E isolate |
| | IBV/Ck/Can/18-048192T | | HCoV-229E/BN1/GER/2015 |
| MN512437 | Infectious bronchitis virus isolate | KU361188 | Infectious bronchitis virus isolate |
| | IBV/Ck/Can/18-048430 | | CK/CH/2014/QL1403 |
| MN512438 | Infectious bronchitis virus isolate | KU558922 | Betacoronavirus 1 isolate Buffalo |
| | IBV/Ck/Can/18-049707 | | coronavirus B1-24F |
| MN096598 | Infectious bronchitis virus strain | KU558923 | Betacoronavirus 1 isolate Buffalo |
| | ck/CH/YNSL/160501 | | coronavirus B1-28F |
| MK581202 | Infectious bronchitis virus strain | KU729220 | Transmissible gastroenteritis virus |
| | gammaCoV/Ck/Poland/80/1989 | | strain TH-98 |
| MK581204 | Infectious bronchitis virus strain | KU762338 | Rousettus bat coronavirus isolate |
| | gammaCoV/Ck/Poland/255/1997 | | GCCDC1 356 |
| MK329221 | Infectious bronchitis virus isolate | KU886219 | Bovine coronavirus strain BCV-AKS- |
| | CK-CH-GX-YL17 | | 01 |
| MK309398 | Infectious bronchitis virus isolate | KU973692 | UNVERIFIED: SARS-related |
| | CK/CH/GD/HY16 | | coronavirus isolate F46 |
| MK217372 | Infectious bronchitis virus strain | KX083668 | Transmissible gastroenteritis virus |
| | I0221/17 | | isolate HE-1 |
| MK217373 | Infectious bronchitis virus strain | KX185057 | Infectious bronchitis virus strain |
| | I0725/17 | | ck/CH/LHLJ/95I |
| MK217374 | Infectious bronchitis virus strain | KX252791 | Infectious bronchitis virus strain |
| | I0916/16 | | ck/CH/LLN/98I |
| MK217375 | Infectious bronchitis virus strain | KX266757 | Infectious bronchitis virus isolate |
| | I1209/16 | | 3575/08 |
| MK574042 | Infectious bronchitis virus strain | KX344031 | Human coronavirus OC43 isolate |
| | ck/CH/LHB/110615 | | LRTI_238 |
| MK574043 | Infectious bronchitis virus strain | KX432213 | Canine respiratory coronavirus strain |
| | ck/CH/LHB/110617 | | BJ232 |
| MK142676 | Infectious bronchitis virus isolate | KX499468 | Transmissible gastroenteritis virus |
| | ahysx-1 | | strain TGEV AHHF |
| MK878536 | Infectious bronchitis virus isolate | KX512809 | Ferret enteric coronavirus strain |
| | GA9977/2019 | | FECV1 |
| MH924835 | Infectious bronchitis virus strain | KX512810 | Ferret systemic coronavirus strain |
| | gammaCoV/ck/China/I0636/16 | | FSCV6 |
| MH878976 | Infectious bronchitis virus isolate | KX538964 | Human coronavirus OC43 isolate MY- |
| | VFAR-047 | | U002/12 |
| MG021194 | Infectious bronchitis virus isolate | KX538965 | Human coronavirus OC43 isolate MY- |
| | gammaCoV/AvCov/Ck/Italy/624I/ | | U208/12 |
| | 96 | | |
| MH021175 | Avian coronavirus strain D274 | KX538966 | Human coronavirus OC43 isolate MY- |
| | | | U236/12 |
| MG448607 | Infectious bronchitis virus strain | KX538967 | Human coronavirus OC43 isolate MY- |
| | ck/CH/LHB/121042 | | U413/12 |
| MF882923 | Infectious bronchitis virus isolate | KX538968 | Human coronavirus OC43 isolate MY- |
| | CK/CH/HB/2016 | | U464/12 |
| MF421319 | Infectious bronchitis virus isolate | KX538969 | Human coronavirus OC43 isolate MY- |
| | UY/09/CA/01 | | U523/12 |
| KX252791 | Infectious bronchitis virus strain | KX538970 | Human coronavirus OC43 isolate MY- |
| | ck/CH/LLN/98I | | U710/12 |
| KX185057 | Infectious bronchitis virus strain | KX538971 | Human coronavirus OC43 isolate MY- |
| | ck/CH/LHLJ/95I | | U732/12 |
| KX266757 | Infectious bronchitis virus isolate | KX538972 | Human coronavirus OC43 isolate MY- |
| | 3575/08 | | U774/12 |
| KU361188 | Infectious bronchitis virus isolate | KX538973 | Human coronavirus OC43 isolate MY- |
| | CK/CH/2014/QL1403 | | U868/12 |
| KR608272 | Infectious bronchitis virus isolate | KX538974 | Human coronavirus OC43 isolate MY- |
| | LDT3-A | | U945/12 |
| KT852992 | Infectious bronchitis virus isolate | KX538975 | Human coronavirus OC43 isolate MY- |
| | tl/CH/LDT3/03 | | U1024/12 |
| KR822424 | European turkey coronavirus | KX538976 | Human coronavirus OC43 isolate MY- |
| | 080385d | | U1057/12 |
| KP780179 | Infectious bronchitis virus strain | KX538977 | Human coronavirus OC43 isolate MY- |
| | gammaCoV/Ck/Italy/I2022/13 | | U1140/12 |
| KM454473 | Duck coronavirus isolate | KX538978 | Human coronavirus OC43 isolate MY- |
| | DK/GD/27/2014 | | U1758/13 |
| KF460437 | Infectious bronchitis virus isolate | KX538979 | Human coronavirus OC43 isolate MY- |
| | VicS-v | | U1975/13 |
| KF931628 | Infectious bronchitis virus isolate | KX574227 | Bat coronavirus isolate |
| | VicS-del | | PREDICT/PDF-2180 |
| KM213963 | Infectious bronchitis virus isolate | KX722529 | Feline coronavirus isolate UG-FH8 |
| | CK/CH/XDC- 2/2013 | | |
| KF663559 | Infectious bronchitis virus isolate | KX722530 | Feline coronavirus isolate Cat 1 |
| | ck/CH/IBTZ/2012 | | Karlslunde |
| KF668605 | Infectious bronchitis virus isolate | KX900393 | Transmissible gastroenteritis virus |
| | CK/CH/SD09/005 | | strain TGEV/USA/Z/1986 |
| JX897900 | UNVERIFIED: Infectious | KX900394 | Transmissible gastroenteritis virus |
| | bronchitis virus isolate GX- | | strain TGEV/USA/HB/1988 |
| | NN09032 | | |
| JQ977697 | Infectious bronchitis virus isolate | KX900395 | Transmissible gastroenteritis virus |
| | SNU8067 | | strain TGEV/USA/Minnesota138/2006 |
| GU393332 | Infectious bronchitis virus serotype | KX900396 | Transmissible gastroenteritis virus |
| | Delaware 072 | | strain TGEV/USA/Illinois139/2006 |
| JF330898 | Infectious bronchitis virus strain | KX900397 | Transmissible gastroenteritis virus |
| | ck/CH/LHB/100801 | | strain |
| | | | TGEV/USA/NorthCarolina140/2007 |
| GQ504722 | Infectious bronchitis virus strain | KX900398 | Transmissible gastroenteritis virus |
| | Georgia 1998 pass8 | | strain TGEV/USA/Minnesota141/2007 |
| GQ504723 | Infectious bronchitis virus strain | KX900399 | Transmissible gastroenteritis virus |
| | Georgia 1998 Vaccine | | strain |
| | | | TGEV/USA/NorthCarolina142/2007 |
| GQ427173 | Turkey coronavirus strain | KX900400 | Transmissible gastroenteritis virus |
| | TCoV/VA-74/03 | | strain TGEV/USA/Iowa143/2008 |
| GQ427174 | Turkey coronavirus strain | KX900401 | Transmissible gastroenteritis virus |
| | TCoV/TX-GL/01 | | strain TGEV/USA/Tennessee144/2008 |
| GQ427175 | Turkey coronavirus strain | KX900402 | Transmissible gastroenteritis virus |
| | TCoV/IN-517/94 | | strain TGEV/Mex/145/2008 |
| GQ427176 | Turkey coronavirus strain | KX900403 | Transmissible gastroenteritis virus |
| | TCoV/TX-1038/98 | | strain TGEV/USA/Illinois146/2008 |
| EU022525 | Turkey coronavirus isolate | KX900404 | Transmissible gastroenteritis virus |
| | TCoV-540 | | strain TGEV/USA/Oklahoma147/2012 |
| EU022526 | Turkey coronavirus isolate | KX900405 | Transmissible gastroenteritis virus |
| | TCoV-ATCC | | strain TGEV/USA/Minnesota148/2013 |
| EU095850 | Turkey coronavirus isolate MG10 | KX900406 | Transmissible gastroenteritis virus |
| | | | strain TGEV/USA/Illinois149/2013 |
| EU526388 | Infectious bronchitis virus strain | KX900407 | Transmissible gastroenteritis virus |
| | A2 | | strain TGEV/USA/Minnesota150/2013 |
| EU111742 | Coronavirus SW1 | KX900408 | Transmissible gastroenteritis virus |
| | | | strain TGEV/USA/Wisconsin151/2014 |
| AY646283 | Avian infectious bronchitis virus | KX900409 | Transmissible gastroenteritis virus |
| | partridge/GD/S14/2003 | | strain TGEV/USA/Minnesota152/2014 |
| AY319651 | Avian infectious bronchitis virus | KX900410 | Transmissible gastroenteritis virus |
| | isolate BJ | | strain TGEV/USA/Minnesota153/2014 |
| MH532440 | Quail deltacoronavirus strain | KX900411 | Transmissible gastroenteritis virus |
| | G032/2015 | | strain |
| | | | TGEV/USA/SouthDakota154/2014 |
| MG812377 | Sparrow deltacoronavirus strain | KX964649 | Coronavirus AcCoV-JC34 |
| | ISU42824 | | |
| MG812378 | Sparrow deltacoronavirus strain | KX982264 | Bovine coronavirus isolate |
| | ISU73347 | | BCoV_2014_13 |
| JQ065044 | White-eye coronavirus HKU16 | KY014281 | Human coronavirus OC43 isolate |
| | strain HKU16-6847 | | 2002-04 |
| JQ065045 | Sparrow coronavirus HKU17 | KY014282 | Human coronavirus OC43 isolate |
| | strain HKU17-6124 | | 2007-09 |
| JQ065046 | Magpie-robin coronavirus HKU18 | KY073744 | NL63-related bat coronavirus strain |
| | strain HKU18-chu3 | | BtKYNL63-9a |
| JQ065047 | Night-heron coronavirus HKU19 | KY073745 | NL63-related bat coronavirus strain |
| | strain HKU19-6918 | | BtKYNL63-9b |
| JQ065048 | Wigeon coronavirus HKU20 strain | KY073746 | NL63-related bat coronavirus strain |
| | HKU20-9243 | | BtKYNL63-15 |
| JQ065049 | Common-moorhen coronavirus | KY073747 | 229E-related bat coronavirus strain |
| | HKU21 strain HKU21-8295 | | BtKY229E-1 |
| FJ376620 | Bulbul coronavirus HKU11-796 | KY073748 | 229E-related bat coronavirus strain |
| | | | BtKY229E-8 |
| FJ376621 | Thrush coronavirus HKU12-600 | KY292377 | Feline coronavirus strain |
| | | | HLJ/DQ/2016/01 |
| FJ376622 | Munia coronavirus HKU13-3514 | KY352407 | Severe acute respiratory syndrome- |
| | | | related coronavirus strain BtKY72 |
| AF220295 | Bovine coronavirus strain Quebec | KY369905 | Human coronavirus OC43 strain |
| | | | SC831 |
| AF391542 | Bovine coronavirus isolate BCoV- | KY369906 | Human coronavirus OC43 strain |
| | LUN | | SC622 |
| AY319651 | Avian infectious bronchitis virus | KY369907 | Human coronavirus OC43 strain |
| | isolate BJ | | SC9741 |
| AY391777 | Human coronavirus OC43 | KY369908 | Human coronavirus 229E strain SC579 |
| AY585228 | Human coronavirus OC43 strain | KY369909 | Human coronavirus 229E strain SC677 |
| | ATCC VR-759 | | |
| AY585229 | Human coronavirus OC43 serotype | KY369910 | Human coronavirus 229E strain |
| | OC43-Paris | | SC1143 |
| AY646283 | Avian infectious bronchitis virus | KY369911 | Human coronavirus 229E strain |
| | partridge/GD/S14/2003 | | SC1212 |
| AY884001 | Human coronavirus HKU1 | KY369912 | Human coronavirus 229E strain |
| | genotype B | | SC9731 |
| AY903459 | Human coronavirus OC43 strain | KY369913 | Human coronavirus 229E strain |
| | 87309 Belgium 2003 | | SC1073 |
| AY903460 | Human coronavirus OC43 strain | KY369914 | Human coronavirus 229E strain |
| | 19572 Belgium 2004 | | SC9773 |
| DQ011855 | Porcine hemagglutinating | KY417142 | Bat SARS-like coronavirus isolate |
| | encephalomyelitis virus strain | | As6526 |
| | VW572 | | |
| DQ022305 | Bat SARS coronavirus HKU3-1 | KY417143 | Bat SARS-like coronavirus isolate |
| | | | Rs4081 |
| DQ071615 | Bat SARS coronavirus Rp3 | KY417144 | Bat SARS-like coronavirus isolate |
| | | | Rs4084 |
| DQ084199 | bat SARS coronavirus HKU3-2 | KY417145 | Bat SARS-like coronavirus isolate |
| | | | Rf4092 |
| DQ084200 | bat SARS coronavirus HKU3-3 | KY417147 | Bat SARS-like coronavirus isolate |
| | | | Rs4237 |
| DQ201447 | Transmissible gastroenteritis virus | KY417148 | Bat SARS-like coronavirus isolate |
| | strain TS | | Rs4247 |
| DQ339101 | Human coronavirus HKU1 strain | KY417149 | Bat SARS-like coronavirus isolate |
| | N5P8 genotype A/B recombinant | | Rs4255 |
| DQ412042 | Bat SARS coronavirus Rf1 | KY417150 | Bat SARS-like coronavirus isolate |
| | | | Rs4874 |
| DQ412043 | Bat SARS coronavirus Rm1 | KY417151 | Bat SARS-like coronavirus isolate |
| | | | Rs7327 |
| DQ415897 | Human coronavirus HKU1 strain | KY419103 | Porcine hemagglutinating |
| | N20 genotype C | | encephalomyelitis virus strain PHEV |
| | | | CoV USA-15TOSU25049 |
| DQ415898 | Human coronavirus HKU1 strain | KY419104 | Porcine hemagglutinating |
| | N21 genotype C | | encephalomyelitis virus strain PHEV |
| | | | CoV USA-15TOSU0331 |
| DQ415899 | Human coronavirus HKU1 strain | KY419105 | Porcine hemagglutinating |
| | N22 genotype C | | encephalomyelitis virus strain PHEV |
| | | | CoV USA-15TOSU0582 |
| DQ415902 | Human coronavirus HKU1 strain | KY419106 | Porcine hemagglutinating |
| | N25 genotype B | | encephalomyelitis virus strain PHEV |
| | | | CoV USA-15TOSU1785 |
| DQ415911 | Human coronavirus HKU1 strain | KY419107 | Porcine hemagglutinating |
| | N15 genotype B | | encephalomyelitis virus strain PHEV |
| | | | CoV USA-15TOSU1209 |
| DQ415912 | Human coronavirus HKU1 strain | KY419109 | Porcine hemagglutinating |
| | N16 genotype C | | encephalomyelitis virus strain PHEV |
| | | | CoV USA-15TOSU1655 |
| DQ415913 | Human coronavirus HKU1 strain | KY419110 | Porcine hemagglutinating |
| | N17 genotype C | | encephalomyelitis virus strain PHEV |
| | | | CoV USA-15TOSU1362 |
| DQ443743 | Transmissible gastroenteritis virus | KY419112 | Porcine hemagglutinating |
| | strain SC-Y | | encephalomyelitis virus strain PHEV |
| | | | CoV USA-15TOSU1765 |
| DQ646405 | Infectious bronchitis virus isolate | KY419113 | Porcine hemagglutinating |
| | TW2575/98 | | encephalomyelitis virus strain PHEV |
| | | | CoV USA-15TOSU1582 |
| DQ648856 | Bat coronavirus (BtCoV/273/2005) | KY554972 | Human coronavirus OC43 strain N07- |
| | | | 1541B_433X |
| DQ648857 | Bat coronavirus (BtCoV/279/2005) | KY554973 | Human coronavirus OC43 strain N07- |
| | | | 1689B_116X |
| DQ811784 | Bovine coronavirus DB2 | KY554974 | Human coronavirus OC43 strain N08- |
| | | | 33B_360X |
| DQ811785 | TGEV Miller M6 | KY554975 | Human coronavirus OC43 strain N09- |
| | | | 382B |
| DQ811786 | TGEV Miller M60 | KY566209 | Feline coronavirus strain |
| | | | HLJ/HRB/2016/10 |
| DQ811788 | TGEV Purdue P115 | KY566210 | Feline coronavirus strain |
| | | | HLJ/HRB/2016/11 |
| DQ811789 | TGEV virulent Purdue | KY566211 | Feline coronavirus strain |
| | | | HLJ/HRB/2016/13 |
| DQ848678 | Feline coronavirus strain FCoV | KY621348 | Human coronavirus 229E strain SC379 |
| | C1Je | | |
| DQ915164 | Bovine coronavirus isolate Alpaca | KY674914 | Human coronavirus 229E strain SC399 |
| EF065513 | Bat coronavirus HKU9-1 | KY674917 | Human coronavirus OC43 strain N07- |
| | | | 1609B |
| EF065514 | Bat coronavirus HKU9-2 | KY674918 | Human coronavirus OC43 strain N07- |
| | | | 1647B |
| EF065516 | Bat coronavirus HKU9-4 | KY674920 | Human coronavirus OC43 strain N09- |
| | | | 595B |
| EF203064 | Bat coronavirus HKU2 strain | KY674921 | Human coronavirus HKU1 genotype B |
| | HKU2/GD/430/2006 | | strain N08-87 |
| EF203065 | Bat coronavirus HKU2 strain | KY684759 | Human coronavirus OC43 strain |
| | HKU2/HK/46/2006 | | SC2269 |
| EF203066 | Bat coronavirus HKU2 strain | KY684760 | Human coronavirus 229E strain |
| | HKU2/HK/298/2006 | | SC2282 |
| EF203067 | Bat coronavirus HKU2 strain | KY770858 | Bat coronavirus isolate Anlong-103 |
| | HKU2/HK/33/2006 | | |
| EF424615 | Bovine coronavirus E-AH65 | KY770859 | Bat coronavirus isolate Anlong-112 |
| EF424616 | Bovine coronavirus E-AH65-TC | KY770860 | Bat coronavirus isolate Jiyuan-84 |
| EF424617 | Bovine coronavirus R-AH65 | KY799179 | Myotis lucifugus coronavirus |
| EF424618 | Bovine coronavirus R-AH65-TC | KY938558 | Bat coronavirus strain 16BO133 |
| EF424619 | Bovine coronavirus E-AH187 | KY967356 | Human coronavirus OC43 strain |
| | | | SC2924 |
| EF424620 | Bovine coronavirus R-AH187 | KY967357 | Human coronavirus 229E strain |
| | | | SC2872 |
| EF424621 | Sable antelope coronavirus | KY967358 | Human coronavirus OC43 strain |
| | US/OH1/2003 | | SC2770 |
| EF424622 | Giraffe coronavirus US/OH3- | KY967359 | Human coronavirus OC43 strain |
| | TC/2006 | | SC2730 |
| EF424623 | Giraffe coronavirus US/OH3/2003 | KY967360 | Human coronavirus OC43 strain |
| | | | SC2476 |
| EF424624 | Calf-giraffe coronavirus | KY967361 | Human coronavirus OC43 strain |
| | US/OH3/2006 | | SC2345 |
| EF446615 | Equine coronavirus strain NC99 | KY983583 | Human coronavirus OC43 strain |
| | | | SC2481 |
| EU022525 | Turkey coronavirus isolate TCoV- | KY983585 | Human coronavirus OC43 strain |
| | 540 | | SC2854 |
| EU022526 | Turkey coronavirus isolate TCoV- | KY983587 | Human coronavirus 229E strain |
| | ATCC | | SC3112 |
| EU074218 | Transmissible gastroenteritis virus | KY983588 | Human coronavirus OC43 strain |
| | strain attenuated H | | SC3118 |
| EU095850 | Turkey coronavirus isolate MG10 | KY994645 | Porcine hemagglutinating |
| | | | encephalomyelitis virus strain JL/2008 |
| EU111742 | Coronavirus SW1 | KY996417 | Human coronavirus 229E strain |
| | | | 229E/UF-1/2016 |
| EU186072 | Feline coronavirus isolate Black | MF083115 | Porcine hemagglutinating |
| | | | encephalomyelitis virus strain CC14 |
| EU420137 | Bat coronavirus 1B strain | MF094685 | Swine acute diarrhea syndrome related |
| | AFCD307 | | coronavirus isolate 8462 |
| EU420138 | Miniopterus bat coronavirus 1 | MF094686 | Swine acute diarrhea syndrome related |
| | | | coronavirus isolate 8495 |
| EU420139 | Bat coronavirus HKU8 strain | MF113046 | Alphacoronavirus Mink/China/1/2016 |
| | AFCD77 | | |
| EU526388 | Infectious bronchitis virus strain | MF314143 | Human coronavirus OC43 isolate |
| | A2 | | HCoV-OC43/USA/ACRI_0052/2016 |
| FJ376619 | Bulbul coronavirus HKU11-934 | MF374983 | Human coronavirus OC43 isolate |
| | | | HCoV-OC43/USA/TCNP_0070/2016 |
| FJ376620 | Bulbul coronavirus HKU11-796 | MF374984 | Human coronavirus OC43 isolate |
| | | | HCoV-OC43/USA/TCNP_00204/2017 |
| FJ376621 | Thrush coronavirus HKU12-600 | MF374985 | Human coronavirus OC43 isolate |
| | | | HCoV-OC43/USA/TCNP_00212/2017 |
| FJ376622 | Munia coronavirus HKU13-3514 | MF421319 | Infectious bronchitis virus isolate |
| | | | UY/09/CA/01 |
| FJ415324 | Human enteric coronavirus 4408 | MF542265 | Human coronavirus 229E strain |
| | | | 229E/Haiti-1/2016 |
| FJ425184 | Waterbuck coronavirus US/OH- | MF577027 | Porcine epidemic diarrhea virus strain |
| | WD358-TC/1994 | | PEDV/Belgorod/dom/2008 |
| FJ425185 | Waterbuck coronavirus US/OH- | MF593268 | Middle East respiratory syndrome- |
| | WD358-GnC/1994 | | related coronavirus strain |
| | | | Neoromicia/5038 |
| FJ425186 | Waterbuck coronavirus US/OH- | MF882923 | Infectious bronchitis virus isolate |
| | WD358/1994 | | CK/CH/HB/2016 |
| FJ425187 | White-tailed deer coronavirus | MG021194 | Infectious bronchitis virus isolate |
| | US/OH-WD470/1994 | | gammaCoV/AvCov/Ck/Italy/624I/96 |
| FJ425188 | Sambar deer coronavirus US/OH- | MG021451 | Middle East respiratory syndrome- |
| | WD388-TC/1994 | | related coronavirus isolate NL13845 |
| FJ425189 | Sambar deer coronavirus US/OH- | MG021452 | Middle East respiratory syndrome- |
| | WD388/1994 | | related coronavirus isolate NL140422 |
| FJ425190 | Sambar deer coronavirus US/OH- | MG197709 | Human coronavirus OC43 strain BJ- |
| | WD388-TC/1994 calf-passaged | | 112 |
| FJ588686 | Bat SARS CoV Rs672/2006 | MG197710 | Human coronavirus OC43 strain BJ- |
| | | | 124 |
| FJ647223 | Murine coronavirus MHV-1 | MG197711 | Human coronavirus OC43 strain BJ- |
| | | | 164 |
| FJ755618 | Transmissible gastroenteritis virus | MG197712 | Human coronavirus OC43 strain BJ- |
| | strain H16 | | 165 |
| FJ938051 | Feline coronavirus RM | MG197713 | Human coronavirus OC43 strain BJ- |
| | | | 221 |
| FJ938052 | Feline coronavirus UU11 | MG197714 | Human coronavirus OC43 strain CC- |
| | | | 23 |
| FJ938053 | Feline coronavirus UU7 | MG197715 | Human coronavirus OC43 strain |
| | | | GZYF-26 |
| FJ938054 | Feline coronavirus UU4 | MG197716 | Human coronavirus OC43 strain WZ- |
| | | | 303 |
| FJ938055 | Feline coronavirus UU8 | MG197717 | Human coronavirus OC43 strain WZ- |
| | | | 522 |
| FJ938056 | Feline coronavirus UU5 | MG197718 | Human coronavirus OC43 strain YC- |
| | | | 55 |
| FJ938057 | Feline coronavirus UU15 | MG197719 | Human coronavirus OC43 strain YC- |
| | | | 67 |
| FJ938058 | Feline coronavirus UU16 | MG197720 | Human coronavirus OC43 strain YC- |
| | | | 68 |
| FJ938059 | Feline coronavirus UU10 | MG197721 | Human coronavirus OC43 strain YC- |
| | | | 72 |
| FJ938060 | Feline coronavirus UU2 | MG197722 | Human coronavirus OC43 strain YC- |
| | | | 207 |
| FJ938061 | Feline coronavirus UU3 | MG197723 | Human coronavirus OC43 strain HZ- |
| | | | 459 |
| FJ938062 | Feline coronavirus UU9 | MG448607 | Infectious bronchitis virus strain |
| | | | ck/CH/LHB/121042 |
| FJ938063 | Bovine coronavirus E-DB2-TC | MG518518 | Water deer coronavirus isolate W17-18 |
| FJ938064 | Bovine coronavirus E-AH187-TC | MG596802 | Middle East respiratory syndrome- |
| | | | related coronavirus isolate Bat- |
| | | | CoV/H.savii/Italy/206645-40/2011 |
| FJ938065 | Bovine respiratory coronavirus | MG596803 | Middle East respiratory syndrome- |
| | AH187 | | related coronavirus isolate Bat- |
| | | | CoV/P.khulii/Italy/206645-63/2011 |
| FJ938066 | Bovine respiratory coronavirus | MG757138 | Bovine coronavirus isolate ICSA21- |
| | bovine/US/OH-440-TC/1996 | | LBA |
| FJ938067 | Human enteric coronavirus strain | MG757139 | Bovine coronavirus isolate ICSA16- |
| | 4408 | | EN |
| FJ938068 | Rat coronavirus Parker | MG757140 | Bovine coronavirus isolate ICSA16- |
| | | | LBA |
| GQ152141 | Feline coronavirus strain | MG757141 | Bovine coronavirus isolate ICSA-pool- |
| | FCoV/NTU156/P/2007 | | EN |
| GQ153539 | Bat SARS coronavirus HKU3-4 | MG757142 | Bovine coronavirus isolate ICSA-pool- |
| | | | LBA |
| GQ153540 | Bat SARS coronavirus HKU3-5 | MG762674 | Rousettus bat coronavirus HKU9 |
| | | | isolate Rousettus spp/Jinghong/2009 |
| GQ153541 | Bat SARS coronavirus HKU3-6 | MG772933 | Bat SARS-like coronavirus isolate bat- |
| | | | SL-CoVZC45 |
| GQ153542 | Bat SARS coronavirus HKU3-7 | MG772934 | Bat SARS-like coronavirus isolate bat- |
| | | | SL-CoVZXC21 |
| GQ153543 | Bat SARS coronavirus HKU3-8 | MG812377 | Sparrow deltacoronavirus strain |
| | | | ISU42824 |
| GQ153544 | Bat SARS coronavirus HKU3-9 | MG812378 | Sparrow deltacoronavirus strain |
| | | | ISU73347 |
| GQ153545 | Bat SARS coronavirus HKU3-10 | MG893511 | Feline coronavirus isolate Felix |
| GQ153546 | Bat SARS coronavirus HKU3-11 | MG916901 | Bat coronavirus BtCoV/Rh/YN2012 |
| | | | isolate BtCoV/Rh/YN2012_Rs3376 |
| GQ153547 | Bat SARS coronavirus HKU3-12 | MG916902 | Bat coronavirus BtCoV/Rh/YN2012 |
| | | | isolate BtCoV/Rh/YN2012_Rs4125 |
| GQ153548 | Bat SARS coronavirus HKU3-13 | MG916903 | Bat coronavirus BtCoV/Rh/YN2012 |
| | | | isolate BtCoV/Rh/YN2012_Rs4259 |
| GQ427173 | Turkey coronavirus strain | MG916904 | Bat coronavirus BtCoV/Rh/YN2012 |
| | TCoV/VA-74/03 | | isolate BtCoV/Rh/YN2012_Ra13591 |
| GQ427174 | Turkey coronavirus strain | MG977444 | Human coronavirus OC43 isolate TNP |
| | TCoV/TX-GL/01 | | F1778_2 |
| GQ427175 | Turkey coronavirus strain | MG977445 | Human coronavirus OC43 isolate TNP |
| | TCoV/IN-517/94 | | F1790_2 |
| GQ427176 | Turkey coronavirus strain | MG977447 | Human coronavirus OC43 isolate TNP |
| | TCoV/TX-1038/98 | | F1832_2 |
| GQ477367 | Canine coronavirus strain | MG977449 | Human coronavirus OC43 isolate TNP |
| | CCoV/NTU336/F/2008 | | F1834_2 |
| GQ504722 | Infectious bronchitis virus strain | MG977451 | Human coronavirus OC43 isolate TNP |
| | Georgia 1998 pass8 | | 12636 |
| GQ504723 | Infectious bronchitis virus strain | MG977452 | Human coronavirus OC43 isolate TNP |
| | Georgia 1998 Vaccine | | 12643 |
| GU190215 | Bat coronavirus BM48- | MH021175 | Avian coronavirus strain D274 |
| | 31/BGR/2008 | | |
| GU393332 | Infectious bronchitis virus serotype | MH043952 | Bovine coronavirus isolate 4-17-03 |
| | Delaware 072 | | |
| GU553361 | Feline coronavirus UU22 isolate | MH043953 | Bovine coronavirus isolate 4-17-25 |
| | TCVSP-ROTTIER-00022 | | |
| GU553362 | Feline coronavirus UU23 isolate | MH043954 | Bovine coronavirus isolate 4-17-08 |
| | TCVSP-ROTTIER-00023 | | |
| HM211099 | Bat coronavirus HKU9-5-2 | MH043955 | Bovine coronavirus isolate 7-16-23 |
| HM211101 | Bat coronavirus HKU9-10-2 | MH121121 | Human coronavirus OC43 isolate |
| | | | HCoV-OC43/USA/ACRI_0213/2016 |
| HM245925 | Mink coronavirus strain WD1127 | MH532440 | Quail deltacoronavirus strain |
| | | | G032/2015 |
| HM245926 | Mink coronavirus strain WD1133 | MH687935 | Alphacoronavirus sp. strain |
| | | | VZ_AlphaCoV_16715_24 |
| HM776941 | Transmissible gastroenteritis virus | MH687936 | Alphacoronavirus sp. strain |
| | isolate AYU | | VZ_AlphaCoV_16715_31 |
| HQ012367 | Feline coronavirus UU17 | MH687937 | Alphacoronavirus sp. strain |
| | | | VZ_AlphaCoV_16715_32 |
| HQ012368 | Feline coronavirus UU18 | MH687939 | Alphacoronavirus sp. strain |
| | | | VZ_AlphaCoV_16715_39_c2 |
| HQ012369 | Feline coronavirus UU21 | MH687940 | Alphacoronavirus sp. strain |
| | | | VZ_AlphaCoV_16715_45 |
| HQ012370 | Feline coronavirus UU24 | MH687942 | Alphacoronavirus sp. strain |
| | | | VZ_AlphaCoV_16715_47_c2 |
| HQ012371 | Feline coronavirus UU31 | MH687943 | Alphacoronavirus sp. strain |
| | | | VZ_AlphaCoV_16715_5 |
| HQ012372 | Feline coronavirus UU34 | MH687944 | Alphacoronavirus sp. strain |
| | | | VZ_AlphaCoV_16715_53 |
| HQ392469 | Feline coronavirus UU40 | MH687947 | Alphacoronavirus sp. strain |
| | | | VZ_AlphaCoV_16715_63 |
| HQ392470 | Feline coronavirus UU19 | MH687949 | Alphacoronavirus sp. strain |
| | | | VZ_AlphaCoV_16715_76 |
| HQ392471 | Feline coronavirus UU20 | MH687950 | Alphacoronavirus sp. strain |
| | | | VZ_AlphaCoV_16715_77 |
| HQ392472 | Feline coronavirus UU30 | MH687951 | Alphacoronavirus sp. strain |
| | | | VZ_AlphaCoV_16715_78 |
| HQ462571 | Transmissible gastroenteritis virus | MH687952 | Alphacoronavirus sp. strain |
| | strain WH-1 | | VZ_AlphaCoV_16715_84 |
| JF330898 | Infectious bronchitis virus strain | MH687953 | Alphacoronavirus sp. strain |
| | ck/CH/LHB/100801 | | VZ_AlphaCoV_16715_86 |
| JF792616 | Rat coronavirus isolate 681 | MH687955 | Alphacoronavirus sp. strain |
| | | | VZ_AlphaCoV_16845_47 |
| JF792617 | Rat coronavirus isolate 8190 | MH687956 | Alphacoronavirus sp. strain |
| | | | VZ_AlphaCoV_16845_53 |
| JN129834 | Human coronavirus OC43 strain | MH687957 | Alphacoronavirus sp. strain |
| | HK04-01 | | VZ_AlphaCoV_16845_64 |
| JN129835 | Human coronavirus OC43 strain | MH687958 | Alphacoronavirus sp. strain |
| | HK04-02 | | VZ_AlphaCoV_16845_87 |
| JN183882 | Feline coronavirus UU47 | MH687959 | Alphacoronavirus sp. strain |
| | | | VZ_AlphaCoV_17819_17 |
| JN183883 | Feline coronavirus UU54 | MH687960 | Alphacoronavirus sp. strain |
| | | | VZ_AlphaCoV_17819_22 |
| JN856008 | Canine coronavirus strain A76 | MH687961 | Alphacoronavirus sp. strain |
| | | | VZ_AlphaCoV_17819_4 |
| JQ065044 | White-eye coronavirus HKU16 | MH687962 | Alphacoronavirus sp. strain |
| | strain HKU16-6847 | | VZ_AlphaCoV_17819_50 |
| JQ065045 | Sparrow coronavirus HKU17 | MH687965 | Alphacoronavirus sp. strain |
| | strain HKU17-6124 | | VZ_AlphaCoV_20745_17 |
| JQ065046 | Magpie-robin coronavirus HKU18 | MH687966 | Alphacoronavirus sp. strain |
| | strain HKU18-chu3 | | VZ_AlphaCoV_20745_6 |
| JQ065047 | Night-heron coronavirus HKU19 | MH687968 | Betacoronavirus sp. strain |
| | strain HKU19-6918 | | VZ_BetaCoV_16715_52 |
| JQ065048 | Wigeon coronavirus HKU20 strain | MH687970 | Betacoronavirus sp. strain |
| | HKU20-9243 | | VZ_BetaCoV_20724_34_c12 |
| JQ065049 | Common-moorhen coronavirus | MH810163 | Yak coronavirus strain |
| | HKU21 strain HKU21-8295 | | YAK/HY24/CH/2017 |
| JQ977697 | Infectious bronchitis virus isolate | MH817484 | Feline Alphacoronavirus 1 strain |
| | SNU8067 | | FCoV-SB22 |
| JQ989270 | Rousettus bat coronavirus HKU10 | MH878976 | Infectious bronchitis virus isolate |
| | isolate 183A | | VFAR-047 |
| JQ989271 | Rousettus bat coronavirus HKU10 | MH924835 | Infectious bronchitis virus strain |
| | isolate 175A | | gammaCoV/ck/China/I0636/16 |
| JQ989272 | Hipposideros bat coronavirus | MH938448 | Alphacoronavirus Bat- |
| | HKU10 isolate TLC1343A | | CoV/P.kuhlii/Italy/206645-41/2011 |
| JQ989273 | Hipposideros bat coronavirus | MH938449 | Alphacoronavirus Bat- |
| | HKU10 isolate TLC1347A | | CoV/P.kuhlii/Italy/3398-19/2015 |
| JX860640 | Canine respiratory coronavirus | MH938450 | Alphacoronavirus Bat- |
| | strain K37 | | CoV/P.kuhlii/Italy/206679-3/2010 |
| JX897900 | UNVERIFIED: Infectious | MH940245 | Human coronavirus HKU1 isolate |
| | bronchitis virus isolate GX- | | SI17244 |
| | NN09032 | | |
| JX993987 | Bat coronavirus Rp/Shaanxi2011 | MK142676 | Infectious bronchitis virus isolate |
| | | | ahysx-1 |
| JX993988 | Bat coronavirus Cp/Yunnan2011 | MK211369 | Coronavirus BtSk- |
| | | | AlphaCoV/GX2018A |
| KC869678 | Coronavirus Neoromicia/PML- | MK211370 | Coronavirus BtSk- |
| | PHE1/RSA/2011 | | AlphaCoV/GX2018B |
| KC881005 | Bat SARS-like coronavirus | MK211372 | Coronavirus BtSk- |
| | RsSHC014 | | AlphaCoV/GX2018D |
| KC881006 | Bat SARS-like coronavirus | MK211373 | Coronavirus BtRs-AlphaCoV/YN2018 |
| | Rs3367 | | |
| KC962433 | Transmissible gastroenteritis virus | MK211374 | Coronavirus BtRl-BetaCoV/SC2018 |
| | isolate TGEV-HX | | |
| KF367457 | Bat SARS-like coronavirus WIV1 | MK211375 | Coronavirus BtRs-BetaCoV/YN2018A |
| KF430219 | Bat coronavirus | MK211376 | Coronavirus BtRs-BetaCoV/YN2018B |
| | CDPHE15/USA/2006 | | |
| KF460437 | Infectious bronchitis virus isolate | MK211377 | Coronavirus BtRs-BetaCoV/YN2018C |
| | VicS-v | | |
| KF530060 | Human coronavirus OC43 strain | MK211378 | Coronavirus BtRs-BetaCoV/YN2018D |
| | OC43/human/USA/851-15/1985 | | |
| KF530061 | Human coronavirus OC43 strain | MK211379 | Coronavirus BtRt-BetaCoV/GX2018 |
| | OC43/human/USA/901-43/1990 | | |
| KF530063 | Human coronavirus OC43 strain | MK217372 | Infectious bronchitis virus strain |
| | OC43/human/USA/9612-48/1996 | | I0221/17 |
| KF530064 | Human coronavirus OC43 strain | MK217373 | Infectious bronchitis virus strain |
| | OC43/human/USA/9612-9/1996 | | I0725/17 |
| KF530065 | Human coronavirus OC43 strain | MK217374 | Infectious bronchitis virus strain |
| | OC43/human/USA/901-41/1990 | | I0916/16 |
| KF530066 | Human coronavirus OC43 strain | MK217375 | Infectious bronchitis virus strain |
| | OC43/human/USA/901-33/1990 | | I1209/16 |
| KF530067 | Human coronavirus OC43 strain | MK303619 | Human coronavirus OC43 isolate |
| | OC43/human/USA/912-10/1991 | | MDS6 |
| KF530068 | Human coronavirus OC43 strain | MK303620 | Human coronavirus OC43 isolate |
| | OC43/human/USA/007-11/2000 | | MDS2 |
| KF530069 | Human coronavirus OC43 strain | MK303621 | Human coronavirus OC43 isolate |
| | OC43/human/USA/982-4/1998 | | MDS4 |
| KF530070 | Human coronavirus OC43 strain | MK303622 | Human coronavirus OC43 isolate |
| | OC43/human/USA/991-19/1999 | | MDS11 |
| KF530071 | Human coronavirus OC43 strain | MK303623 | Human coronavirus OC43 isolate |
| | OC43/human/USA/925-1/1992 | | MDS12 |
| KF530072 | Human coronavirus OC43 strain | MK303624 | Human coronavirus OC43 isolate |
| | OC43/human/USA/9712-13/1997 | | MDS14 |
| KF530073 | Human coronavirus OC43 strain | MK303625 | Human coronavirus OC43 isolate |
| | OC43/human/USA/8912-37/1989 | | MDS16 |
| KF530074 | Human coronavirus OC43 strain | MK309398 | Infectious bronchitis virus isolate |
| | OC43/human/USA/9212-33/1992 | | CK/CH/GD/HY16 |
| KF530075 | Human coronavirus OC43 strain | MK329221 | Infectious bronchitis virus isolate CK- |
| | OC43/human/USA/953-23/1995 | | CH-GX-YL17 |
| KF530076 | Human coronavirus OC43 strain | MK472067 | Alphacoronavirus sp. isolate WA1087 |
| | OC43/human/USA/911-11/1991 | | |
| KF530077 | Human coronavirus OC43 strain | MK472068 | Alphacoronavirus sp. isolate WA2028 |
| | OC43/human/USA/873-16/1987 | | |
| KF530078 | Human coronavirus OC43 strain | MK472069 | Alphacoronavirus sp. isolate WA3301 |
| | OC43/human/USA/9612-29/1996 | | |
| KF530079 | Human coronavirus OC43 strain | MK472070 | Alphacoronavirus sp. isolate WA3607 |
| | OC43/human/USA/913-29/1991 | | |
| KF530080 | Human coronavirus OC43 strain | MK472071 | Alphacoronavirus sp. isolate WAAlc1 |
| | OC43/human/USA/9712-31/1997 | | |
| KF530081 | Human coronavirus OC43 strain | MK492263 | Bat coronavirus strain BtCoV92 |
| | OC43/human/USA/991-5/1999 | | |
| KF530082 | Human coronavirus OC43 strain | MK574042 | Infectious bronchitis virus strain |
| | OC43/human/USA/912-11/1991 | | ck/CH/LHB/110615 |
| KF530083 | Human coronavirus OC43 strain | MK574043 | Infectious bronchitis virus strain |
| | OC43/human/USA/873-19/1987 | | ck/CH/LHB/110617 |
| KF530084 | Human coronavirus OC43 strain | MK581202 | Infectious bronchitis virus strain |
| | OC43/human/USA/951-18/1995 | | gammaCoV/Ck/Poland/80/1989 |
| KF530085 | Human coronavirus OC43 strain | MK581204 | Infectious bronchitis virus strain |
| | OC43/human/USA/871-25/1987 | | gammaCoV/Ck/Poland/255/1997 |
| KF530086 | Human coronavirus OC43 strain | MK720944 | Tylonycteris bat coronavirus HKU33 |
| | OC43/human/USA/872-5/1987 | | strain GZ151867 |
| KF530087 | Human coronavirus OC43 strain | MK720945 | Rhinolophus bat coronavirus HKU32 |
| | OC43/human/USA/873-6/1987 | | strain TLC26A |
| KF530088 | Human coronavirus OC43 strain | MK720946 | Rhinolophus bat coronavirus HKU32 |
| | OC43/human/USA/901-54/1990 | | strain TLC28A |
| KF530089 | Human coronavirus OC43 strain | MK878536 | Infectious bronchitis virus isolate |
| | OC43/human/USA/911-66/1991 | | GA9977/2019 |
| KF530090 | Human coronavirus OC43 strain | MK907286 | Erinaceus hedgehog coronavirus |
| | OC43/human/USA/931-85/1993 | | HKU31 strain F6 |
| KF530091 | Human coronavirus OC43 strain | MK907287 | Erinaceus hedgehog coronavirus |
| | OC43/human/USA/911-58/1991 | | HKU31 strain Rs13 |
| KF530092 | Human coronavirus OC43 strain | MN026164 | Human coronavirus OC43 isolate |
| | OC43/human/USA/008-5/2000 | | OC43_KLF_01_2018 |
| KF530094 | Human coronavirus OC43 strain | MN096598 | Infectious bronchitis virus strain |
| | OC43/human/USA/912-36/1991 | | ck/CH/YNSL/160501 |
| KF530095 | Human coronavirus OC43 strain | MN165107 | Feline coronavirus isolate XXN |
| | OC43/human/USA/912-6/1991 | | |
| KF530096 | Human coronavirus OC43 strain | MN197549 | Infectious bronchitis virus isolate |
| | OC43/human/USA/911-38/1991 | | CK/CH/GD/GDTS13 |
| KF530097 | Human coronavirus OC43 strain | MN306036 | Human coronavirus OC43 strain |
| | OC43/human/USA/9211-43/1992 | | SC0682 |
| KF530098 | Human coronavirus OC43 strain | MN306041 | Human coronavirus OC43 strain |
| | OC43/human/USA/965-6/1996 | | SC0810 |
| KF530099 | Human coronavirus OC43 strain | MN306042 | Human coronavirus OC43 strain |
| | OC43/human/USA/971-5/1997 | | SC0839 |
| KF530123 | Feline coronavirus strain Felis | MN306043 | Human coronavirus OC43 strain |
| | catus/NLD/UU88/2010 | | SC0841 |
| KF569996 | Rhinolophus affinis coronavirus | MN306046 | Human coronavirus 229E strain |
| | isolate LYRa11 | | SC0865 |
| KF636752 | Bat Hp- | MN306053 | Human coronavirus OC43 strain |
| | betacoronavirus/Zhejiang2013 | | SC9430 |
| KF663559 | Infectious bronchitis virus isolate | MN310476 | Human coronavirus OC43 strain |
| | ck/CH/IBTZ/2012 | | SC9428 |
| KF668605 | Infectious bronchitis virus isolate | MN310478 | Human coronavirus OC43 strain |
| | CK/CH/SD09/005 | | SC0776 |
| KF906249 | Dromedary camel coronavirus | MN369046 | Human coronavirus 229E strain |
| | HKU23 strain HKU23-265F | | SC9724 |
| KF906250 | Dromedary camel coronavirus | MN512434 | Infectious bronchitis virus isolate |
| | HKU23 strain HKU23-362F | | IBV/Ck/Can/17-035614 |
| KF923886 | Human coronavirus OC43 isolate | MN512435 | Infectious bronchitis virus isolate |
| | 1908A/2010 | | IBV/Ck/Can/17-036989 |
| KF923887 | Human coronavirus OC43 isolate | MN512436 | Infectious bronchitis virus isolate |
| | 1997A/2010 | | IBV/Ck/Can/18-048192T |
| KF923888 | Human coronavirus OC43 isolate | MN512437 | Infectious bronchitis virus isolate |
| | 2145A/2010 | | IBV/Ck/Can/18-048430 |
| KF923889 | Human coronavirus OC43 isolate | MN512438 | Infectious bronchitis virus isolate |
| | 1926/2006 | | IBV/Ck/Can/18-049707 |
| KF923890 | Human coronavirus OC43 isolate | MN514962 | Dromedary camel coronavirus HKU23 |
| | 39A/2007 | | isolate DcCoV- |
| | | | HKU23/camel/Ethiopia/CAC1019/2015 |
| KF923891 | Human coronavirus OC43 isolate | MN514963 | Dromedary camel coronavirus HKU23 |
| | 5240/2007 | | isolate DcCoV- |
| | | | HKU23/camel/Morocco/CAC2586/2016 |
| KF923892 | Human coronavirus OC43 isolate | MN514964 | Dromedary camel coronavirus HKU23 |
| | 5345/2007 | | isolate DcCoV- |
| | | | HKU23/camel/Nigeria/NV1010/2015 |
| KF923893 | Human coronavirus OC43 isolate | MN514965 | Dromedary camel coronavirus HKU23 |
| | 2151A/2010 | | isolate DcCoV- |
| | | | HKU23/camel/Nigeria/NV1092/2015 |
| KF923894 | Human coronavirus OC43 isolate | MN514966 | Dromedary camel coronavirus HKU23 |
| | 5352/2007 | | isolate DcCoV- |
| | | | HKU23/camel/Nigeria/NV1097/2015 |
| KF923895 | Human coronavirus OC43 isolate | MN514967 | Dromedary camel coronavirus HKU23 |
| | 10285/2010 | | isolate DcCoV- |
| | | | HKU23/camel/Nigeria/NV1385/2016 |
| KF923896 | Human coronavirus OC43 isolate | MN611517 | Rousettus aegyptiacus bat coronavirus |
| | 3074A/2012 | | 229E-related isolate 5425 |
| KF923897 | Human coronavirus OC43 isolate | MN611518 | Miniopterus pusillus bat coronavirus |
| | 3269A/2012 | | HKU8-related isolate 6610 |
| KF923898 | Human coronavirus OC43 isolate | MN611521 | Scotophilus kuhlii bat coronavirus 512- |
| | 3184A/2012 | | related isolate HK140714 |
| KF923899 | Human coronavirus OC43 isolate | MN611522 | Rhinolophus affinis bat coronavirus |
| | 3582/2006 | | HKU2-related isolate 160660 |
| KF923900 | Human coronavirus OC43 isolate | MN611524 | Miniopterus schreibersii bat |
| | 3647/2006 | | coronavirus 1-related isolate 161454 |
| KF923901 | Human coronavirus OC43 isolate | MN611525 | Hipposideros pomona bat coronavirus |
| | 5472/2007 | | CHB25 isolate CHB0025 |
| KF923902 | Human coronavirus OC43 isolate | NC_003045 | Bovine coronavirus |
| | 12689/2012 | | |
| KF923903 | Human coronavirus OC43 isolate | NC_038861 | Transmissible gastroenteritis virus |
| | 12691/2012 | | complete genome |
| KF923904 | Human coronavirus OC43 isolate | NC_045512 | Wuhan seafood market pneumonia |
| | 12694/2012 | | virus isolate Wuhan-Hu-1 |
| KF923905 | Human coronavirus OC43 isolate | U00735 | Bovine coronavirus strain Mebus |
| | 229/2005 | | |
| KF923906 | Human coronavirus OC43 isolate | PDF_0663 | #N/A |
| | 3194A/2012 | | |
| KF923907 | Human coronavirus OC43 isolate | GCS_003 | #N/A |
| | 5370/2007 | | |
| KF923908 | Human coronavirus OC43 isolate | GCS_019 | #N/A |
| | 5414/2007 | | |
| KF923909 | Human coronavirus OC43 isolate | PDF_2560 | #N/A |
| | 5442/2007 | | |
| KF923910 | Human coronavirus OC43 isolate | PDF_3316 | #N/A |
| | 5445/2007 | | |
| KF923911 | Human coronavirus OC43 isolate | GCS_242 | #N/A |
| | 5479/2007 | | |
| KF923912 | Human coronavirus OC43 isolate | GCS_264a | #N/A |
| | 5484/2007 | | |
| KF923913 | Human coronavirus OC43 isolate | GCS_264b | #N/A |
| | 5485/2007 | | |
| KF923914 | Human coronavirus OC43 isolate | GCS_265a | #N/A |
| | 5508/2007 | | |
| KF923915 | Human coronavirus OC43 isolate | GCS_265b | #N/A |
| | 5517/2007 | | |
| KF923916 | Human coronavirus OC43 isolate | SBB_122 | #N/A |
| | 5519/2007 | | |
| KF923917 | Human coronavirus OC43 isolate | PDF_1704 | #N/A |
| | 5566/2007 | | |
| KF923918 | Human coronavirus OC43 isolate | GCS_008 | #N/A |
| | 10108/2010 | | |
| KF923919 | Human coronavirus OC43 isolate | SBB_943 | #N/A |
| | 5595/2007 | | |
| KF923920 | Human coronavirus OC43 isolate | SBB_797 | #N/A |
| | 5617/2007 | | |
| KF923921 | Human coronavirus OC43 isolate | PDF_2370 | #N/A |
| | 69A/2007 | | |
| KF923922 | Human coronavirus OC43 isolate | PDF_2386 | #N/A |
| | 8164/2009 | | |
| KF923923 | Human coronavirus OC43 isolate | GCS_017 | #N/A |
| | 892A/2008 | | |
| KF923924 | Human coronavirus OC43 isolate | PRD_0038 | #N/A |
| | 10290/2010 | | |
| KF923925 | Human coronavirus OC43 isolate | PDF_2180 | #N/A |
| | 10574/2010 | | |
| KF931628 | Infectious bronchitis virus isolate | SBB_120 | #N/A |
| | VicS-del | | |
| KJ473795 | BtMf-AlphaCoV/AH2011 | SBB_041 | #N/A |
| KJ473796 | BtMf-AlphaCoV/JX2012 | SBB_053 | #N/A |
| KJ473797 | BtMf-AlphaCoV/GD2012 | SBB_010 | #N/A |
| KJ473798 | BtMf-AlphaCoV/HuB2013 | SBB_024 | #N/A |
| KJ473799 | BtMf-AlphaCoV/FJ2012 | SBB_726 | #N/A |
| KJ473800 | BtMf-AlphaCoV/HeN2013 | SBB_747 | #N/A |
| KJ473806 | BtMr-AlphaCoV/SAX2011 | SBB_788 | #N/A |
| KJ473807 | BtRf-AlphaCoV/HuB2013 | SBB_130 | #N/A |
| KJ473808 | BtRf-AlphaCoV/YN2012 | GCS_011 | #N/A |
| KJ473809 | BtNv-AlphaCoV/SC2013 | GCS_211 | #N/A |
| KJ473810 | BtMs-AlphaCoV/GS2013 | GCS_227 | #N/A |
| KJ473811 | BtRf-BetaCoV/JL2012 | NC_009020 | Pipistrellus bat coronavirus HKU5 |
| KJ473812 | BtRf-BetaCoV/HeB2013 | EF065508 | Bat coronavirus HKU4-4 |
| KJ473813 | BtRf-BetaCoV/SX2013 | EF065505 | Bat coronavirus HKU4-1 |
| KJ473814 | BtRs-BetaCoV/HuB2013 | EF065506 | Bat coronavirus HKU4-2 |
| KJ473815 | BtRs-BetaCoV/GX2013 | EF065507 | Bat coronavirus HKU4-3 |
| KJ473816 | BtRs-BetaCoV/YN2013 | NC_009019 | Tylonycteris bat coronavirus HKU4 |
| KJ473821 | BtVs-BetaCoV/SC2013 | MH002339 | Tylonycteris bat coronavirus HKU4 |
| KJ958218 | Human coronavirus OC43 strain | MH002337 | Tylonycteris bat coronavirus HKU4 |
| | LY341 | | |
| KJ958219 | Human coronavirus OC43 strain | MH002338 | Tylonycteris bat coronavirus HKU4 |
| | LY342 | | |
| KM213963 | Infectious bronchitis virus isolate | MW218395 | Tylonycteris bat coronavirus HKU4 |
| | CK/CH/XDC- 2/2013 | | |
| KM347965 | Ferret coronavirus isolate FRCoV- | MN611519 | Tylonycteris pachypus bat coronavirus |
| | NL-2010 | | HKU4-related isolate GZ131656 |
| KM454473 | Duck coronavirus isolate | MH002342 | Pipistrellus bat coronavirus HKU5 |
| | DK/GD/27/2014 | | |
| KP143507 | Feline coronavirus isolate 27C | MN611520 | Pipistrellus abramus bat coronavirus |
| | | | HKU5-related isolate BY140568 |
| KP143508 | Feline coronavirus isolate 28O | MH002340 | Pipistrellus bat coronavirus HKU5 |
| KP143509 | Feline coronavirus isolate 65F | EF065510 | Bat coronavirus HKU5-2 |
| KP143510 | Feline coronavirus isolate 67F | EF065511 | Bat coronavirus HKU5-3 |
| KP143511 | Feline coronavirus isolate 80F | EF065512 | Bat coronavirus HKU5-5 |
| KP143512 | Feline coronavirus isolate 26M | EF065509 | Bat coronavirus HKU5-1 |
| KP198610 | Human coronavirus OC43 strain | MH002341 | Pipistrellus bat coronavirus HKU5 |
| | 2058A/10 | | |
| KP198611 | Human coronavirus OC43 strain | | |
| | 1783A/10 | | |

### Example 2. RNA Preparation and Tagmentation Enrichment of RNAs of Interest in Wastewater Samples

RNA sequencing (RNA-Seq) with next-generation sequencing (NGS) is a powerful method for discovering, profiling, and quantifying RNA transcripts. Targeted RNA-Seq analyzes expression in a focused set of genes. Enrichment enables cost-effective RNA exome analysis using sequence-specific capture of the coding regions of the transcriptome. It is ideal for low-quality samples.

This tagmentation enrichment uses on-bead tagmentation followed by a single 90-minute hybridization step to provide a rapid workflow. On-bead tagmentation features enrichment Bead-Linked Transposomes (eBLT) optimized for RNA (eBLTL) that mediate a uniform tagmentation reaction. In addition to manual preparation, RNA Preparation and Tagmentation Enrichment is designed to be compatible with liquid-handling platforms for an automated workflow, providing highly reproducible sample handling, reduced risk of human error, and less hands-on time.

A. cDNA Synthesis and Tagmentation

Wastewater is collected for evaluation of viral RNA. RNA collected from wastewater is denatured and then random hexamers are annealed. The random hexamers prime the sample for cDNA synthesis. The hexamer-primed RNA fragments are then reverse transcribed to produce first strand cDNA. Enrichment Bead-Linked Transposomes are used to tagment double-stranded cDNA.

### B. Amplification and Purification

After tagmentation, the fragments are purified and amplified to add index adapter sequences for dual indexing and P7 and P5 sequences for clustering. Next, magnetic beads are implemented to purify the tagmented library. Then the purified library is quantified and normalized.

### C. Enrichment

After normalization, the library is combined into one pool for one- or three-plex enrichment. Results are optimized for 200 ng of each library. Following quantification and normalization, the magnetic beads are implemented to capture probes hybridized to the targeted library fragments of interest. Using heated washes, nonspecific binding is removed from the beads. The enriched library is then eluted from the beads. The enriched library is then amplified using a PCR program. In some embodiments, the PCR program is 14 cycles. After amplification, magnetic beads are used purify the enriched library.

### D. Evaluation

The enriched library is then evaluated using either or both of the following methods: (1) analyzing 1 μl of the enriched library with the Qubit dsDNA HS Assay kit (Illumina) to quantify library concentration (ng/μl); and/or (2) analyzing 1 μl of the enriched library with the Agilent 2100 Bioanalyzer System and a DNA 1000 Kit to qualify.

After diluting to the starting concentration depending on the sequence system, libraries are denatured and diluted to the final loading concentration. Paired-end runs are used for sequencing. The number of cycles per index read is 10, and the number of cycles per read varies depending on the sequencing system.

### Example 3. Enrichment Using a Solid Support

A solid support, such as a flowcell, is prepared for enrichment. Oligonucleotides are prepared corresponding to desired RNA, and these oligonucleotides are immobilized to a solid support. For example, oligonucleotides comprising sequences complementary to desired RNA (e.g., RNA sequences associated with coronaviruses) are immobilized to a solid support to allow for enrichment. A flowcell with such immobilized oligonucleotides may be termed an enrichment flowcell.

A cDNA library is prepared using the probe sets described above in Example 1 from a wastewater sample comprising RNA. Library fragments are then be added to the enrichment flowcell. Library fragments prepared from desired RNA bind to the enrichment flowcell, and the fluid that does not bind to the enrichment flowcell (comprising library fragments not prepared from desired RNA) is siphoned to a waste container. The bound library fragments are denatured, collected, and sequenced (with optional amplification before sequencing). In this way, the library that is sequenced is enriched for library fragments prepared from desired RNA.

### Example 4. Pan-Coronavirus Panel for Genomic Surveillance of Coronaviruses Using Target Enrichment NGS

The performance of a panel comprising coronavirus enrichment probes described herein (Pan-CoV panel) was evaluated in an enrichment assay (Illumina RNA Prep with Enrichment or “IRPE”) followed by Illumina SBS sequencing using synthetic controls representative of four different coronaviruses. The Pan-CoV panel contains probes to >200 human and animal coronaviruses. Compared to untargeted sequencing, enrichment using the Pan-CoV panel provides much more depth of genome coverage and sensitivity (>100-fold enrichment at low viral loads). Mixing studies demonstrated that different controls of Coronaviruses can be detected together, revealing the robustness of this assay. In addition, the design strategy of Pan-CoV panel and IRPE chemistry can also tolerate a certain level of mismatches such that diverse coronaviruses sequences including new variants, as indicated by the identification of BA.2 variant of SARS-COV-2 that emerged after the panel development was complete.

To further demonstrate the applications of this assay in real-world samples, enrichment using the Pan-CoV panel was performed on 48 RNA samples extracted from bat feces collected from Africa followed by sequencing. When performing assembly analysis, it was found that enriched libraries showed more and longer contigs compared to untargeted sequencing. Alignment of these contigs to widely used databases showed similarity to currently characterized Bat and Human Coronaviruses but have significant divergence in regions of the genome, seemingly due to the novelty of the Coronaviruses sequenced. To provide a comprehensive analysis solution, a DRAGEN Microbial Enrichment App was developed, which provides users with consensus genomes and contigs, enabling novel coronaviruses discovery and characterization, and using these two methods together provides benefits. Thus, the panel comprising coronavirus enrichment probes is a powerful tool for monitoring the spread of coronaviruses to prevent the next pandemic.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A method of enriching a sample for one or more target viral nucleic acids comprising the steps of:
a. providing a probe set comprising at least two nucleic acid probes complementary to one or more target viral nucleic acids, wherein the probe set comprises at least two of SEQ ID NOs: 1-22909;
b. allowing the probes in the probe set to hybridize to the target viral nucleic acids; and
c. enriching the sample for the one or more target viral nucleic acids by amplifying the target viral nucleic acids and/or separating the target viral nucleic acids from the sample.

2. A method of enriching a sample for one or more target coronavirus nucleic acids comprising the steps of:
a. providing a probe set comprising at least two nucleic acid probes complementary to one or more target coronavirus nucleic acids, wherein the nucleic acid probes are affixed to a support;
b. capturing one or more target coronavirus nucleic acids on a support;
c. using the one or more captured target coronavirus nucleic acids as a template strand to produce one or more nucleic acid duplexes immobilized on the support, wherein the at least one target coronavirus nucleic acids hybridize to one or more probes in a probe set on the support;
d. contacting a transposase and transposon with the one or more nucleic acid duplexes under conditions wherein the one or more nucleic acid duplexes and transposon composition undergo a transposition reaction to produce one or more tagged nucleic acid duplexes, wherein the transposon composition comprises a double stranded nucleic acid molecule comprising a transferred strand and a non-transferred strand;
e. contacting the one or more tagged nucleic acid duplexes with a nucleic acid modifying enzyme under conditions to extend a 3′ end of the immobilized strand to a 5′ end of the template strand to produce one or more end-extended tagged nucleic acid duplexes;
f. amplifying the one or more end-extended tagged nucleic acid duplexes to produce a plurality of tagged nucleic acid strands;
g. contacting the plurality of tagged nucleic acid strands with a probe set to create an enriched library; and
h. amplifying the enriched library.

3. The method of claim 2, wherein the sample comprises a sample from a mammal.

4. The method of claim 2, wherein the sample comprises a blood sample, a serum sample, a whole blood sample, a tissue sample, a fecal sample, a urine sample, a mucus sample, a saliva sample, a lymph sample, a vaginal fluid sample, a semen sample, an amniotic sample, and/or a sweat sample.

5. The method of claim 2, wherein the sample comprises a freshwater sample, a wastewater sample, a saline water sample, or a combination thereof.

6. The method of claim 2, wherein the probe set is biotinylated.

7. The method of claim 2, wherein the one or more target coronavirus nucleic acids are coronavirus RNA molecules.

8. The method of claim 2, wherein the probe set further comprises at least two DNA probes that each hybridize to at least one target coronavirus molecule of an Alphacoronavirus, Betacoronavirus, Deltacoronavirus, Gammacoronavirus, and/or Bafinivirus genus.

9. The method of claim 2, wherein the probe set further comprises at least two DNA probes that each hybridize to at least one target coronavirus molecule selected from Table 2.

10. The method of claim 2, wherein the probe set further comprises at least two DNA probes that comprise any one of SEQ ID NOs: 22917-23376.

11. The method of claim 2, wherein the method further comprises depleting unwanted nucleic acid molecules from a nucleic acid sample by depleting unwanted cDNA library fragments from a library of cDNA fragments prepared from RNA, wherein the unwanted cDNA library fragments comprise those prepared from unwanted RNA sequences, further comprising:
a. preparing a solid support comprising at least one immobilized oligonucleotide, wherein each immobilized oligonucleotide comprises a nucleic acid sequence corresponding to an unwanted RNA sequence or its complement;
b. adding the library of fragments to the solid support and hybridizing the library fragments to at least one immobilized oligonucleotide to allow binding of unwanted library fragments to at least one immobilized oligonucleotide; and
c. collecting library fragments not bound to at least one immobilized oligonucleotide.

12. The method of claim 11 wherein the at least one immobilized oligonucleotide comprises a sequence comprising any one or more of SEQ ID NOs: 23377-24507 or its complement.

13. The method of claim 11, wherein the depleting unwanted nucleic acid molecules comprises depleting off-target RNA nucleic acid molecules from a nucleic acid sample comprises:
a. contacting a nucleic acid sample comprising at least one RNA or DNA target sequence and at least one off-target RNA molecule from a first species with a probe set comprising at least two DNA probes complementary to discontiguous sequences along the full length of the at least one off-target RNA molecule from a second species, thereby hybridizing the DNA probes to the off-target RNA molecules to form DNA: RNA hybrids, wherein each DNA: RNA hybrid is at least 5 bases apart, or at least 10 bases apart, along a given off-target RNA molecule sequence from any other DNA: RNA hybrid, wherein the off-target RNA comprises at least one small noncoding RNA chosen from RN7SK, RN7SL1, RN7SL2, RN7SL5P, RPPH1, SNORD3A;
b. contacting the DNA: RNA hybrids with a ribonuclease that degrades the RNA from the DNA: RNA hybrids, thereby degrading the off-target RNA molecules in the nucleic acid sample to form a degraded mixture;
c. separating the degraded RNA from the degraded mixture;
d. sequencing the remaining RNA from the sample;
e. evaluating the remaining RNA sequences for the presence of off-target RNA molecules from the first species, thereby determining gap sequence regions; and
f. supplementing the probe set with additional DNA probes complementary to discontiguous sequences in one or more of the gap sequence regions.

14. The method of claim 2, wherein the probe set comprises any one or more of SEQ ID NOs: 22917-23376.

15. The method of claim 2, wherein the method further comprises depleting unwanted cDNA library fragments from a library of cDNA fragments prepared from RNA, wherein the unwanted cDNA library fragments comprise those prepared from unwanted RNA sequences.

16. A composition comprising a probe set comprising at least one DNA probe comprising at least one sequence of SEQ ID NOs: 1-22909.

17. The composition of claim 16, comprising at least 5, at least at least 10, at least 50, at least 100, at least 250, at least 500, at least 750, at least 1000, at least 1500, or at least 2000 sequences of SEQ ID NOs: 1-22909.

18. A kit comprising a probe set comprising:
a. at least one DNA probe comprising at least one sequence comprising at least one of SEQ ID NOs: 1-22909; and
b. a buffer.

19. The kit of claim 18, further comprising:
a. a ribonuclease;
b. a DNase; and
c. RNA purification beads.

20. The kit of claim 19, further comprising a nucleic acid destabilizing chemical comprising formamide.
